SEQUENCE LISTING (629 sequences)

SEQ ID NO: 1 - protein, 108 aa, synthetic construct
  REGION 1..108: note = ULBP2/5/6_VL-1
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIK

SEQ ID NO: 2 - protein, 120 aa, synthetic construct
  REGION 1..120: note = ULBP2/5/6_VH-1
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSS

SEQ ID NO: 3 - protein, 108 aa, synthetic construct
  REGION 1..108: note = ULBP2/5/6_VL-2
EIVLTQSPATLSLSPGERATLSCRASQSIGTSIHWYQDKPGQAPRLLIYFASESISGIPARFSGSGSGTDFTLTISSLEPEDFAVYYCQQSNSWPLYTFGGGTKVEIK

SEQ ID NO: 4 - protein, 120 aa, synthetic construct
  REGION 1..120: note = ULBP2/5/6_VH-2
QVQLVQSGAEVKKPGASVKVSCKASGYTFTNYDINWVRKAPGQGLEWMGWIYPGDGSTYYNEKFKGRVTMTRDTSTSTVYMELSSLRSEDTAVYYCARSGDYGLYYFDYWGQGTLVTVSS

SEQ ID NO: 5 - protein, 5 aa, synthetic construct
  REGION 1..5: note = ULBP2/5/6_CDRH1-1_Kabat
NYDIN

SEQ ID NO: 6 - protein, 7 aa, synthetic construct
  REGION 1..7: note = ULBP2/5/6_CDRH1-1_Chothia
GYTFTNY

SEQ ID NO: 7 - protein, 17 aa, synthetic construct
  REGION 1..17: note = ULBP2/5/6_CDRH2-1_Kabat
WIYPGDGSTYYNEKFKG

SEQ ID NO: 8 - protein, 6 aa, synthetic construct
  REGION 1..6: note = ULBP2/5/6_CDRH2-1_Chothia
YPGDGS

SEQ ID NO: 9 - protein, 11 aa, synthetic construct
  REGION 1..11: note = ULBP2/5/6_CDRH3-1
SGDYGLYYFDY

SEQ ID NO: 10 - protein, 11 aa, synthetic construct
  REGION 1..11: note = ULBP2/5/6_CDRL1-1
RASQSIGTSIH

SEQ ID NO: 11 - protein, 7 aa, synthetic construct
  REGION 1..7: note = ULBP2/5/6_CDRL2-1
FASESIS

SEQ ID NO: 12 - protein, 10 aa, synthetic construct
  REGION 1..10: note = ULBP2/5/6_CDRL3-1
QQSNSWPLYT

SEQ ID NO: 13 - protein, 125 aa, synthetic construct
  REGION 1..125: note = CD3_VH-1
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 14 - protein, 125 aa, synthetic construct
  REGION 1..125: note = CD3_VH-2
EVQLLESGGGLVQPGGSLKLSCAASGFTFNTYAMNWVRDAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 15 - protein, 125 aa, synthetic construct
  REGION 1..125: note = CD3_VH-3
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSS

SEQ ID NO: 16 - protein, 125 aa, synthetic construct
  REGION 1..125: note = CD3_VH-4
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSS

SEQ ID NO: 17 - protein, 125 aa, synthetic construct
  REGION 1..125: note = CD3_VH-5
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 18 - protein, 125 aa, synthetic construct
  REGION 1..125: note = CD3_VH-6
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSS

SEQ ID NO: 19 - protein, 125 aa, synthetic construct
  REGION 1..125: note = CD3_VH-7
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSS

SEQ ID NO: 20 - protein, 125 aa, synthetic construct
  REGION 1..125: note = CD3_VH-8
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSS

SEQ ID NO: 21 - protein, 125 aa, synthetic construct
  REGION 1..125: note = CD3_VH-9
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 22 - protein, 109 aa, synthetic construct
  REGION 1..109: note = CD3_VL-1
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVL

SEQ ID NO: 23 - protein, 109 aa, synthetic construct
  REGION 1..109: note = CD3_VL-2
ELVVTQEPSLTVSPGGTVTLTCRSSTGAVTTSNYANWVQKKPGQAPRGLIGGTNKRAPGTPARFSGSLLGGKAALTLSGVQPEDEAEYYCALWYSNLWVFGGGTKLTVL

SEQ ID NO: 24 - protein, 109 aa, synthetic construct
  REGION 1..109: note = CD3_VL-3
ELVVTQEPSLTVSPGGTVTLTCRSSTGAVTTSNYANWVQEKPGQAPRGLIGGTNKRAPGTPARFSGSLLGGKAALTLSGVQPEDEAEYYCALWYSNLWVFGGGTKLTVL

SEQ ID NO: 25 - protein, 109 aa, synthetic construct
  REGION 1..109: note = CD3_VL-4
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWDSNLWVFGGGTKLTVL

SEQ ID NO: 26 - protein, 109 aa, synthetic construct
  REGION 1..109: note = CD3_VL-5
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSQLWVFGGGTKLTVL

SEQ ID NO: 27 - protein, 109 aa, synthetic construct
  REGION 1..109: note = CD3_VL-6
ELVVTQEPSLTVSPGGTVTLTCRSSTGAVTTSNYANWVQDKPGQAPRGLIGDTNKRAPGTPARFSGSLLGGKAALTLSGVQPEDEAEYYCALWYSNLWVFGGGTKLTVL

SEQ ID NO: 28 - protein, 109 aa, synthetic construct
  REGION 1..109: note = CD3_VL-7
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSDLWVFGGGTKLTVL

SEQ ID NO: 29 - protein, 5 aa, synthetic construct
  REGION 1..5: note = CD3_CDRH1-1_Kabat
TYAMN

SEQ ID NO: 30 - protein, 5 aa, synthetic construct
  REGION 1..5: note = CD3_CDRH1-2_Kabat
DYAMN

SEQ ID NO: 31 - protein, 5 aa, synthetic construct
  REGION 1..5: note = CD3_CDRH1-3_Kabat
TYAMD

SEQ ID NO: 32 - protein, 7 aa, synthetic construct
  REGION 1..7: note = CD3_CDRH1-1_Chothia
GFTFNTY

SEQ ID NO: 33 - protein, 7 aa, synthetic construct
  REGION 1..7: note = CD3_CDRH1-2_Chothia
GFTFNDY

SEQ ID NO: 34 - protein, 19 aa, synthetic construct
  REGION 1..19: note = CD3_CDRH2-1_Kabat
RIRSKYNNYATYYADSVKD

SEQ ID NO: 35 - protein, 19 aa, synthetic construct
  REGION 1..19: note = CD3_CDRH2-2_Kabat
KIRSKYNNYATYYADSVKD

SEQ ID NO: 36 - protein, 8 aa, synthetic construct
  REGION 1..8: note = CD3_CDRH2-1_Chothia
RSKYNNYA

SEQ ID NO: 37 - protein, 14 aa, synthetic construct
  REGION 1..14: note = CD3_CDRH3-1
HGNFGNSYVSWFAY

SEQ ID NO: 38 - protein, 14 aa, synthetic construct
  REGION 1..14: note = CD3_CDRH3-2
HGNFGDSYVSWFAY

SEQ ID NO: 39 - protein, 14 aa, synthetic construct
  REGION 1..14: note = CD3_CDRH3-3
HGNFGDDYVSWFAY

SEQ ID NO: 40 - protein, 14 aa, synthetic construct
  REGION 1..14: note = CD3_CDRH3-4
HGSFGNSYVSWFAY

SEQ ID NO: 41 - protein, 14 aa, synthetic construct
  REGION 1..14: note = CD3_CDRH3-5
HGTFGNSYVSWFAY

SEQ ID NO: 42 - protein, 14 aa, synthetic construct
  REGION 1..14: note = CD3_CDRL1
RSSTGAVTTSNYAN

SEQ ID NO: 43 - protein, 7 aa, synthetic construct
  REGION 1..7: note = CD3_CDRL2-1
GTNKRAP

SEQ ID NO: 44 - protein, 7 aa, synthetic construct
  REGION 1..7: note = CD3_CDRL2-2
DTNKRAP

SEQ ID NO: 45 - protein, 9 aa, synthetic construct
  REGION 1..9: note = CD3_CDRL3-1
ALWYSNLWV

SEQ ID NO: 46 - protein, 9 aa, synthetic construct
  REGION 1..9: note = CD3_CDRL3-2
ALWDSNLWV

SEQ ID NO: 47 - protein, 9 aa, synthetic construct
  REGION 1..9: note = CD3_CDRL3-3
ALWYSQLWV

SEQ ID NO: 48 - protein, 9 aa, synthetic construct
  REGION 1..9: note = CD3_CDRL3-4
ALWYSDLWV

SEQ ID NO: 49 - protein, 188 aa, synthetic construct
  REGION 1..188: note = CD58_ECD
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 50 - protein, 94 aa, synthetic construct
  REGION 1..94: note = CD58_VD
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLE

SEQ ID NO: 51 - protein, 152 aa, synthetic construct
  REGION 1..152: note = IL17
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 52 - protein, 5 aa, synthetic construct
  REGION 1..5: note = Linker-1
GGGGS

SEQ ID NO: 53 - protein, 10 aa, synthetic construct
  REGION 1..10: note = Linker-2
GGGGSGGGGS

SEQ ID NO: 54 - protein, 15 aa, synthetic construct
  REGION 1..15: note = Linker-3
GGGGSGGGGSGGGGS

SEQ ID NO: 55 - protein, 215 aa, synthetic construct
  REGION 1..215: note = EIP0174_ULBP2/5/6 light chain
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 56 - protein, 448 aa, synthetic construct
  REGION 1..448: note = EIP0174_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 215 aa, synthetic construct
  REGION 1..215: note = EIP0174_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLCPPSSEELQANKATLVCLIDDFYPGAVTVAWKADGSPVKAGVETTKPSKQSNNKYAASSYLSLTPEQWKSHRSYSCQVTHEGSTVEKTVAPTESS

SEQ ID NO: 58 - protein, 453 aa, synthetic construct
  REGION 1..453: note = EIP0174_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPCAPSSKSTSGGTAALGCLVKDYFPEPVTVSWNSGALTSGVHTFPAVLQSSGLYSLSSKVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 59 - protein, 215 aa, synthetic construct
  REGION 1..215: note = EIP0175_ULBP2/5/6 light chain
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIKRTVAAPSVCIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGES

SEQ ID NO: 60 - protein, 448 aa, synthetic construct
  REGION 1..448: note = EIP0175_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKCTSGGTAALGCLVDDYFPEPVTVSWNSGALTSGVHTFPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 61 - protein, 215 aa, synthetic construct
  REGION 1..215: note = EIP0175_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKATLVCLIDDFYPGAVTVAWKADGSPVKAGVETTKPSKQSNNKYAASSYLSLTPEQWKSHRSYSCQVTHEGSTVEKTVAPTECS

SEQ ID NO: 62 - protein, 453 aa, synthetic construct
  REGION 1..453: note = EIP0175_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 63 - protein, 215 aa, synthetic construct
  REGION 1..215: note = EIP0187_ULBP2/5/6 light chain
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIKRTVAAPCVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGES

SEQ ID NO: 64 - protein, 448 aa, synthetic construct
  REGION 1..448: note = EIP0187_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTCGGTAALGCLVDDYFPEPVTVSWNSGALTSGVHTFPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 65 - protein, 215 aa, synthetic construct; identical to SEQ ID NO: 61
  REGION 1..215: note = EIP0187_CD3 light chain

SEQ ID NO: 66 - protein, 453 aa, synthetic construct; identical to SEQ ID NO: 62
  REGION 1..453: note = EIP0187_CD3 heavy chain

SEQ ID NO: 67 - protein, 215 aa, synthetic construct; identical to SEQ ID NO: 55
  REGION 1..215: note = EIP0205_ULBP2/5/6 light chain

SEQ ID NO: 68 - protein, 448 aa, synthetic construct; identical to SEQ ID NO: 56
  REGION 1..448: note = EIP0205_ULBP2/5/6 heavy chain

SEQ ID NO: 69 - protein, 215 aa, synthetic construct
  REGION 1..215: note = EIP0205_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 70 - protein, 453 aa, synthetic construct
  REGION 1..453: note = EIP0205_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 71 - protein, 215 aa, synthetic construct; identical to SEQ ID NO: 55
  REGION 1..215: note = EIP0206_ULBP2/5/6 light chain

SEQ ID NO: 72 - protein, 448 aa, synthetic construct; identical to SEQ ID NO: 56
  REGION 1..448: note = EIP0206_ULBP2/5/6 heavy chain

SEQ ID NO: 73 - protein, 215 aa, synthetic construct
  REGION 1..215: note = EIP0206_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKATLVCLIDDFYPGAVTVAWKADGSPVKAGVETCKPSKQSNNKYAASSYLSLTPEQWKSHRSYSCQVTHEGSTVEKTVAPTESS

SEQ ID NO: 74 - protein, 453 aa, synthetic construct
  REGION 1..453: note = EIP0206_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 75 - protein, 215 aa, synthetic construct; identical to SEQ ID NO: 63
  REGION 1..215: note = EIP0207_ULBP2/5/6 light chain

SEQ ID NO: 76 - protein, 448 aa, synthetic construct
  REGION 1..448: note = EIP0207_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPCSKSTSGGTAALGCLVDDYFPEPVTVSWNSGALTSGVHTFPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 77 - protein, 215 aa, synthetic construct; identical to SEQ ID NO: 61
  REGION 1..215: note = EIP0207_CD3 light chain

SEQ ID NO: 78 - protein, 453 aa, synthetic construct; identical to SEQ ID NO: 62
  REGION 1..453: note = EIP0207_CD3 heavy chain

SEQ ID NO: 79 - protein, 215 aa, synthetic construct
  REGION 1..215: note = EIP0208_ULBP2/5/6 light chain
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIKRTVAAPSVFIFPPSDEQLKSGTASVVCLLNKFYPREAKVQWKVDNALQSGNSQECVTEQDSKDSTYSLSSTLTLSKADYEKHKVYACEVTHQGLSSPVTKSFNRGES

SEQ ID NO: 80 - protein, 448 aa, synthetic construct
  REGION 1..448: note = EIP0208_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVKDYFPEPVTVSWNSGALTSGVHTCPAVLQSSGLYSLSSVVDVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 215 aa, synthetic construct; identical to SEQ ID NO: 61
  REGION 1..215: note = EIP0208_CD3 light chain

SEQ ID NO: 82 - protein, 453 aa, synthetic construct; identical to SEQ ID NO: 62
  REGION 1..453: note = EIP0208_CD3 heavy chain

SEQ ID NO: 83 - protein, 215 aa, synthetic construct; identical to SEQ ID NO: 79
  REGION 1..215: note = EIP0294_ULBP2/5/6 light chain

SEQ ID NO: 84 - protein, 448 aa, synthetic construct
  REGION 1..448: note = EIP0294_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVKDYFPEPVTVSWNSGALTSGVHTFCAVLQSSGLYSLSSVVDVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 85 - protein, 215 aa, synthetic construct
  REGION 1..215: note = EIP0294_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKATLVCLIKDFYPGAVTVAWKADGSPVKAGVETTKPSKQSNNKYAASSYLELTPEQWKSHRSYSCQVTHEGSTVEKTVAPTECS

SEQ ID NO: 86 - protein, 453 aa, synthetic construct
  REGION 1..453: note = EIP0294_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSEVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 87 - protein, 215 aa, synthetic construct; identical to SEQ ID NO: 79
  REGION 1..215: note = EIP0295_ULBP2/5/6 light chain

SEQ ID NO: 88 - protein, 448 aa, synthetic construct; identical to SEQ ID NO: 84
  REGION 1..448: note = EIP0295_ULBP2/5/6 heavy chain

SEQ ID NO: 89 - protein, 215 aa, synthetic construct; identical to SEQ ID NO: 61
  REGION 1..215: note = EIP0295_CD3 light chain

SEQ ID NO: 90 - protein, 453 aa, synthetic construct; identical to SEQ ID NO: 62
  REGION 1..453: note = EIP0295_CD3 heavy chain

SEQ ID NO: 91 - protein, 215 aa, synthetic construct
  REGION 1..215: note = EIP0306_ULBP2/5/6 light chain
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIKRTVAAPSVFIFPPSDEQLKSGTASVVCLLNNFYPREAKVQWKVDNALQSGNSQECVTEQDSKDSTYSLSSTLRLSKADYEKHKVYACEVTHQGLSSPVTKSFNRGEC

SEQ ID NO: 92 - protein, 448 aa, synthetic construct
  REGION 1..448: note = EIP0306_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVDDYFPEPVTVSWNSGALTSGVHTFCAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 93 - protein, 215 aa, synthetic construct; identical to SEQ ID NO: 85
  REGION 1..215: note = EIP0306_CD3 light chain

SEQ ID NO: 94 - protein, 453 aa, synthetic construct; identical to SEQ ID NO: 86
  REGION 1..453: note = EIP0306_CD3 heavy chain

SEQ ID NO: 95 - protein, 215 aa, synthetic construct; identical to SEQ ID NO: 91
  REGION 1..215: note = EIP0307_ULBP2/5/6 light chain

SEQ ID NO: 96 - protein, 448 aa, synthetic construct; identical to SEQ ID NO: 92
  REGION 1..448: note = EIP0307_ULBP2/5/6 heavy chain

SEQ ID NO: 97 - protein, 215 aa, synthetic construct; identical to SEQ ID NO: 61
  REGION 1..215: note = EIP0307_CD3 light chain

SEQ ID NO: 98 - protein, 453 aa, synthetic construct; identical to SEQ ID NO: 62
  REGION 1..453: note = EIP0307_CD3 heavy chain

SEQ ID NO: 99 - protein, 215 aa, synthetic construct; identical to SEQ ID NO: 91
  REGION 1..215: note = EIP0318_ULBP2/5/6 light chain

SEQ ID NO: 100 - protein, 448 aa, synthetic construct; identical to SEQ ID NO: 92
  REGION 1..448: note = EIP0318_ULBP2/5/6 heavy chain

SEQ ID NO: 101 - protein, 215 aa, synthetic construct
  REGION 1..215: note = EIP0318_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKADLVCLIDDFYPGAVTVAWKADGSPVKAGVETTKPSKQSNNKYAASSYLSLTPEQWKSHRSYSCQVTHEGSTVEKTVAPTECS

SEQ ID NO: 102 - protein, 453 aa, synthetic construct; identical to SEQ ID NO: 62
  REGION 1..453: note = EIP0318_CD3 heavy chain

SEQ ID NO: 103 - protein, 215 aa, synthetic construct
  REGION 1..215: note = EIP0340_ULBP2/5/6 light chain
EIVLTQSPATLSLSPGERATLSCRASQSIGTSIHWYQDKPGQAPRLLIYFASESISGIPARFSGSGSGTDFTLTISSLEPEDFAVYYCQQSNSWPLYTFGGGTKVEIKRTVAAPCVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGES

SEQ ID NO: 104 - protein, 448 aa, synthetic construct
  REGION 1..448: note = EIP0340_ULBP2/5/6 heavy chain
QVQLVQSGAEVKKPGASVKVSCKASGYTFTNYDINWVRKAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTCGGTAALGCLVDDYFPEPVTVSWNSGALTSGVHTFPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 105 - protein, 215 aa, synthetic construct
  REGION 1..215: note = EIP0340_CD3 light chain
ELVVTQEPSLTVSPGGTVTLTCRSSTGAVTTSNYANWVQKKPGQAPRGLIGGTNKRAPGTPARFSGSLLGGKAALTLSGVQPEDEAEYYCALWYSNLWVFGGGTKLTVLGQPKANPTVTLFPPSSEELQANKATLVCLIKDFYPGAVTVAWKADGSPVKAGVETTKPSKQSNNKYAASSYLSLTPEQWKSHRSYSCQVTHEGSTVEKTVAPTECS

SEQ ID NO: 106 - protein, 453 aa, synthetic construct
  REGION 1..453: note = EIP0340_CD3 heavy chain
EVQLLESGGGLVQPGGSLKLSCAASGFTFNTYAMNWVRDAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSEVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 107 - protein, 215 aa, synthetic construct
  REGION 1..215: note = EIP0342_ULBP2/5/6 light chain
EIVLTQSPATLSLSPGERATLSCRASQSIGTSIHWYQDKPGQAPRLLIYFASESISGIPARFSGSGSGTDFTLTISSLEPEDFAVYYCQQSNSWPLYTFGGGTKVEIKRTVAAPSVFIFPPSDEQLKSGTASVVCLLNKFYPREAKVQWKVDNALQSGNSQECVTEQDSKDSTYSLSSTLTLSKADYEKHKVYACEVTHQGLSSPVTKSFNRGES

SEQ ID NO: 108 - protein, 448 aa, synthetic construct
  REGION 1..448: note = EIP0342_ULBP2/5/6 heavy chain
QVQLVQSGAEVKKPGASVKVSCKASGYTFTNYDINWVRKAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVKDYFPEPVTVSWNSGALTSGVHTFCAVLQSSGLYSLSSVVDVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 109 - protein, 215 aa, synthetic construct; identical to SEQ ID NO: 105
  REGION 1..215: note = EIP0342_CD3 light chain

SEQ ID NO: 110 - protein, 453 aa, synthetic construct; identical to SEQ ID NO: 106
  REGION 1..453: note = EIP0342_CD3 heavy chain

SEQ ID NO: 111 - protein, 215 aa, synthetic construct; identical to SEQ ID NO: 107
  REGION 1..215: note = EIP0354_ULBP2/5/6 light chain

SEQ ID NO: 112 - protein, 448 aa, synthetic construct; identical to SEQ ID NO: 108
  REGION 1..448: note = EIP0354_ULBP2/5/6 heavy chain

SEQ ID NO: 113 - protein, 215 aa, synthetic construct
  REGION 1..215: note = EIP0354_CD3 light chain
ELVVTQEPSLTVSPGGTVTLTCRSSTGAVTTSNYANWVQKKPGQAPRGLIGGTNKRAPGTPARFSGSLLGGKAALTLSGVQPEDEAEYYCALWYSNLWVFGGGTKLTVLGQPKANPTVTLFPPSSEELQANKATLVCLIDDFYPGAVTVAWKADGSPVKAGVETTKPSKQSNNKYAASSYLSLTPEQWKSHRSYSCQVTHEGSTVEKTVAPTECS

SEQ ID NO: 114 - protein, 453 aa, synthetic construct
  REGION 1..453: note = EIP0354_CD3 heavy chain
EVQLLESGGGLVQPGGSLKLSCAASGFTFNTYAMNWVRDAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 115 - protein, 215 aa, synthetic construct; identical to SEQ ID NO: 107
  REGION 1..215: note = EIP0356_ULBP2/5/6 light chain

SEQ ID NO: 116 - protein, 448 aa, synthetic construct
  REGION 1..448: note = EIP0356_ULBP2/5/6 heavy chain
QVQLVQSGAEVKKPGASVKVSCKASGYTFTNYDINWVRKAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVKDYFPEPVTVSWNSGALTSGVHTCPAVLQSSGLYSLSSVVDVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 117 - protein, 215 aa, synthetic construct
  REGION 1..215: note = EIP0356_CD3 light chain
ELVVTQEPSLTVSPGGTVTLTCRSSTGAVTTSNYANWVQKKPGQAPRGLIGGTNKRAPGTPARFSGSLLGGKAALTLSGVQPEDEAEYYCALWYSNLWVFGGGTKLTVLGQPKANPTVTLFPPSSEELQANKADLVCLIKDFYPGAVTVAWKADGSPVKAGVETTKPSKQSNNKYAASSYLELTPEQWKSHRSYSCQVTHEGSTVEKTVAPTECS

SEQ ID NO: 118 - protein, 453 aa, synthetic construct
  REGION 1..453: note = EIP0356_CD3 heavy chain
EVQLLESGGGLVQPGGSLKLSCAASGFTFNTYAMNWVRDAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCSVKDYFPEPVTVSWNSGALTSGVHTFPAVLQSSGLYSLSSEVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 119 - protein, 215 aa, synthetic construct; identical to SEQ ID NO: 91
  REGION 1..215: note = EIP0367_ULBP2/5/6 light chain

SEQ ID NO: 120 - protein, 448 aa, synthetic construct
  REGION 1..448: note = EIP0367_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVDDYFPEPVTVSWNSGALTSGVHTCPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 121 - protein, 215 aa, synthetic construct; identical to SEQ ID NO: 61
  REGION 1..215: note = EIP0367_CD3 light chain

SEQ ID NO: 122 - protein, 453 aa, synthetic construct; identical to SEQ ID NO: 62
  REGION 1..453: note = EIP0367_CD3 heavy chain

SEQ ID NO: 123 - protein, 215 aa, synthetic construct; identical to SEQ ID NO: 79
  REGION 1..215: note = EIP0377_ULBP2/5/6 light chain

SEQ ID NO: 124 - protein, 448 aa, synthetic construct; identical to SEQ ID NO: 80
  REGION 1..448: note = EIP0377_ULBP2/5/6 heavy chain

SEQ ID NO: 125 - protein, 215 aa, synthetic construct
  REGION 1..215: note = EIP0377_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKAKLVCLISDFYPGAVTVAWKADGSPVKAGVETTKPSKQSNNKYAASSYLSLTPEQWKSHRSYSCQVTHEGSTVEKTVAPTECS

SEQ ID NO: 126 - protein, 453 aa, synthetic construct
  REGION 1..453: note = EIP0377_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 127 - protein, 215 aa, synthetic construct; identical to SEQ ID NO: 63
  REGION 1..215: note = EIP0404_ULBP2/5/6 light chain

SEQ ID NO: 128 - protein, 448 aa, synthetic construct; identical to SEQ ID NO: 64
  REGION 1..448: note = EIP0404_ULBP2/5/6 heavy chain

SEQ ID NO: 129 - protein, 215 aa, synthetic construct
  REGION 1..215: note = EIP0404_CD3 light chain
ELVVTQEPSLTVSPGGTVTLTCRSSTGAVTTSNYANWVQEKPGQAPRGLIGGTNKRAPGTPARFSGSLLGGKAALTLSGVQPEDEAEYYCALWYSNLWVFGGGTKLTVLGQPKANPTVTLFPPSSEELQANKADLVCLIKDFYPGAVTVAWKADGSPVKAGVETTKPSKQSNNKYAASSYLSLTPEQWKSHRSYSCQVTHEGSTVEKTVAPTECS

SEQ ID NO: 130 - protein, 453 aa, synthetic construct; identical to SEQ ID NO: 86
  REGION 1..453: note = EIP0404_CD3 heavy chain

SEQ ID NO: 131 - protein, 215 aa, synthetic construct
  REGION 1..215: note = EIP0406_ULBP2/5/6 light chain
EIVLTQSPATLSLSPGERATLSCRASQSIGTSIHWYQKKPGQAPRLLIYFASESISGIPARFSGSGSGTDFTLTISSLEPEDFAVYYCQQSNSWPLYTFGGGTKVEIKRTVAAPCVFIFPPSDEQLKSGTASVVCLLNKFYPREAKVQWKVDNALQSGNSQESVTEQDSKDSTYSLSSTLTLSKADYEKHKVYACEVTHQGLSSPVTKSFNRGES

SEQ ID NO: 132 - protein, 448 aa, synthetic construct
  REGION 1..448: note = EIP0406_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTCGGTAALGCLVKDYFPEPVTVSWNSGALTSGVHTSPAVLQSSGLYSLSSVVDVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 133 - protein, 215 aa, synthetic construct; identical to SEQ ID NO: 129
  REGION 1..215: note = EIP0406_CD3 light chain

SEQ ID NO: 134 - protein, 453 aa, synthetic construct; identical to SEQ ID NO: 86
  REGION 1..453: note = EIP0406_CD3 heavy chain

SEQ ID NO: 135 - protein, 215 aa, synthetic construct; identical to SEQ ID NO: 107
  REGION 1..215: note = EIP0473_ULBP2/5/6 light chain

SEQ ID NO: 136 - protein, 448 aa, synthetic construct; identical to SEQ ID NO: 108
  REGION 1..448: note = EIP0473_ULBP2/5/6 heavy chain

SEQ ID NO: 137 - protein, 215 aa, synthetic construct
  REGION 1..215: note = EIP0473_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSNLWVFGGGTKLTVLGQPKANPTVTLFPPSSEELQANKAKLVCLIKDFYPGAVTVAWKADGSPVKAGVETTKPSKQSNNKYAASSYLSLTPEQWKSHRSYSCQVTHEGSTVEKTVAPTECS

SEQ ID NO: 138 - protein, 453 aa, synthetic construct
  REGION 1..453: note = EIP0473_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVDDYFPEPVTVSWNSGALTSGVHTFPAVLQSSGLYSLSSDVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 139 - protein, 215 aa, synthetic construct; identical to SEQ ID NO: 55
  REGION 1..215: note = EIP0598_ULBP2/5/6 light chain

SEQ ID NO: 140 - protein, 448 aa, synthetic construct; identical to SEQ ID NO: 56
  REGION 1..448: note = EIP0598_ULBP2/5/6 heavy chain

SEQ ID NO: 141 - protein, 215 aa, synthetic construct; identical to SEQ ID NO: 69
  REGION 1..215: note = EIP0598_CD3 light chain

SEQ ID NO: 142 - protein, 453 aa, synthetic construct
  REGION 1..453: note = EIP0598_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 143 - protein, 215 aa, synthetic construct; identical to SEQ ID NO: 55
  REGION 1..215: note = EIP0527_ULBP2/5/6 light chain

SEQ ID NO: 144 - protein, 448 aa, synthetic construct; identical to SEQ ID NO: 56
  REGION 1..448: note = EIP0527_ULBP2/5/6 heavy chain

SEQ ID NO: 145 - protein, 215 aa, synthetic construct
  REGION 1..215: note = EIP0527_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWDSNLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 146 - protein, 453 aa, synthetic construct; identical to SEQ ID NO: 70
  REGION 1..453: note = EIP0527_CD3 heavy chain

SEQ ID NO: 147 - protein, 215 aa, synthetic construct; identical to SEQ ID NO: 55
  REGION 1..215: note = EIP0624_ULBP2/5/6 light chain

SEQ ID NO: 148 - protein, 448 aa, synthetic construct; identical to SEQ ID NO: 56
  REGION 1..448: note = EIP0624_ULBP2/5/6 heavy chain

SEQ ID NO: 149 - protein, 215 aa, synthetic construct
  REGION 1..215: note = EIP0624_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSQLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 150 - protein, 453 aa, synthetic construct
  REGION 1..453: note = EIP0624_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 151 - protein, 215 aa, synthetic construct; identical to SEQ ID NO: 55
  REGION 1..215: note = EIP0486_ULBP2/5/6 light chain

SEQ ID NO: 152 - protein, 448 aa, synthetic construct; identical to SEQ ID NO: 56
  REGION 1..448: note = EIP0486_ULBP2/5/6 heavy chain

SEQ ID NO: 153 - protein, 215 aa, synthetic construct
  REGION 1..215: note = EIP0486_CD3 light chain
ELVVTQEPSLTVSPGGTVTLTCRSSTGAVTTSNYANWVQDKPGQAPRGLIGDTNKRAPGTPARFSGSLLGGKAALTLSGVQPEDEAEYYCALWYSNLWVFGGGTKLTVLGQPKANPTVTLFPPSSEELQANKADLVCLISDFYPGAVTVAWKADGSPVKAGVETCKPSKQSNNKYAASSYLSLTPEQWKSHRSYSCQVTHEGSTVEKTVAPTESS

SEQ ID NO: 154 - protein, 453 aa, synthetic construct; identical to SEQ ID NO: 70
  REGION 1..453: note = EIP0486_CD3 heavy chain

SEQ ID NO: 155 - protein, 215 aa, synthetic construct; identical to SEQ ID NO: 55
  REGION 1..215: note = EIP0626_ULBP2/5/6 light chain

SEQ ID NO: 156 - protein, 448 aa, synthetic construct; identical to SEQ ID NO: 56
  REGION 1..448: note = EIP0626_ULBP2/5/6 heavy chain

SEQ ID NO: 157 - protein, 215 aa, synthetic construct; identical to SEQ ID NO: 149
  REGION 1..215: note = EIP0626_CD3 light chain

SEQ ID NO: 158 - protein, 453 aa, synthetic construct
  REGION 1..453: note = EIP0626_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 159 - protein, 215 aa, synthetic construct; identical to SEQ ID NO: 55
  REGION 1..215: note = EIP0483_ULBP2/5/6 light chain

SEQ ID NO: 160 - protein, 448 aa, synthetic construct; identical to SEQ ID NO: 56
  REGION 1..448: note = EIP0483_ULBP2/5/6 heavy chain

SEQ ID NO: 161 - protein, 215 aa, synthetic construct; identical to SEQ ID NO: 69
  REGION 1..215: note = EIP0483_CD3 light chain

SEQ ID NO: 162 - protein, 453 aa, synthetic construct
  REGION 1..453: note = EIP0483_CD3 heavy chain
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 163 - protein, 215 aa, synthetic construct; identical to SEQ ID NO: 55
  REGION 1..215: note = EIP0623_ULBP2/5/6 light chain

SEQ ID NO: 164 - protein, 448 aa, synthetic construct; identical to SEQ ID NO: 56
  REGION 1..448: note = EIP0623_ULBP2/5/6 heavy chain

SEQ ID NO: 165 - protein, 215 aa, synthetic construct; identical to SEQ ID NO: 149
  REGION 1..215: note = EIP0623_CD3 light chain

SEQ ID NO: 166 - protein, 453 aa, synthetic construct
  REGION 1..453: note = EIP0623_CD3 heavy chain
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 167 - protein, 215 aa, synthetic construct; identical to SEQ ID NO: 55
  REGION 1..215: note = EIP0621_ULBP2/5/6 light chain

SEQ ID NO: 168 - protein, 448 aa, synthetic construct; identical to SEQ ID NO: 56
  REGION 1..448: note = EIP0621_ULBP2/5/6 heavy chain

SEQ ID NO: 169 - protein, 215 aa, synthetic construct; identical to SEQ ID NO: 149
  REGION 1..215: note = EIP0621_CD3 light chain

SEQ ID NO: 170 - protein, 453 aa, synthetic construct
  REGION 1..453: note = EIP0621_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 171 - protein, 215 aa, synthetic construct; identical to SEQ ID NO: 55
  REGION 1..215: note = EIP0625_ULBP2/5/6 light chain

SEQ ID NO: 172 - protein, 448 aa, synthetic construct; identical to SEQ ID NO: 56
  REGION 1..448: note = EIP0625_ULBP2/5/6 heavy chain

SEQ ID NO: 173 - protein, 215 aa, synthetic construct; identical to SEQ ID NO: 69
  REGION 1..215: note = EIP0625_CD3 light chain

SEQ ID NO: 174 - protein, 453 aa, synthetic construct; identical to SEQ ID NO: 166
  REGION 1..453: note = EIP0625_CD3 heavy chain

SEQ ID NO: 175 - protein, 215 aa, synthetic construct; identical to SEQ ID NO: 55
  REGION 1..215: note = EIP0622_ULBP2/5/6 light chain

SEQ ID NO: 176 - protein, 448 aa, synthetic construct; identical to SEQ ID NO: 56
  REGION 1..448: note = EIP0622_ULBP2/5/6 heavy chain

SEQ ID NO: 177 - protein, 215 aa, synthetic construct; identical to SEQ ID NO: 149
  REGION 1..215: note = EIP0622_CD3 light chain

SEQ ID NO: 178 - protein, 453 aa, synthetic construct
  REGION 1..453: note = EIP0622_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 179 - protein, 215 aa, synthetic construct; identical to SEQ ID NO: 55
  REGION 1..215: note = EIP0525_ULBP2/5/6 light chain

SEQ ID NO: 180 - protein, 448 aa, synthetic construct; identical to SEQ ID NO: 56
  REGION 1..448: note = EIP0525_ULBP2/5/6 heavy chain

SEQ ID NO: 181 - protein, 215 aa, synthetic construct; identical to SEQ ID NO: 69
  REGION 1..215: note = EIP0525_CD3 light chain

SEQ ID NO: 182 - protein, 453 aa, synthetic construct
  REGION 1..453: note = EIP0525_CD3 heavy chain
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 183 - protein, 215 aa, synthetic construct; identical to SEQ ID NO: 55
  REGION 1..215: note = EIP0630_ULBP2/5/6 light chain

SEQ ID NO: 184 - protein, 448 aa, synthetic construct; identical to SEQ ID NO: 56
  REGION 1..448: note = EIP0630_ULBP2/5/6 heavy chain

SEQ ID NO: 185 - protein, 215 aa, synthetic construct; identical to SEQ ID NO: 145
  REGION 1..215: note = EIP0630_CD3 light chain

SEQ ID NO: 186 - protein, 453 aa, synthetic construct; identical to SEQ ID NO: 142
  REGION 1..453: note = EIP0630_CD3 heavy chain

SEQ ID NO: 187 - protein, 215 aa, synthetic construct; identical to SEQ ID NO: 55
  REGION 1..215: note = EIP0540_ULBP2/5/6 light chain

SEQ ID NO: 188 - protein, 448 aa, synthetic construct; identical to SEQ ID NO: 56
  REGION 1..448: note = EIP0540_ULBP2/5/6 heavy chain

SEQ ID NO: 189 - protein, 215 aa, synthetic construct; identical to SEQ ID NO: 149
  REGION 1..215: note = EIP0540_CD3 light chain

SEQ ID NO: 190 - protein, 453 aa, synthetic construct
  REGION 1..453: note = EIP0540_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 191 - protein, 215 aa, synthetic construct; identical to SEQ ID NO: 55
  REGION 1..215: note = EIP0628_ULBP2/5/6 light chain

SEQ ID NO: 192 - protein, 448 aa, synthetic construct; identical to SEQ ID NO: 56
  REGION 1..448: note = EIP0628_ULBP2/5/6 heavy chain

SEQ ID NO: 193 - protein, 215 aa, synthetic construct; identical to SEQ ID NO: 153
  REGION 1..215: note = EIP0628_CD3 light chain

SEQ ID NO: 194 - protein, 453 aa, synthetic construct; identical to SEQ ID NO: 142
  REGION 1..453: note = EIP0628_CD3 heavy chain

SEQ ID NO: 195 - protein, 215 aa, synthetic construct; identical to SEQ ID NO: 55
  REGION 1..215: note = EIP0542_ULBP2/5/6 light chain

SEQ ID NO: 196 - protein, 448 aa, synthetic construct; identical to SEQ ID NO: 56
  REGION 1..448: note = EIP0542_ULBP2/5/6 heavy chain

SEQ ID NO: 197 - protein, 215 aa, synthetic construct; identical to SEQ ID NO: 149
  REGION 1..215: note = EIP0542_CD3 light chain

SEQ ID NO: 198 - protein, 453 aa, synthetic construct
  REGION 1..453: note = EIP0542_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 199 - protein, 215 aa, synthetic construct; identical to SEQ ID NO: 55
  REGION 1..215: note = EIP0627_ULBP2/5/6 light chain

SEQ ID NO: 200 - protein, 448 aa, synthetic construct; identical to SEQ ID NO: 56
  REGION 1..448: note = EIP0627_ULBP2/5/6 heavy chain

SEQ ID NO: 201 - protein, 215 aa, synthetic construct; identical to SEQ ID NO: 69
  REGION 1..215: note = EIP0627_CD3 light chain

SEQ ID NO: 202 - protein, 453 aa, synthetic construct
  REGION 1..453: note = EIP0627_CD3 heavy chain
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 203 - protein, 215 aa, synthetic construct; identical to SEQ ID NO: 55
  REGION 1..215: note = EIP0515_ULBP2/5/6 light chain

SEQ ID NO: 204 - protein, 448 aa, synthetic construct; identical to SEQ ID NO: 56
  REGION 1..448: note = EIP0515_ULBP2/5/6 heavy chain

SEQ ID NO: 205 - protein, 215 aa, synthetic construct; identical to SEQ ID NO: 149
  REGION 1..215: note = EIP0515_CD3 light chain

SEQ ID NO: 206 - protein, 453 aa, synthetic construct
  REGION 1..453: note = EIP0515_CD3 heavy chain
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 207 - protein, 215 aa, synthetic construct; identical to SEQ ID NO: 55
  REGION 1..215: note = EIP0477_ULBP2/5/6 light chain

SEQ ID NO: 208 - protein, 448 aa, synthetic construct; identical to SEQ ID NO: 56
  REGION 1..448: note = EIP0477_ULBP2/5/6 heavy chain

SEQ ID NO: 209 - protein, 215 aa, synthetic construct; identical to SEQ ID NO: 149
  REGION 1..215: note = EIP0477_CD3 light chain

SEQ ID NO: 210 - protein, 453 aa, synthetic construct
  REGION 1..453: note = EIP0477_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 211 - protein, 215 aa, synthetic construct; identical to SEQ ID NO: 55
  REGION 1..215: note = EIP0541_ULBP2/5/6 light chain

SEQ ID NO: 212 - protein, 448 aa, synthetic construct; identical to SEQ ID NO: 56
  REGION 1..448: note = EIP0541_ULBP2/5/6 heavy chain

SEQ ID NO: 213 - protein, 215 aa, synthetic construct; identical to SEQ ID NO: 69
  REGION 1..215: note = EIP0541_CD3 light chain

SEQ ID NO: 214 - protein, 453 aa, synthetic construct; identical to SEQ ID NO: 206
  REGION 1..453: note = EIP0541_CD3 heavy chain

SEQ ID NO: 215 - protein, 215 aa, synthetic construct; identical to SEQ ID NO: 55
  REGION 1..215: note = EIP0513_ULBP2/5/6 light chain

SEQ ID NO: 216 - protein, 448 aa, synthetic construct; identical to SEQ ID NO: 56
  REGION 1..448: note = EIP0513_ULBP2/5/6 heavy chain

SEQ ID NO: 217 - protein, 215 aa, synthetic construct; identical to SEQ ID NO: 149
  REGION 1..215: note = EIP0513_CD3 light chain

SEQ ID NO: 218 - protein, 453 aa, synthetic construct
  REGION 1..453: note = EIP0513_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 219 - protein, 215 aa, synthetic construct; identical to SEQ ID NO: 55
  REGION 1..215: note = EIP0629_ULBP2/5/6 light chain

SEQ ID NO: 220 - protein, 448 aa, synthetic construct; identical to SEQ ID NO: 56
  REGION 1..448: note = EIP0629_ULBP2/5/6 heavy chain

SEQ ID NO: 221 - protein, 215 aa, synthetic construct; identical to SEQ ID NO: 69
  REGION 1..215: note = EIP0629_CD3 light chain

SEQ ID NO: 222 - protein, 453 aa, synthetic construct
  REGION 1..453: note = EIP0629_CD3 heavy chain
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 223 - protein, 215 aa, synthetic construct; identical to SEQ ID NO: 55
  REGION 1..215: note = EIP0359_ULBP2/5/6 light chain

SEQ ID NO: 224 - protein, 448 aa, synthetic construct; identical to SEQ ID NO: 56
  REGION 1..448: note = EIP0359_ULBP2/5/6 heavy chain

SEQ ID NO: 225 - protein, 215 aa, synthetic construct; identical to SEQ ID NO: 69
  REGION 1..215: note = EIP0359_CD3 light chain

SEQ ID NO: 226 - protein, 653 aa, synthetic construct
  REGION 1..653: note = EIP0359_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 227 - protein, 215 aa, synthetic construct; identical to SEQ ID NO: 55
  REGION 1..215: note = EIP0360_ULBP2/5/6 light chain

SEQ ID NO: 228 - protein, 448 aa, synthetic construct; identical to SEQ ID NO: 56
  REGION 1..448: note = EIP0360_ULBP2/5/6 heavy chain

SEQ ID NO: 229 - protein, 215 aa, synthetic construct; identical to SEQ ID NO: 69
  REGION 1..215: note = EIP0360_CD3 light chain

SEQ ID NO: 230 - protein, 559 aa, synthetic construct
  REGION 1..559: note = EIP0360_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLE

SEQ ID NO: 231 - protein, 215 aa, synthetic construct; identical to SEQ ID NO: 55
  REGION 1..215: note = EIP0545_ULBP2/5/6 light chain

SEQ ID NO: 232 - protein, 448 aa, synthetic construct; identical to SEQ ID NO: 56
  REGION 1..448: note = EIP0545_ULBP2/5/6 heavy chain

SEQ ID NO: 233 - protein, 215 aa, synthetic construct; identical to SEQ ID NO: 69
  REGION 1..215: note = EIP0545_CD3 light chain

SEQ ID NO: 234 - protein, 653 aa, synthetic construct; identical to SEQ ID NO: 226
  REGION 1..653: note = EIP0545_CD3 heavy chain

SEQ ID NO: 235 - protein, 215 aa, synthetic construct; identical to SEQ ID NO: 55
  REGION 1..215: note = EIP0544_ULBP2/5/6 light chain

SEQ ID NO: 236 - protein, 448 aa, synthetic construct; identical to SEQ ID NO: 56
  REGION 1..448: note = EIP0544_ULBP2/5/6 heavy chain

SEQ ID NO: 237 - protein, 215 aa, synthetic construct; identical to SEQ ID NO: 69
  REGION 1..215: note = EIP0544_CD3 light chain

SEQ ID NO: 238 - protein, 559 aa, synthetic construct; identical to SEQ ID NO: 230
  REGION 1..559: note = EIP0544_CD3 heavy chain

SEQ ID NO: 239 - protein, 215 aa, synthetic construct; identical to SEQ ID NO: 55
  REGION 1..215: note = EIP0566_ULBP2/5/6 light chain

SEQ ID NO: 240 - protein, 448 aa, synthetic construct; identical to SEQ ID NO: 56
  REGION 1..448: note = EIP0566_ULBP2/5/6 heavy chain

SEQ ID NO: 241 - protein, 215 aa, synthetic construct; identical to SEQ ID NO: 145
  REGION 1..215: note = EIP0566_CD3 light chain

SEQ ID NO: 242 - protein, 653 aa, synthetic construct; identical to SEQ ID NO: 226
  REGION 1..653: note = EIP0566_CD3 heavy chain

SEQ ID NO: 243 - protein, 215 aa, synthetic construct; identical to SEQ ID NO: 55
  REGION 1..215: note = EIP0634_ULBP2/5/6 light chain

SEQ ID NO: 244 - protein, 448 aa, synthetic construct; identical to SEQ ID NO: 56
  REGION 1..448: note = EIP0634_ULBP2/5/6 heavy chain

SEQ ID NO: 245 - protein, 215 aa, synthetic construct; identical to SEQ ID NO: 149
  REGION 1..215: note = EIP0634_CD3 light chain

SEQ ID NO: 246 - protein, 653 aa, synthetic construct
  REGION 1..653: note = EIP0634_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 247 - protein, 215 aa, synthetic construct; identical to SEQ ID NO: 55
  REGION 1..215: note = EIP0562_ULBP2/5/6 light chain

SEQ ID NO: 248 - protein, 448 aa, synthetic construct; identical to SEQ ID NO: 56
  REGION 1..448: note = EIP0562_ULBP2/5/6 heavy chain

SEQ ID NO: 249 - protein, 215 aa, synthetic construct; identical to SEQ ID NO: 153
  REGION 1..215: note = EIP0562_CD3 light chain

SEQ ID NO: 250 - protein, 653 aa, synthetic construct; identical to SEQ ID NO: 226
  REGION 1..653: note = EIP0562_CD3 heavy chain

SEQ ID NO: 251 - protein, 215 aa, synthetic construct; identical to SEQ ID NO: 55
  REGION 1..215: note = EIP0636_ULBP2/5/6 light chain

SEQ ID NO: 252 - protein, 448 aa, synthetic construct; identical to SEQ ID NO: 56
  REGION 1..448: note = EIP0636_ULBP2/5/6 heavy chain

SEQ ID NO: 253 - protein, 215 aa, synthetic construct; identical to SEQ ID NO: 149
  REGION 1..215: note = EIP0636_CD3 light chain

SEQ ID NO: 254 - protein, 653 aa, synthetic construct
  REGION 1..653: note = EIP0636_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 255 - protein, 215 aa, synthetic construct; identical to SEQ ID NO: 55
  REGION 1..215: note = EIP0561_ULBP2/5/6 light chain

SEQ ID NO: 256 - protein, 448 aa, synthetic construct; identical to SEQ ID NO: 56
  REGION 1..448: note = EIP0561_ULBP2/5/6 heavy chain

SEQ ID NO: 257 - protein, 215 aa, synthetic construct; identical to SEQ ID NO: 69
  REGION 1..215: note = EIP0561_CD3 light chain

SEQ ID NO: 258 - protein, 653 aa, synthetic construct
  REGION 1..653: note = EIP0561_CD3 heavy chain
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 259 - protein, 215 aa, synthetic construct; identical to SEQ ID NO: 55
  REGION 1..215: note = EIP0633_ULBP2/5/6 light chain

SEQ ID NO: 260 - protein, 448 aa, synthetic construct; identical to SEQ ID NO: 56
  REGION 1..448: note = EIP0633_ULBP2/5/6 heavy chain

SEQ ID NO: 261 - protein, 215 aa, synthetic construct; identical to SEQ ID NO: 149
  REGION 1..215: note = EIP0633_CD3 light chain

SEQ ID NO: 262 - protein, 653 aa, synthetic construct
  REGION 1..653: note = EIP0633_CD3 heavy chain
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 263 - protein, 215 aa, synthetic construct; identical to SEQ ID NO: 55
  REGION 1..215: note = EIP0631_ULBP2/5/6 light chain

SEQ ID NO: 264 - protein, 448 aa, synthetic construct; identical to SEQ ID NO: 56
  REGION 1..448: note = EIP0631_ULBP2/5/6 heavy chain

SEQ ID NO: 265 - protein, 215 aa, synthetic construct; identical to SEQ ID NO: 149
  REGION 1..215: note = EIP0631_CD3 light chain

SEQ ID NO: 266 - protein, 653 aa, synthetic construct
  REGION 1..653: note = EIP0631_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 267 - protein, 215 aa, synthetic construct; identical to SEQ ID NO: 55
  REGION 1..215: note = EIP0635_ULBP2/5/6 light chain

SEQ ID NO: 268 - protein, 448 aa, synthetic construct; identical to SEQ ID NO: 56
  REGION 1..448: note = EIP0635_ULBP2/5/6 heavy chain

SEQ ID NO: 269 - protein, 215 aa, synthetic construct; identical to SEQ ID NO: 69
  REGION 1..215: note = EIP0635_CD3 light chain

SEQ ID NO: 270 - protein, 653 aa, synthetic construct; identical to SEQ ID NO: 262
  REGION 1..653: note = EIP0635_CD3 heavy chain

SEQ ID NO: 271 - protein, 215 aa, synthetic construct; identical to SEQ ID NO: 55
  REGION 1..215: note = EIP0632_ULBP2/5/6 light chain

SEQ ID NO: 272 - protein, 448 aa, synthetic construct; identical to SEQ ID NO: 56
  REGION 1..448: note = EIP0632_ULBP2/5/6 heavy chain

SEQ ID NO: 273 - protein, 215 aa, synthetic construct; identical to SEQ ID NO: 149
  REGION 1..215: note = EIP0632_CD3 light chain

SEQ ID NO: 274 - protein, 653 aa, synthetic construct
  REGION 1..653: note = EIP0632_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 275 - protein, 215 aa, synthetic construct; identical to SEQ ID NO: 55
  REGION 1..215: note = EIP0565_ULBP2/5/6 light chain

SEQ ID NO: 276 - protein, 448 aa, synthetic construct; identical to SEQ ID NO: 56
  REGION 1..448: note = EIP0565_ULBP2/5/6 heavy chain

SEQ ID NO: 277 - protein, 215 aa, synthetic construct; identical to SEQ ID NO: 69
  REGION 1..215: note = EIP0565_CD3 light chain

SEQ ID NO: 278 - protein, 653 aa, synthetic construct
  REGION 1..653: note = EIP0565_CD3 heavy chain
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 279 - protein, 215 aa, synthetic construct; identical to SEQ ID NO: 55
  REGION 1..215: note = EIP0599_ULBP2/5/6 light chain

SEQ ID NO: 280 - protein, 448 aa, synthetic construct; identical to SEQ ID NO: 56
  REGION 1..448: note = EIP0599_ULBP2/5/6 heavy chain

SEQ ID NO: 281 - protein, 215 aa, synthetic construct; identical to SEQ ID NO: 69
  REGION 1..215: note = EIP0599_CD3 light chain

SEQ ID NO: 282 - protein, 653 aa, synthetic construct
  REGION 1..653: note = EIP0599_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 283 - protein, 215 aa, synthetic construct; identical to SEQ ID NO: 55
  REGION 1..215: note = EIP0640_ULBP2/5/6 light chain

SEQ ID NO: 284 - protein, 448 aa, synthetic construct; identical to SEQ ID NO: 56
  REGION 1..448: note = EIP0640_ULBP2/5/6 heavy chain

SEQ ID NO: 285 - protein, 215 aa, synthetic construct; identical to SEQ ID NO: 145
  REGION 1..215: note = EIP0640_CD3 light chain

SEQ ID NO: 286 - protein, 653 aa, synthetic construct; identical to SEQ ID NO: 282
  REGION 1..653: note = EIP0640_CD3 heavy chain

SEQ ID NO: 287 - protein, 215 aa, synthetic construct; identical to SEQ ID NO: 55
  REGION 1..215: note = EIP0567_ULBP2/5/6 light chain

SEQ ID NO: 288 - protein, 448 aa, synthetic construct; identical to SEQ ID NO: 56
  REGION 1..448: note = EIP0567_ULBP2/5/6 heavy chain

SEQ ID NO: 289 - protein, 215 aa, synthetic construct; identical to SEQ ID NO: 149
  REGION 1..215: note = EIP0567_CD3 light chain

SEQ ID NO: 290 - protein, 653 aa, synthetic construct
  REGION 1..653: note = EIP0567_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 291 - protein, 215 aa, synthetic construct; identical to SEQ ID NO: 55
  REGION 1..215: note = EIP0638_ULBP2/5/6 light chain

SEQ ID NO: 292 - protein, 448 aa, synthetic construct; identical to SEQ ID NO: 56
  REGION 1..448: note = EIP0638_ULBP2/5/6 heavy chain

SEQ ID NO: 293 - protein, 215 aa, synthetic construct; identical to SEQ ID NO: 153
  REGION 1..215: note = EIP0638_CD3 light chain

SEQ ID NO: 294 - protein, 653 aa, synthetic construct; identical to SEQ ID NO: 282
  REGION 1..653: note = EIP0638_CD3 heavy chain

SEQ ID NO: 295 - protein, 215 aa, synthetic construct; identical to SEQ ID NO: 55
  REGION 1..215: note = EIP0568_ULBP2/5/6 light chain

SEQ ID NO: 296 - protein, 448 aa, synthetic construct; identical to SEQ ID NO: 56
  REGION 1..448: note = EIP0568_ULBP2/5/6 heavy chain

SEQ ID NO: 297 - protein, 215 aa, synthetic construct; identical to SEQ ID NO: 149
  REGION 1..215: note = EIP0568_CD3 light chain

SEQ ID NO: 298 - protein, 653 aa, synthetic construct
  REGION 1..653: note = EIP0568_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 299 - protein, 215 aa, synthetic construct; identical to SEQ ID NO: 55
  REGION 1..215: note = EIP0637_ULBP2/5/6 light chain

SEQ ID NO: 300 - protein, 448 aa, synthetic construct; identical to SEQ ID NO: 56
  REGION 1..448: note = EIP0637_ULBP2/5/6 heavy chain

SEQ ID NO: 301 - protein, 215 aa, synthetic construct; identical to SEQ ID NO: 69
  REGION 1..215: note = EIP0637_CD3 light chain

SEQ ID NO: 302 - protein, 653 aa, synthetic construct
  REGION 1..653: note = EIP0637_CD3 heavy chain
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 303 - protein, 215 aa, synthetic construct; identical to SEQ ID NO: 55
  REGION 1..215: note = EIP0564_ULBP2/5/6 light chain

SEQ ID NO: 304 - protein, 448 aa, synthetic construct; identical to SEQ ID NO: 56
  REGION 1..448: note = EIP0564_ULBP2/5/6 heavy chain

SEQ ID NO: 305 - protein, 215 aa, synthetic construct; identical to SEQ ID NO: 149
  REGION 1..215: note = EIP0564_CD3 light chain

SEQ ID NO: 306 - protein, 653 aa, synthetic construct
  REGION 1..653: note = EIP0564_CD3 heavy chain
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 307 - protein, 215 aa, synthetic construct; identical to SEQ ID NO: 55
  REGION 1..215: note = EIP0560_ULBP2/5/6 light chain

SEQ ID NO: 308 - protein, 448 aa, synthetic construct; identical to SEQ ID NO: 56
  REGION 1..448: note = EIP0560_ULBP2/5/6 heavy chain

SEQ ID NO: 309 - protein, 215 aa, synthetic construct; identical to SEQ ID NO: 149
  REGION 1..215: note = EIP0560_CD3 light chain

SEQ ID NO: 310 - protein, 653 aa, synthetic construct
  REGION 1..653: note = EIP0560_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 311 - protein, 215 aa, synthetic construct; identical to SEQ ID NO: 55
  REGION 1..215: note = EIP0662_ULBP2/5/6 light chain

SEQ ID NO: 312 - protein, 448 aa, synthetic construct; identical to SEQ ID NO: 56
  REGION 1..448: note = EIP0662_ULBP2/5/6 heavy chain

SEQ ID NO: 313 - protein, 215 aa, synthetic construct; identical to SEQ ID NO: 69
  REGION 1..215: note = EIP0662_CD3 light chain

SEQ ID NO: 314 - protein, 653 aa, synthetic construct; identical to SEQ ID NO: 306
  REGION 1..653: note = EIP0662_CD3 heavy chain

SEQ ID NO: 315 - protein, 215 aa, synthetic construct; identical to SEQ ID NO: 55
  REGION 1..215: note = EIP0563_ULBP2/5/6 light chain

SEQ ID NO: 316 - protein, 448 aa, synthetic construct; identical to SEQ ID NO: 56
  REGION 1..448: note = EIP0563_ULBP2/5/6 heavy chain

SEQ ID NO: 317 - protein, 215 aa, synthetic construct; identical to SEQ ID NO: 149
  REGION 1..215: note = EIP0563_CD3 light chain

SEQ ID NO: 318 - protein, 653 aa, synthetic construct
  REGION 1..653: note = EIP0563_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 319 - protein, 215 aa, synthetic construct; identical to SEQ ID NO: 55
  REGION 1..215: note = EIP0639_ULBP2/5/6 light chain

SEQ ID NO: 320 - protein, 448 aa, synthetic construct; identical to SEQ ID NO: 56
  REGION 1..448: note = EIP0639_ULBP2/5/6 heavy chain

SEQ ID NO: 321 - protein, 215 aa, synthetic construct; identical to SEQ ID NO: 69
  REGION 1..215: note = EIP0639_CD3 light chain

SEQ ID NO: 322 - protein, 653 aa, synthetic construct
  REGION 1..653: note = EIP0639_CD3 heavy chain
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 323 - protein, 215 aa, synthetic construct; identical to SEQ ID NO: 55
  REGION 1..215: note = EIP0363_ULBP2/5/6 light chain

SEQ ID NO: 324 - protein, 448 aa, synthetic construct; identical to SEQ ID NO: 56
  REGION 1..448: note = EIP0363_ULBP2/5/6 heavy chain

SEQ ID NO: 325 - protein, 215 aa, synthetic construct; identical to SEQ ID NO: 69
  REGION 1..215: note = EIP0363_CD3 light chain

SEQ ID NO: 326 - protein, 617 aa, synthetic construct
  REGION 1..617: note = EIP0363_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 327 - protein, 215 aa, synthetic construct; identical to SEQ ID NO: 55
  REGION 1..215: note = EIP0663_ULBP2/5/6 light chain

SEQ ID NO: 328 - protein, 448 aa, synthetic construct; identical to SEQ ID NO: 56
  REGION 1..448: note = EIP0663_ULBP2/5/6 heavy chain

SEQ ID NO: 329 - protein, 215 aa, synthetic construct; identical to SEQ ID NO: 69
  REGION 1..215: note = EIP0663_CD3 light chain

SEQ ID NO: 330 - protein, 617 aa, synthetic construct
  REGION 1..617: note = EIP0663_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 331 - protein, 215 aa, synthetic construct; identical to SEQ ID NO: 63
  REGION 1..215: note = EIP0641_ULBP2/5/6 light chain

SEQ ID NO: 332 - protein, 448 aa, synthetic construct; identical to SEQ ID NO: 64
  REGION 1..448: note = EIP0641_ULBP2/5/6 heavy chain

SEQ ID NO: 333 - protein, 215 aa, synthetic construct; identical to SEQ ID NO: 61
  REGION 1..215: note = EIP0641_CD3 light chain

SEQ ID NO: 334 - protein, 653 aa, synthetic construct
  REGION 1..653: note = EIP0641_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 335 - protein, 215 aa, synthetic construct; identical to SEQ ID NO: 63
  REGION 1..215: note = EIP0642_ULBP2/5/6 light chain

SEQ ID NO: 336 - protein, 448 aa, synthetic construct; identical to SEQ ID NO: 64
  REGION 1..448: note = EIP0642_ULBP2/5/6 heavy chain

SEQ ID NO: 337 - protein, 215 aa, synthetic construct
  REGION 1..215: note = EIP0642_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWDSNLWVFGGGTKLTVLGQPKANPTVTLFPPSSEELQANKATLVCLIDDFYPGAVTVAWKADGSPVKAGVETTKPSKQSNNKYAASSYLSLTPEQWKSHRSYSCQVTHEGSTVEKTVAPTECS

SEQ ID NO: 338 - protein, 653 aa, synthetic construct; identical to SEQ ID NO: 334
  REGION 1..653: note = EIP0642_CD3 heavy chain

SEQ ID NO: 339 - protein, 215 aa, synthetic construct; identical to SEQ ID NO: 63
  REGION 1..215: note = EIP0643_ULBP2/5/6 light chain

SEQ ID NO: 340 - protein, 448 aa, synthetic construct; identical to SEQ ID NO: 64
  REGION 1..448: note = EIP0643_ULBP2/5/6 heavy chain

SEQ ID NO: 341 - protein, 215 aa, synthetic construct
  REGION 1..215: note = EIP0643_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSQLWVFGGGTKLTVLGQPKANPTVTLFPPSSEELQANKATLVCLIDDFYPGAVTVAWKADGSPVKAGVETTKPSKQSNNKYAASSYLSLTPEQWKSHRSYSCQVTHEGSTVEKTVAPTECS

SEQ ID NO: 342 - protein, 653 aa, synthetic construct
  REGION 1..653: note = EIP0643_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 343 - protein, 215 aa, synthetic construct; identical to SEQ ID NO: 63
  REGION 1..215: note = EIP0644_ULBP2/5/6 light chain

SEQ ID NO: 344 - protein, 448 aa, synthetic construct; identical to SEQ ID NO: 64
  REGION 1..448: note = EIP0644_ULBP2/5/6 heavy chain

SEQ ID NO: 345 - protein, 215 aa, synthetic construct
  REGION 1..215: note = EIP0644_CD3 light chain
ELVVTQEPSLTVSPGGTVTLTCRSSTGAVTTSNYANWVQDKPGQAPRGLIGDTNKRAPGTPARFSGSLLGGKAALTLSGVQPEDEAEYYCALWYSNLWVFGGGTKLTVLGQPKANPTVTLFPPSSEELQANKATLVCLIDDFYPGAVTVAWKADGSPVKAGVETTKPSKQSNNKYAASSYLSLTPEQWKSHRSYSCQVTHEGSTVEKTVAPTECS

SEQ ID NO: 346 - protein, 653 aa, synthetic construct; identical to SEQ ID NO: 334
  REGION 1..653: note = EIP0644_CD3 heavy chain

SEQ ID NO: 347 - protein, 215 aa, synthetic construct; identical to SEQ ID NO: 63
  REGION 1..215: note = EIP0645_ULBP2/5/6 light chain

SEQ ID NO: 348 - protein, 448 aa, synthetic construct; identical to SEQ ID NO: 64
  REGION 1..448: note = EIP0645_ULBP2/5/6 heavy chain

SEQ ID NO: 349 - protein, 215 aa, synthetic construct; identical to SEQ ID NO: 341
  REGION 1..215: note = EIP0645_CD3 light chain

SEQ ID NO: 350 - protein, 653 aa, synthetic construct
  REGION 1..653: note = EIP0645_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 351 - protein, 215 aa, synthetic construct; identical to SEQ ID NO: 63
  REGION 1..215: note = EIP0646_ULBP2/5/6 light chain

SEQ ID NO: 352 - protein, 448 aa, synthetic construct; identical to SEQ ID NO: 64
  REGION 1..448: note = EIP0646_ULBP2/5/6 heavy chain

SEQ ID NO: 353 - protein, 215 aa, synthetic construct; identical to SEQ ID NO: 61
  REGION 1..215: note = EIP0646_CD3 light chain

SEQ ID NO: 354 - protein, 653 aa, synthetic construct
  REGION 1..653: note = EIP0646_CD3 heavy chain
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 355 - protein, 215 aa, synthetic construct; identical to SEQ ID NO: 63
  REGION 1..215: note = EIP0647_ULBP2/5/6 light chain

SEQ ID NO: 356 - protein, 448 aa, synthetic construct; identical to SEQ ID NO: 64
  REGION 1..448: note = EIP0647_ULBP2/5/6 heavy chain

SEQ ID NO: 357 - protein, 215 aa, synthetic construct; identical to SEQ ID NO: 341
  REGION 1..215: note = EIP0647_CD3 light chain

SEQ ID NO: 358 - protein, 653 aa, synthetic construct
  REGION 1..653: note = EIP0647_CD3 heavy chain
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 359 - protein, 215 aa, synthetic construct; identical to SEQ ID NO: 63
  REGION 1..215: note = EIP0648_ULBP2/5/6 light chain

SEQ ID NO: 360 - protein, 448 aa, synthetic construct; identical to SEQ ID NO: 64
  REGION 1..448: note = EIP0648_ULBP2/5/6 heavy chain

SEQ ID NO: 361 - protein, 215 aa, synthetic construct; identical to SEQ ID NO: 341
  REGION 1..215: note = EIP0648_CD3 light chain

SEQ ID NO: 362 - protein, 653 aa, synthetic construct
  REGION 1..653: note = EIP0648_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 363 - protein, 215 aa, synthetic construct; identical to SEQ ID NO: 63
  REGION 1..215: note = EIP0649_ULBP2/5/6 light chain

SEQ ID NO: 364 - protein, 448 aa, synthetic construct; identical to SEQ ID NO: 64
  REGION 1..448: note = EIP0649_ULBP2/5/6 heavy chain

SEQ ID NO: 365 - protein, 215 aa, synthetic construct; identical to SEQ ID NO: 61
  REGION 1..215: note = EIP0649_CD3 light chain

SEQ ID NO: 366 - protein, 653 aa, synthetic construct; identical to SEQ ID NO: 358
  REGION 1..653: note = EIP0649_CD3 heavy chain

SEQ ID NO: 367 - protein, 215 aa, synthetic construct; identical to SEQ ID NO: 63
  REGION 1..215: note = EIP0650_ULBP2/5/6 light chain

SEQ ID NO: 368 - protein, 448 aa, synthetic construct; identical to SEQ ID NO: 64
  REGION 1..448: note = EIP0650_ULBP2/5/6 heavy chain

SEQ ID NO: 369 - protein, 215 aa, synthetic construct; identical to SEQ ID NO: 341
  REGION 1..215: note = EIP0650_CD3 light chain

SEQ ID NO: 370 - protein, 653 aa, synthetic construct
  REGION 1..653: note = EIP0650_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 371 - protein, 215 aa, synthetic construct; identical to SEQ ID NO: 63
  REGION 1..215: note = EIP0651_ULBP2/5/6 light chain

SEQ ID NO: 372 - protein, 448 aa, synthetic construct; identical to SEQ ID NO: 64
  REGION 1..448: note = EIP0651_ULBP2/5/6 heavy chain

SEQ ID NO: 373 - protein, 215 aa, synthetic construct; identical to SEQ ID NO: 61
  REGION 1..215: note = EIP0651_CD3 light chain

SEQ ID NO: 374 - protein, 653 aa, synthetic construct
  REGION 1..653: note = EIP0651_CD3 heavy chain
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSKVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 375 - protein, 215 aa, synthetic construct; identical to SEQ ID NO: 79
  REGION 1..215: note = EIP0547_ULBP2/5/6 light chain

SEQ ID NO: 376 - protein, 449 aa, synthetic construct
  REGION 1..449: note = EIP0547_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVKDYFPEPVTVSWNSGALTSGVHTCPAVLQSSGLYSLSSVVDVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 377 - protein, 215 aa, synthetic construct; identical to SEQ ID NO: 125
  REGION 1..215: note = EIP0547_CD3 light chain

SEQ ID NO: 378 - protein, 653 aa, synthetic construct
  REGION 1..653: note = EIP0547_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 379 - protein, 215 aa, synthetic construct; identical to SEQ ID NO: 79
  REGION 1..215: note = EIP0652_ULBP2/5/6 light chain

SEQ ID NO: 380 - protein, 448 aa, synthetic construct; identical to SEQ ID NO: 80
  REGION 1..448: note = EIP0652_ULBP2/5/6 heavy chain

SEQ ID NO: 381 - protein, 215 aa, synthetic construct
  REGION 1..215: note = EIP0652_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWDSNLWVFGGGTKLTVLGQPKANPTVTLFPPSSEELQANKAKLVCLISDFYPGAVTVAWKADGSPVKAGVETTKPSKQSNNKYAASSYLSLTPEQWKSHRSYSCQVTHEGSTVEKTVAPTECS

SEQ ID NO: 382 - protein, 653 aa, synthetic construct; identical to SEQ ID NO: 378
  REGION 1..653: note = EIP0652_CD3 heavy chain

SEQ ID NO: 383 - protein, 215 aa, synthetic construct; identical to SEQ ID NO: 79
  REGION 1..215: note = EIP0653_ULBP2/5/6 light chain

SEQ ID NO: 384 - protein, 448 aa, synthetic construct; identical to SEQ ID NO: 80
  REGION 1..448: note = EIP0653_ULBP2/5/6 heavy chain

SEQ ID NO: 385 - protein, 215 aa, synthetic construct
  REGION 1..215: note = EIP0653_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSQLWVFGGGTKLTVLGQPKANPTVTLFPPSSEELQANKAKLVCLISDFYPGAVTVAWKADGSPVKAGVETTKPSKQSNNKYAASSYLSLTPEQWKSHRSYSCQVTHEGSTVEKTVAPTECS

SEQ ID NO: 386 - protein, 653 aa, synthetic construct
  REGION 1..653: note = EIP0653_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 387 - protein, 215 aa, synthetic construct; identical to SEQ ID NO: 79
  REGION 1..215: note = EIP0454_ULBP2/5/6 light chain

SEQ ID NO: 388 - protein, 448 aa, synthetic construct; identical to SEQ ID NO: 80
  REGION 1..448: note = EIP0454_ULBP2/5/6 heavy chain

SEQ ID NO: 389 - protein, 215 aa, synthetic construct
  REGION 1..215: note = EIP0454_CD3 light chain
ELVVTQEPSLTVSPGGTVTLTCRSSTGAVTTSNYANWVQDKPGQAPRGLIGDTNKRAPGTPARFSGSLLGGKAALTLSGVQPEDEAEYYCALWYSNLWVFGGGTKLTVLGQPKANPTVTLFPPSSEELQANKAKLVCLISDFYPGAVTVAWKADGSPVKAGVETTKPSKQSNNKYAASSYLSLTPEQWKSHRSYSCQVTHEGSTVEKTVAPTECS

SEQ ID NO: 390 - protein, 653 aa, synthetic construct; identical to SEQ ID NO: 378
  REGION 1..653: note = EIP0454_CD3 heavy chain

SEQ ID NO: 391 - protein, 215 aa, synthetic construct; identical to SEQ ID NO: 79
  REGION 1..215: note = EIP0655_ULBP2/5/6 light chain

SEQ ID NO: 392 - protein, 448 aa, synthetic construct; identical to SEQ ID NO: 80
  REGION 1..448: note = EIP0655_ULBP2/5/6 heavy chain

SEQ ID NO: 393 - protein, 215 aa, synthetic construct
  REGION 1..215: note = EIP0655_CD3 light chain
ELVVTQEPSLTVSPGGTVTLTCRSSTGAVTTSNYANWVQDKPGQAPRGLIGGTNKRAPGTPARFSGSLLGGKAALTLSGVQPEDEAEYYCALWYSDLWVFGGGTKLTVLGQPKANPTVTLFPPSSEELQANKAKLVCLISDFYPGAVTVAWKADGSPVKAGVETTKPSKQSNNKYAASSYLSLTPEQWKSHRSYSCQVTHEGSTVEKTVAPTECS

SEQ ID NO: 394 - protein, 653 aa, synthetic construct
  REGION 1..653: note = EIP0655_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 395 - protein, 215 aa, synthetic construct; identical to SEQ ID NO: 79
  REGION 1..215: note = EIP0656_ULBP2/5/6 light chain

SEQ ID NO: 396 - protein, 448 aa, synthetic construct; identical to SEQ ID NO: 80
  REGION 1..448: note = EIP0656_ULBP2/5/6 heavy chain

SEQ ID NO: 397 - protein, 215 aa, synthetic construct; identical to SEQ ID NO: 125
  REGION 1..215: note = EIP0656_CD3 light chain

SEQ ID NO: 398 - protein, 653 aa, synthetic construct
  REGION 1..653: note = EIP0656_CD3 heavy chain
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 399 - protein, 215 aa, synthetic construct; identical to SEQ ID NO: 79
  REGION 1..215: note = EIP0657_ULBP2/5/6 light chain

SEQ ID NO: 400 - protein, 448 aa, synthetic construct; identical to SEQ ID NO: 80
  REGION 1..448: note = EIP0657_ULBP2/5/6 heavy chain

SEQ ID NO: 401 - protein, 215 aa, synthetic construct; identical to SEQ ID NO: 385
  REGION 1..215: note = EIP0657_CD3 light chain

SEQ ID NO: 402 - protein, 653 aa, synthetic construct
  REGION 1..653: note = EIP0657_CD3 heavy chain
EVQLLESGGGLVQPGGSLKLSCAASGFTFNTYAMNWVRKAPGKGLEWVAKIRSKYNNYATYYADSVKDRFTISRDDSKNTAYLQMNNLKTEDTAVYYCVRHGNFGDSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 403 - protein, 215 aa, synthetic construct; identical to SEQ ID NO: 79
  REGION 1..215: note = EIP0658_ULBP2/5/6 light chain

SEQ ID NO: 404 - protein, 448 aa, synthetic construct; identical to SEQ ID NO: 80
  REGION 1..448: note = EIP0658_ULBP2/5/6 heavy chain

SEQ ID NO: 405 - protein, 215 aa, synthetic construct; identical to SEQ ID NO: 385
  REGION 1..215: note = EIP0658_CD3 light chain

SEQ ID NO: 406 - protein, 653 aa, synthetic construct
  REGION 1..653: note = EIP0658_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGSFGNSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 407 - protein, 215 aa, synthetic construct; identical to SEQ ID NO: 79
  REGION 1..215: note = EIP0659_ULBP2/5/6 light chain

SEQ ID NO: 408 - protein, 448 aa, synthetic construct; identical to SEQ ID NO: 80
  REGION 1..448: note = EIP0659_ULBP2/5/6 heavy chain

SEQ ID NO: 409 - protein, 215 aa, synthetic construct; identical to SEQ ID NO: 125
  REGION 1..215: note = EIP0659_CD3 light chain

SEQ ID NO: 410 - protein, 653 aa, synthetic construct; identical to SEQ ID NO: 402
  REGION 1..653: note = EIP0659_CD3 heavy chain

SEQ ID NO: 411 - protein, 215 aa, synthetic construct; identical to SEQ ID NO: 79
  REGION 1..215: note = EIP0660_ULBP2/5/6 light chain

SEQ ID NO: 412 - protein, 448 aa, synthetic construct; identical to SEQ ID NO: 80
  REGION 1..448: note = EIP0660_ULBP2/5/6 heavy chain

SEQ ID NO: 413 - protein, 215 aa, synthetic construct; identical to SEQ ID NO: 385
  REGION 1..215: note = EIP0660_CD3 light chain

SEQ ID NO: 414 - protein, 653 aa, synthetic construct
  REGION 1..653: note = EIP0660_CD3 heavy chain
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGTFGNSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 415 - protein, 215 aa, synthetic construct; identical to SEQ ID NO: 79
  REGION 1..215: note = EIP0661_ULBP2/5/6 light chain

SEQ ID NO: 416 - protein, 448 aa, synthetic construct; identical to SEQ ID NO: 80
  REGION 1..448: note = EIP0661_ULBP2/5/6 heavy chain

SEQ ID NO: 417 - protein, 215 aa, synthetic construct; identical to SEQ ID NO: 125
  REGION 1..215: note = EIP0661_CD3 light chain

SEQ ID NO: 418 - protein, 653 aa, synthetic construct
  REGION 1..653: note = EIP0661_CD3 heavy chain
EVQLLESGGGLVQPGGSLKLSCAASGFTFNTYAMDWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCSVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 419 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 420 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 421 - protein, 246 aa, Homo sapiens
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 422 - protein, 334 aa, Homo sapiens
MAAAASPAFLLRLPLLLLLSSWCRTGLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPRATATTLILCCLLIMCLLICSRHSLTQSHGHHPQSLQPPPHPPLLHPTWLLRRVLWSDSYQIAKRPLSGGHVTRVTLPIIGDDSHSLPCPLALYTINNGAARYSEPLQVSIS

SEQ ID NO: 423 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 424 - protein, 108 aa, synthetic construct
  REGION 1..108: note = ULBP2_VL_E12
EIVLTQSPATLSLSPGERATLSCRASQSIGTSIHWYQQKPGQAPRLLIYFASESISGIPARFSGSGSGTDFTLTISSLEPEDFAVYYCQQSNSWPLYTFGGGTKVEIK

SEQ ID NO: 425 - protein, 120 aa, synthetic construct
  REGION 1..120: note = ULBP2_VH_E12
QVQLVQSGAEVKKPGASVKVSCKASGYTFTNYDINWVRQAPGQGLEWMGWIYPGDGSTYYNEKFKGRVTMTRDTSTSTVYMELSSLRSEDTAVYYCARSGDYGLYYFDYWGQGTLVTVSS

SEQ ID NO: 426 - protein, 108 aa, synthetic construct
  REGION 1..108: note = ULBP2_VL_A06
EIVMTQSPATLSLSPGERITLSCSASSSIHSNYLHWYQQKPGQAPRLLIYRTSNLASGIPARFSGSGSGTDFTLTISSLQPEDFAVYYCQQGFSIPFTFGQGTKLEIK

SEQ ID NO: 427 - protein, 120 aa, synthetic construct
  REGION 1..120: note = ULBP2_VH_A06
EVQLVQSGAEVKKPGESLKISCKGSGYAFSTSWMNWVRQMPGKGLEWMGRIYPGDGDTNYNGNFKGQVTISADKSISTAYLQWSSLKASDTAMYYCARGGELRLEAMDSWGQGTLVTVSS

SEQ ID NO: 428 - protein, 5 aa, synthetic construct
  REGION 1..5: note = ULBP2_VH_A06_CDRH1 Kabat
TSWMN

SEQ ID NO: 429 - protein, 7 aa, synthetic construct
  REGION 1..7: note = ULBP2_VH_A06_CDRH1 Chothia
GYAFSTS

SEQ ID NO: 430 - protein, 17 aa, synthetic construct
  REGION 1..17: note = ULBP2_VH_A06_CDRH2 Kabat
RIYPGDGDTNYNGNFKG

SEQ ID NO: 431 - protein, 6 aa, synthetic construct
  REGION 1..6: note = ULBP2_VH_A06_CDRH2 Chothia
YPGDGD

SEQ ID NO: 432 - protein, 11 aa, synthetic construct
  REGION 1..11: note = ULBP2_VH_A06_CDRH3
GGELRLEAMDS

SEQ ID NO: 433 - protein, 12 aa, synthetic construct
  REGION 1..12: note = ULBP2_VH_A06_CDRL1
SASSSIHSNYLH

SEQ ID NO: 434 - protein, 7 aa, synthetic construct
  REGION 1..7: note = ULBP2_VH_A06_CDRL2
RTSNLAS

SEQ ID NO: 435 - protein, 9 aa, synthetic construct
  REGION 1..9: note = ULBP2_VH_A06_CDRL3
QQGFSIPFT

SEQ ID NO: 436 - protein, 215 aa, synthetic construct
  REGION 1..215: note = EIP bispecific L2
  VARIANT 38: note = Xaa can be any naturally occurring amino acid
  VARIANT 115: note = Xaa can be any naturally occurring amino acid
  VARIANT 117: note = Xaa can be any naturally occurring amino acid
  VARIANT 139: note = Xaa can be any naturally occurring amino acid
  VARIANT 163: note = Xaa can be any naturally occurring amino acid
  VARIANT 181: note = Xaa can be any naturally occurring amino acid
  VARIANT 215: note = Xaa can be any naturally occurring amino acid
EIVLTQSPATLSLSPGERATLSCRASQSIGTSIHWYQXKPGQAPRLLIYFASESISGIPARFSGSGSGTDFTLTISSLEPEDFAVYYCQQSNSWPLYTFGGGTKVEIKRTVAAPXVXIFPPSDEQLKSGTASVVCLLNXFYPREAKVQWKVDNALQSGNSQEXVTEQDSKDSTYSLSSTLXLSKADYEKHKVYACEVTHQGLSSPVTKSFNRGEX

SEQ ID NO: 437 - protein, 448 aa, synthetic construct
  REGION 1..448: note = EIP bispecific H2
  VARIANT 39: note = Xaa can be any naturally occurring amino acid
  VARIANT 134: note = Xaa can be any naturally occurring amino acid
  VARIANT 137: note = Xaa can be any naturally occurring amino acid
  VARIANT 139: note = Xaa can be any naturally occurring amino acid
  VARIANT 150: note = Xaa can be any naturally occurring amino acid
  VARIANT 173..174: note = Xaa can be any naturally occurring amino acid
  VARIANT 190: note = Xaa can be any naturally occurring amino acid
  VARIANT 223: note = Xaa can be any naturally occurring amino acid
QVQLVQSGAEVKKPGASVKVSCKASGYTFTNYDINWVRXAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPXSKXTXGGTAALGCLVXDYFPEPVTVSWNSGALTSGVHTXXAVLQSSGLYSLSSVVXVPSSSLGTQTYICNVNHKPSNTKVDKKVEPKSXDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 438 - protein, 215 aa, synthetic construct
  REGION 1..215: note = EIP bispecific L1
  VARIANT 40: note = Xaa can be any naturally occurring amino acid
  VARIANT 121: note = Xaa can be any naturally occurring amino acid
  VARIANT 134: note = Xaa can be any naturally occurring amino acid
  VARIANT 140: note = Xaa can be any naturally occurring amino acid
  VARIANT 165: note = Xaa can be any naturally occurring amino acid
  VARIANT 182: note = Xaa can be any naturally occurring amino acid
  VARIANT 214: note = Xaa can be any naturally occurring amino acid
ELVVTQEPSLTVSPGGTVTLTCRSSTGAVTTSNYANWVQXKPGQAPRGLIGGTNKRAPGTPARFSGSLLGGKAALTLSGVQPEDEAEYYCALWYSNLWVFGGGTKLTVLGQPKANPTVTLXPPSSEELQANKAXLVCLIXDFYPGAVTVAWKADGSPVKAGVETXKPSKQSNNKYAASSYLXLTPEQWKSHRSYSCQVTHEGSTVEKTVAPTEXS

SEQ ID NO: 439 - protein, 453 aa, synthetic construct
  REGION 1..453: note = EIP bispecific H1
  VARIANT 39: note = Xaa can be any naturally occurring amino acid
  VARIANT 136: note = Xaa can be any naturally occurring amino acid
  VARIANT 153: note = Xaa can be any naturally occurring amino acid
  VARIANT 155: note = Xaa can be any naturally occurring amino acid
  VARIANT 178: note = Xaa can be any naturally occurring amino acid
  VARIANT 181: note = Xaa can be any naturally occurring amino acid
  VARIANT 193: note = Xaa can be any naturally occurring amino acid
  VARIANT 228: note = Xaa can be any naturally occurring amino acid
EVQLLESGGGLVQPGGSLKLSCAASGFTFNTYAMNWVRXAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPXAPSSKSTSGGTAALGCXVXDYFPEPVTVSWNSGALTSGVHTXPAXLQSSGLYSLSSXVTVPSSSLGTQTYICNVNHKPSNTKVDKKVEPKSXDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 440 - protein, 126 aa, synthetic construct
  REGION 1..126: note = SP34 VH1 Framework
  VARIANT 31: note = Xaa can be any naturally occurring amino acid
  VARIANT 35: note = Xaa can be any naturally occurring amino acid
  VARIANT 39: note = Xaa can be any naturally occurring amino acid
  VARIANT 50: note = Xaa can be any naturally occurring amino acid
  VARIANT 103: note = Xaa can be any naturally occurring amino acid
  VARIANT 107..108: note = Xaa can be any naturally occurring amino acid
EVQLLESGGGLVQPGGSLKLSCAASGFTFNXYAMXWVRXAPGKGLEWVAXIRSKYNNYATYYADSVKDRFTISRDDSKNTAYLQMNNLKTEDTAVYYCVRHGXFGNXXYVSWFAYWGQGTLVTVSS

SEQ ID NO: 441 - protein, 109 aa, synthetic construct
  REGION 1..109: note = SP34 VL1 Framework
  VARIANT 40: note = Xaa can be any naturally occurring amino acid
  VARIANT 52: note = Xaa can be any naturally occurring amino acid
  VARIANT 96: note = Xaa can be any naturally occurring amino acid
  VARIANT 94: note = Xaa can be any naturally occurring amino acid
ELVVTQEPSLTVSPGGTVTLTCRSSTGAVTTSNYANWVQXKPGQAPRGLIGXTNKRAPGTPARFSGSLLGGKAALTLSGVQPEDEAEYYCALWXSXLWVFGGGTKLTVL

SEQ ID NO: 442 - protein, 215 aa, synthetic construct; identical to SEQ ID NO: 63
  REGION 1..215: note = EIP0641_ULBP2/5/6 light chain

SEQ ID NO: 443 - protein, 449 aa, synthetic construct
  REGION 1..449: note = EIP0641_ULBP2/5/6 heavy chain
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTCGGTAALGCLVDDYFPEPVTVSWNSGALTSGVHTFPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 444 - protein, 215 aa, synthetic construct; identical to SEQ ID NO: 61
  REGION 1..215: note = EIP0641_CD3 light chain

SEQ ID NO: 445 - protein, 653 aa, synthetic construct; identical to SEQ ID NO: 334
  REGION 1..653: note = EIP0641_CD3 heavy chain

SEQ ID NO: 446-449 - skipped (4 entries)

SEQ ID NO: 450 - protein, 214 aa, synthetic construct
DILLTQSPVILSVSPGERVSFSCRASQSIGTNIHWYQKRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 451 - protein, 448 aa, synthetic construct
QVQLKQSGPGLVQPSQSLSITCTVSGFSLTNYGVHWVRDSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 452 - protein, 215 aa, synthetic construct; identical to SEQ ID NO: 69

SEQ ID NO: 453 - protein, 454 aa, synthetic construct
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 454 - protein, 214 aa, synthetic construct; identical to SEQ ID NO: 450

SEQ ID NO: 455 - protein, 448 aa, synthetic construct; identical to SEQ ID NO: 451

SEQ ID NO: 456 - protein, 215 aa, synthetic construct; identical to SEQ ID NO: 69

SEQ ID NO: 457 - protein, 658 aa, synthetic construct
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 458 - protein, 214 aa, synthetic construct
DIQMTQSPSSLSASVGDRVTITCSASQDISNYLNWYQKKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 459 - protein, 450 aa, synthetic construct
QVQLVQSGAEVKKPGSSVKVSCKASGGTFSNYWMHWVRDAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 460 - protein, 215 aa, synthetic construct; identical to SEQ ID NO: 69

SEQ ID NO: 461 - protein, 454 aa, synthetic construct; identical to SEQ ID NO: 453

SEQ ID NO: 462 - protein, 214 aa, synthetic construct; identical to SEQ ID NO: 458

SEQ ID NO: 463 - protein, 450 aa, synthetic construct; identical to SEQ ID NO: 459

SEQ ID NO: 464 - protein, 215 aa, synthetic construct; identical to SEQ ID NO: 69

SEQ ID NO: 465 - protein, 658 aa, synthetic construct; identical to SEQ ID NO: 457

SEQ ID NO: 466 - skipped

SEQ ID NO: 467 - protein, 471 aa, synthetic construct
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 468 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 469 - protein, 449 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 470 - skipped

SEQ ID NO: 471 - protein, 471 aa, synthetic construct; identical to SEQ ID NO: 467

SEQ ID NO: 472 - protein, 214 aa, synthetic construct; identical to SEQ ID NO: 468

SEQ ID NO: 473 - protein, 646 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 474 - skipped

SEQ ID NO: 475 - protein, 471 aa, synthetic construct
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 476 - protein, 214 aa, synthetic construct; identical to SEQ ID NO: 468

SEQ ID NO: 477 - protein, 449 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 478 - skipped

SEQ ID NO: 479 - protein, 471 aa, synthetic construct; identical to SEQ ID NO: 475

SEQ ID NO: 480 - protein, 214 aa, synthetic construct; identical to SEQ ID NO: 468

SEQ ID NO: 481 - protein, 647 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 482 - skipped

SEQ ID NO: 483 - protein, 344 aa, synthetic construct
EVQLVESGGGLVQPGGSLRLSCAASGFSFSDFWMSWVRQAPGKGLEWVATISQAGSEKDYVDSVKGRFTISRDNAKKSLYLQMNSLRAEDTAVYYCASGVYSFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 484 - protein, 214 aa, synthetic construct; identical to SEQ ID NO: 468

SEQ ID NO: 485 - protein, 449 aa, synthetic construct; identical to SEQ ID NO: 477

SEQ ID NO: 486 - skipped

SEQ ID NO: 487 - protein, 344 aa, synthetic construct; identical to SEQ ID NO: 483

SEQ ID NO: 488 - protein, 214 aa, synthetic construct; identical to SEQ ID NO: 468

SEQ ID NO: 489 - protein, 647 aa, synthetic construct; identical to SEQ ID NO: 481

SEQ ID NO: 490 - skipped

SEQ ID NO: 491 - protein, 485 aa, synthetic construct
QLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSSGGGGSGGGGSQVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 492 - protein, 214 aa, synthetic construct; identical to SEQ ID NO: 468

SEQ ID NO: 493 - protein, 449 aa, synthetic construct; identical to SEQ ID NO: 477

SEQ ID NO: 494 - skipped

SEQ ID NO: 495 - protein, 485 aa, synthetic construct; identical to SEQ ID NO: 491

SEQ ID NO: 496 - protein, 214 aa, synthetic construct; identical to SEQ ID NO: 468

SEQ ID NO: 497 - protein, 647 aa, synthetic construct; identical to SEQ ID NO: 481

SEQ ID NO: 498 - protein, 213 aa, synthetic construct
EIVLTQSPATLSLSPGERATLSCSASSSVSYMHWYQKKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 499 - protein, 449 aa, synthetic construct
QVQLQESGPGLVKPSQTLSLTCTVSGGSISTSGMGVGWIRDHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 500 - protein, 215 aa, synthetic construct; identical to SEQ ID NO: 69

SEQ ID NO: 501 - protein, 454 aa, synthetic construct; identical to SEQ ID NO: 453

SEQ ID NO: 502 - protein, 213 aa, synthetic construct; identical to SEQ ID NO: 498

SEQ ID NO: 503 - protein, 449 aa, synthetic construct; identical to SEQ ID NO: 499

SEQ ID NO: 504 - protein, 215 aa, synthetic construct; identical to SEQ ID NO: 69

SEQ ID NO: 505 - protein, 656 aa, synthetic construct
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 506 - protein, 219 aa, synthetic construct
DIVMTQSPATLSLSPGERATLSCRSSKSLQNVNGNTYLYWFQKKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 507 - protein, 450 aa, synthetic construct
EVQLVESGGGLVKPGGSLKLSCAASGYTFTSYVMHWVRDAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 508 - protein, 215 aa, synthetic construct; identical to SEQ ID NO: 69

SEQ ID NO: 509 - protein, 454 aa, synthetic construct; identical to SEQ ID NO: 453

SEQ ID NO: 510 - protein, 219 aa, synthetic construct; identical to SEQ ID NO: 506

SEQ ID NO: 511 - protein, 450 aa, synthetic construct; identical to SEQ ID NO: 507

SEQ ID NO: 512 - protein, 215 aa, synthetic construct; identical to SEQ ID NO: 69

SEQ ID NO: 513 - protein, 656 aa, synthetic construct; identical to SEQ ID NO: 505

SEQ ID NO: 514 - protein, 215 aa, synthetic construct
EIVLTQSPGTLSLSPGERATFSCRSSHSIRSRRVAWYQKKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLRLSKADYEKHKVYACEVTHQGLSSPVTKSFNRGEC

SEQ ID NO: 515 - protein, 456 aa, synthetic construct
QVQLVQSGAEVKKPGASVKVSCQASGYRFSNFVIHWVRDAPGQRQEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 516 - protein, 215 aa, synthetic construct; identical to SEQ ID NO: 69

SEQ ID NO: 517 - protein, 454 aa, synthetic construct; identical to SEQ ID NO: 453

SEQ ID NO: 518 - protein, 215 aa, synthetic construct; identical to SEQ ID NO: 514

SEQ ID NO: 519 - protein, 456 aa, synthetic construct; identical to SEQ ID NO: 515

SEQ ID NO: 520 - protein, 215 aa, synthetic construct; identical to SEQ ID NO: 149

SEQ ID NO: 521 - protein, 656 aa, synthetic construct
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 522 - protein, 215 aa, synthetic construct; identical to SEQ ID NO: 514

SEQ ID NO: 523 - protein, 456 aa, synthetic construct; identical to SEQ ID NO: 515

SEQ ID NO: 524 - protein, 215 aa, synthetic construct; identical to SEQ ID NO: 69

SEQ ID NO: 525 - protein, 656 aa, synthetic construct
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 526-529 - skipped (4 entries)

SEQ ID NO: 530 - protein, 19 aa, synthetic construct
VPSTPPTPSPSTPPTPSPS

SEQ ID NO: 531 - protein, 6 aa, synthetic construct
VPPPPP

SEQ ID NO: 532 - protein, 58 aa, synthetic construct
ESPKAQASSVPTAQPQAEGSLAKATTAPATTRNTGRGGEEKKKEKEKEEQEERETKTP

SEQ ID NO: 533 - protein, 15 aa, synthetic construct
EPKSCDKTHTCPPCP

SEQ ID NO: 534 - protein, 12 aa, synthetic construct
ERKCCVECPPCP

SEQ ID NO: 535 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 536 - protein, 12 aa, synthetic construct
ESKYGPPCPSCP

SEQ ID NO: 537 - protein, 12 aa, synthetic construct
ESKYGPPCPPCP

SEQ ID NO: 538 - protein, 4 aa, synthetic construct
CPPC

SEQ ID NO: 539 - protein, 4 aa, synthetic construct
CPSC

SEQ ID NO: 540 - protein, 4 aa, synthetic construct
CPRC

SEQ ID NO: 541 - protein, 4 aa, synthetic construct
SPPC

SEQ ID NO: 542 - protein, 4 aa, synthetic construct
CPPS

SEQ ID NO: 543 - protein, 4 aa, synthetic construct
SPPS

SEQ ID NO: 544 - protein, 8 aa, synthetic construct
DKTHTCAA

SEQ ID NO: 545 - protein, 11 aa, synthetic construct
DKTHTCPPCPA

SEQ ID NO: 546 - protein, 18 aa, synthetic construct
DKTHTCPPCPATCPPCPA

SEQ ID NO: 547 - protein, 25 aa, synthetic construct
DKTHTCPPCPATCPPCPATCPPCPA

SEQ ID NO: 548 - protein, 30 aa, synthetic construct
DKTHTCPPCPAGKPTLYNSLVMSDTAGTCY

SEQ ID NO: 549 - protein, 31 aa, synthetic construct
DKTHTCPPCPAGKPTHVNVSVVMAEVDGTCY

SEQ ID NO: 550 - protein, 15 aa, synthetic construct
DKTHTCCVECPPCPA

SEQ ID NO: 551 - protein, 26 aa, synthetic construct
DKTHTCPRCPEPKSCDTPPPCPRCPA

SEQ ID NO: 552 - protein, 11 aa, synthetic construct
DKTHTCPSCPA

SEQ ID NO: 553 - protein, 5 aa, synthetic construct
ADAAP

SEQ ID NO: 554 - protein, 11 aa, synthetic construct
ADAAPTVSIFP

SEQ ID NO: 555 - protein, 12 aa, synthetic construct
ADAAPTVSIFPP

SEQ ID NO: 556 - protein, 6 aa, synthetic construct
AKTTAP

SEQ ID NO: 557 - protein, 13 aa, synthetic construct
AKTTAPSVYPLAP

SEQ ID NO: 558 - protein, 17 aa, synthetic construct
AKTTPKLEEGEFSEARV

SEQ ID NO: 559 - protein, 9 aa, synthetic construct
AKTTPKLGG

SEQ ID NO: 560 - protein, 6 aa, synthetic construct
AKTTPP

SEQ ID NO: 561 - protein, 13 aa, synthetic construct
AKTTPPSVTPLAP

SEQ ID NO: 562 - protein, 6 aa, synthetic construct
ASTKGP

SEQ ID NO: 563 - protein, 13 aa, synthetic construct
ASTKGPSVFPLAP

SEQ ID NO: 564 - protein, 26 aa, synthetic construct
ASTKGPSVFPLAPASTKGPSVFPLAP

SEQ ID NO: 565 - protein, 14 aa, synthetic construct
EGKSSGSGSESKST

SEQ ID NO: 566 - protein, 15 aa, synthetic construct
GEGESGEGESGEGES

SEQ ID NO: 567 - protein, 20 aa, synthetic construct
GEGESGEGESGEGESGEGES

SEQ ID NO: 568 - protein, 15 aa, synthetic construct
GEGGSGEGGSGEGGS

SEQ ID NO: 569 - protein, 15 aa, synthetic construct
GENKVEYAPALMALS

SEQ ID NO: 570 - protein, 15 aa, synthetic construct
GGEGSGGEGSGGEGS

SEQ ID NO: 571 - protein, 15 aa, synthetic construct
GGGESGGEGSGEGGS

SEQ ID NO: 572 - protein, 15 aa, synthetic construct
GGGESGGGESGGGES

SEQ ID NO: 573 - protein, 6 aa, synthetic construct
GGGGSN

SEQ ID NO: 574 - protein, 10 aa, synthetic construct
GGGGSGGGGS

SEQ ID NO: 575 - protein, 15 aa, synthetic construct
GGGGSGGGGSGGGGS

SEQ ID NO: 576 - protein, 20 aa, synthetic construct
GGGGSGGGGSGGGGSGGGGS

SEQ ID NO: 577 - protein, 15 aa, synthetic construct
GGGKSGGGKSGGGKS

SEQ ID NO: 578 - protein, 15 aa, synthetic construct
GGGKSGGKGSGKGGS

SEQ ID NO: 579 - protein, 15 aa, synthetic construct
GGKGSGGKGSGGKGS

SEQ ID NO: 580 - protein, 5 aa, synthetic construct
GGSGG

SEQ ID NO: 581 - protein, 9 aa, synthetic construct
GGSGGGGSG

SEQ ID NO: 582 - protein, 13 aa, synthetic construct
GGSGGGGSGGGGS

SEQ ID NO: 583 - protein, 15 aa, synthetic construct
GHEAAAVMQVQYPAS

SEQ ID NO: 584 - protein, 15 aa, synthetic construct
GKGGSGKGGSGKGGS

SEQ ID NO: 585 - protein, 15 aa, synthetic construct
GKGKSGKGKSGKGKS

SEQ ID NO: 586 - protein, 20 aa, synthetic construct
GKGKSGKGKSGKGKSGKGKS

SEQ ID NO: 587 - protein, 15 aa, synthetic construct
GKPGSGKPGSGKPGS

SEQ ID NO: 588 - protein, 20 aa, synthetic construct
GKPGSGKPGSGKPGSGKPGS

SEQ ID NO: 589 - protein, 15 aa, synthetic construct
GPAKELTPLKEAKVS

SEQ ID NO: 590 - protein, 12 aa, synthetic construct
GSAGSAAGSGEF

SEQ ID NO: 591 - protein, 14 aa, synthetic construct
IRPRAIGGSKPRVA

SEQ ID NO: 592 - protein, 18 aa, synthetic construct
KESGSVSSEQLAQFRSLD

SEQ ID NO: 593 - protein, 15 aa, synthetic construct
KTTPKLEEGEFSEAR

SEQ ID NO: 594 - protein, 6 aa, synthetic construct
QPKAAP

SEQ ID NO: 595 - protein, 13 aa, synthetic construct
QPKAAPSVTLFPP

SEQ ID NO: 596 - protein, 9 aa, synthetic construct
RADAAAAGS

SEQ ID NO: 597 - protein, 12 aa, synthetic construct
RADAAAAGGPGS

SEQ ID NO: 598 - protein, 6 aa, synthetic construct
RADAAP

SEQ ID NO: 599 - protein, 9 aa, synthetic construct
RADAAPTVS

SEQ ID NO: 600 - protein, 6 aa, synthetic construct
SAKTTP

SEQ ID NO: 601 - protein, 18 aa, synthetic construct
SAKTTPKLEEGEFSEARV

SEQ ID NO: 602 - protein, 10 aa, synthetic construct
SAKTTPKLGG

SEQ ID NO: 603 - protein, 14 aa, synthetic construct
STAGDTHLGGEDFD

SEQ ID NO: 604 - protein, 5 aa, synthetic construct
TVAAP

SEQ ID NO: 605 - protein, 12 aa, synthetic construct
TVAAPSVFIFPP

SEQ ID NO: 606 - protein, 24 aa, synthetic construct
TVAAPSVFIFPPTVAAPSVFIFPP

SEQ ID NO: 607-609 - skipped (3 entries)

SEQ ID NO: 610 - protein, 215 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 611 - protein, 457 aa, synthetic construct
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 612 - protein, 215 aa, synthetic construct; identical to SEQ ID NO: 55

SEQ ID NO: 613 - protein, 449 aa, synthetic construct
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 614 - protein, 215 aa, synthetic construct; identical to SEQ ID NO: 69

SEQ ID NO: 615 - protein, 454 aa, synthetic construct
EVQLLESGGGLVQPGGSLKLSCAASGFTFDTYAMNWVRKAPGKGLEWVAKIRSKYNNYATYYADSVKDRFTISRDDSKNTAYLQMNNLKTEDTAVYYCVRHGAFGDSYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 616 - protein, 215 aa, synthetic construct; identical to SEQ ID NO: 59

SEQ ID NO: 617 - protein, 449 aa, synthetic construct
QVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKCTSGGTAALGCLVDDYFPEPVTVSWNSGALTSGVHTFPAVLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 618 - protein, 215 aa, synthetic construct; identical to SEQ ID NO: 153

SEQ ID NO: 619 - protein, 454 aa, synthetic construct
EVQLLESGGGLVQPGGSLKLSCAASGFTFNTYAMNWVRKAPGKGLEWVARIRSKYNNYATYYADSVKDRFTISRDDSKNTAYLQMNNLKTEDTAVYYCVRHGNFGDDYVSWFAYWGQGTLVTVSSASTKGPSVFPLAPSSKSTSGGTAALGCLVKDYFPEPVTVSWNSGALTSGVHTSPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 620 - skipped

SEQ ID NO: 621 - protein, 449 aa, synthetic construct
EVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 622 - protein, 651 aa, synthetic construct
EVQLLESGGGLVQPGGSLKLSCAASGFTFNDYAMNWVRKAPGKGLEWVARIRSKYNNYATYYADSVKDRFTISRDDSKNTAYLQMNNLKTEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLSSVVTVPSSSLGTQTYICNVNHKPSNTKVDKKVEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRY

SEQ ID NO: 623 - skipped

SEQ ID NO: 624 - protein, 250 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 625 - protein, 187 aa, synthetic construct
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 626 - protein, 187 aa, synthetic construct
BSQQIYGVJYGNVTFHVPSNOPLKEVLWKKQKDKVAELENSEFRAFSSFKNRVYLDTUSGSLTIYNLTSSDEDEYEMESPNITDXMKFFLYVZESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 627 - protein, 44 aa, synthetic construct
SQQIYGVVYGNVTFHVPSNVPLKEVLWKKQKDKVAELENSEFRA

SEQ ID NO: 628 - protein, 44 aa, synthetic construct
SQQIYGVJYGNVTFHVPSNOPLKEVLWKKQKDKVAELENSEFRA

SEQ ID NO: 629 - protein, 120 aa, synthetic construct
EVQLVQSGAEVKKPGASVKVSCKASGYTFTNYDINWVRDAPGQGLEWMGWIYPGDGSTYYNEKFKGRVTMTRDTSTSTVYMELSSLRSEDTAVYYCARSGDYGLYYFDYWGQGTLVTVSS

(19) United States
(12) Patent Application Publication
(10) Pub. No.: US 2025/0197496 A1
(43) Pub. Date: Jun. 19, 2025

(54) BISPECIFIC ANTIBODY FUSION MOLECULES AND METHODS OF USE THEREOF

(71) Applicant: Evolvelmmune Therapeutics, Inc., Branford, CT (US)

(72) Inventors: Mohosin Sarkar, Woodbridge, CT (US); Sonali Dhindwal, Branford, CT (US); Jeremy S. Myers, Orange, CT (US); Eric M. Tam, Cooperstown, NY (US); Guixian Jin, Suffern, NY (US); Shu Shien Chin, Hastings-On-Hudson, NY (US); Parvez Rashid, Foster City, CA (US); Hayretin Rafet Yumerefendi, Belmont, MA (US)

(73) Assignee: Evolvelmmune Therapeutics, Inc., Branford, CT (US)

(21) Appl. No.: 18/847,825

(22) PCT Filed: Mar. 20, 2023
(86) PCT No.: PCT/US2023/064728

§ 371 Date: Sep. 17, 2024

Related U.S. Application Data
(60) Provisional application No. 63/368,852, filed on Jul. 19, 2022.
(60) Provisional application No. 63/330,250, filed on Apr. 12, 2022.
(60) Provisional application No. 63/321,563, filed on Mar. 18, 2022.

(51) Int. Cl. C07K 16/28; A61K 9/127; A61K 39/00; A61P 35/00; C07K 14/54; C07K 14/705

(52) U.S. Cl. CPC C07K 16/2809; A61K 9/127; A61P 35/00; C07K 14/5418; C07K 14/70528; A61K 2039/505; C07K 2317/24; C07K 2317/31; C07K 2317/522; C07K 2317/524; C07K 2317/53; C07K 2317/565; C07K 2319/30

(57) ABSTRACT

Described herein are compositions and methods for the efficient production of a heteromultimeric antibodies, such as a bispecific antibodies. The compositions and methods improve assembly of the heteromultimeric antibodies at higher yield and efficiency than conventional methods. Antibodies are optionally fused with a cytokine or costimulatory peptide or portions thereof. Also described herein are monoclonal antibodies and antigen binding fragments, variants, multimeric versions, or bispecific antibodies thereof that specifically bind CD3. Described herein are methods of making and using the anti-CD3 antibodies and antigen binding fragments thereof in a variety of therapeutic, diagnostic and prophylactic indications.

### RELATED APPLICATIONS

This application is a national stage filing under 35 U.S.C. § 371 of international PCT application PCT/US2023/064728, filed Mar. 20, 2023, which claims priority under 35 U.S.C. § 119(e) to U.S. Provisional Application No. 63/368,852, filed on Jul. 19, 2022, U.S. Provisional Application No. 63/330,250, filed on Apr. 12, 2022, and U.S. Provisional Application No. 63/321,563, filed on Mar. 18, 2022, the entire contents of each of which are incorporated by reference herein.

### REFERENCE TO AN ELECTRONIC SEQUENCE LISTING

The contents of the electronic sequence listing (E070170000US03-SEQ-KZM.xml; Size: 883,751 bytes; and Date of Creation: Sep. 13, 2024) is herein incorporated by reference in its entirety.

### FIELD

Described herein are compositions and methods for the efficient production of antibodies or antigen binding fragments thereof. Antibodies may be capable of specifically binding to more than one target molecule or different epitopes on a single target molecule. The compositions and methods improve antibody assembly and production resulting in higher efficiency and yield compared to conventional methods.

### BACKGROUND OF THE INVENTION

Monoclonal antibodies of the IgG type contain two identical antigen-binding arms and a constant domain (Fc). Antibodies with a differing specificity in their binding arms usually do not occur in nature and, therefore, have to be crafted with the help of chemical engineering (e.g., chemical cross-linking, etc.), recombinant DNA and/or cell-fusion technology.

Bispecific antibodies can bind simultaneously two different antigens. This property enables the development of therapeutic strategies that are not possible with conventional monoclonal antibodies. Another class of multispecific molecules is recombinant fusion proteins. Recombinant fusion proteins consisting of the extracellular domain of immunoregulatory proteins and the constant (Fc) domain of immunoglobulin (Ig) represent a growing class of human therapeutics.

The manufacturing of clinical grade material remains challenging for antibodies generally and especially for the multispecific antibodies. There are many paths to the production of molecules with mixed binding arms, i.e., binding arms that are not identical to each other. But each of these methods have significant drawbacks.

Chemical cross-linking is labor intensive as the relevant species may yet need to be purified from homodimers and other undesirable by-products resulting in additional step to separate undesirable products from desired products. In addition, the chemical modification steps can alter the integrity of the proteins thus leading to poor stability. Thus, this method is often inefficient and can lead to loss of antibody activity.

Cell-fusion technology (e.g., hybrid hybridomas) express two heavy and two light chains that assemble randomly leading to the generation of 10 antibody combinations when two cells each expressing a separate antibody are fused. The desired heteromultimeric antibodies are only a small fraction of the antibodies thus produced. Purification of the desired heteromultimeric proteins dramatically reduces production yields and increases manufacturing costs.

Recombinant DNA techniques have been used to generate various heteromultimeric formats. e.g., single chain Fv, diabodies, etc., that do not comprise an Fc domain. A major drawback for this type of antibody molecule is the lack of the Fc domain and thus the ability of the antibody to trigger an effector function and extend serum half-life (e.g., complement activation, Fc-receptor binding etc.). Thus, a bispecific antibody comprising a functional Fc domain is desired.

Recombinant DNA techniques have also been used to generate ‘knob into hole’ bispecific antibodies. One constraint of this strategy is that the light chains of the two parent antibodies have to be identical to prevent mispairing and formation of undesired and/or inactive molecules when expressed in the same cell.

In addition, one of the limiting events during annealing and purification is the redox efficiency. Following reduction, oxidized heterodimer typically only make up 70-80% of the protein after this step as indicated by BioAnalyzer and MS-TOF analysis of intact mass species following oxidation. The remaining 20-30% of antibody is dimeric and lacks a covalent linkage (SEC-MALS). This can be removed but significantly impacts overall yields.

Thus, there remains a need to improve the overall yield in antibody production, especially heterodimeric antibodies such as bispecific antibodies. Described herein are methods that can improve overall yield of bispecific antibodies, heterodimers and the like. These and other aspects and advantages of the invention will be apparent from the description of the invention provided herein.

### SUMMARY OF THE INVENTION

The disclosure provides an antibody comprising the following structure: a. a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), b. a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2), wherein i. the amino acid at positions 39 (Kabat numbering) of the VH1 and VH2 are charged or polar amino acid residues and the amino acid at positions 38 (Kabat numbering) of the VL1 and VL2 are an oppositely charged or polar amino acid residue compared to the amino acids at positions 39 of the VH1 and the VH2; or the amino acid at positions 100 of the VH1 and VH2 (Kabat numbering) are charged or polar amino acid residues and the amino acid at positions 44 (Kabat numbering) of the VL1 and VL2 are an oppositely charged or polar amino acid residue compared to the amino acids at positions 100 of the VH1 and the VH2 (Kabat numbering); ii. the amino acid at positions 147 of the CH1_{H1} and the CH1_{H2} (EU numbering) are charged or polar amino acid residues and one of the amino acids at positions 131, 179 or 180 of the CL1 or CL2 (EU numbering) is an oppositely charged or polar amino acid residue compared to the amino acids at positions 147 of the CH1_{H1} and the CH1_{H2} (EU numbering); iii. the amino acid at positions 185 of the CH1_{H1} and the CH1_{H2} (EU numbering) are charged or polar amino acid residues and the amino acid at positions 137 of the CL1 and CL2 (EU numbering) are an oppositely charged or polar amino acid residue compared to the amino acids at positions 185 of the CH1_{H1} and the CH1_{H2} (EU numbering); or the amino acid at positions 187 of the CHI_{H1} and the CH1_{H2} (EU numbering) are charged or polar amino acid residues and one of the amino acids at positions 137 or 138 of the CL1 and CL2 (EU numbering) is an oppositely charged or polar amino acid residue compared to the amino acids at positions 187 of the CHI_{H1} and the CH1_{H2} (EU numbering); and iv. the amino acid at positions 145 of the CH1_{H1} and the CH1_{H2} (EU numbering) are charged or polar amino acid residues and the amino acids at position 131 of the CL1 and CL2 (EU numbering) are oppositely charged or polar amino acid residues compared to the amino acids at positions 145 of the CH1_{H1} and the CH1_{H2}. (EU numbering).

In some embodiments, the charged amino acid residue is a naturally occurring amino acid or a non-naturally occurring amino acid. In some embodiments, the naturally occurring charged amino acid residue is an arginine, a lysine, a histidine, a glutamic acid or an aspartic acid.

In some embodiments, the H1 amino acids at position 39, 100, 147, 185, 187 or 145 are positively charged and the L1 amino acids at positions 38, 44, 131, 179, 180, 137 or 138 are negatively charged; and the H2 amino acids at position 39, 100, 147, 185, 187 or 145 are negatively charged and the L2 amino acids at positions 38, 44, 131, 179, 180, 137 or 138 are positively charged. In some embodiments, the H1 amino acids at position 39, 100, 147, 185, 187 and 145 are negatively charged and the L1 amino acids at positions 38, 44, 131, 179, 180, 137 or 138 are positively charged; and the H2 amino acids at position 39, 100, 147, 185, 187 or 145 are positively charged and the L2 amino acids at positions 38, 44, 131, 179, 180, 137 or 138 are negatively charged.

In some embodiments, L1 and L2 are lambda or kappa light chains. In some embodiments, L1 is a lambda light chain and L2 is a kappa light chain. In some embodiments, L1 is a kappa light chain and L2 is a lambda light chain.

In some embodiments, the antibody has an IgG₁, an IgG₂, or an IgG₄ isotype. In some embodiments, the antibody is a chimeric antibody, a human antibody, or a humanized antibody. In some embodiments, the antibody is a bispecific antibody.

In some embodiments, the bispecific antibody comprises: i) a first antigen binding domain that binds to a cell surface antigen, wherein the cell surface antigen is expressed on a T-cell, a NK cell, a neutrophil, a B cell or a dendritic cell engager; and ii) a second antigen binding domain that binds to a disease associated antigen (DAA). In some embodiments, the cell surface antigen is expressed on a T-cell.

In some embodiments, the cell surface antigen expressed on a T-cell is a CD3. In some embodiments, the cell surface antigen expressed on a T-cell is a CD38.

In some embodiments, the DAA is a UL16 Binding Protein 2 (ULBP2). In some embodiments, the DAA is a UL16 Binding Protein 5 (ULBP5) In some embodiments, the DAA is a UL16 Binding Protein 6 (ULBP6).

In some embodiments a polypeptide is fused to the N-terminus or the C-terminus of H1 and/or H2. In some embodiments a polypeptide is fused to the N-terminus of the H1. In some embodiments a polypeptide is fused to the N-terminus of the H2. In some embodiments a polypeptide is fused to the C-terminus of the H1. In some embodiments a polypeptide is fused to the C-terminus of the H2. In some embodiments, the polypeptide is a CD58, a IL-7 or a fragment thereof. In some embodiments, the polypeptide is a CD58 extracellular domain (CD58-ECD). In some embodiments, the polypeptide is a CD58-variable domain (CD58v*).

In some embodiments, the polypeptide is fused via a linker peptide. In some embodiments, the linker peptide is at least 5, at least 6, at least 7, at least 8, at least 9, at least 10, at least 11, at least 12, at least 13, at least 14, at least 15, at least 16, at least 17, at least 18, at least 19 or at least 20 amino acid residues in length. In some embodiments, the linker peptide comprises the amino acid sequence of SEQ ID NOS: 52-54.

In some embodiments, the antibody comprises: i) the amino acid at position 87 (Kabat numbering) of the VH1 and/or VH2 is a G; and ii) the amino acid at position 45 (Kabat numbering) of the VL1 and/or VL2 is a W.

In some embodiments, the antibody comprises i) the CH1_{H3} has a C at position 349, an S at position 366, an A at position 368 and a V at position 407 (EU numbering); and the CH2_{H3} has a C at position 354 and a W at position 366 (EU numbering); ii) the CH2_{H3} has a C at position 349, an S at position 366, an A at position 368 and a V at position 407 (EU numbering); and the CH1_{H3} has a C at position 354 and a W at position 366 (EU numbering); iii) the CH1_{H3} has a C at position 354, an S at position 366, an A at position 368 and a V at position 407 (EU numbering); and the CH2_{H3} has a C at position 349 and a W at position 366 (EU numbering); or iv) the CH2_{H3} has a C at position 354, an S at position 366, an A at position 368 and a V at position 407 (EU numbering); and the CH1_{H3} has a C at position 349 and a W at position 366 (EU numbering).

In some embodiments, the amino acid at position 447 (EU numbering) of the CH1_{H3} and/or of the CH2_{H3} is deleted.

In some embodiments, i) the H1H and/or the H2H has an A at positions 234 and 235 (EU numbering); ii) the H1H and/or the H2H has an A at positions 234, 235 and 237 (EU numbering); or iii) the H1H and/or the H2H has an A at positions 234 and 235 and G at position 329 (EU numbering).

In some embodiments, the antibody comprises i) the CH1_{H3} and/or the CH2_{H3} has an A at position 297 (EU numbering); ii) the CH1_{H3} and/or the CH2_{H3} has a G at position 297 (EU numbering); or iii) the CH1_{H3} and/or the CH2_{H3} has a S at position 297 (EU numbering). In some embodiments, the CH1_{H3} and/or the CH2_{H3} has an S at position 331 (EU numbering).

In some embodiments, the antibody comprises i) the CH1_{H2} and/or the CH2_{H2} has an C at position 370 (Kabat numbering); and ii) the CH1_{H2} and/or the CH2_{H2} has an C at position 375 (Kabat numbering).

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 185 (EU numbering) of the CH1_{H1} is a K and the amino acid at position 137 (EU numbering) of the CL1 is a D; iii. the amino acid at position 128 (EU numbering) of the CH1_{H1} is a C and the amino acid at position 118 (EU numbering) of the CL1 is a C; and iv. the amino acid at position 220 (EU numbering) in the H1H is a S and the amino acid at position 214 (EU numbering) of the CL1 is a S; and b) the H2 and the L2 comprise the following: i. the amino acid at position 39 (Kabat numbering) of the VH2 is a D and the amino acid at position 38 (Kabat numbering) of the VL2 is a K; and ii. the amino acid at position 147 (EU numbering) of the CH2_{H1} is a D and the amino acid at position 180 (EU numbering) of the CL2 is a R.

In some embodiments, a) the H1 and the L1 comprise the following: i. the amino acid at position 39 (Kabat numbering) of the VH1 is a K and the amino acid at position 38 (Kabat numbering) of the VL1 is a D; and ii. the amino acid at position 185 (EU numbering) of the CH1_{H1} is a K and the amino acid at position 137 (EU numbering) of the CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 147 (EU numbering) of the CH2_{H1} is a D and the amino acid at position 180 (EU numbering) of the CL2 is a R; iii. the amino acid at position 134 (EU numbering) of the CH2_{H1} is a C and the amino acid at position 116 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; and ii. the amino acid at position 185 (EU numbering) of the CH1_{H1} is a K and the amino acid at position 137 (EU numbering) of the CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 147 (EU numbering) of the CH2_{H1} is a D and the amino acid at position 180 (EU numbering) of the CL2 is a R; iii. the amino acid at position 136 (EU numbering) of the CH2_{H1} is a C and the amino acid at position 114 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 147 (EU numbering) of the CH1_{H1} is a K and the amino acid at position 131 (EU numbering) of the CL1 is a D; iii. the amino acid at position 173 (EU numbering) of the CH1_{H1} is a C and the amino acid at position 162 (EU numbering) of the CL1 is a C; iv. the amino acid at position 220 (EU numbering) in the H1H is a S and the amino acid at position 214 (EU numbering) of the CL1 is an S; and b) the H2 and the L2 comprise the following: i. the amino acid at position 39 (Kabat numbering) of the VH2 is a D and the amino acid at position 38 (Kabat numbering) of the VL2 is a K; and ii. the amino acid at position 147 (EU numbering) of the CH2_{H1} is a D and the amino acid at position 180 (EU numbering) of the CL2 is a R.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 185 (EU numbering) of the CHI_{H1} is a K and the amino acid at position 137 (EU numbering) of the CL1 is a D; iii. the amino acid at position 173 (EU numbering) of the CH1_{H1} is a C and the amino acid at position 162 (EU numbering) CL1 is a C; and iv. the amino acid at position 220 (EU numbering) in the H1H is a S and the amino acid at position 214 (EU numbering) of the CL1 is a S; and b) the H2 and the L2 comprise the following: i. the amino acid at position 39 (Kabat numbering) of the VH2 is a D and the amino acid at position 38 (Kabat numbering) of the VL2 is a K; and ii. the amino acid at position 147 (EU numbering) of the CH2_{H1} is a D and the amino acid at position 180 (EU numbering) of the CL2 is a R.

In some embodiments, a) the H1 and the L1 comprise the following: i. the amino acid at position 39 (Kabat numbering) of the VH1 is a K and the amino acid at position 38 (Kabat numbering) of the VL1 is a D; and ii. the amino acid at position 185 (EU numbering) of the CH1_{H1} is a K and the amino acid at position 137 (EU numbering) CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 147 (EU numbering) of the CH2_{H1} is a D and the amino acid at position 180 (EU numbering) of the CL2 is a R; iii. the amino acid at position 131 (EU numbering) of the CH2_{H1} is a C and the amino acid at position 114 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; and ii. the amino acid at position 185 (EU numbering) of the CH1_{H1} is a K and the amino acid at position 137 (EU numbering) of the CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 138 (EU numbering) of the CL2 is a K; iii. the amino acid at position 170 (EU numbering) of the CH2_{H1} is a C and the amino acid at position 162 EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 185 (EU numbering) of the CH1_{H1} is a E, the amino acid at position 137 (EU numbering) of the CL1 is a K; and iii. the amino acid at position 179 (EU numbering) of the CL1 is a E; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 138 (EU numbering) of the CL2 is a K; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; and ii. the amino acid at position 185 (EU numbering) of the CH1_{H1} is a K and the amino acid at position 137 (EU numbering) of the CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 138 (EU numbering) of the CL2 is a K; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 185 (EU numbering) of the CHI_{H1} is a E and the amino acid at position 137 (EU numbering) of the CL1 is a K; and iii. the amino acid at position 179 (EU numbering) of the CL1 is a E; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 147 (EU numbering) of the CH2_{H1} is a D and the amino acid at position 180 (EU numbering) of the CL2 is a R; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; and ii. the amino acid at position 185 (EU numbering) of the CH1_{H1} is a K and the amino acid at position 137 (EU numbering) of the CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 147 (EU numbering) of the CH2_{H1} is a D and the amino acid at position 180 (EU numbering) of the CL2 is a R; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: the amino acid at position 39 (Kabat numbering) of the VH1 is a K and the amino acid at position 38 (Kabat numbering) of the VL1 is a D; the amino acid at position 147 (EU numbering) of the CH1_{H1} is a K and the amino acid at position 131 (EU numbering) of the CL1 is a D; the amino acid at position 185 (EU numbering) of the CH1_{H1} is a K and the amino acid at position 137 (EU numbering) of the CL1 is a D; and b) the H2 and the L2 comprise the following: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 171 (EU numbering) of the CH2_{H1} is a C and the amino acid at position 162 (EU numbering) of the CL2 is a C; and the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a D and the amino acid at position 38 (Kabat numbering) of the VL1 is a K; and ii. the amino acid at position 185 (EU numbering) of the CH1_{H1} is a E and the amino acid at position 137 (EU numbering) of the CL1 is a K; and b) the H2 and the L2 comprise the following: i. the amino acid at position 39 (Kabat numbering) of the VH2 is a K and the amino acid at position 38 (Kabat numbering) of the VL2 is a D; ii. the amino acid at position 187 (EU numbering) of the CH2_{H1} is a D and the amino acid at position 138 (EU numbering) of the CL2 is a K; iii. the amino acid at position 136 (EU numbering) of the CH2_{H1} is a C and the amino acid at position 114 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a D and the amino acid at position 38 (Kabat numbering) of the VL1 is a K; and ii. the amino acid at position 185 (EU numbering) of the CH1_{H1} is a E and the amino acid at position 137 (EU numbering) of the CL1 is a K; and b) the H2 and the L2 comprise the following: i. the amino acid at position 39 (Kabat numbering) of the VH2 is a K and the amino acid at position 38 (Kabat numbering) of the VL2 is a D; ii. the amino acid at position 187 (EU numbering) of the CH2_{H1} is a D and the amino acid at position 138 (EU numbering) of the CL2 is a K; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a D and the amino acid at position 38 (Kabat numbering) of the VL1 is a K; and ii. the amino acid at position 185 (EU numbering) of the CH1_{H1} is a K and the amino acid at position 137 (EU numbering) of the CL1 is a D; and b) the H2 and the L2 comprise the following: i. the amino acid at position 39 (Kabat numbering) of the VH2 is a K and the amino acid at position 38 (Kabat numbering) of the VL2 is a D; ii. the amino acid at position 187 (EU numbering) of the CH2_{H1} is a D and the amino acid at position 138 (EU numbering) of the CL2 is a K; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is an S.

In some embodiments, a) the H1 and the L1 comprise the following: i. the amino acid at position 39 (Kabat numbering) of the VH1 is a D and the amino acid at position 38 (Kabat numbering) of the VL1 is a K; ii. the amino acid at position 147 (EU numbering) of the CH1_{H1} is a K and the amino acid at position 131 (EU numbering) of the CL1 is a D; iii. the amino acid at position 185 (EU numbering) of the CH1_{H1} is a E and the amino acid at position 137 (EU numbering) of the CL1 is a K; and iv. the amino acid at position 145 (EU numbering) of the CH1_{H1} is a S and the amino acid at position 180 (EU numbering) of the CL1 is a E; and b) the H2 and the L2 comprise the following: i. the amino acid at position 39 (Kabat numbering) of the VH2 is a K and the amino acid at position 38 (Kabat numbering) of the VL2 is a D; ii. the amino acid at position 187 (EU numbering) of the CH2_{H1} is a D and the amino acid at position 138 (EU numbering) of the CL2 is a K; iii. the amino acid at position 170 (EU numbering) of the VH2 is a C and the amino acid at position 162 (EU numbering) of the VL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 147 (EU numbering) of the CH1_{H1} is a D and the amino acid at position 131 (EU numbering) of the CL1 is a K; and iii. the amino acid at position 185 (EU numbering) of the CH1_{H1} is a E and the amino acid at position 137 (EU numbering) of the CL1 is a D; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 137 (EU numbering) of the CL2 is a K; iii. the amino acid at position 138 (EU numbering) of the CL2 is a R; iv. the amino acid at position 170 (EU numbering) of the CH2_{H1} is a C and the amino acid at position 162 (EU numbering) of the CL2 is a C; and v. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 147 (EU numbering) of the CHI_{H1} is a D and the amino acid at position 131 (EU numbering) of the CL1 is a K; and iii. the amino acid at position 145 (EU numbering) of the CH1_{H1} is a S; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 138 (EU numbering) of the CL2 is a K; iii. the amino acid at position 170 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a D and the amino acid at position 38 (Kabat numbering) of the VL1 is a K; ii. the amino acid at position 147 (EU numbering) of the CH1_{H1} is a K and the amino acid at position 131 (EU numbering) of the CL1 is a D; and iii. the amino acid at position 185 (EU numbering) of the CH1_{H1} is a E and the amino acid at position 137 (EU numbering) of the CL1 is a K; and b) the H2 and the L2 comprise the following: i. the amino acid at position 39 (Kabat numbering) of the VH2 is a K and the amino acid at position 38 (Kabat numbering) of the VL2 is a D; ii. the amino acid at position 147 (EU numbering) of the CH2_{H1} is a D and the amino acid at position 180 (EU numbering) of the CL2 is a R; iii. the amino acid at position 136 (EU numbering) of the CH2_{H1} is a C and the amino acid at position 114 (EU numbering) of the CL2 is a C; and iv. the amino acid at position 220 (EU numbering) in the H2H is a S and the amino acid at position 214 (EU numbering) of the CL2 is an S.

In some embodiments, a) the H1 and the L1 comprise the following: i. the amino acid at position 39 (Kabat numbering) of the VH1 is a K and the amino acid at position 38 (Kabat numbering) of the VL1 is a E; ii. the amino acid at position 147 (EU numbering) of the CH1_{H1} is a K and the amino acid at position 131 (EU numbering) of the CL1 is a D; and iii. the amino acid at position 185 (EU numbering) of the CH1_{H1} is a E and the amino acid at position 137 (EU numbering) of the CL1 is a K; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 138 (EU numbering) of the CL2 is a K; iii. the amino acid at position 136 (EU numbering) of the CH2_{H1} is a C and the amino acid at position 114 (EU numbering) of the CL2 is a C; and iv. the amino acid at position 220 (EU numbering) in the H2H is a S and the amino acid at position 214 (EU numbering) of the CL2 is a S.

In some embodiments, a) the H1 and the L1 comprise the following: i. the amino acid at position 39 (Kabat numbering) of the VH1 is a K and the amino acid at position 38 (Kabat numbering) of the VL1 is a D; ii. the amino acid at position 147 (EU numbering) of the CH1_{H1} is a D and the amino acid at position 131 (EU numbering) of the CL1 is a K; and iii. the amino acid at position 185 (EU numbering) of the CH1_{H1} is a D and the amino acid at position 137 (EU numbering) of the CL1 is a K; and b) the H2 and the L2 comprise the following: i. the amino acid at position 39 (Kabat numbering) of the VH2 is a D and the amino acid at position 38 (Kabat numbering) of the VL2 is a K; ii. the amino acid at position 187 (EU numbering) of the CH2_{H1} is a D and the amino acid at position 138 (EU numbering) of the CL2 is a K; iii. the amino acid at position 171 (EU numbering) of the CH2_{H1} is a C and the amino acid at position 162 (EU numbering) of the CL2 is a C; and iv. the amino acid at position 220 (EU numbering) in the H2H is a S and the amino acid at position 214 (EU numbering) of the CL2 is a S.

In some embodiments, i) the amino acid at position 87 (Kabat numbering) of the VH1 and/or VH2 is a G; and ii) the amino acid at position 45 (Kabat numbering) of the VL1 and/or VL2 is a W.

In some embodiments, i) the CH1_{H3} has a C at position 349, an S at position 366, an A at position 368 and a V at position 407 (EU numbering); and the CH2_{H3} has a C at position 354 and a W at position 366 (EU numbering); ii) the CH2_{H3} has a C at position 349, an S at position 366, an A at position 368 and a V at position 407 (EU numbering); and the CH1_{H3} has a C at position 354 and a W at position 366 (EU numbering); iii) the CH1_{H3} has a C at position 354, an S at position 366, an A at position 368 and a V at position 407 (EU numbering); and the CH2_{H3} has a C at position 349 and a W at position 366 (EU numbering); or iv) the CH2_{H3} has a C at position 354, an S at position 366, an A at position 368 and a V at position 407 (EU numbering); and the CH1_{H3} has a C at position 349 and a W at position 366 (EU numbering). In some embodiments, the amino acid at position 447 (EU numbering) of the CH1_{H3} and/or of the CH2_{H3} is deleted.

In some embodiments, i) the H1H and/or the H2H has an A at positions 234 and 235 (EU numbering); ii) the H1H and/or the H2H has an A at positions 234, 235 and 237 (EU numbering); or iii) the H1H and/or the H2H has an A at positions 234 and 235 and G at position 329 (EU numbering).

In some embodiments, i) the CH1_{H3} and/or the CH2_{H3} has an A at position 297 (EU numbering); ii) the CH1_{H3} and/or the CH2_{H3} has a G at position 297 (EU numbering); or iii) the CH1_{H3} and/or the CH2_{H3} has a S at position 297 (EU numbering). In some embodiments, the CH1_{H3} and/or the CH2_{H3} has an S at position 331 (EU numbering).

In some embodiments, the antibody comprises i) the CH1_{H2} and/or the CH2_{H2} has an C at position 370 (Kabat numbering); and ii) the CH1_{H2} and/or the CH2_{H2} has an C at position 375 (Kabat numbering).

In some embodiments, a) the VH1 comprises the amino acid sequence of SEQ ID NO: 13; and b) the VL1 comprises the amino acid sequence of SEQ ID NO: 22.

In some embodiments, a) the VH1 comprises the amino acid sequence of EVQLLESGGGLVQPGGSLKLSCAASGFTFNX₁YAMX₂WVRX₃APGKGLEWVAX₄IR SKYNNYATYYADSVKDRFTISRDDSKNTAYLQMNNLKTEDTAVYYCVRHGX₅FGN X₆X₇YVSWFAYWGQGTLVTVSS (SEQ ID NO: 440), wherein X1 of SEQ ID NO: 440 is D or T, wherein X2 of SEQ ID NO: 440 is D or N, wherein X3 of SEQ ID NO: 440 is D or K, wherein X4 of SEQ ID NO: 440 is K or R, wherein X5 of SEQ ID NO: 440 is N or T, wherein X6 of SEQ ID NO: 440 is D or N, and wherein X7 of SEQ ID NO: 440 is D or S; and b) the VL1 comprises the amino acid sequence of ELVVTQEPSLTVSPGGTVTLTCRSSTGAVTTSNYANWVQXIKPGQAPRGLIGX₂TNK RAPGTPARFSGSLLGGKAALTLSGVQPEDEAEYYCALWX₃SX₄LWVFGGGTKLTVL (SEQ ID NO: 441), wherein X1 of SEQ ID NO: 441 is E or D, wherein X2 of SEQ ID NO: 441 is D or G, wherein X3 of SEQ ID NO: 441 is D or Y, and wherein X4 of SEQ ID NO: 441 is D, N or Q.

In some embodiments, a) the VH2 comprises: a complementarity determining region 1 (VH2_{CDR1}) comprising the amino acid sequence of SEQ ID NO: 428; a complementarity determining region 2 (VH2_{CDR2}) comprising the amino acid sequence of SEQ ID NO: 430; and a complementarity determining region 3 (VH2_{CDR3}) comprising the amino acid sequence of SEQ ID NO: 432; and b) the VL2 comprises: a complementarity determining region 1 (VL2_{CDR1}) comprising the amino acid sequence of SEQ ID NO: 433; a complementarity determining region 2 (VL2_{CDR2}) comprising the amino acid sequence of SEQ ID NO: 434; and a complementarity determining region 3 (VL2_{CDR3}) comprising the amino acid sequence of SEQ ID NO: 435.

In some embodiments, a) the VH2 comprises: a complementarity determining region 1 (VH2_{CDR1}) comprising the amino acid sequence of SEQ ID NO: 5; a complementarity determining region 2 (VH2_{CDR2}) comprising the amino acid sequence of SEQ ID NO: 7; and a complementarity determining region 3 (VH2_{CDR3}) comprising the amino acid sequence of SEQ ID NO: 9; and b) the VL2 comprises: a complementarity determining region 1 (VL2_{CDR1}) comprising the amino acid sequence of SEQ ID NO: 10; a complementarity determining region 2 (VL2_{CDR2}) comprising the amino acid sequence of SEQ ID NO: 11; and a complementarity determining region 3 (VL2_{CDR3}) comprising the amino acid sequence of SEQ ID NO: 12.

In some embodiments, the CD58 comprises the amino acid sequence of SEQ ID NO: 49-50. In some embodiments, the IL-7 comprises the amino acid sequence of SEQ ID NO: 51.

The disclosure provides a polynucleotide comprising a nucleic acid sequence encoding any one of the antibodies disclosed herein. The disclosure also provides a vector comprising the polynucleotide of the disclosure.

The disclosure also provides a pharmaceutical composition comprising any one of the antibodies, the polynucleotides, or the vectors of the disclosure and a pharmaceutically acceptable carrier. In some embodiments, the pharmaceutical composition is packaged in a liposome or a lipid nanoparticle.

The disclosure provides a method of treating cancer in a subject in need thereof comprising administering a therapeutically effective amount of any one of the pharmaceutical compositions of the disclosure. The disclosure also provides a method of T-cell re-targeting in a subject in need thereof comprising administering a therapeutically effective amount of any one of the pharmaceutical compositions of the disclosure. The disclosure also provides a method of T-cell activation in a subject in need thereof comprising administering a therapeutically effective amount of any one of the pharmaceutical compositions of the disclosure.

In some embodiments, the subject has a cancer. In some embodiments, the cancer is a ULBP2 positive cancer. In some embodiments, the cancer is a primary tumor, a metastatic cancer, a multiply resistant cancer, a progressive tumor or recurrent cancer. In some embodiments, the cancer is a solid tumor. In some embodiments, the cancer is a urothelial cancer, a lung cancer, a brain cancer, a head and neck cancer, a breast cancer, a skin cancer, a melanoma, a liver cancer, a pancreatic cancer, a stomach cancer, a colon cancer, a rectal cancer, a uterine cancer, a cervical cancer, an ovarian cancer, a prostate cancer, a testicular cancer, a skin cancer or an esophageal cancer.

In some embodiments, the method further comprises administering a therapeutically effective amount of a ligand or a cytokine or an agnostic antibody that binds to the receptors of the ligands and the cytokines. In some embodiments, the ligand is CD48, CD58, CD86, TNFSF9, OX40L, 4-1BBL, GITL, CD70, CD80, MR1, TNFSF4, ICOSL, ICOSLG, MICA, MICB, ULBP1, ULBP2, ULBP3, RAET1G and RAET1L. In some embodiments, the cytokine is a IL-2, IL-7, IL-10, IL-12, IL-15, IL-18 or IL-21.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1A-D depicts schematic diagrams of antibodies with charged pair mutations, disulfide bond repositioning and knob into hole mutations. Grey shaded domains represent a first heavy chain polypeptide (H1) having a heavy chain variable region (VH1), having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1). White shaded domains represent a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2). The + and − symbols between the antigen binding domains represent the charged pair mutations. Lines between the CH1_{H1} and CL1 domain and CH2_{H1} and CL2 domain represent disulfide bonds, where a solid line represents an endogenous disulfide bond and a dashed line represents a repositioned disulfide bond. The protrusion and dent between the CH1_{H3} and CH2_{H3} domain represent knob into hole mutations. Charged pair mutations, disulfide bond repositioning and knob into hole mutations provide increased heavy chain and light chain heterodimerization, which is advantageous for production and purification of bispecific antibodies of the disclosure.

FIGS. 2A-2B are two graphs depicting biophysical characterization of light chain pairing bispecific antibodies EIP0187 (light chain pairing C), EIP0205 (light chain pairing D), EIP0356 (light chain pairing O) and EIP0377 (light chain pairing P) compared to EIP0112 Crossmab control antibody. FIG. 2A is a size exclusion chromatogram obtained from a protein A and size exclusion chromatography tandem purification of light chain pairing bispecific antibodies. FIG. 2B depicts differential scanning calorimetry analysis of light chain pairing bispecific antibodies.

FIG. 3A-3B are NuPAGE gel analyses of representative variants of light chain pairing bispecific antibodies depicted in FIGS. 2A-2B. FIG. 3A is a non-reduced NuPAGE analysis. FIG. 3B is a reduced NuPAGE analysis, together showing an intact bispecific antibody with expected protein masses of the heavy chain and light chain.

FIG. 4A-4B are a series of line graphs showing antigen binding of light chain pairing bispecific antibodies depicted in FIGS. 2A-2B compared to isotype and bispecific antibody controls. FIG. 4A is a line graph depicting antigen binding of light chain pairing bispecific antibody variants via sandwich ELISA where antibodies were captured on the plate coated with CD3ε. FIG. 4B is a line graph depicting antigen binding of light chain pairing bispecific antibody variants via sandwich ELISA where antibodies were captured on the plate coated with recombinant ULBP2.

FIG. 5A is mass spectrometry analysis of EIP0205 showing intact mass after PNGase F deglycosylation in non-reduced condition and chromatographic separation using reverse phase C4 column

FIG. 5B is mass spectrometry analysis of EIP0205 showing reduced mass of heavy chains after Rapid PNGase F deglycosylation in reduced condition and chromatographic separation using reverse phase C4 column.

FIG. 5C is mass spectrometry analysis of EIP0205 showing reduced mass of light chains after Rapid PNGase F deglycosylation in reduced condition and chromatographic separation using reverse phase C4 column.

FIG. 5D is mass spectrometry analysis of EIP0187 showing intact mass after PNGase F deglycosylation in non-reduced condition and chromatographic separation using reverse phase C4 column.

FIG. 5E is mass spectrometry analysis of EIP0187 showing reduced mass of heavy chains after Rapid PNGase F deglycosylation in reduced condition and chromatographic separation using reverse phase C4 column.

FIG. 5F is mass spectrometry analysis of EIP0187 showing reduced mass of light chains after Rapid PNGase F deglycosylation in reduced condition and chromatographic separation using reverse phase C4 column.

FIG. 6 is a line graph depicting functional evaluation (cytotoxicity) light chain pairing bispecific antibodies depicted in FIGS. 2A-2Bin a tumor cell and T cell co-culture assay compared to bispecific control antibody (EIP0112)

FIG. 7A are chromatograms of bispecific antibody variants obtained from tandem purification.

FIG. 7B is a non-reduced NuPAGE analysis showing protein mass of intact bispecific antibody variants.

FIG. 7C is a reduced NuPAGE analysis showing protein mass of bispecific antibody variant heavy and light chains.

FIG. 7D is a line graph depicting antigen binding of light chain pairing bispecific antibodies via sandwich ELISA, where antibodies were captured on a plate coated with CD3ε.

FIG. 7E is a line graph depicting antigen binding of light chain pairing bispecific antibodies via sandwich ELISA, where antibodies were captured on the plate coated with antigen.

FIG. 8A is a line graph depicting binding of αULBP2-αCD3 bispecific antibody variants to human CD3 epsilon by ELISA.

FIG. 8B is a line graph depicting binding of αULBP2-αCD3 bispecific variants to cynomolgus CD3 epsilon by ELISA.

FIGS. 9A-9B are a series of line graphs depicting luciferase activity in co-cultures of tumor cells and Jurkat NFAT luciferase reporter cells in the presence of αULBP2-αCD3 bispecific antibody variants. FIG. 9A depicts co-cultures of SiHa tumor cells.

FIG. 9B depicts co-culture of HCT116 tumor cells.

FIGS. 10A-10C are a series of line graphs depicting T-cell mediated cytotoxicity of three tumor cell lines in the presence of αULBP2-αCD3 bispecific antibody variants of FIGS. 9A-9B. FIG. 10A depicts cytotoxicity of HCT116 tumor cells. FIG. 10B depicts cytotoxicity of MDA-MB-231 GFP tumor cells.

FIG. 10C depicts cytotoxicity of SiHa tumor cells in the presence of αULBP2-αCD3 bispecific antibody variants.

FIGS. 11A-11C are a series of line graphs depicting secretion of cytokines from from activated T cells in co-culture with SiHa tumor cells in the presence of αULBP2-αCD3 bispecific affinity variants of FIGS. 9A-9B. FIG. 11A depicts secretion of IFNγ.

FIG. 11B depicts secretion of IL-2.

FIG. 11C depicts secretion of TNFα.

FIG. 12A depicts a bispecific antibody variant with no CD58 fusion.

FIG. 12B depicts a bispecific antibody variant with a CD58 fusion to the carboxyl terminal of CH1_{H3}.

FIG. 12C depicts a bispecific antibody variant with a CD58 fusion to the carboxyl terminal of CH2_{H3}.

FIG. 12D depicts a bispecific antibody variant with a CD58 fusion to the carboxyl terminal of CH1_{H3} and a CD58 fusion to the carboxyl terminal of CH2_{H3}.

FIG. 12E depicts a bispecific antibody variant with an amino terminal fusion to CD58 on VL2.

FIG. 12F depicts a bispecific antibody variant with an amino terminal fusion to CD58 on VH2.

FIG. 12G depicts a bispecific antibody variant with an amino terminal fusion to CD58 on VL1.

FIG. 12H depicts a bispecific antibody variant with an amino terminal fusion to CD58 on VH1.

FIG. 12L depicts a bispecific antibody variant with a CD58 fusion to the carboxyl terminal of CL2

FIG. 12J depicts a bispecific antibody variant with a CD58 fusion to the carboxyl terminal of CL1

FIG. 12K depicts a bispecific antibody variant with a CD58 fusion to the carboxyl terminal of CL1 and CL2

FIG. 13 are chromatograms obtained from tandem purification of costimulatory ligand or cytokine fusion bispecific variants engineered with light chain pairing technology (EIP0205, EIP0359, EIP0360, EIP0363).

FIG. 14 depicts differential scanning calorimetry analysis of costimulatory ligand or cytokine fusion αULBP2-αCD3 bispecific variants (EIP0205, EIP0359, EIP0363).

FIG. 15A is a line graph depicting antigen binding of costimulatory ligand or cytokine fusion bispecific antibody variants to a plate coated with recombinant CD3ε via sandwich ELISA.

FIG. 15B is a line graph depicting antigen binding of costimulatory ligand or cytokine fusion bispecific antibody variants to a plate coated with recombinant ULBP2 protein via sandwich ELISA.

FIG. 16A depicts cytolysis of MDA-MB-231 GFP tumor cells in the presence of αULBP2-αCD3 bispecific variants after 7-day incubation with naïve T cells.

FIG. 16B shows brightfield and fluorescent microscopy representative images of naïve T cell activation and MDA-MB-231 cell death

FIG. 16C depicts cytolysis of ULBP2-deficient MDA-MB-231 GFP tumor cells in the presence of αULBP2-αCD3 bispecific antibody variants after 7-day incubation with naïve T cells.

FIGS. 17A-17B are a series of line graphs depicting secretion of IFNγ from naïve T cells after 24 hour incubation with MDA-MB-231 GFP tumor cells and ULBP2-deficient MDA-MB-231 GFP tumor cells in the presence of bispecific antibody variants of FIGS. 16A-16C. FIG. 17A depicts MDA-MB-231 GFP tumor cells.

FIG. 17B depicts ULBP2-deficient MDA-MB-231 GFP tumor cells.

FIGS. 18A-18B are a series of line graphs depicting secretion of IL-2 from naïve T cells after 24 hour incubation with MDA-MB-231 GFP tumor cells and ULBP2-deficient MDA-MB-231 GFP tumor cells in the presence of bispecific antibody variants of FIGS. 16A-16C. FIG. 18A depicts MDA-MB-231 GFP tumor cells.

FIG. 18B depicts ULBP2-deficient MDA-MB-231 GFP tumor cells.

FIGS. 19A-19B are a series of line graphs depicting secretion of IFNγ from naïve T cells after 24 hour incubation with MDA-MB-231 GFP tumor cells and ULBP2-deficient MDA-MB-231 GFP tumor cells in the presence of bispecific antibody variants of FIGS. 16A-16C. FIG. 19A depicts MDA-MB-231 GFP tumor cells.

FIG. 19B depicts ULBP2-deficient MDA-MB-231 GFP tumor cells.

FIG. 20A depicts cytolysis of SiHa cells in the presence of αULBP2-αCD3 bispecific and αULBP2-αCD3-CD58 bispecific variants after 48 hour incubation with activated T cells.

FIG. 20B depicts cytolysis of SiHa cells in the presence of αULBP2-αCD3 bispecific and αULBP2-αCD3-CD58 bispecific variants after 48 hour incubation with activated T cells.

FIG. 21A depicts cytolysis of SiHa cells in the presence of αULBP2-αCD3 bispecific and αULBP2-αCD3-CD58 bispecific variants after 48 hour incubation with activated T cells.

FIG. 21B depicts cytolysis of SiHa cells in the presence of αULBP2-αCD3 bispecific and αULBP2-αCD3-CD58 bispecific variants after 48 hour incubation with activated T cells.

FIG. 22A is a line graph depicting secretion of IFNγ after 48 hours of activated T cells in co-culture with SiHa cells in the presence of αULBP2-αCD3 bispecific and αULBP2-αCD3-CD58 bispecific antibody variants.

FIG. 22B is a line graph depicting secretion of IFNγ after 48 hours of activated T cells in co-culture with SiHa cells in the presence of αULBP2-αCD3 bispecific and αULBP2-αCD3-CD58 bispecific antibody variants.

FIG. 23A is a line graph depicting secretion of IFNγ after 48 hours of activated T cells in co-culture with SiHa cells in the presence of αULBP2-αCD3 bispecific and αULBP2-αCD3-CD58 bispecific antibody variants.

FIG. 23B is a line graph depicting secretion of IFNγ after 48 hours of activated T cells in co-culture with SiHa cells in the presence of αULBP2-αCD3 bispecific and αULBP2-αCD3-CD58 bispecific antibody variants.

FIG. 24A is a line graph depicting secretion of IL-2 after 48 hours of activated T cells in co-culture with SiHa cells in the presence of αULBP2-αCD3 bispecific and αULBP2-αCD3-CD58 bispecific antibody variants.

FIG. 24B is a line graph depicting secretion of IL-2 after 48 hours of activated T cells in co-culture with SiHa cells in the presence of αULBP2-αCD3 bispecific and αULBP2-αCD3-CD58 bispecific antibody variants.

FIG. 25A is a line graph depicting secretion of IL-2 after 48 hours of activated T cells in co-culture with SiHa cells in the presence of αULBP2-αCD3 bispecific and αULBP2-αCD3-CD58 bispecific antibody variants.

FIG. 25B is a line graph depicting secretion of IL-2 after 48 hours of activated T cells in co-culture with SiHa cells in the presence of αULBP2-αCD3 bispecific and αULBP2-αCD3-CD58 bispecific antibody variants.

FIG. 26A is a line graph depicting secretion of TNFα after 48 hours of activated T cells in co-culture with SiHa cells in the presence of αULBP2-αCD3 bispecific and αULBP2-αCD3-CD58 bispecific antibody variants.

FIG. 26B is a line graph depicting secretion of TNFα after 48 hours of activated T cells in co-culture with SiHa cells in the presence of αULBP2-αCD3 bispecific and αULBP2-αCD3-CD58 bispecific antibody variants.

FIG. 27A is a line graph depicting secretion of TNFα after 48 hours of activated T cells in co-culture with SiHa cells in the presence of αULBP2-αCD3 bispecific and αULBP2-αCD3-CD58 bispecific antibody variants.

FIG. 27B is a line graph depicting secretion of TNFα after 48 hours of activated T cells in co-culture with SiHa cells in the presence of αULBP2-αCD3 bispecific and αULBP2-αCD3-CD58 bispecific antibody variants.

FIG. 28 is a line graph depicting cytolysis of MDA-MB-231 GFP cells after 7 day incubation with PBMCs in the presence of αULBP2-αCD3, αULBP2-αCD3-CD58 bispecific αULBP2-αCD3-CD58 variable domain fusion bispecific antibody variants.

FIG. 29A is a line graph depicting cytolysis of MDA-MB-231 cells in the presence of bispecific antibody variants.

FIG. 29B is a line graph depicting secretion of IFNγ from MDA-MB-231 cells in the presence of bispecific antibody variants.

FIG. 30 are representative microscopy images of cytolysis of MDA-MB-231-GFP cells following 48 hour incubation with naïve T cells, round 3 and round 5 exhausted T cells in the presence of bispecific antibody variants.

FIG. 31 is a radar plot depicting normalized levels of the T-cell markers IL2, IFNγ, CD25, CD69, GZMB, CD2, PD-1, CD3ε and TIM3 present after 72 hour incubation of PBMCs with MDA-MB-231 cells in the presence of bispecific antibody variants.

FIGS. 32A-32D are a series of graphs depicting improved tumor growth inhibition, pharmacokinetics and survival of humanized mice following treatment with bispecific antibody variants. FIG. 32A is a line graph depicting growth inhibition of SiHa tumors over time after adoptive transfer of human T cells and dosing with bispecific antibody variants EIP0542, EIP0205 and EIP0359. FIG. 32B is a survival curve after adoptive transfer of human T cells and dosing with bispecific antibody variants of FIG. 32A. FIG. 32C is a line graph depicting pharmacokinetics of bispecific antibody variants of FIG. 32A. FIG. 32D is a graph depicting pharmacokinetics of EIP0561 following administration of a 10 mg/kg dosage to humanized mice.

FIGS. 33A-33C are a series of histograms from flow cytometry analysis of tumor infiltrating lymphocytes in SiHa tumors 3 days post-engraftment and treated with bispecific antibody variants FIG. 33A is analysis of Granzyme B showing increased tumor cytolysis by bispecific antibody variants. FIG. 33B is analysis of CD25 showing increased T cell activation by bispecific antibody variants. FIG. 33C is analysis of CD3ε showing decreased T cell exhaustion by bispecific antibody variants.

FIGS. 34A-34C are a series of structural renderings modeling A06 and E12 binding to ULBP2. FIG. 34A is a homology model of the ULBP2-NKG2D complex. FIG. 34B is a docked model of ULBP2 and A06 antibody. FIG. 34C is a docked model of ULBP2 and E12 antibody. Residue R106 is shown in stick model in all the panels. ULBP2 is shown in dark gray while NKG2D, A06 and E12 are shown in light gray.

FIG. 35 is a line graph depicting growth inhibition of CORL-105 tumors over time after co-engraftment of human T cells and dosing with bispecifics and bispecific CD58 fusions with various CD3 affinities.

FIGS. 36A-36F are a series of graphs depicting cytolysis of tumor cells in the presence of bispecific CD58 fusions after 48 hour incubation with activated T cells. FIG. 36A shows cytolysis of HCT116 cells. FIG. 36B shows cytolysis of U266B1 cells. FIG. 36C shows cytolysis of JeKo-1 cells. FIG. 36D shows cytolysis of PSMA-low LNCAP prostate cancer cells (LNCAP-vL). FIG. 36E shows cytolysis of MM1s cells. FIG. 36F shows cytolysis of Raji cells.

FIG. 37 is a graph depicting a chromatogram obtained from tandem purification of an exemplary antibody with and without exemplary disulfide stabilization mutations.

FIGS. 38A-38D are two graphs and microscopy images depicting cytolysis of MDA-MB-231 GFP tumor cells and ULBP2-deficient MDA-MB-231 GFP tumor cells in the presence of αULBP2-αCD3 bispecific antibody variants after 5-day incubation at a ratio of 1:10 with naïve T cells. FIG. 38A shows tumor cells incubated with EIP0205 at various concentrations. FIG. 38B shows brightfield and fluorescent microscopy representative images of naïve T cell activation and MDA-MB-231 cell death with EIP0205. FIG. 38C shows tumor cells incubated with EIP0359 at various concentrations. FIG. 38D shows brightfield and fluorescent microscopy representative images of naïve T cell activation and MDA-MB-231 cell death with EIP0359.

FIG. 39 is a graph depicting cytolysis of MDA-MB-231 GFP tumor cells in the presence of αULBP2-αCD3 bispecific antibody variants after 5-day incubation at a ratio of 1:10 with naïve T cells.

FIGS. 40A-40C are a series of line graphs depicting cytolysis of tumor cells and cytokine secretion in the presence of αULBP2-αCD3 bispecific and αULBP2-αCD3-CD58 bispecific variants after 5 day incubation at an E:T ratio of 10:1 with naïve T cells. FIG. 40A depicts cytolysis of tumor cells. FIG. 40B depicts secretion of IFNγ after 48 hours of activated T cells in co-culture with tumor cells. FIG. 40C depicts secretion of IL-2 after 48 hours of activated T cells in co-culture with tumor cells.

FIG. 41 is a graph depicting killing of MDA-MB-231 tumor cells in the presence of activated T cells T cells in the presence of αULBP2-αCD3-CD58 bispecific variants after 48 hour incubation at an E:T ratio of 5:1 with activated T cells.

FIGS. 42A-42F are a series of graphs depicting cytolysis of tumor cells in the presence of activated T cells in the presence of αCD3 bispecific antibody variants comprising light chain pairing of the present disclosure after 2 days compared to controls. FIG. 42A shows JeKo-1 tumor cells at an effector to target cell (E:T) ratio of 5:1. FIG. 42B shows Ramos cells at an effector to target cell (E:T) ratio of 10:1. FIG. 42C shows Raji cells at an effector to target cell (E:T) ratio of 10:1. FIG. 42D shows SUDHL10 cells at an effector to target cell (E:T) ratio of 10:1. FIG. 42E shows MV411 cells at an effector to target cell (E:T) ratio of 5:1. FIG. 42F shows OCI-AML2 cells at an effector to target cell (E:T) ratio of 5:1.

FIGS. 43A-43B are a series of graphs depicting lysis of tumor cells in the presence of naive T cells at an effector to target cell (E:T) ratio of 7.5:1 in the presence of αCD3 bispecific antibody variants comprising light chain pairing with and without CD58 fusion molecules of the present disclosure after 3 days compared to controls. FIG. 43A shows tumor lysis of JeKo-1 cells. FIG. 43B shows tumor lysis of MV411 cells.

FIGS. 44A-44B are a series of graphs depicting cytolysis of tumor cells in the presence of activated T cells in the presence of αBCMA-αCD3 and αBCMA-αCD3-CD58 bispecific variants after 48 hour incubation compared to no antibody control. FIG. 43A shows NCI929 tumor cells at an effector to target cell (E:T) ratio of 2:1. FIG. 43B shows U266B1 cells at an effector to target (E:T) cell ratio of 1.6:1.

### DETAILED DESCRIPTION

Production of heteromultimeric antibodies (e.g. bispecific antibodies) using conventional techniques provides challenges including the production of a mis-paired antibodies, reduced yield and decreased/elimination of effector function among others. In addition, aggregation and precipitation often occur during the preparation and assembly of the heteromultimeric antibody. Mismatching of heavy chains and light chains, aggregation and precipitation can greatly reduce the yield of the desired heteromultimeric antibody. Thus, there exists a need for the compositions and methods herein to produce heteromultimeric antibodies more efficiently and at higher levels.

Disclosed herein are compositions and efficient production processes/methods for economical production of heteromultimeric antibodies (e.g., bispecific antibodies), by using or modulating one or more of the following including without limitation: oppositely charged pairs of mutations on light chains and heavy chains, disulfide bond repositioning mutations or knob in hole mutations. The inventive methods described herein result in reduced mismatching of heavy chains and light chains, decreased loss of protein to precipitation and/or aggregation and improved the overall yield of heteromultimeric antibody production, such as the production of bispecific antibodies.

T cell retargeting (or T cell redirecting) bispecific antibodies is a novel class of therapeutics, capable of recruiting T cells to tumor cells and inducing tumor-specific (but MHC-independent) activation of T cell effector activities. Typically, T cell retargeting bispecific antibodies contain an antigen binding domain that targets CD3 portion of the T cell receptor for T cell recruitment, and an antigen binding domain that targets a disease-associated antigen (DAA). This targeting design promotes the recruitment of T cell and positions it in close contact with a target tumor cell, resulting in the formation of an immunological synapse, local T cell activation and the subsequent destruction of the target cell by perforin and granzyme released from T cell cytotoxic granules.

As the CD3 binding affinity of the T-cell retargeting bispecific antibodies is crucial for recruitment of T cells, the present invention also relates to the generation of a panel of antibodies that bind to human CD3 and that are cross reactive with cynomolgus CD3. Cross-reactivity with cynomolgus CD3 is an important feature in order to facilitate preclinical development of T cell retargeting bispecific antibodies that incorporate the anti-CD3ε antibodies described herein. Furthermore, these anti-CD3ε antibodies display different binding affinities. The affinity of the CD3 arm of a bispecific antibody can significantly modify the functional activity of the bispecific antibody. Thus, it is desirable and advantageous to have anti-CD3 antibodies with varied affinities.

Additionally, bispecific antibodies disclosed herein may have a cytokine or costimulatory molecule fusion peptide that acts as antagonist to inhibit or block deleterious interactions or as an agonist to mimic or enhance physiological responses. Physiological responses include but are not limited to T-cell activation, T-cell proliferation and prevention of T-cell exhaustion. These properties are advantageous over conventional CD3-bispecific antibodies or tumor targeted co-stimulatory receptor agonists which do not optimally activate T-cells and induce (or promote) T-cell dysfunction. In accordance, cytokine and/or costimulatory fusion peptides are advantageous for enhancing the therapeutic potential of bispecific antibodies.

Altogether, these characteristics (e.g. oppositely charged pairs of mutations on light chains and heavy chains, disulfide bond repositioning mutations, knob into hole mutations, CD3 affinity variants, cytokine or costimulatory molecule fusion peptides) present considerable advantages from a development perspective. For example, it provides co-stimulation of T-cells and prevents T-cell exhaustion, Further, bispecific T cell retargeting agents share the drug-like properties of human monoclonal antibodies. Further, T-cell retargeting bispecific antibodies are advantageous over other existing therapies (e.g. CAR-T therapies) because it provides an off-the-shelf product with a high safety profile (e.g. mitigation of cytokine release syndrome and reduced levels of tonic signaling leading to T-cell dysfunction) and the possibility of dose titration and escalation.

### Antibody Compositions and Structures

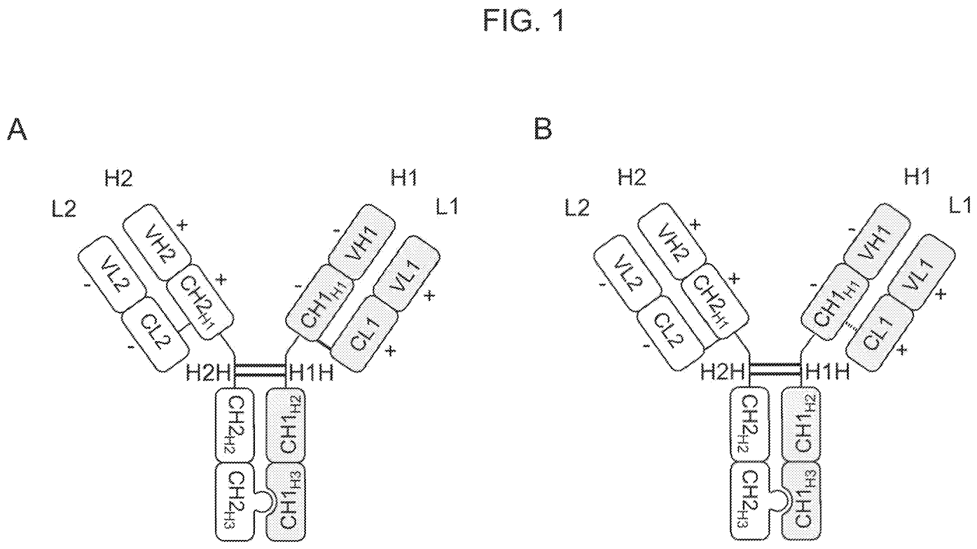

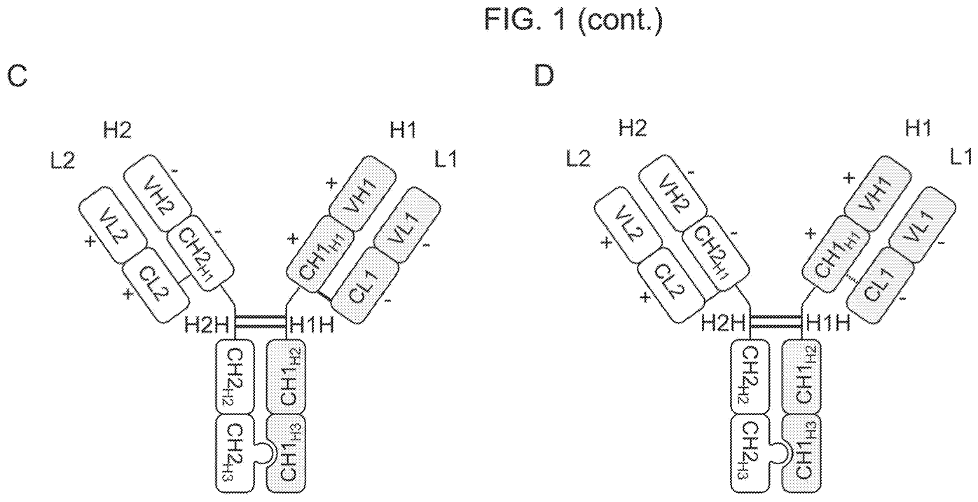

The present disclosure provides an antibody comprising the following domain structure: a) a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and b) a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2). A schematic diagram of the antibody structure of the disclosure is shown in FIGS. 1A-1D.

As used herein, the term “antibody” refers to an immunoglobulin (Ig) molecule and immunologically active portions of an immunoglobulin molecule, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶). Antibodies include, but are not limited to, polyclonal antibodies, monoclonal antibodies, chimeric antibodies. The antibody may be from recombinant sources and/or produced in transgenic animals.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, IgG4 and others. Furthermore, in humans, the light chain may be a kappa chain or a lambda chain. Accordingly, in one embodiment, the antibody disclosed herein is an IgG antibody.

Antibodies may be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “antibody fragment” as used herein is intended to include without limitation, Fv, Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Techniques can be adapted for the production of single-chain antibodies specific to an antigenic protein of the disclosure (see e.g., U.S. Pat. No. 4,946,778). In addition, methods can be adapted for the construction of Fab expression libraries (see e.g., Huse, et al., 1989 Science 246:1275-1281) to allow rapid and effective identification of monoclonal Fab fragments with the desired specificity for a protein or derivatives, fragments, analogs or homologs thereof.

As used herein, the term “epitope” refers to the site on an antigen that is recognized by the antibodies and fragments disclosed herein. The term “epitope” includes any protein determinant capable of specific binding to an immunoglobulin. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is <1 micromolar; e.g., <100 nM, preferably <10 nM and more preferably <1 nM.

Bispecific antibodies are antibodies that have binding specificities for at least two different antigens. The present disclosure provides a bispecific antibody having a first antigen binding region that binds to a first antigen (e.g. CD3) and a second antigen binding region that binds to a second antigen (e.g. disease associated antigen)

Antibodies with more than two valencies are also contemplated. For example, trispecific antibodies can be prepared. Tutt et al., J. Immunol. 147:60 (1991).

### Antibody Variants

In certain embodiments, amino acid sequence variants of the antibodies provided herein are contemplated. For example, it may be desirable to improve the heavy chain heterodimerization, light chain heterodimerization, binding affinity, and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics (e.g., light chain heterodimerization, heavy chain heterodimerization, antigen binding).

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic (negatively charged): Asp, Glu;
(4) basic (positively charged): His, Lys, Arg;
(5) residues that influence chain orientation: Gly, Pro;
(6) aromatic: Trp, Tyr, Phe.

Functional variants of the antibody or antigen-binding fragments described herein are also encompassed by the present disclosure. The term “functional variant” as used herein includes modifications or chemical equivalents of the amino acid and nucleic acid sequences disclosed herein that perform substantially the same function as the polypeptides or nucleic acid molecules disclosed herein in substantially the same way. For example, functional variants of polypeptides disclosed herein include, without limitation, conservative amino acid substitutions.

A “conservative amino acid substitution” as used herein, is one in which one amino acid residue is replaced with another amino acid residue that change an amino acid to a different amino acid with similar biochemical properties (e.g. charge, hydrophobicity and size). Variants of polypeptides also include additions and deletions to the polypeptide sequences disclosed herein. In addition, variant nucleotide sequences include analogs and derivatives thereof. A variant of the binding proteins disclosed herein include proteins that bind to the same antigen or epitope as the binding proteins.

In some embodiments, the charged amino acid residue is a naturally occurring amino acid or a non-naturally occurring amino acid. In some embodiments, the naturally occurring charged amino acid residue is an arginine, a lysine, a histidine, a glutamic acid or an aspartic acid.

### Light Chain and Heavy Chain Substitution Variants

To generate a substantially homogeneous population of bispecific antibodies with the correct pairing of heavy chain and light chains (i.e. cognate pairing or heterodimerization of a light chain with the heavy chain necessary to form the variable domain or antigen binding domain of the original antibody), the first heavy chain polypeptide (H1) has a strong preference for binding with the first light chain polypeptide (L1) relative to the second light chain polypeptide (L2); and the second heavy chain polypeptide (H2) has a strong preference for binding with the second light chain polypeptide (L2) relative to first light chain polypeptide (L1). In addition, the first heavy chain polypeptide (H1) and the second heavy chain polypeptide (H2) have a stronger preference for heterodimerization than homodimerization (i.e. heavy chain heterodimerization).

Antibody variants having one or more amino acid substitutions are provided herein. Exemplary substitutional mutagenesis sites include the charged substitution pairs shown in Tables 1.1-1.3 and 2-6.

For the bispecific antibodies of the disclosure, it is advantageous to use Fab heterodimerization strategies to permit the correct association of Fab domains belonging to the same arm and minimize aberrant pairing of Fab domains belonging to different arm. Exemplary Fab heterodimerization strategies include but are not limited to those shown in Table 1.1 and Table 1.2. Antibodies domains as listed in Tables 1.1 and Table 1.2 correspond to domains of the present disclosure as depicted in FIG. 1.

TABLE 1.1
Fab Heterodimerization Strategies

| Name | STRATEGY | VH | CH1 | VL | CL | REFERENCE |
|---|---|---|---|---|---|---|
| F1 | CrossMabCH1-CL | WT | CL | WT | CH1 | Schaefer et al., 2011, |
| | | | domain | | domain | Cancer Cell 2011; |
| | | | | | | 20: 472-86; |
| | | | | | | PMID: 22014573. |
| F2 | orthogonal Fab | 39K, | H172A, | 1R, 38D, | L135Y, | Lewis et al., 2014, Nat |
| | VHVRD1CH1CRD2 - | 62E | F174G | (36F) | S176W | Biotechnol 32: 191-8 |
| | VLVRD1CλCR D2 | | | | | |
| F3 | orthogonal Fab | 39Y | WT | 38R | WT | Lewis et al., 2014, Nat |
| | VHVRD2CH1wt - | | | | | Biotechnol 32: 191-8 |
| | VLVRD2Cλwt | | | | | |
| F4 | TCR CαCβ | 39K | TCRCα | 38D | TCR Cβ | Wu et al., 2015, MAbs |
| | | | | | | 7: 364-76 |
| F5 | CR3 | WT | T192E | WT | N137K, | Golay at al., 2016, J |
| | | | | | S114A | Immunol 196: 3199-211. |
| F6 | MUT4 | WT | L143Q, | WT | V133T, | Golay at al., 2016, J |
| | | | S188V | | S176V | Immunol 196: 3199-211. |
| F7 | DuetMab | WT | F126C | WT | S121C | Mazor et al., 2015, |
| | | | | | | MAbs 7: 377-89; |
| | | | | | | Mazor et al., 2015, |
| | | | | | | MAbs 7: 461-669. |

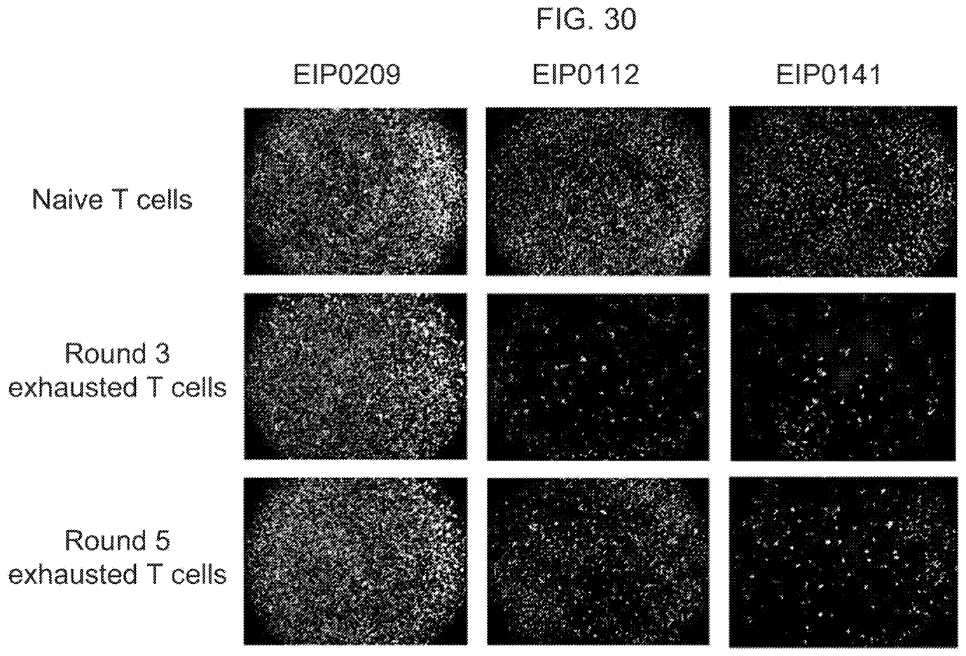

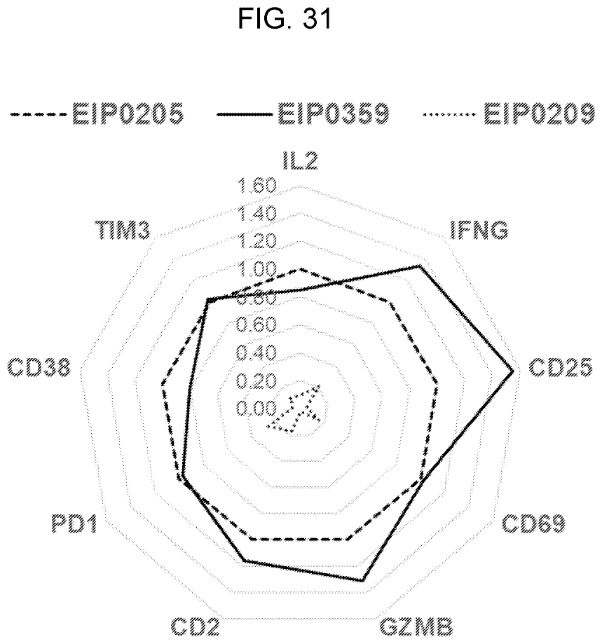

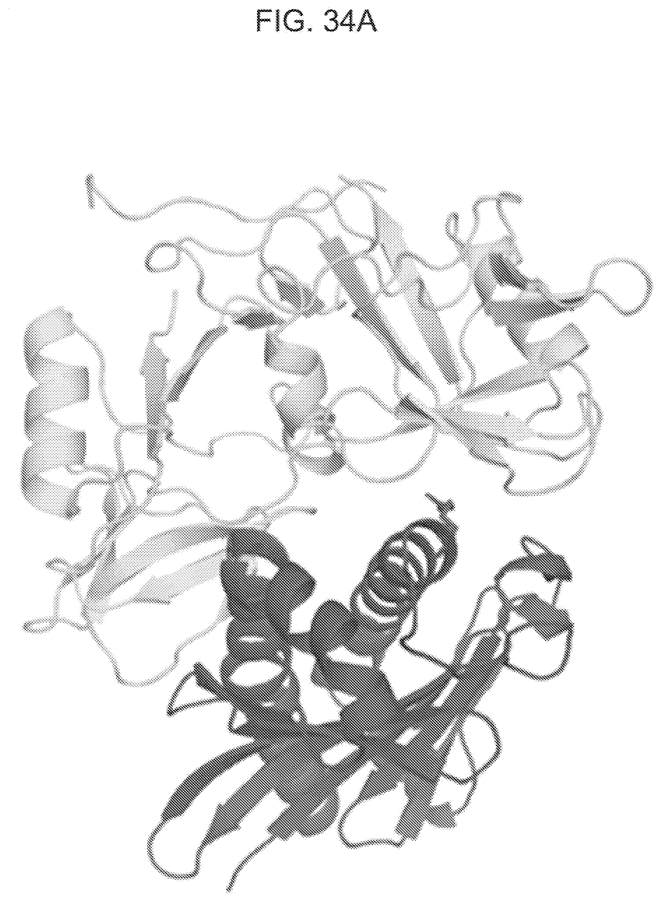

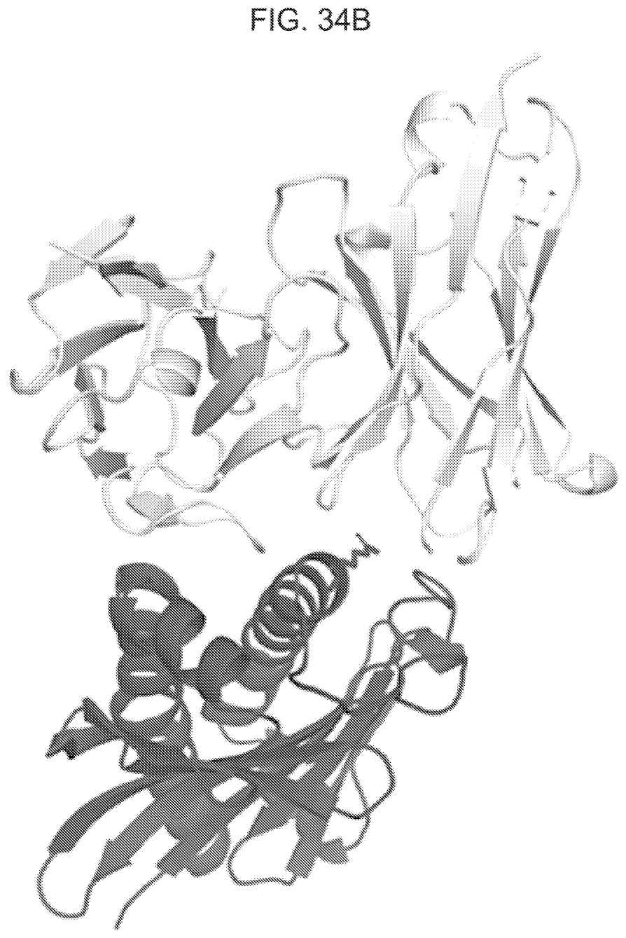

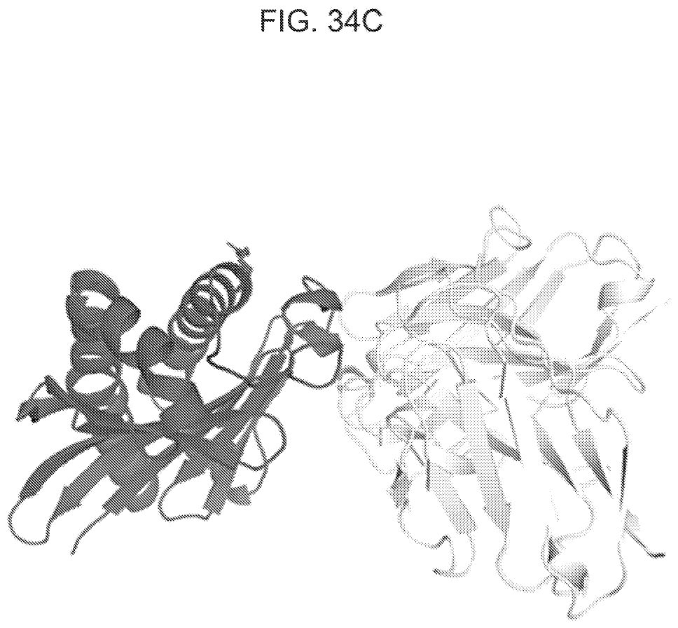

TABLE 1.2
Fc Heterodimerization Strategies

| | | CH3 DOMAIN | CH3 DOMAIN | |
|---|---|---|---|---|
| NO. | STRATEGY | 1 | 2 | REFERENCES |
| Fc 1 | knobs-into- | T366Y | Y407T | Ridgway et al., 1996, Protein Eng |
| | holes (Y-T) | | | 9: 617-21 |
| Fc 2 | knobs-into- | S354C, T366W | Y349C, T366S, | Atwell et al., 1997, J Mol Biol. |
| | holes (CW- | | L368A, Y407V | 270(1): 26- 35; Merchant et al., |
| | CSAV) | | | 1998, Nat Biotechnol 16: 677-681 |
| Fc 3 | HA-TF | S364H, F405A | Y349T, T394F | Moore et al., 2011, MAbs |
| | | | | 3(6): 546-57 |
| Fc 4 | ZW1 (VYAV- | T350V, L351Y, | T350V, T366L, | Von Kreudenstein et al., 2013, |
| | VLLW) | F405A, Y407V | K392L, T394W | MAbs 5: 646-54 |
| Fc 5 | CH3 charge | K392D, K409D | E356K, D399K | Gunasekaran et al., 2010, J Biol |
| | pairs (DD-KK) | | | Chem 285: 19637-46 |
| Fc 6 | IgG1 | IgG1: D221E, | IgG1: D221R, | Strop et al., 2012, J Mol Biol |
| | hinge, CH3 | P228E, L368E | P228R, K409R | 420: 204-19 |
| | charge pairs | | | |
| | (EEE-RRR) | | | |
| Fc 7 | IgG2 | IgG2: C223E, | IgG2: C223R, | Strop et al., 2012, J Mol Biol |
| | hinge, CH3 | P228E, L368E | E225R, P228R, | 420: 204-19 |
| | charge pairs | | K409R | |
| | (EEE-RRRR) | | | |
| Fc 8 | EW-RVT | K360E, | Q347R, D399V, | Choi et al., 2013, Mol Cancer Ther |
| | | K409W, | F405T | 12: 2748-59 |
| Fc 9 | EW-RVTS-S | K360E, K409W, | Q347R, D399V, | Choi et al., 2015, Mol Immunol |
| | | Y349C | F405T, S354C | 65: 377-83 |
| Fc 10 | Biclonic | 366K (+351K) | 351D or E or D | Geuijen et al., 2014, Journal of |
| | | | at 349, 368, | Clinical Oncology 32: suppl: 560 |
| | | | 349, or 349 + | |
| | | | 355 | |
| Fc 11 | DuoBody (L-R) | F405L | K409R | Labrijn et al., 2013, Proc Natl |
| | | | | Acad Sci USA 110:5145-50 |
| Fc 12 | SEEDbody | IgG/A chimera | IgG/A chimera | Davis et al., 2010, Protein Eng Des |
| | | | | Sel 23: 195-202 |
| Fc 13 | BEAT | residues from | residues from | Moretti et al., 2013, BMC |
| | | TCRα interface | TCRβ interface | Proceedings 7(Suppl 6): O9 |
| Fc 14 | 7.8.60 (DMA- | K360D, D399M, | E345R, Q347R, | Leaver-Fey et al., Structure |
| | RRVV) | Y407A | T366V, K409V | 24: 641-51 |
| Fc 15 | 20.8.34 | Y349S, K370Y, | E356G, E357D, | Leaver-Fey et al., Structure |
| | (SYMV- | T366M, K409V | S364Q, Y407A | 24: 641-51 |
| | GDQA) | | | |
| Fc 16 | Skew variant | None | none | FIG. 34 of US 2016/0355600 |
| | 12757 | | | |
| Fc 17 | Skew variant | L368D, K370S | S364K | FIG. 34 of US 2016/0355600 |
| | 12758 | | | |
| Fc 18 | Skew variant | L368D, K370S | S364K, E357L | FIG. 34 of US 2016/0355600 |
| | 12759 | | | |
| Fc 19 | Skew variant | L368D, K370S | S364K, E357Q | FIG. 34 of US 2016/0355600 |
| | 12760 | | | |
| Fc 20 | Skew variant | T411E, K360E, | D401K | FIG. 34 of US 2016/0355600 |
| | 12761 | Q362E | | |
| Fc 21 | Skew variant | L368E, K370S | S364K | FIG. 34 of US 2016/0355600 |
| | 12496 | | | |
| Fc 22 | Skew variant | K370S | S364K | FIG. 34 of US 2016/0355600 |
| | 12511 | | | |
| Fc 23 | Skew variant | L368E, K370S | S364K, E357Q | FIG. 34 of US 2016/0355600 |
| | 12840 | | | |
| Fc 24 | Skew variant | K370S | S364K, E357Q | FIG. 34 of US 2016/0355600 |
| | 12841 | | | |
| Fc 25 | Skew variant | L368E, K370S | S364K | FIG. 34 of US 2016/0355600 |
| | 12894 | | | |
| Fc 26 | Skew variant | K370S | S364K | FIG. 34 of US 2016/0355600 |
| | 12895 | | | |
| Fc 27 | Skew variant | L368E, K370S | S364K, E357Q | FIG. 34 of US 2016/0355600 |
| | 12896 | | | |
| Fc 28 | Skew variant | K370S | S364K, E357Q | FIG. 34 of US 2016/0355600 |
| | 12901 | | | |
| Fc 29 | pl_IS0(−) | 1199T, N203D, | | FIG. 31 of US 2016/0355600 |
| | | K274Q, R355Q, | | |
| | | N384S, K392N, | | |
| | | V397M, Q419E, | | |
| | | DEL447 | | |
| Fc 30 | pl_(−)_Isosteric_A | N208D, Q295E, | | FIG. 31 of US 2016/0355600 |
| | | N384D, Q418E, | | |
| | | N421D | | |
| Fc 31 | pl_(−)_isosteric_B | N208D, Q295E, | | FIG. 31 of US 2016/0355600 |
| | | Q418E, N421D | | |
| Fc 32 | pl_IS0(+RR) | Q196K, I199T, | | FIG. 31 of US 2016/0355600 |
| | | P217R, P228R, | | |
| | | N276K | | |
| Fc 33 | pl_IS0(+) | Q196K, I199T, | | FIG. 31 of US 2016/0355600 |
| | | N276K | | |
| Fc 34 | pl_(+) | E269Q, E272Q, | | FIG. 31 of US 2016/0355600 |
| | isosteric_A | E283Q, E357Q, | | |
| Fc 35 | pl_(+)_isosteric_B | E269Q, E272Q, | | FIG. 31 of US 2016/0355600 |
| | | E283Q | | |
| Fc 36 | pl_(+) | E269Q, E272Q | | FIG. 31 of US 2016/0355600 |
| | isosteric_E269Q, | | | |
| | E272Q | | | |
| Fc 37 | pl_(+)_isosteric_E | E269Q, E283Q | | FIG. 31 of US 2016/0355600 |
| | 269Q, E283Q | | | |
| Fc 38 | pl_(+) | E272Q, E283Q | | FIG. 31 of US 2016/0355600 |
| | isosteric_E2720, | | | |
| | E283Q | | | |
| Fc 39 | pl_(+)_isosteric_E | E269Q | | FIG. 31 of US 2016/0355600 |
| | 269Q | | | |
| Fc 40 | Heterodimerization | F405A | T394F | FIG. 30A of US 2016/0355600 |
| Fc 41 | Heterodimerization | S364D | Y349K | FIG. 30A of US 2016/0355600 |
| Fc 42 | Heterodimerization | S364E | L368K | FIG. 30A of US 2016/0355600 |
| Fc 43 | Heterodimerization | S364E | Y349K | FIG. 30A of US 2016/0355600 |
| Fc 44 | Heterodimerization | S364F | K370G | FIG. 30A of US 2016/0355600 |
| Fc 45 | Heterodimerization | S364H | Y349K | FIG. 30A of US 2016/0355600 |
| Fc 46 | Heterodimerization | S364H | Y349T | FIG. 30A of US 2016/0355600 |
| Fc 47 | Heterodimerization | S364Y | K370G | FIG. 30A of US 2016/0355600 |
| Fc 48 | Heterodimerization | T411K | K370E | FIG. 30A of US 2016/0355600 |
| Fc 49 | Heterodimerization | V397S, F405A | T394F | FIG. 30A of US 2016/0355600 |
| Fc 50 | Heterodimerization | K370R, T411K | K370E, T411E | FIG. 30A of US 2016/0355600 |
| Fc 51 | Heterodimerization | L351E, S364D | Y349K, L351K | FIG. 30A of US 2016/0355600 |
| Fc 52 | Heterodimerization | L351E, S364E | Y349K, L351K | FIG. 30A of US 2016/0355600 |
| Fc 53 | Heterodimerization | L351E, T366D | L351K, T366K | FIG. 30A of US 2016/0355600 |
| Fc 54 | Heterodimerization | P395T, V397S, | T394F | FIG. 30A of US 2016/0355600 |
| | | F405A | | |
| Fc 55 | Heterodimerization | S364D, K370G | S364Y, K370R | FIG. 30A of US 2016/0355600 |
| Fc 56 | Heterodimerization | S364D, T394F | Y349K, F405A | FIG. 30A of US 2016/0355600 |
| Fc 57 | Heterodimerization | S364E, F405A | Y349K, T394F | FIG. 30A of US 2016/0355600 |
| Fc 58 | Heterodimerization | S364E, F405S | Y349K, T394Y | FIG. 30A of US 2016/0355600 |
| Fc 59 | Heterodimerization | S364E, T411E | Y349K, D401K | FIG. 30A of US 2016/0355600 |
| | | | US | |
| Fc 60 | Heterodimerization | S364H, D401K | Y349T, T411E | FIG. 30A of US 2016/0355600 |
| Fc 61 | Heterodimerization | S364H, F405A | Y349T, T394F | FIG. 30A of US 2016/0355600 |
| Fc 62 | Heterodimerization | S364H, T394F | Y349T, F405A | FIG. 30A of US 2016/0355600 |
| Fc 63 | Heterodimerization | Y349C, S364E | Y349K, S354C | FIG. 30A of US 2016/0355600 |
| Fc 64 | Heterodimerization | L351E, S364D, | Y349K, L351K, | FIG. 30A of US 2016/0355600 |
| | | F405A | T394F | |
| Fc 65 | Heterodimerization | L351K, S364H, | Y349T, L351E, | FIG. 30A of US 2016/0355600 |
| | | D401K | T411E | |
| Fc 66 | Heterodimerization | S364E, T411E, | Y349K, T394F, | FIG. 30A of US 2016/0355600 |
| | | F405A | D401K | |
| Fc 67 | Heterodimerization | S364H, D401K, | Y349T, T394F, | FIG. 30A of US 2016/0355600 |
| | | F405A | T411E | |
| Fc 68 | Heterodimerization | S364H, F405A, | Y349T, T394F, | FIG. 30A of US 2016/0355600 |
| | | T411E | D401K | |
| Fc 69 | Heterodimerization | T411E, K360E, | D401K | FIG. 30C of US 2016/0355600 |
| | | N390D | | |
| Fc 70 | Heterodimerization | T411E, Q362E, | D401K | FIG. 30C of US 2016/0355600 |
| | | N390D | | |
| Fc 71 | Heterodimerization | T411E, Q347R | D401K, K360D | FIG. 30C of US 2016/0355600 |
| Fc 72 | Heterodimerization | T411E, Q347R | D401K, K360E | FIG. 30C of US 2016/0355600 |
| Fc 73 | Heterodimerization | T411E, K360 | D401K, Q347K | FIG. 30C of US 2016/0355600 |
| Fc 74 | Heterodimerization | T411E, K360D | D401K, Q347R | FIG. 30C of US 2016/0355600 |
| Fc 75 | Heterodimerization | T411E, K360E | D401K, Q347K | FIG. 30C of US 2016/0355600 |
| Fc 76 | Heterodimerization | T411E, K360E | D401K, Q347R | FIG. 30C of US 2016/0355600 |
| Fc 77 | Heterodimerization | T411E, S364K | D401K, K370S | FIG. 30C of US 2016/0355600 |
| Fc 78 | Heterodimerization | T411E, K370S | D401K, S364K | FIG. 30C of US 2016/0355600 |
| Fc 79 | Heterodimerization | Q347E | E357Q | FIG. 30C of US 2016/0355600 |
| Fc 80 | Heterodimerization | Q347E | E357Q, Q362K | FIG. 30C of US 2016/0355600 |
| Fc 81 | Heterodimerization | K360D, Q362E | Q347R | FIG. 30C of US 2016/0355600 |
| Fc 82 | Heterodimerization | K360D, Q362E | D401K | FIG. 30C of US 2016/0355600 |
| Fc 83 | Heterodimerization | K360D, Q362E | Q347R, D401K | FIG. 30C of US 2016/0355600 |
| Fc 84 | Heterodimerization | K360E, Q362E | Q347R | FIG. 30C of US 2016/0355600 |
| Fc 85 | Heterodimerization | K360E, Q362E | D401K | FIG. 30C of US 2016/0355600 |
| Fc 86 | Heterodimerization | K360E, Q362E | Q347R, D401K | FIG. 30C of US 2016/0355600 |
| Fc 87 | Heterodimerization | Q362E, N390D | D401K | FIG. 30C of US 2016/0355600 |
| Fc 88 | Heterodimerization | Q347E, K360D | D401N | FIG. 30C of US 2016/0355600 |
| Fc 89 | Heterodimerization | K360D | Q347R, N390K | FIG. 30C of US 2016/0355600 |
| Fc 90 | Heterodimerization | K360D | N390K, D401N | FIG. 30C of US 2016/0355600 |
| Fc 91 | Heterodimerization | K360E | Y349H | FIG. 30C of US 2016/0355600 |
| Fc 92 | Heterodimerization | K370S, Q347E | S364K | FIG. 30C of US 2016/0355600 |
| Fc 93 | Heterodimerization | K370S, E357L | S364K | FIG. 30C of US 2016/0355600 |
| Fc 94 | Heterodimerization | K370S, E357Q | S364K | FIG. 30C of US 2016/0355600 |
| Fc 95 | Heterodimerization | K370S, Q347E, | S364K | FIG. 30C of US 2016/0355600 |
| | | E357L | | |
| Fc 96 | Heterodimerization | K370S, Q347E, | S364K | FIG. 30C of US 2016/0355600 |
| | | E357Q | | |
| Fc 97 | Heterodimerization | L368D, K370S, | S364K | FIG. 30D of US 2016/0355600 |
| | | Q347E | | |
| Fc 98 | Heterodimerization | L368D, K370S, | S364K | FIG. 30D of US 2016/0355600 |
| | | E357L | | |
| Fc 99 | Heterodimerization | L368D, K370S, | S364K | FIG. 30D of US 2016/0355600 |
| | | E357Q | | |
| Fc 100 | Heterodimerization | L368D, K370S, | S364K | FIG. 30D of US 2016/0355600 |
| | | Q347E, E357L | | |
| Fc 101 | Heterodimerization | L368D, K370S, | S364K | FIG. 30D of US 2016/0355600 |
| | | Q347E, E357Q | | |
| Fc 102 | Heterodimerization | L368E, K370S, | S364K | FIG. 30D of US 2016/0355600 |
| | | Q347E | | |
| Fc 103 | Heterodimerization | L368E, K370S, | S364K | FIG. 30D of US 2016/0355600 |
| | | E357L | | |
| Fc 104 | Heterodimerization | L368E, K370S, | S364K | FIG. 30D of US 2016/0355600 |
| | | E357Q | | |
| Fc 105 | Heterodimerization | L368E, K370S, | S364K | FIG. 30D of US 2016/0355600 |
| | | Q347E, E357L | | |
| Fc 106 | Heterodimerization | L368E, K370S, | S364K | FIG. 30D of US 2016/0355600 |
| | | Q347E, E357Q | | |
| Fc 107 | Heterodimerization | L368D, K370T, | S364K | FIG. 30D of US 2016/0355600 |
| | | Q347E | | |
| Fc 108 | Heterodimerization | L368D, K370T, | S364K | FIG. 30D of US 2016/0355600 |
| | | E357L | | |
| Fc 109 | Heterodimerization | L368D, K370T, | S364K | FIG. 30D of US 2016/0355600 |
| | | E357Q | | |
| Fc 110 | Heterodimerization | L368D, K370T, | S364K | FIG. 30D of US 2016/0355600 |
| | | Q347E, E357L | | |
| Fc 111 | Heterodimerization | L368D, K370T, | S364K | FIG. 30D of US 2016/0355600 |
| | | Q347E, E357Q | | |
| Fc 112 | Heterodimerization | L368E, K370T, | S364K | FIG. 30D of US 2016/0355600 |
| | | Q347E | | |
| Fc 113 | Heterodimerization | L368E, K370T, | S364K | FIG. 30D of US 2016/0355600 |
| | | E357L | | |
| Fc 114 | Heterodimerization | L368E, K370T, | S364K | FIG. 30D of US 2016/0355600 |
| | | E357Q | | |
| Fc 115 | Heterodimerization | L368E, K370T, | S364K | FIG. 30D of US 2016/0355600 |
| | | Q347E, E357L | | |
| Fc 116 | Heterodimerization | L368E, K370T, | S364K | FIG. 30D of US 2016/0355600 |
| | | Q347E, E357Q | | |
| Fc 117 | Heterodimerization | T411E, Q362E | D401K, T411K | FIG. 30D of US 2016/0355600 |
| Fc 118 | Heterodimerization | T411E, N390D | D401K, T411K | FIG. 30D of US 2016/0355600 |
| Fc 119 | Heterodimerization | T411E, Q362E | D401R, T411R | FIG. 30D of US 2016/0355600 |
| Fc 120 | Heterodimerization | T411E, N390D | D401R, T411R | FIG. 30D of US 2016/0355600 |
| Fc 121 | Heterodimerization | Y407T | T366Y | FIG. 30D of US 2016/0355600 |
| Fc 122 | Heterodimerization | F405A | T394W | FIG. 30D of US 2016/0355600 |
| Fc 123 | Heterodimerization | T366Y, F405A | T394W, Y407T | FIG. 30D of US 2016/0355600 |
| Fc 124 | Heterodimerization | T3665, L368A, | T366W | FIG. 30D of US 2016/0355600 |
| | | Y407V | | |
| Fc 125 | Heterodimerization | T366S, L368A, | T366W, S354C | FIG. 30D of US 2016/0355600 |
| | | Y407V, Y349C | | |
| Fc 126 | Heterodimerization | K392D, K409D | E356K, D399K | FIG. 30E of US 2016/0355600 |
| Fc 127 | Heterodimerization | K370D, K392D, | E356K, E357K, | FIG. 30E of US 2016/0355600 |
| | | K409D | D399K | |
| Fc 128 | Heterodimerization | 1199T, N203D, | Q196K, L99T, | FIG. 30E of US 2016/0355600 |
| | | K247Q, R355Q, | P217R, P228R, | |
| | | N384S, K392N, | N276K | |
| | | V397M, Q419E, | | |
| | | K447 | | |
| Fc 129 | Heterodimerization | 1199T, N203D, | Q196K, L99T, | FIG. 30E of US 2016/0355600 |
| | | K247Q, R355Q, | N276K | |
| | | N384S, K392N, | | |
| | | V397M, Q419E, | | |
| | | K447 | | |
| Fc 130 | Heterodimerization | N384S, K392N, | N276K | FIG. 30E of US 2016/0355600 |
| | | V397M, Q419E | | |
| Fc 131 | Heterodimerization | D221E, P228E, | D221R, P228R, | FIG. 30E of US 2016/0355600 |
| | | L368E | K409R | |
| Fc 132 | Heterodimerization | C220E, P228E, | C220R, E224R, | FIG. 30E of US 2016/0355600 |
| | | L368E | P228R, K409R | |
| Fc 133 | Heterodimerization | F405L | K409R Figure | FIG. 30E of US 2016/0355600 |
| Fc 134 | Heterodimerization | T3661, K392M, | F405A, Y407V | FIG. 30E of US 2016/0355600 |
| | | T394W | | |
| Fc 135 | Heterodimerization | T366V, K409F | L351Y, Y407A | FIG. 30E of US 2016/0355600 |
| Fc 136 | Heterodimerization | T366A, K392E, | D399R, S400R, | FIG. 30E of US 2016/0355600 |
| | | K409F, T411E | Y407A | |
| Fc 137 | Heterodimerization | L351K | L351E | FIG. 30E of US 2016/0355600 |
| Fc 138 | Heterodimerization | I199T, N203D, | Q196K, L199T, | FIG. 30E of US 2016/0355600 |
| | | K247Q, R355Q, | P217R, P228R, | |
| | | Q419E, K447 | N276K | |
| Fc 139 | Heterodimerization | I199T, N203D, | Q196K, I199T, | FIG. 30E of US 2016/0355600 |
| | | K247Q, R355Q, | N276K | |
| | | Q419E, K447 | | |
| Fc 140 | Heterodimerization | I199T, N203D, | | FIG. 30E of US 2016/0355600 |
| | | K274Q, R355Q, | | |
| | | N384S, K392N, | | |
| | | V397M, Q419E | | |
| | | DEL447 | | |
| Fc 141 | Heterodimerization | N208D, Q295E | | FIG. 30E of US 2016/0355600 |
| | | N384D, Q418E | | |
| | | N421D | | |
| Fc 142 | Heterodimerization | N208D, Q295E | | FIG. 30E of US 2016/0355600 |
| | | Q418E, N421D | | |
| Fc 143 | Heterodimerization | Q196K, I199T | | FIG. 30E of US 2016/0355600 |
| | | P217R, P228R | | |
| | | N276K | | |
| Fc 144 | Heterodimerization | Q196K, I199T | | FIG. 30E of US 2016/0355600 |
| | | N276K | | |
| Fc 145 | Heterodimerization | E269Q, E272Q | | FIG. 30E of US 2016/0355600 |
| | | E283Q, E357Q | | |
| Fc 146 | Heterodimerization | E269Q, E272Q | | FIG. 30E of US 2016/0355600 |
| | | E283Q, | | |
| Fc 147 | Heterodimerization | E269Q, E272Q | | FIG. 30E of US 2016/0355600 |
| Fc 148 | Heterodimerization | E269Q, E283Q | | FIG. 30E of US 2016/0355600 |
| Fc 149 | Heterodimerization | E272Q, E283Q | | FIG. 30E of US 2016/0355600 |
| Fc 150 | Heterodimerization | E269Q | | FIG. 30E of US 2016/0355600 |

TABLE 1.3
Kappa Light Chain and Heavy Chain -
Constant Domain Mutations Pairs

| | Heavy Chain | Light Chain |
|---|---|---|
| Mutation Type | Constant Domain | Constant Domain |
| Charged pair | K147D | S131K |
| Charged pair | K147D | T180R |
| Charged pair | K147(WT) | T180E |
| Charged pair | T187D | N137K/N138R |
| Charged pair | T187D | N138R |
| Charged pair | T187D | N138K |
| Charged pair | K147(WT) | S131D |
| Charged pair | L145S | S131K |

All position information is reported using the EU numbering scheme
Wild type (WT) indicates the natural amino acid at the indicated position
Charge pairs with negative and positive charge residues could be reversed between heavy and light chains, where D or E (negative charge) are replaced by K or R (positive charge) and cognate chain K or R (positive charge) are replaced by D or E (negative charge).

TABLE 2
Kappa Light Chain and Heavy Chain -
Variable Domain Mutations Pairs

| | Heavy Chain Variable | Light Chain Variable |
|---|---|---|
| Mutation Type | Domain | Domain |
| Charged pair | Q39K | Q38D |
| Charged pair | Q39K | Q38E |
| Charged pair | Q39D | Q38K |
| Charged pair | Q39E | Q38K |
| Charged pair | G100K | G44D |
| Knob-into-Hole | Y87G | L45W |

All position information is reported using the Kabat numbering scheme
Wild type (WT) indicates the natural amino acid at the indicated position
Charged pairs with negative and positive charged residues could be reversed between heavy and light chains, where D or E (negative charged) are replaced by K or R (positive charged) and cognate chain K or R (positive charged) are replaced by D or E (negative charged).

TABLE 3
Lambda Light Chain and Heavy Chain -
Constant Domain Mutations Pairs

| | Heavy Chain Constant | Light Chain Constant |
|---|---|---|
| Mutation Type | Domain | Domain |
| Charged pair | V185K | S137D |
| Charged pair | V185E | S137K |
| Charged pair | K147D | T131R |
| Charged pair | K147D | S179R |
| Charged pair | K147D | S179K |
| Charged pair | K147D | T131K |
| Charged pair | L145S | T131K |
| Charged pair | K147(WT) | T131D |
| Charged pair | K147(WT) | S179E |

All position information is reported using the EU numbering scheme
Charge pairs with negative and positive charge residues could be reversed between heavy and light chains, where D or E (negative charge) are replaced by K or R (positive charge) and cognate chain K or R (positive charge) are replaced by D or E (negative charge).

TABLE 4
Lambda Light Chain and Heavy Chain -
Variable Domain Mutations Pairs

| | Heavy Chain Variable | Light Chain Variable |
|---|---|---|
| Mutation Type | Domain | Domain |
| Charged pair | Q39K | Q38D |
| Charged pair | Q39K | Q38E |
| Charged pair | Q39D | Q38K |
| Charged pair | Q39E | Q38K |
| Charged pair | G100D | G44K |
| Knob-into-Hole | Y87G | L45W |

All position information is reported using the Kabat numbering scheme
Charge pairs with negative and positive charge residues could be reversed between heavy and light chains, where D or E (negative charge) are replaced by K or R (positive charge) and cognate chain K or R (positive charge) are replaced by D or E (negative charge).

TABLE 5
Kappa Constant Chain Cysteine Mutation Pairs

| | Heavy Chain Constant | Light Chain Constant |
|---|---|---|
| Mutation Type | Domain | Domain |
| Cys-Cys | S131C | S114C |
| Cys-Cys | S136C | S114C |
| Cys-Cys | S134C | F116C |
| Cys-Cys | F170C | S162C |
| Cys-Cys | P171C | S162C |

All position information is reported using the EU numbering scheme

TABLE 6
Lambda Constant Chain Cysteine Mutation Pairs

| | Heavy Chain Constant | Light Chain Constant |
|---|---|---|
| Mutation Type | Domain | Domain |
| Cys-Cys | V173C | T162C |
| Cys-Cys | L128C | S118C |
| Cys-Cys | S134C | T116C |

All position information is reported using the EU numbering scheme

In certain embodiments, antibody variants comprise the following substitutions: i) the amino acid at positions 39 (Kabat numbering) of the VH1 and VH2 are charged or polar amino acid residues and the amino acid at positions 38 (Kabat numbering) of the VL1 and VL2 are an oppositely charged or polar amino acid residue compared to the amino acids at positions 39 of the VH1 and the VH2; or the amino acid at positions 100 of the VH1 and VH2 (Kabat numbering) are charged or polar amino acid residues and the amino acid at positions 44 (Kabat numbering) of the VL1 and VL2 are an oppositely charged or polar amino acid residue compared to the amino acids at positions 100 of the VH1 and the VH2; ii) the amino acid at positions 147 of the CH1_{H1} and the CH1_{H2} (EU numbering) are charged or polar amino acid residues and one of the amino acids at positions 131, 179 or 180 of the CL1 or CL2 (EU numbering) is an oppositely charged or polar amino acid residue compared to the amino acids at positions 147 of the CH1_{H1} and the CH1_{H2}; iii) the amino acid at positions 185 of the CH1_{H1} and the CH1_{H2} (EU numbering) are charged or polar amino acid residues and the amino acid at positions 137 of the CL1 and CL2 (EU numbering) are an oppositely charged or polar amino acid residue compared to the amino acids at positions 185 of the CHI_{H1} and the CH1_{H2}; or the amino acid at positions 187 of the CH1_{H1} and the CH1_{H2} (EU numbering) are charged or polar amino acid residues and one of the amino acids at positions 137 or 138 of the CL1 and CL2 (EU numbering) is an oppositely charged or polar amino acid residue compared to the amino acids at positions 187 of the CH1_{H1} and the CH1_{H2} (EU numbering); and iv) the amino acid at positions 145 of the CH1_{H1} and the CH1_{H2} (EU numbering) are charged or polar amino acid residues and the amino acids at position 131 of the CL1 and CL2 (EU numbering) are oppositely charged or polar amino acid residues compared to the amino acids at positions 145 of the CH1_{H1} and the CH1_{H2}.

In certain embodiments, antibody variants comprise the following substitutions: the H1 amino acids at position 39, 100, 147, 185, 187 or 145 are positively charged and the L1 amino acids at positions 38, 44, 131, 179, 180, 137 or 138 are negatively charged; and the H2 amino acids at position 39, 100, 147, 185, 187 or 145 are negatively charged and the L2 amino acids at positions 38, 44, 131, 179, 180, 137 or 138 are positively charged.

In certain embodiments, antibody variants comprise the following substitutions: the H1 amino acids at position 39, 100, 147, 185, 187 and 145 are negatively charged and the L1 amino acids at positions 38, 44, 131, 179, 180, 137 or 138 are positively charged; and the H2 amino acids at position 39, 100, 147, 185, 187 or 145 are positively charged and the L2 amino acids at positions 38, 44, 131, 179, 180, 137 or 138 are negatively charged.

In certain embodiments, the antibody variant comprises the “light chain pairing mutation set A” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 185 (EU numbering) of the CH1_{H1} is a K and the amino acid at position 137 (EU numbering) of the CL1 is a D; iii) the amino acid at position 128 (EU numbering) of the CH1_{H1} is a C and the amino acid at position 118 (EU numbering) of the CL1 is a C; and iv) the amino acid at position 220 (EU numbering) in the H1H is a S and the amino acid at position 214 (EU numbering) of the CL1 is a S; and b) the H2 and the L2 comprise the following: i) the amino acid at position 39 (Kabat numbering) of the VH2 is a D and the amino acid at position 38 (Kabat numbering) of the VL2 is a K; and ii) the amino acid at position 147 (EU numbering) of the CH2_{H1} is a D and the amino acid at position 180 (EU numbering) of the CL2 is a R.

In certain embodiments, the antibody variant comprises the “light chain pairing mutation set B” comprising the following substitutions: a) the H1 and the L1 comprise the following: i) the amino acid at position 39 (Kabat numbering) of the VH1 is a K and the amino acid at position 38 (Kabat numbering) of the VL1 is a D; and ii) the amino acid at position 185 (EU numbering) of the CH1_{H1} is a K and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 147 (EU numbering) of the CH2_{H1} is a D and the amino acid at position 180 (EU numbering) of the CL2 is a R; iii) the amino acid at position 134 (EU numbering) of the CH2_{H1} is a C and the amino acid at position 116 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set C” comprising the following substitutions: a) the H1 and the L1 comprise the following: i) the amino acid at position 39 (Kabat numbering) of the VH1 is a K and the amino acid at position 38 (Kabat numbering) of the VL1 is a D; and ii) the amino acid at position 185 (EU numbering) of the CH1_{H1} is a K and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 147 (EU numbering) of the CH2_{H1} is a D and the amino acid at position 180 (EU numbering) of the CL2 is a R; iii) the amino acid at position 136 (EU numbering) of the CH2_{H1} is a C and the amino acid at position 114 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set D” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a K and the amino acid at position 131 (EU numbering) of the CL1 is a D; iii) the amino acid at position 173 (EU numbering) of the CH1_{H1} is a C and the amino acid at position 162 (EU numbering) of the CL1 is a C; iv) the amino acid at position 220 (EU numbering) in the H1H is a S and the amino acid at position 214 (EU numbering) of the CL1 is a S; and b) the H2 and the L2 comprise the following: i) the amino acid at position 39 (Kabat numbering) of the VH2 is a D and the amino acid at position 38 (Kabat numbering) of the VL2 is a K; and ii) the amino acid at position 147 (EU numbering) of the CH2_{H1} is a D and the amino acid at position 180 (EU numbering) of the CL2 is a R.

In certain embodiments, the antibody variant comprises the “light chain pairing mutation set E” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 185 (EU numbering) of the CH1_{H1} is a K and the amino acid at position 137 (EU numbering) of the CL1 is a D; iii) the amino acid at position 173 (EU numbering) of the CH1_{H1} is a C and the amino acid at position 162 (EU numbering) CL1 is a C; and iv) the amino acid at position 220 (EU numbering) in the H1H is a S and the amino acid at position 214 (EU numbering) of the CL1 is a S; and b) the H2 and the L2 comprise the following: i) the amino acid at position 39 (Kabat numbering) of the VH2 is a D and the amino acid at position 38 (Kabat numbering) of the VL2 is a K; and ii) the amino acid at position 147 (EU numbering) of the CH2_{H1} is a D and the amino acid at position 180 (EU numbering) of the CL2 is a R.

In certain embodiments, the antibody variant comprises the “light chain pairing mutation set F” comprising the following substitutions: a) the H1 and the L1 comprise the following: i) the amino acid at position 39 (Kabat numbering) of the VH1 is a K and the amino acid at position 38 (Kabat numbering) of the VL1 is a D; and ii) the amino acid at position 185 (EU numbering) of the CH1_{H1} is a K and the amino acid at position 137 (EU numbering) CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 147 (EU numbering) of the CH2_{H1} is a D and the amino acid at position 180 (EU numbering) of the CL2 is a R; iii) the amino acid at position 131 (EU numbering) of the CH2_{H1} is a C and the amino acid at position 114 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set G” comprising the following substitutions: a) the H1 and the L1 comprise the following: i) the amino acid at position 39 (Kabat numbering) of the VH1 is a K and the amino acid at position 38 (Kabat numbering) of the VL1 is a D; and ii) the amino acid at position 185 (EU numbering) of the CH1_{H1} is a K and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH2_{H1} is a D and the amino acid at position 138 (EU numbering) of the CL2 is a K; ii) the amino acid at position 170 (EU numbering) of the CH2_{H1} is a C and the amino acid at position 162 EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set H” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 185 (EU numbering) of the CH1_{H1} is a E, the amino acid at position 137 (EU numbering) of the CL1 is a K; and iii) the amino acid at position 179 (EU numbering) of the CL1 is a E; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH2_{H1} is a D and the amino acid at position 138 (EU numbering) of the CL2 is a K; iii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set I” comprising the following substitutions: a) the H1 and the L1 comprise the following: i) the amino acid at position 39 (Kabat numbering) of the VH1 is a K and the amino acid at position 38 (Kabat numbering) of the VL1 is a D; and ii) the amino acid at position 185 (EU numbering) of the CH1_{H1} is a K and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH2_{H1} is a D and the amino acid at position 138 (EU numbering) of the CL2 is a K; iii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set J” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 185 (EU numbering) of the CH1_{H1} is a E and the amino acid at position 137 (EU numbering) of the CL1 is a K; and iii) the amino acid at position 179 (EU numbering) of the CL1 is a E; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 147 (EU numbering) of the CH2_{H1} is a D and the amino acid at position 180 (EU numbering) of the CL2 is a R; iii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set K” comprising the following substitutions: a) the H1 and the L1 comprise the following: i) the amino acid at position 39 (Kabat numbering) of the VH1 is a K and the amino acid at position 38 (Kabat numbering) of the VL1 is a D; and ii) the amino acid at position 185 (EU numbering) of the CHI_{H1} is a K and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 147 (EU numbering) of the CH2_{H1} is a D and the amino acid at position 180 (EU numbering) of the CL2 is a R; iii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set L” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a K and the amino acid at position 131 (EU numbering) of the CL1 is a D; iii) the amino acid at position 185 (EU numbering) of the CH1_{H1} is a K and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 147 (EU numbering) of the CH2_{H1} is a D and the amino acid at position 180 (EU numbering) of the CL2 is a R; ii) the amino acid at position 171 (EU numbering) of the CH2_{H1} is a C and the amino acid at position 162 (EU numbering) of the CL2 is a C; and the amino acid at position 220 (EU numbering) in the H2H is a S and the amino acid at position 214 (EU numbering) of the CL2 is a S.

In certain embodiments, the antibody variant comprises the “light chain pairing mutation set 0340” comprising the following substitutions: a) the H1 and the L1 comprise the following: i) the amino acid at position 39 (Kabat numbering) of the VH1 is a D and the amino acid at position 38 (Kabat numbering) of the VL1 is a K; and i) the amino acid at position 185 (EU numbering) of the CH1_{H1} is a E and the amino acid at position 137 (EU numbering) of the CL1 is a K; and b) the H2 and the L2 comprise the following: i) the amino acid at position 39 (Kabat numbering) of the VH2 is a K and the amino acid at position 38 (Kabat numbering) of the VL2 is a D; ii) the amino acid at position 187 (EU numbering) of the CH2_{H1} is a D and the amino acid at position 138 (EU numbering) of the CL2 is a K; iii) the amino acid at position 136 (EU numbering) of the CH2_{H1} is a C and the amino acid at position 114 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set M” comprising the following substitutions: a) the H1 and the L1 comprise the following: i) the amino acid at position 39 (Kabat numbering) of the VH1 is a D and the amino acid at position 38 (Kabat numbering) of the VL1 is a K; and i) the amino acid at position 185 (EU numbering) of the CH1_{H1} is a E and the amino acid at position 137 (EU numbering) of the CL1 is a K; and b) the H2 and the L2 comprise the following: i) the amino acid at position 39 (Kabat numbering) of the VH2 is a K and the amino acid at position 38 (Kabat numbering) of the VL2 is a D; ii) the amino acid at position 187 (EU numbering) of the CH2_{H1} is a D and the amino acid at position 138 (EU numbering) of the CL2 is a K; ii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set N” comprising the following substitutions: a) the H1 and the L1 comprise the following: i) the amino acid at position 39 (Kabat numbering) of the VH1 is a D and the amino acid at position 38 (Kabat numbering) of the VL1 is a K; and ii) the amino acid at position 185 (EU numbering) of the CH1_{H1} is a K and the amino acid at position 137 (EU numbering) of the CL1 is a D; and b) the H2 and the L2 comprise the following: i) the amino acid at position 39 (Kabat numbering) of the VH2 is a K and the amino acid at position 38 (Kabat numbering) of the VL2 is a D; i) the amino acid at position 187 (EU numbering) of the CH2_{H1} is a D and the amino acid at position 138 (EU numbering) of the CL2 is a K; iii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is an S.

In certain embodiments, the antibody variant comprises the “light chain pairing mutation set O” comprising the following substitutions: a) the H1 and the L1 comprise the following: i) the amino acid at position 39 (Kabat numbering) of the VH1 is a D and the amino acid at position 38 (Kabat numbering) of the VL1 is a K; ii) the amino acid at position 147 (EU numbering) of the CH1_{H1} is a K and the amino acid at position 131 (EU numbering) of the CL1 is a D; iii) the amino acid at position 185 (EU numbering) of the CH1_{H1} is a E and the amino acid at position 137 (EU numbering) of the CL1 is a K; and iv) the amino acid at position 145 (EU numbering) of the CH1_{H1} is a S and the amino acid at position 180 (EU numbering) of the CL1 is a E; and b) the H2 and the L2 comprise the following: i) the amino acid at position 39 (Kabat numbering) of the VH2 is a K and the amino acid at position 38 (Kabat numbering) of the VL2 is a D; ii) the amino acid at position 187 (EU numbering) of the CH2_{H1} is a D and the amino acid at position 138 (EU numbering) of the CL2 is a K; iii) the amino acid at position 170 (EU numbering) of the VH2 is a C and the amino acid at position 162 (EU numbering) of the VL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set 367” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a D and the amino acid at position 131 (EU numbering) of the CL1 is a K; and iii) the amino acid at position 185 (EU numbering) of the CH1_{H1} is a E and the amino acid at position 137 (EU numbering) of the CL1 is a D; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH2_{H1} is a D and the amino acid at position 137 (EU numbering) of the CL2 is a K; iii) the amino acid at position 138 (EU numbering) of the CL2 is a R; iv) the amino acid at position 170 (EU numbering) of the CH2_{H1} is a C and the amino acid at position 162 (EU numbering) of the CL2 is a C; and v) the amino acid at position 220 (EU numbering) in the H2H is a S and the amino acid at position 214 (EU numbering) of the CL2 is a S.

In certain embodiments, the antibody variant comprises the “light chain pairing mutation set P” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a D and the amino acid at position 131 (EU numbering) of the CL1 is a K; and iii) the amino acid at position 145 (EU numbering) of the CH1_{H1} is a S; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH2_{H1} is a D and the amino acid at position 138 (EU numbering) of the CL2 is a K; iii) the amino acid at position 170 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set 404” comprising the following substitutions: a) the H1 and the L1 comprise the following: i) the amino acid at position 39 (Kabat numbering) of the VH1 is a D and the amino acid at position 38 (Kabat numbering) of the VL1 is a K; ii) the amino acid at position 147 (EU numbering) of the CH1_{H1} is a K and the amino acid at position 131 (EU numbering) of the CL1 is a D; and iii) the amino acid at position 185 (EU numbering) of the CH1_{H1} is a E and the amino acid at position 137 (EU numbering) of the CL1 is a K; and b) the H2 and the L2 comprise the following: i) the amino acid at position 39 (Kabat numbering) of the VH2 is a K and the amino acid at position 38 (Kabat numbering) of the VL2 is a D; ii) the amino acid at position 147 (EU numbering) of the CH2_{H1} is a D and the amino acid at position 180 (EU numbering) of the CL2 is a R; i) the amino acid at position 136 (EU numbering) of the CH2_{H1} is a C and the amino acid at position 114 (EU numbering) of the CL2 is a C; and iv) the amino acid at position 220 (EU numbering) in the H2H is a S and the amino acid at position 214 (EU numbering) of the CL2 is an S.

In certain embodiments, the antibody variant comprises the “light chain pairing mutation set 406” comprising the following substitutions: a) the H1 and the L1 comprise the following: i) the amino acid at position 39 (Kabat numbering) of the VH1 is a K and the amino acid at position 38 (Kabat numbering) of the VL1 is a E; ii) the amino acid at position 147 (EU numbering) of the CH1_{H1} is a K and the amino acid at position 131 (EU numbering) of the CL1 is a D; and iii) the amino acid at position 185 (EU numbering) of the CH1_{H1} is a E and the amino acid at position 137 (EU numbering) of the CL1 is a K; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH2_{H1} is a D and the amino acid at position 138 (EU numbering) of the CL2 is a K; iii) the amino acid at position 136 (EU numbering) of the CH2_{H1} is a C and the amino acid at position 114 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set 473” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 147 (EU numbering) of the CH1_{H1} is a D and the amino acid at position 131 (EU numbering) of the CL1 is a K; and iii) the amino acid at position 185 (EU numbering) of the CH1_{H1} is a D and the amino acid at position 137 (EU numbering) of the CL1 is a K; and b) the H2 and the L2 comprise the following: i) the amino acid at position 39 (Kabat numbering) of the VH2 is a D and the amino acid at position 38 (Kabat numbering) of the VL2 is a K; ii) the amino acid at position 187 (EU numbering) of the CH21 is a D and the amino acid at position 138 (EU numbering) of the CL2 is a K; iii) the amino acid at position 171 (EU numbering) of the CH2_{H1} is a C and the amino acid at position 162 (EU numbering) of the CL2 is a C; and iv) the amino acid at position 220 (EU numbering) in the H2H is a S and the amino acid at position 214 (EU numbering) of the CL2 is a S.

In certain embodiments, the antibody variant comprises the “light chain pairing mutation set Q” comprising the following substitutions: a) the H1 and the L1 comprise the following: i) the amino acid at position 39 (Kabat numbering) of the VH1 is a K and the amino acid at position 38 (Kabat numbering) of the VL1 is a D; ii) the amino acid at position 170 (EU numbering) of the CH1_{H1} is a S and the amino acid at position 131 (EU numbering) of the CL1 is a D; iii) the amino acid at position 173 (EU numbering) of the CH1_{H1} is a C and the amino acid at position 162 (EU numbering) of the CL1 is a C; iv) the amino acid at position 220 (EU numbering) in the H1H is a S and the amino acid at position 214 (EU numbering) of the CL1 is a S; and b) the H2 and the L2 comprise the following: i) the amino acid at position 39 (Kabat numbering) of the VH2 is a D and the amino acid at position 38 (Kabat numbering) of the VL2 is a K; and ii) the amino acid at position 147 (EU numbering) of the CH2_{H1} is a D and the amino acid at position 180 (EU numbering) of the CL2 is a R.

It can be desirable to modify an antibody disclosed herein with respect to effector function, so as to enhance, e.g., the effectiveness of the antibody in treating diseases and disorders. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J Exp Med., 176:1191-1195 (1992) and Shopes, J. Immunol., 148:2918-2922. (1992)). Alternatively, an antibody can be engineered that has dual Fc regions and can thereby have enhanced complement lysis and ADCC capabilities. (See Stevenson et al., Anti-Cancer Drug Design, 3:219-230 (1989)).

Certain antibody variants with improved or diminished binding to FcRs are described. (See, e.g., U.S. Pat. No. 6,737,056; WO 2004/056312, and Shields et al., J. Biol. Chem. 9 (2):6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent

Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, antibodies may comprise a substitution mutation in the Fc region that reduces effector function. In some embodiments, the substitution mutation is an aglycosylation site mutation. In some embodiments, the aglycosylation site mutation is at amino acid residue 297 and amino acid substitutions at residues 234, 235, 265 and 331 (EU numbering) to disrupt the Fc receptor binding interface. In some embodiments, the aglycosylation site mutation reduces effector function of the antibody.

In some embodiments, i) the CH1_{H3} and/or the CH2_{H3} has an A at position 297 (EU numbering) ii) the CH1_{H3} and/or the CH2_{H3} has a G at position 297 (EU numbering); or iii) the CH1_{H3} and/or the CH2_{H3} has a S at position 297 (EU numbering). In some embodiments, the CH1_{H3} and/or the CH2_{H3} has an S at position 331 (EU numbering).

In some embodiments, i) the H1H and/or the H2H has an A at positions 234 and 235 (EU numbering); or ii) the H1H and/or the H2H has an A at positions 234, 235 and 237 (EU numbering) iii) the H1H and/or the H2H has an A at positions 234 and 235 and G at position 329 (EU numbering).

In some embodiments, the antibodies may comprise as substitution mutation in the Fc region that can improve expression titers and increased homogeneity of the antibody post-purification. In some embodiments, the antibody comprises a variant human IgG4 Fc domain. In some embodiments, i) the CH1_{H2} and/or the CH2_{H2} has an C at position 370 (Kabat numbering); and ii) the CH1_{H2} and/or the CH2_{H2} has an C at position 375 (Kabat numbering).

The use of knobs into holes as a method of producing multispecific antibodies is well known in the art. See U.S. Pat. No. 5,731,168 granted 24 Mar. 1998 assigned to Genentech, PCT Pub. No. WO2009089004 published 16 Jul. 2009 and assigned to Amgen, and US Pat. Pub. No. 20090182127 published 16 Jul. 2009 and assigned to Novo Nordisk A/S. See also Marvin and Zhu, Acta Pharmacologica Sincia (2005) 26(6):649-658 and Kontermann (2005) Acta Pharacol. Sin., 26:1-9.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimeric antibody, and thereby favor heteromultimeric antibody formation over homomultimeric antibody formation, for example. The protuberance may exist in the original interface or may be introduced synthetically (e.g. by altering nucleic acid encoding the interface). Normally, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The upper limit for the number of original residues which are replaced is the total number of residues in the interface of the first polypeptide.

The preferred import residues for the formation of a protuberance are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the protuberance has a small side chain volume, such as alanine, asparagine, aspartic acid, glycine, serine, threonine or valine. Exemplary amino acid substitutions in the CH1_{H3} or CH2_{H3} domain for forming the protuberance include without limitation the T366W substitution.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). Normally, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide. The side chain volumes of the various amino residues are shown in Table 3 above. The preferred import residues for the formation of a cavity are usually naturally occurring amino acid residues and are preferably selected from alanine (A), serine(S), threonine (T) and valine (V). Most preferred are serine, alanine or threonine. In one embodiment, the original residue for the formation of the cavity has a large side chain volume, such as tyrosine, arginine, phenylalanine or tryptophan. Exemplary amino acid substitutions in the CH1_{H3} or CH2_{H3} domain for generating the cavity include without limitation the T366S, L368A, Y407A, Y407T and Y407V substitutions. In certain embodiments, the knob half-antibody comprises T366W substitution, and the hole half-antibody comprises the T366S/L368A/Y407V substitutions.

In certain embodiments, the antibody variant comprises the following substitutions: the CH1_{H3} has a C at position 349, an S at position 366, an A at position 368 and a V at position 407 (EU numbering); and the CH2_{H3} has a C at position 354 and a W at position 366 (EU numbering).

In certain embodiments, the antibody variant comprises the following substitutions: the CH2_{H3} has a C at position 349, an S at position 366, an A at position 368 and a V at position 407 (EU numbering); and the CH1_{H3} has a C at position 354 and a W at position 366 (EU numbering).

In certain embodiments, the antibody variant comprises the following substitutions: the CH1_{H3} has a C at position 354, an S at position 366, an A at position 368 and a V at position 407 (EU numbering); and the CH2_{H3} has a C at position 349 and a W at position 366 (EU numbering);

In certain embodiments, the antibody variant comprises the following substitutions: the CH2_{H3} has a C at position 354, an S at position 366, an A at position 368 and a V at position 407 (EU numbering); and the CH1_{H3} has a C at position 349 and a W at position 366 (EU numbering).

### T-Cell Surface Antigens

The present disclosure provides an antibody comprising a first antigen binding domain that binds to a cell surface antigen expressed on a T-cell, a NK cell, a neutrophil, a B cell or a dendritic cell engager cell, and a second antigen binding domain that binds to a disease associated antigen (DAA). In some embodiments, the cell surface antigen is expressed on a T-cell. Exemplary T-cell surface antigens include but are not limited to CD3. In some embodiments, the T-cell surface antigen is CD3. In some embodiments, the T-cell surface antigen is CD38.

### Cluster of Differentiation 3 (CD3)

In some embodiments, the invention is based, in part, on anti-CD3 antibodies. In certain embodiments, the anti-CD3 antibodies are multispecific (e.g., bispecific) and bind, in addition to CD3 or a fragment thereof, a second biological molecule (e.g., a cell surface antigen, e.g., a disease associated antigen). Antibodies of the invention are useful, for example, for treating or delaying the progression of a cell proliferative disorder (e.g., cancer) or an autoimmune disorder, or for enhancing immune function in a subject having such a disorder.

The term “cluster of differentiation 3” or “CD3,” as used herein, refers to any native CD3 from any vertebrate source, including mammals such as primates (e.g. humans, cynomolgus monkey) and rodents (e.g., mice and rats), unless otherwise indicated, including, for example, CD3ε, CD3γ, CD3α, and CD3β chains. CD3 is a cell surface complex expressed on T cells in association with the T cell receptor. The CD3 complex is required for the activation of CD8+ and CD4+ T lymphocytes. It is formed of three different but highly related chains: one CD3 gamma chain, one CD3 delta chain, and two CD3 epsilon chains, which associate with each other to form a CD3 epsilon/gamma heterodimer, and a CD3 epsilon/delta heterodimer. The two CD3 heterodimers, together with the T cell receptor (TCR) and the signal-transducing zeta chain homodimer form the T cell receptor complex.

The term encompasses “full-length” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length.

In some embodiments, the invention provides isolated antibodies that bind to CD3. In some embodiments, the invention provides antibodies that bind to CD3ε. In some instances the anti-CD3ε antibody binds to a human CD3ε polypeptide or a cynomolgus monkey (cyno) CD3ε polypeptide. In some instances, the human CD3 polypeptide or the cyno CD3 polypeptide is a human CD3ε polypeptide (SEQ ID NO: 419) or a cyno CD3ε polypeptide (SEQ ID NO: 420), respectively. In some instances, the anti-CD3 antibody binds to an epitope within a fragment of CD3ε (e.g., human CD3ε) consisting of amino acid residues 1-26 or amino acid residues 1-27 of human CD3ε (SEQ ID NO: 419).

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

### Anti-CD3ε Antibodies

Provided herein are anti-CD3ε antibodies. In some embodiments, alanine scanning mutagenesis was performed on the “SP34” anti-CD3ε antibody to produce affinity modulated anti-CD3ε antibodies of the invention.

In some embodiments, a anti-CD3ε antibody of the disclosure comprises any one of the VH and VL sequences listed in Table 7. In Table 7, the underlined sequences are CDR sequence according to Kabat and the bolded sequences are CDR sequences according to Chothia.

In some embodiments, a anti-CD3 antibody of the disclosure comprises: a) a heavy chain variable region (VH) comprising a VH complementarity determining region 1 (VH_{CDR1}), a VH complementarity determining region 2 (VH_{CDR2}) and a VH complementarity determining region 3 (VH_{CDR3}); and b) a light chain variable region (VL) comprising a VL complementarity determining region 1 (VL_{CDR1}), a VL complementarity determining region 2 (VL_{CDR2}) and a VL complementarity determining region 3 (VL_{CDR3}). Tables 8 and 9 provide exemplary of CDR sequences of the anti-CD3 antibodies provided herein.

TABLE 7
Anti-CD3 Variable Heavy Chain and Variable Light Chain Domains

| Ab | Domain | Amino Acid Sequence | SEQ ID NO |
|---|---|---|---|
| CD3-01 | CD3_VL-1 | ELVVTQEPSLTVSPGGTVTLTCRSSTGAVTTSNYANWVQD | SEQ ID |
| (SP34) | | KPGQAPRGLIGGTNKRAPGTPARFSGSLLGGKAALTLSGV | NO: 22 |
| | | QPEDEAEYYCALWYSNLWVFGGGTKLTVL | |
| | CD3_VH-1 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 13 |
| | | AYLQMNNLKTEDTAVYYCVRHGNFGNSYVSWFAYWGQGTL | |
| | | VTVSS | |
| CD3-A1 | CD3_VL-4 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 25 |
| | | QPEDEAEYYCALWDSNLWVFGGGTKLTVL | |
| | CD3_VH-1 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 13 |
| | | AYLQMNNLKTEDTAVYYCVRHGNFGNSYVSWFAYWGQGTL | |
| | | VTVSS | |
| CD3-A2 | CD3_VL-6 | ELVVTQEPSLTVSPGGTVTLTCRSSTGAVTTSNYANWVQD | SEQ ID |
| | | KPGQAPRGLIGDTNKRAPGTPARFSGSLLGGKAALTLSGV | NO: 27 |
| | | QPEDEAEYYCALWYSNLWVFGGGTKLTVL | |
| | CD3_VH-1 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 13 |
| | | AYLQMNNLKTEDTAVYYCVRHGNFGNSYVSWFAYWGQGTL | |
| | | VTVSS | |
| CD3-A3 | CD3_VL-2 | ELVVTQEPSLTVSPGGTVTLTCRSSTGAVTTSNYANWVQK | SEQ ID |
| | | KPGQAPRGLIGGTNKRAPGTPARFSGSLLGGKAALTLSGV | NO: 23 |
| | | QPEDEAEYYCALWYSNLWVFGGGTKLTVL | |
| | CD3_VH-2 | EVQLLESGGGLVQPGGSLKLSCAASGFTFNTYAMNWVRDA | SEQ ID |
| | | PGKGLEWVARIRSKYNNYATYYADSVKDRFTISRDDSKNT | NO: 14 |
| | | AYLQMNNLKTEDTAVYYCVRHGNFGNSYVSWFAYWGQGTL | |
| | | VTVSS | |
| CD3-A4 | CD3_VL-5 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 26 |
| | | QPEDEAEYYCALWYSQLWVFGGGTKLTVL | |
| | CD3_VH-3 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 15 |
| | | AYLQMNNLKTEDTAVYYCVRHGNFGDSYVSWFAYWGQGTL | |
| | | VTVSS | |
| CD3-A5 | CD3_VL-5 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 26 |
| | | QPEDEAEYYCALWYSQLWVFGGGTKLTVL | |
| | CD3_VH-4 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 16 |
| | | AYLQMNNLKTEDTAVYYCVRHGNFGDDYVSWFAYWGQGTL | |
| | | VTVSS | |
| CD3-A6 | CD3_VL-1 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 22 |
| | | QPEDEAEYYCALWYSNLWVFGGGTKLTVL | |
| | CD3_VH-5 | EVQLLESGGGLVQPGGSLKLSCAASGFTFNDYAMNWVRKA | SEQ ID |
| | | PGKGLEWVARIRSKYNNYATYYADSVKDRFTISRDDSKNT | NO: 17 |
| | | AYLQMNNLKTEDTAVYYCVRHGNFGNSYVSWFAYWGQGTL | |
| | | VTVSS | |
| CD3-A7 | CD3_VL-1 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 22 |
| | | QPEDEAEYYCALWYSNLWVFGGGTKLTVL | |
| | CD3_VH-6 | EVQLLESGGGLVQPGGSLKLSCAASGFTFNTYAMNWVRKA | SEQ ID |
| | | PGKGLEWVAKIRSKYNNYATYYADSVKDRFTISRDDSKNT | NO: 18 |
| | | AYLQMNNLKTEDTAVYYCVRHGNFGDSYVSWFAYWGQGTL | |
| | | VTVSS | |
| CD3-A8 | CD3_VL-5 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 26 |
| | | QPEDEAEYYCALWYSQLWVFGGGTKLTVL | |
| | CD3_VH-6 | EVQLLESGGGLVQPGGSLKLSCAASGFTFNTYAMNWVRKA | SEQ ID |
| | | PGKGLEWVAKIRSKYNNYATYYADSVKDRFTISRDDSKNT | NO: 18 |
| | | AYLQMNNLKTEDTAVYYCVRHGNFGDSYVSWFAYWGQGTL | |
| | | VTVSS | |
| CD3-A9 | CD3_VL-5 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 26 |
| | | QPEDEAEYYCALWYSQLWVFGGGTKLTVL | |
| | CD3_VH-7 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 19 |
| | | AYLQMNNLKTEDTAVYYCVRHGSFGNSYVSWFAYWGQGTL | |
| | | VTVSS | |
| CD3-A10 | CD3_VL-5 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 26 |
| | | QPEDEAEYYCALWYSQLWVFGGGTKLTVL | |
| | CD3_VH-8 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 20 |
| | | AYLQMNNLKTEDTAVYYCVRHGTFGNSYVSWFAYWGQGTL | |
| | | VTVSS | |
| CD3-A11 | CD3_VL-1 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 22 |
| | | QPEDEAEYYCALWYSNLWVFGGGTKLTVL | |
| | CD3_VH-9 | EVQLLESGGGLVQPGGSLKLSCAASGFTFNTYAMDWVRKA | SEQ ID |
| | | PGKGLEWVARIRSKYNNYATYYADSVKDRFTISRDDSKNT | NO: 21 |
| | | AYLQMNNLKTEDTAVYYCVRHGNFGNSYVSWFAYWGQGTL | |
| | | VTVSS | |
| CD3-A12 | CD3_VL-3 | ELVVTQEPSLTVSPGGTVTLTCRSSTGAVTTSNYANWVQE | SEQ ID |
| | | KPGQAPRGLIGGTNKRAPGTPARFSGSLLGGKAALTLSGV | NO: 24 |
| | | QPEDEAEYYCALWYSNLWVFGGGTKLTVL | |
| | CD3_VH-1 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 13 |
| | | AYLQMNNLKTEDTAVYYCVRHGNFGNSYVSWFAYWGQGTL | |
| | | VTVSS | |
| CD3-A13 | CD3_VL-7 | ELVVTQEPSLTVSPGGTVTLTCRSSTGAVTTSNYANWVQD | SEQ ID |
| | | KPGQAPRGLIGGTNKRAPGTPARFSGSLLGGKAALTLSGV | NO: 28 |
| | | QPEDEAEYYCALWYSDLWVFGGGTKLTVL | |
| | CD3_VH-4 | EVQLLESGGGLVQPGGSLKLSCAASGFTFNTYAMNWVRKA | SEQ ID |
| | | PGKGLEWVARIRSKYNNYATYYADSVKDRFTISRDDSKNT | NO: 16 |
| | | AYLQMNNLKTEDTAVYYCVRHGNFGDDYVSWFAYWGQGTL | |
| | | VTVSS | |

TABLE 8
Anti-CD3 Heavy Chain CDRs

| | CDRH1 | CDRH2 | CDRH3 |
|---|---|---|---|
| CD3-01 | TYAMN (SEQ ID NO: 29) | RIRSKYNNYATYYADSVKD (SEQ | HGNFGNSYVSW |
| (SP34) | (Kabat) | ID NO: 34) (Kabat) | FAY (SEQ ID |
| | GFTFNTY (SEQ ID NO: | RSKYNNYA (SEQ ID NO: 36) | NO: 37) |
| | 32) (Chothia) | (Chothia) | |
| CD3-A1 | TYAMN (SEQ ID NO: 29) | RIRSKYNNYATYYADSVKD (SEQ | HGNFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 37) |
| | 32) (Chothia) | (Chothia) | |
| CD3-A2 | TYAMN (SEQ ID NO: 29) | RIRSKYNNYATYYADSVKD (SEQ | HGNFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 37) |
| | 32) (Chothia) | (Chothia) | |
| CD3-A3 | TYAMN (SEQ ID NO: 29) | RIRSKYNNYATYYADSVKD (SEQ | HGNFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 37) |
| | 32) (Chothia) | (Chothia) | |
| CD3-A4 | TYAMN (SEQ ID NO: 29) | RIRSKYNNYATYYADSVKD (SEQ | HGNFGDSYVSW |
| | (Kabat) | ID NO: 34) (Kabat) | FAY (SEQ ID |
| | GFTFNTY (SEQ ID NO: | RSKYNNYA (SEQ ID NO: 36) | NO: 38) |
| | 32) (Chothia) | (Chothia) | |
| CD3-A5 | TYAMN (SEQ ID NO: 29) | RIRSKYNNYATYYADSVKD (SEQ | HGNFGDDYVSW |
| | (Kabat) | ID NO: 34) (Kabat) | FAY (SEQ ID |
| | GFTFNTY (SEQ ID NO: | RSKYNNYA (SEQ ID NO: 36) | NO: 39) |
| | 32) (Chothia) | (Chothia) | |
| CD3-A6 | DYAMN (SEQ ID NO: 30) | RIRSKYNNYATYYADSVKD (SEQ | HGNFGNSYVSW |
| | (Kabat) | ID NO: 34) (Kabat) | FAY (SEQ ID |
| | GFTFNDY (SEQ ID NO: | RSKYNNYA (SEQ ID NO: 36) | NO: 37) |
| | 33) (Chothia) | (Chothia) | |
| CD3-A7 | TYAMN (SEQ ID NO: 29) | KIRSKYNNYATYYADSVKD (SEQ | HGNFGDSYVSW |
| | (Kabat) | ID NO: 35) (Kabat ) | FAY (SEQ ID |
| | GFTFNTY (SEQ ID NO: | RSKYNNYA (SEQ ID NO: 36) | NO: 38) |
| | 32) (Chothia) | (Chothia) | |
| CD3-A8 | TYAMN (SEQ ID NO: 29) | KIRSKYNNYATYYADSVKD (SEQ | HGNFGDSYVSW |
| | (Kabat) | ID NO: 35) (Kabat) | FAY (SEQ ID |
| | GFTFNTY (SEQ ID NO: | RSKYNNYA (SEQ ID NO: 36) | NO: 38) |
| | 32) (Chothia) | (Chothia) | |
| CD3-A9 | TYAMN (SEQ ID NO: 29) | RIRSKYNNYATYYADSVKD (SEQ | HGSFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 40) |
| | 32) (Chothia) | (Chothia) | |
| CD3-A10 | TYAMN (SEQ ID NO: 29) | RIRSKYNNYATYYADSVKD (SEQ | HGTFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 41) |
| | 32) (Chothia) | (Chothia) | |
| CD3-A11 | TYAMD (SEQ ID NO: 31) | RIRSKYNNYATYYADSVKD (SEQ | HGNFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 37) |
| | 32) (Chothia) | (Chothia) | |
| CD3-A12 | TYAMN (SEQ ID NO: 29) | RIRSKYNNYATYYADSVKD (SEQ | HGNFGNSYVSW |
| | (Kabat) | ID NO: 34) (Kabat) | FAY (SEQ ID |
| | GFTFNTY (SEQ ID NO: | RSKYNNYA (SEQ ID NO: 36) | NO: 37) |
| | 32) (Chothia) | (Chothia) | |
| CD3-A13 | TYAMN (SEQ ID NO: 29) | RIRSKYNNYATYYADSVKD (SEQ | HGNFGDDYVSW |
| | (Kabat) | ID NO: 34) (Kabat) | FAY (SEQ ID |
| | GFTFNTY (SEQ ID NO: | RSKYNNYA (SEQ ID NO: 36) | NO: 39) |
| | 32) (Chothia) | (Chothia) | |

TABLE 9
Anti-CD3 Light Chain CDRs

| | CDRL1 | CDRL2 | CDRL3 |
|---|---|---|---|
| CD3-01 | RSSTGAVTTSNYAN | GTNKRAP (SEQ ID NO: | ALWYSNLWV (SEQ |
| (SP34) | (SEQ ID NO: 42) | 43) | ID NO: 45) |
| CD3-A1 | RSSTGAVTTSNYAN | GTNKRAP (SEQ ID NO: | ALWDSNLWV (SEQ |
| | (SEQ ID NO: 42) | 43) | ID NO: 46) |
| CD3-A2 | RSSTGAVTTSNYAN | DTNKRAP (SEQ ID NO: | ALWYSNLWV (SEQ |
| | (SEQ ID NO: 42) | 44) | ID NO: 45) |
| CD3-A3 | RSSTGAVTTSNYAN | GTNKRAP (SEQ ID NO: | ALWYSNLWV (SEQ |
| | (SEQ ID NO: 42) | 43) | ID NO: 45) |
| CD3-A4 | RSSTGAVTTSNYAN | GTNKRAP (SEQ ID NO: | ALWYSQLWV (SEQ |
| | (SEQ ID NO: 42) | 43) | ID NO: 47) |
| CD3-A5 | RSSTGAVTTSNYAN | GTNKRAP (SEQ ID NO: | ALWYSQLWV (SEQ |
| | (SEQ ID NO: 42) | 43) | ID NO: 47) |
| CD3-A6 | RSSTGAVTTSNYAN | GTNKRAP (SEQ ID NO: | ALWYSNLWV (SEQ |
| | (SEQ ID NO: 42) | 43) | ID NO: 45) |
| CD3-A7 | RSSTGAVTTSNYAN | GTNKRAP (SEQ ID NO: | ALWYSNLWV (SEQ |
| | (SEQ ID NO: 42) | 43) | ID NO: 45) |
| CD3-A8 | RSSTGAVTTSNYAN | GTNKRAP (SEQ ID NO: | ALWYSQLWV (SEQ |
| | (SEQ ID NO: 42) | 43) | ID NO: 47) |
| CD3-A9 | RSSTGAVTTSNYAN | GTNKRAP (SEQ ID NO: | ALWYSQLWV (SEQ |
| | (SEQ ID NO: 42) | 43) | ID NO: 47) |
| CD3-A10 | RSSTGAVTTSNYAN | GTNKRAP (SEQ ID NO: | ALWYSQLWV (SEQ |
| | (SEQ ID NO: 42) | 43) | ID NO: 47) |
| CD3-A11 | RSSTGAVTTSNYAN | GTNKRAP (SEQ ID NO: | ALWYSNLWV (SEQ |
| | (SEQ ID NO: 42) | 43) | ID NO: 45) |
| CD3-A12 | RSSTGAVTTSNYAN | GTNKRAP (SEQ ID NO: | ALWYSNLWV (SEQ |
| | (SEQ ID NO: 42) | 43) | ID NO: 45) |
| CD3-A13 | RSSTGAVTTSNYAN | GTNKRAP (SEQ ID NO: | ALWYSDLWV (SEQ |
| | (SEQ ID NO: 42) | 43) | ID NO: 48) |

In some embodiments, the disclosure provides an antibody (e.g. including antibody fragments, such as single chain variable fragments (scFvs) which specifically bind to CD3ε, wherein the antibody comprises a) a heavy chain variable region (VH) comprising a i) a VH complementarity determining region 1 (VH_{CDR1}) comprising the amino acid sequence of SEQ ID NO: 29, 30, 31, 32 or 33, ii) a VH complementarity determining region 2 (VH_{CDR2}) comprising the amino acid sequence of SEQ ID NO: 34, 35 or 36, iii) a VH complementarity determining region 3 (VH_{CDR3}) comprising the amino acid sequence of SEQ ID NO: 37, 38, 39, 40 or 41; and b) a light chain variable region (VL) comprising a i) i) a VL complementarity determining region 1 (VL_{CDR1}) comprising the amino acid sequence of SEQ ID NO: 42, ii) a VL complementarity determining region 2 (VL_{CDR2}) comprising the amino acid sequence of SEQ ID NO: 43 or 44, iii) a VL complementarity determining region 3 (VL_{CDR3}) comprising the amino acid sequence of SEQ ID NO: 45, 46, 47 or 48.

Exemplary anti-CD3 antibodies of the invention include CD3-A1, CD3-A2, CD3-A3, CD3-A4, CD3-A5, CD3-A6, CD3-A7, CD3-A8, CD3-A9, CD3-A10, CD3-A11, CD3-A12 and CD3-A13.

In some embodiments, the anti-CD3 antibody CD3-A1 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 37; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 46.

In some embodiments, the anti-CD3 antibody CD3-A1 comprises a VH region comprising the amino acid sequence shown in SEQ ID NO: 13 and a VL region comprising the amino acid sequence shown in SEQ ID NO: 25.

In some embodiments, the anti-CD3 antibody CD3-A2 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 37; and a VL region comprising a VL_{CDR1} comprising the amino acid sequence of SEQ ID NO: 42, a VL_{CDR2} comprising the amino acid sequence of SEQ ID NO: 44, and a VL_{CDR3} comprising the amino acid sequence of SEQ ID NO: 45.

In some embodiments, the anti-CD3 antibody CD3-A2 comprises a VH region comprising the amino acid sequence shown in SEQ ID NO: 13 and a VL region comprising the amino acid sequence shown in SEQ ID NO: 27.

In some embodiments, the anti-CD3 antibody CD3-A3 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 37; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 45.

In some embodiments, the anti-CD3 antibody CD3-A3 comprises a VH region comprising the amino acid sequence shown in SEQ ID NO: 14 and a VL region comprising the amino acid sequence shown in SEQ ID NO: 23.

In some embodiments, the anti-CD3 antibody CD3-A4 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 38; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 47. In some embodiments, the anti-CD3 antibody CD3-A4 comprises a VH region comprising the amino acid sequence shown in SEQ ID NO: 15 and a VL region comprising the amino acid sequence shown in SEQ ID NO: 26.

In some embodiments, the anti-CD3 antibody CD3-A5 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 39; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 47.

In some embodiments, the anti-CD3 antibody CD3-A5 comprises a VH region comprising the amino acid sequence shown in SEQ ID NO: 16 and a VL region comprising the amino acid sequence shown in SEQ ID NO: 26.

In some embodiments, the anti-CD3 antibody CD3-A6 comprises a VH region comprising a VH_{CDR1} comprising the amino acid sequence of SEQ ID NO: 30, a VH_{CDR2} comprising the amino acid sequence of SEQ ID NO: 34, and a VH_{CDR3} comprising the amino acid sequence of SEQ ID NO: 37; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 45.

In some embodiments, the anti-CD3 antibody CD3-A6 comprises a VH region comprising the amino acid sequence shown in SEQ ID NO: 17 and a VL region comprising the amino acid sequence shown in SEQ ID NO: 22.

In some embodiments, the anti-CD3 antibody CD3-A7 comprises a VH region comprising a VH_{CDR1} comprising the amino acid sequence of SEQ ID NO: 29, a VH_{CDR2} comprising the amino acid sequence of SEQ ID NO: 35, and a VH_{CDR3} comprising the amino acid sequence of SEQ ID NO: 38; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 45.

In some embodiments, the anti-CD3 antibody CD3-A7 comprises a VH region comprising the amino acid sequence shown in SEQ ID NO: 18 and a VL region comprising the amino acid sequence shown in SEQ ID NO: 22.

In some embodiments, the anti-CD3 antibody CD3-A8 comprises a VH region comprising a VH_{CDR1} comprising the amino acid sequence of SEQ ID NO: 29, a VH_{CDR2} comprising the amino acid sequence of SEQ ID NO: 35, and a VH_{CDR3} comprising the amino acid sequence of SEQ ID NO: 38; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 47.

In some embodiments, the anti-CD3 antibody CD3-A8 comprises a VH region comprising the amino acid sequence shown in SEQ ID NO: 18 and a VL region comprising the amino acid sequence shown in SEQ ID NO: 26.

In some embodiments, the anti-CD3 antibody CD3-A9 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 40; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 47.

In some embodiments, the anti-CD3 antibody CD3-A9 comprises a VH region comprising the amino acid sequence shown in SEQ ID NO: 19 and a VL region comprising the amino acid sequence shown in SEQ ID NO: 26.

In some embodiments, the anti-CD3 antibody CD3-A10 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 41; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 47.

In some embodiments, the anti-CD3 antibody CD3-A10 comprises a VH region comprising the amino acid sequence shown in SEQ ID NO: 20 and a VL region comprising the amino acid sequence shown in SEQ ID NO: 26.

In some embodiments, the anti-CD3 antibody CD3-A11 comprises a VH region comprising a VH_{CDR1} comprising the amino acid sequence of SEQ ID NO: 31, a VH_{CDR2} comprising the amino acid sequence of SEQ ID NO: 34, and a VH_{CDR3} comprising the amino acid sequence of SEQ ID NO: 37; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 45.

In some embodiments, the anti-CD3 antibody CD3-A11 comprises a VH region comprising the amino acid sequence shown in SEQ ID NO: 21 and a VL region comprising the amino acid sequence shown in SEQ ID NO: 22.

In some embodiments, the anti-CD3 antibody CD3-A12 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 37; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 45.

In some embodiments, the anti-CD3 antibody CD3-A12 comprises a VH region comprising the amino acid sequence shown in SEQ ID NO: 13 and a VL region comprising the amino acid sequence shown in SEQ ID NO: 24.

In some embodiments, the anti-CD3 antibody CD3-A13 comprises a VH region comprising a VH_{CDR1} comprising the amino acid sequence of SEQ ID NO: 29, a VH_{CDR2} comprising the amino acid sequence of SEQ ID NO: 34, and a VH_{CDR3} comprising the amino acid sequence of SEQ ID NO: 39; and a VL region comprising a VL_{CDR1} comprising the amino acid sequence of SEQ ID NO: 42, a VL_{CDR2} comprising the amino acid sequence of SEQ ID NO: 43, and a VL_{CDR3} comprising the amino acid sequence of SEQ ID NO: 48.

In some embodiments, the anti-CD3 antibody CD3-A13 comprises a VH region comprising the amino acid sequence shown in SEQ ID NO: 16 and a VL region comprising the amino acid sequence shown in SEQ ID NO: 28.

### Bispecific Anti-CD3ε Antibodies

Provided herein are bispecific antibodies comprising a first antigen binding domain that binds a first antigen (e.g. CD3ε) and a second antigen binding domain that binds to a second antigen (e.g. disease associated antigen).

In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2).

In some embodiments, the bispecific antibody of the disclosure comprises a first antigen binding domain (e.g. binding to CD3) comprising any one of the VH1 and VL1 sequences listed in Table 7. In Table 7, the underlined sequences are CDR sequence according to Kabat and the bolded sequences are CDR sequences according to Chothia.

In some embodiments, the bispecific antibody of the disclosure comprises a first antigen binding domain (e.g. binding to CD3ε) comprising: a) a heavy chain variable region (VH1) comprising a VH complementarity determining region 1 (VH1_{CDR1}), a VH complementarity determining region 2 (VH1_{CDR2}) and a VH complementarity determining region 3 (VH1_{CDR3}); and b) a light chain variable region (VL) comprising a VL complementarity determining region 1 (VL1_{CDR1}), a VL complementarity determining region 2 (VL1_{CDR2}) and a VL complementarity determining region 3 (VL1_{CDR3}). Tables 8 and 9 provide exemplary of CDR sequences of the anti-CD3 antibodies provided herein.

In some embodiments, the bispecific antibody comprises any one of the anti-CD3 antibodies of the disclosure. Exemplary anti-CD3 antibodies of the invention include CD3-A1, CD3-A2, CD3-A3, CD3-A4, CD3-A5, CD3-A6, CD3-A7, CD3-A8, CD3-A9, CD3-A10, CD3-A11, CD3-A12 and CD3-A13.

In some embodiments, the disclosure provides an isolated antibody (e.g. monospecific antibody or bispecific antibody) which specifically binds to CD3ε and competes with any of the foregoing antibodies.

In some embodiments, the present invention provides an antibody (e.g. monospecific antibody or bispecific antibody) that binds to CD3ε and competes with an antibody as described herein, including CD3-A1, CD3-A2, CD3-A3, CD3-A4, CD3-A5, CD3-A6, CD3-A7, CD3-A8, CD3-A9, CD3-A10, CD3-A11, CD3-A12 and CD3-A13.

In some embodiments, the invention also provides CDR portions of antibodies to CD3ε antibodies based on CDR contact regions. CDR contact regions are regions of an antibody that imbue specificity to the antibody for an antigen. In general, CDR contact regions include the residue positions in the CDRs and Vernier zones which are constrained in order to maintain proper loop structure for the antibody to bind a specific antigen. See, e.g., Makabe et al., J. Biol. Chem., 283:1156-1166, 2007. Determination of CDR contact regions is well within the skill of the art.

The binding affinity (K_{D}) of the anti-CD3ε antibodies of the invention (e.g. monospecific antibody or bispecific antibody) to human CD3ε (such as human CD3ε (e.g., (SEQ ID NO: 419)) can be about 0.001 to about 5000 nM.

In some embodiments, the binding affinity is about any of 5000 nM, 4500 nM, 4000 nM, 3500 nM, 3000 nM, 2500 nM, 2000 nM, 1789 nM, 1583 nM, 1540 nM, 1500 nM, 1490 nM, 1064 nM, 1000 nM, 933 nM, 894 nM, 750 nM, 705 nM, 678 nM, 532 nM, 500 nM, 494 nM, 400 nM, 349 nM, 340 nM, 353 nM, 300 nM, 250 nM, 244 nM, 231 nM, 225 nM, 207 nM, 200 nM, 186 nM, 172 nM, 136 nM, 113 nM, 104 nM, 101 nM, 100 nM, 90 nM, 83 nM, 79 nM, 74 nM, 54 nM, 50 nM, 45 nM, 42 nM, 40 nM, 35 nM, 32 nM, 30 nM, 25 nM, 24 nM, 22 nM, 20 nM, 19 nM, 18 nM, 17 nM, 16 nM, 15 nM, 12 nM, 10 nM, 9 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM, 0.1 nM, 0.01 nM, or 0.001 nM.

In some embodiments, the binding affinity is less than about any of 5000 nM, 4000 nM, 3000 nM, 2000 nM, 1000 nM, 900 nM, 800 nM, 250 nM, 200 nM, 100 nM, 50 nM, 30 nM, 20 nM, 10 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

The binding affinity (K_{D}) of the anti-CD3ε antibodies of the invention (e.g. monospecific antibody or bispecific antibody) to cynomolgus CD3ε (such as cynomolgus CD3ε (e.g., (SEQ ID NO: 422)) can be about 0.001 to about 5000 nM.

In some embodiments, the binding affinity is about any of 5000 nM, 4500 nM, 4000 nM, 3500 nM, 3000 nM, 2500 nM, 2000 nM, 1789 nM, 1583 nM, 1540 nM, 1500 nM, 1490 nM, 1064 nM, 1000 nM, 933 nM, 894 nM, 750 nM, 705 nM, 678 nM, 532 nM, 500 nM, 494 nM, 400 nM, 349 nM, 340 nM, 353 nM, 300 nM, 250 nM, 244 nM, 231 nM, 225 nM, 207 nM, 200 nM, 186 nM, 172 nM, 136 nM, 113 nM, 104 nM, 101 nM, 100 nM, 90 nM, 83 nM, 79 nM, 74 nM, 54 nM, 50 nM, 45 nM, 42 nM, 40 nM, 35 nM, 32 nM, 30 nM, 25 nM, 24 nM, 22 nM, 20 nM, 19 nM, 18 nM, 17 nM, 16 nM, 15 nM, 12 nM, 10 nM, 9 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM, 0.1 nM, 0.01 nM, or 0.001 nM.

In some embodiments, the binding affinity is less than about any of 5000 nM, 4000 nM, 3000 nM, 2000 nM, 1000 nM, 900 nM, 800 nM, 250 nM, 200 nM, 100 nM, 50 nM, 30 nM, 20 nM, 10 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

In some embodiments, the disclosure provides a nucleic acid encoding any of the foregoing isolated anti-CD3ε antibodies (e.g. monospecific antibody or bispecific antibody). In some embodiments, the disclosure provides a vector comprising such a nucleic acid. In some embodiments, the disclosure provides a host cell comprising such a nucleic acid.

### Disease Associated Antigens

Provided herein are bispecific antibodies that have a first antigen binding domain that binds a first antigen (e.g. CD3ε) and a second antigen binding domain that binds to a second antigen (e.g. a cell surface antigen, or DAA).

In some embodiments, the second biological molecule is a cell surface antigen. In some embodiments the second biological molecule is a disease associated antigen. Disease associated antigens include but are not limited to ACVR1, ADAM21, AGL10, ALPPL2, APCDD1, ASPRV1, BCMA, BMPR1B, CD151, CD19, CD22, CD274, CD276, CD33, CD3ε, CD47, CD6, CD70, CD74, CD84, CD180, CDCP1, CDH17, CDH3, CDHR2,CDHR5, CEACAM5, CEACAM6, CEACAM7, CELSR1, CLCA2, CLDN1, CLDN18, CLDN6, CNGB1, CNGB3, COL11A1, COL17A1, CRB1, CPSG4, CTAG2, CTAGE4, CXADR, CXCR4, DCBLD2, DCST1, DLL3, DLL4, DPCR1, DSG3, DSG4, DUOX2, EBI3, EFNA4, EGFR, ENTPD1, ENTPD2, EPCAM, EPHA10, EPHA6, EPHA8, EPHB3, EPS8L1, ERBB2, ERMP1, F11R, FAP, FAT1, FCER2, FCRL3, FER1L6, FGFR2, FLT3, FLVCR1, FN1, FXYD3, GABRA3, GGT2, GGT3P, GJB3, GLG1, GPC1, GPC2, GPNMB, GPC5A, GRIND2D, GUCY2C, HAVCR2, HEPHL1, HHLA1, IGSF3, IGSF9, IL2RB, IL3RA, ITGA2, ITGA6, ITGAV, ITGB4, ITGB6, LCN15, LILRB4, LNPEP, LRFN4, LRRC15, LY6D, LY75, MAL2, MET, MFI2, MICA, MICB, MMP13, MMP14, MPZL2, MS4A1, MSLN, MSTIR, MTDH, MUC1, MUC13, MUC16, MUC17, NAALADL2, NCSTN, NIPAL4, NLGN1, NOTCH3, NOX1, OC90, OR10Q1, OR511, PAEP, PANX3, PCDH15, PDCHA9, PCDHB12, PCDHB2, PKD1L1, PODXL, POLR2J2, PROM1, PSMA, PTK7, PVR, PVRL1, PVRL4, RAETIE, RAET1G, RAET1L, ROR1, ROR2, SDC1, SDC4, SDK2, SHISA8, SIGLEC7, SIT1, SLAMF1, SLAMF6, SLAMF7, SLC11A2, SLC12A2, SLC15A1, SLC1A5, SLC22A25, SLC2A9, SLC34A2, SLC38A2, SLC39A4, SLC6A14, SLC7A11, SLC7A3, SLC7A5, SYT8, TAS2R5, TMEM132A, TMPRSS3, TMPRSS4, TMX1, TNFRSF17, TNFRSF21, TNFRSF9, TNFRSF11, TNFRSF15, TNFRSF4, TNFRSF9, TNMD, TP53111, TPBG, TRPC5, TRPV2, TSPAN10, TSPAN8, UGT2A1,UGT3A2, ULBP1, ULBP2, ULBP3, UMODL1, UPK1B, VANGL1, VANGL2, VASN, VMP1, VSIG4, VTCN1, WNT16, YIF1B, and ZNRF4.

Exemplary disease associated antigens include but are not limited to those shown in Table 22.

TABLE 22
Exemplary Disease Associated Antigens

| Target | Examples of Antibody Name and/or Reference(s) and/or Source |
|---|---|
| ALK | antibodies described in e.g., Mino-Kenudson et al., 2010, Clin Cancer |
| | Res 16(5): 1561-1571 |
| B7H3 | MGA271 (Macrogenics) |
| BCMA | Any BCMA antibody described in WO2012163805, WO200112812, or |
| | WO2003062401. |
| CAIX | Antibody clone 303123 (R&D Systems) |
| CD123 | U.S. Pat. No. 8,852,551; EP2426148; WO2014138819; WO2016028896; |
| | WO2014130635 |
| CD171 | Hong et al., 2014, J Immunother 37(2): 93-104. |
| CD19 | WO2014031687; WO2012079000; WO2014153270; U.S. Pat. No. |
| | 7,741,465; the CD19 binder of Yescarta or Blinatumomab |
| CD20 | Rituximab, Ofatumumab, Ocrelizumab, Veltuzumab, or GA101 |
| CD22 | Haso et al., 2013, Blood, 121(7): 1165-1174; Wayne et al., 2010, Clin |
| | Cancer Res 16(6): 1894-1903; Kato et al., 2013, Leuk Res 37(1): 83-88; |
| | Creative BioMart (creativebiomart.net): MOM-18047-S(P). |
| CD24 | Maliar et al., Gastroenterology 143(5): 1375-1384 (2012) |
| CD30 | Any CD30 antibody described in U.S. Pat. No. 7,090,843 B1, or EP0805871 |
| CD33 | Bross et al., 2001, Clin Cancer Res 7(6): 1490-1496 (Gemtuzumab |
| | Ozogamicin, hP67.6), Caron et al., 1992, Cancer Res 52(24): 6761-6767 |
| | (Lintuzumab, HuM195), Lapusan et al., 2012, Invest New Drugs |
| | 30(3): 1121-1131 (AVE9633), Aigner et al., 2013, Leukemia 27(5): |
| | 1107-1115 (AMG330, CD33 BiTE), Dutour et al., 2012, Adv Hematol |
| | 2012: 683065, or Pizzitola et al., 2014, Leukemia |
| | doi: 10.1038/Lue.2014.62. |
| CD38 | Daratumumab (see, e.g., Groen et al., 2010, Blood 116(21): 1261-1262; |
| | MOR202 (see, e.g., U.S. Pat. No. 8,263,746); or any CD38 antibody |
| | described in U.S. Pat. No. 8,362,211. |
| CD44v6 | Casucci et al., 2013, Blood 122(20): 3461-3472. |
| CD97 | antibodies described in, e.g., U.S. Pat. No. 6,846,911; de Groot et al., |
| | 2009, J Immunol 183(6): 4127-4134; antibody from R&D: MAB373 |
| CEA | Chmielewski et al., 2012, Gastoenterology 143(4): 1095-1107. |
| CLDN6 | WO2015069794; IMAB027, mAb, Ganymed Pharmaceuticals |
| CLL-1 | PE-CLL1-hu Cat# 353604 (BioLegend); and PE-CLL1 (CLEC12A) |
| | Cat# 562566 (BD); WO 2014/051433 A1; US 2016/0368994 A1; US |
| | 2013/0295118 A1; U.S. Pat. No. 8,536,310 B2; Lu et al., 2014, |
| | Angewandte Chemie International Edition 53(37): 9841-9845; Leong et |
| | al., 2017, Blood 129(5): 609-618 |
| CS1 | Elotuzumab (BMS), see e.g., Tai et al., 2008, Blood 112(4): 1329-37; |
| | Tai et al., 2007, Blood. 110(5): 1656-63. |
| EGFR | Cetuximab, panitumumab, zalutumumab, nimotuzumab, or matuzumab |
| EGFRvIII | WO2012138475; WO2014130657 |
| EPCAM | MT110, EpCAM-CD3 bispecific Ab (see, |
| | e.g., clinicaltrials.gov/ct2/show/NCT00635596); Edrecolomab; |
| | 3622W94; ING-1; or adecatumumab (MT201). |
| EphA2 | Yu et al., 2014, Mol Ther 22(1): 102-111. |
| Ephrin B2 | Abengozar et al., 2012, Blood 119(19): 4565-4576. |
| ERBB2 | Trastuzumab or pertuzumab. |
| (Her2/neu) | |
| FAP | Ostermann et al., 2008, Clinical Cancer Research 14: 4584-4592 |
| | (FAP5), US Pat. Publication No. 2009/0304718; sibrotuzumab (see |
| | e.g., Hofheinz et al., 2003, Oncology Research and Treatment |
| | 26(1): 44-48); and Tran et al., 2013, J Exp Med 210(6): 1125-1135. |
| FLT3 | Any FLT3 antibody described in WO2011076922, U.S. Pat. No. |
| | 5,777,084, EP0754230, or US20090297529. |
| Folate | IMGN853, or any folate receptor alpha antibody described in |
| receptor alpha | US20120009181; U.S. Pat. No. 4,851,332, LK26: U.S. Pat. No. 5,952,484. |
| Folate | antibodies described in, e.g., US20100297138; WO2007/067992 |
| receptor beta | |
| GD2 | Mujoo et al., Cancer Res. 47(4): 1098-1104 (1987); Cheung et al., |
| | Cancer Res 45(6): 2642-2649 (1985), Cheung et al., J Clin Oncol |
| | 5(9): 1430-1440 (1987), Cheung et al., J Clin Oncol 16(9): 3053-3060 |
| | (1998), Handgretinger et al., Cancer Immunol Immunother 35(3): 199- |
| | 204 (1992); |
| | mAb 14.18, 14G2a, ch14.18, hu14.18, 3F8, hu3F8, 3G6, 8B6, 60C3, |
| | 10B8, ME36.1, or 8H9 (see e.g., WO2012033885, WO2013040371, |
| | WO2013192294, WO2013061273, WO2013123061, WO2013074916, |
| | and WO201385552). |
| | Any GD2 antibody described in US Publication No.: 20100150910 or |
| | PCT Publication No.: WO 2011160119. |
| GD3 | Any GD3 antibody described in U.S. Pat. No. 7,253,263; U.S. Pat. No. |
| | 8,207,308; US 20120276046; EP1013761; WO2005035577; or U.S. Pat. |
| | No. 6,437,098. |
| GloboH | VK9; Kudryashov et al., 1998, Glycoconj J.15(3): 243-9; Lou et al., |
| | 2014, Proc Natl Acad Sci USA 111(7): 2482-2487; MBr1: Bremer et |
| | al., 1984, J Biol Chem 259: 14773-14777. |
| gp100 | HMB45, NKlbetaB, or any anti-gp100 antibody described in |
| | WO2013165940, or US20130295007 |
| GPRC5D | R&Dsystems: FAB6300A; Lifespan Biosciences: LS-A4180 |
| HMWMAA | antibodies described in, e.g., Kmiecik et al., 2014, Oncoimmunology |
| | 3(1): e27185 (PMID: 24575382) (mAb9.2.27); U.S. Pat. No. 6,528,481; |
| | WO2010033866; US 20140004124 |
| IGF-I receptor | Any IGF-I receptor antibody described in U.S. Pat. No. 8,344,112 B2; EP2322550 |
| | A1; WO 2006/138315, or PCT/US2006/022995. |
| IL-11Ra | Abcam (cat# ab55262) or Novus Biologicals (cat# EPR5446) |
| IL-13Ra2 | Any IL-13Ra2 antibody described in WO2008/146911, |
| | WO2004087758, or WO2004087758 |
| KIT | Any KIT antibody described in U.S. Pat. No. 7,915,391, US20120288506 |
| KLRG2 | ab121563 (Abcam); B-12 or sc-514346 (Santa Cruz); HPA018199 |
| | (Sigma Aldrich) |
| Lewis Y | Kelly et al., Cancer Biother Radiopharm 23(4): 411-423 (2008) |
| | (hu3S193 Ab (scFvs)); Dolezal et al., Protein Engineering 16(1): 47-56 |
| | (2003) (NC10 scFv) |
| LMP2 | Any LMP2 antibody described in U.S. Pat. No. 7,410,640 or US 2005/0129701 |
| LRP6 | WO2009064944, WO2009056634, WO2011119661, WO2011138392, |
| | WO2011138391, WO2013067355, WO2014029752, WO2017093478 |
| Mesothelin | Any mesothelin antibody described in US 20110262448, US |
| | 2012/0107933 or U.S. Pat. No. 9,719,996 |
| MUC1 | SAR566658 |
| NCAM | 2-2B: MAB5324 (EMD Millipore) |
| NY-BR-1 | antibodies described in, e.g., Jager et al., 2007, Appl Immunohitochem |
| | Mol Morphol 15(1): 77-83 |
| o-acetyl-GD2 | 8B6 |
| PDGFR-beta | Abcam ab32570 |
| PLAC1 | antibodies described in, e.g., Ghods et al., 2013, Biotechnol Appl |
| | Biochem doi: 10.1002/bab.1177 |
| Polysialic | antibodies described in e.g., Nagae et al., 2013, J Biol Chem |
| acid | 288(47): 33784-33796 |
| PRSS21 | Any PRSS21 antibody described in U.S. Pat. No.: 8,080,650. |
| PSCA | Morgenroth et al., Prostate 67(10): 1121-1131 (2007) (scFv 7F5); |
| | Nejatollahi et al., J of Oncology 2013(2013), article ID 839831 (scFv |
| | C5-II); or any PSCA antibody described in US Pat Publication No. |
| | 20090311181. |
| PSMA | Parker et al., Protein Expr Purif 89(2): 136-145 (2013), US |
| | 20110268656 (J591 ScFv); Frigerio et al, European J Cancer |
| | 49(9): 2223-2232 (2013) (scFvD2B); WO 2006125481 (mAbs 3/A12, |
| | 3/E7 and 3/F11) or single chain antibody fragments (scFv A5 and D7). |
| ROR1 | Hudecek et al., Clin Cancer Res 19(12): 3153-3164 (2013); or any |
| | ROR1 antibody described in WO 2011159847 or US20130101607. |
| SSEA-4 | MC813 (Cell Signaling) |
| TAG72 | Hombach et al., Gastroenterology 113(4): 1163-1170 (1997) or Abcam |
| | ab691. |
| TEM1/CD248 | antibodies described in, e.g., Marty et al., 2006, Cancer Lett |
| | 235(2): 298-308; Zhao et al., 2011, J Immunol Methods 363(2): 221-232 |
| Tn | Brooks et al., PNAS 107(22): 10056-10061 (2010); Stone et al., |
| | Oncolmmunology 1(6): 863-873(2012); any Tn antibody described in |
| | U.S. Pat. No. 8,440,798 |
| TSHR | antibodies described in, e.g., Marty et al., 2006, Cancer Lett |
| | 235(2): 298-308; Zhao et al., 2011, J Immunol Methods 363(2): 221-232 |
| Tyrosinase | Any tyrosinase antibody described in U.S. Pat. No. 5,843,674 or |
| | US19950504048. |
| VEGFR2 | Chinnasamy et al., J Clin Invest 120(11): 3953-3968 (2010). |

Multispecific antibodies are antibodies (e.g., monoclonal antibodies) that have binding specificities for at least two different sites. In some embodiments, the anti-CD3 CD3ε antibody provided herein is a multispecific antibody (e.g. a bispecific antibody). In certain embodiments, bispecific antibodies may bind to two different epitopes of CD3 (e.g., CD3ε or CD3γ). In certain embodiments, one of the binding specificities is for CD3 (e.g., CD3ε or CD3γ) and the other is for any other antigen (e.g., a second biological molecule, e.g., a cell surface antigen, e.g., a disease associated antigen). Accordingly, a bispecific anti-CD3 antibody may have binding specificities for CD3 and a second biological molecule, such as a second biological molecule (e.g., a disease associated antigen) listed in Table 22 and described in U.S. Pub. No. 2010/0111856 and PCT Publication No. WO2016204966 A1, each of which are incorporated by reference herein in their entirety.

In some instances, the cell surface antigen (e.g. disease associated antigen) may be expressed in low copy number on the target cell (e.g. tumor cell). For example, in some instances, the cell surface antigen is expressed or present at less than 35,000 copies per target cell. In some embodiments, the low copy number cell surface antigen is present between 100 and 35,000 copies per target cell; between 100 and 30,000 copies per target cell; between 100 and 25,000 copies per target cell; between 100 and 20,000 copies per target cell; between 100 and 15,000 copies per target cell; between 100 and 10,000 copies per target cell; between 100 and 5,000 copies per target cell; between 100 and 2,000 copies per target cell; between 100 and 1,000 copies per target cell; or between 100 and 500 copies per target cell. Copy number of the cell surface antigen can be determined, for example, using a standard Scratchcard plot.

### UL16 Binding Proteins

Exemplary UL16 binding proteins include but are not limited to ULBP1, ULBP2, ULBP3, RAETIE (ULBP4), RAET1G (ULBP5) and RAET1L (ULBP6). Defects in the regulation of ULBP1-6 are associated with diseases ranging from autoimmunity to cancer.

UL16 Binding Protein 2 (ULBP2) is a major histocompatibility complex (MHC) class I-related molecule that binds to the NKG2D receptor on natural killer (NK) cells to trigger release of multiple cytokines and chemokines that in turn contribute to the recruitment and activation of NK cells. The encoded protein undergoes further processing to generate the mature protein that is either anchored to membrane via a glycosyl-phosphatidylinositol moiety, or secreted. Many malignant cells secrete the encoded protein to evade immunosurveillance by NK cells. ULBP2 is broadly and differentially expressed in multiple solid tumor indications. In particular, ULBP2 expression in melanoma and breast cancer is associated with poor prognosis and late-stage disease.

Senescence is a stress-induced cellular state that limits tumorigenesis by preventing cell proliferation and promoting immune-mediated clearance of damaged cells through the induction of senescence-associated secretory phenotype (SASP) (Rodier, F. et al. Persistent DNA damage signalling triggers senescence-associated inflammatory cytokine secretion. Nat. Cell Biol. 11, 973-979 (2009)). Senescence is also implicated in age-related tissue pathologies where the accumulation of SASP-positive cells can induce tissue inflammation resulting in tissue dysfunction that manifests in aged patients as arthritis, autoimmunity, diabetes, fibrosis, and delayed wound healing (Childs, B. G. et al. Senescent cells: an emerging target for diseases of ageing. Nat. Rev. Drug Discov. 16, 718-735 (2017)). SASP-positive cells express a complex assortment of both secreted and cell surface proteins including immune-activating cytokines, tissue remodeling matrix metalloprotease, and cell surface proteins that include MHCI-like NKG2D ligands that mediate the recognition and activation of NK and T cell effectors through NKG2D costimulatory receptors. These proteins together promote the elimination of senescent cells. However, age-related decline in immune cell activities and other factors like chemotherapy treatment of cancer accelerates the induction of SASP-positive cells in tissues and limits the clearance of senescent cells by the immune system (Jackola, D. R., Ruger, J. K. & Miller, R. A. Age-associated changes in human T cell phenotype and function. Aging Clin. Exp. Res. 6, 25-34 (1994); Demaria, M. et al. Cellular Senescence Promotes Adverse Effects of Chemotherapy and Cancer Relapse. Cancer Discov. 7, 165-176 (2017)). Therapeutic strategies to eliminate SASP-positive cells provide an opportunity to supplement senescent immunosurveillance and alleviate an underlying etiology of many age-related diseases and lasting side effects of chemotherapy. In addition, the clearance of senescent cells cannot only reduce age-related disease, but these senolytic drugs also have the potential to extend life span (Baker, D. J. et al. Naturally occurring p16Ink4a-positive cells shorten healthy lifespan. Nature 530, 184-189 (2016); Baker, D. J. et al. Clearance of p16Ink4a-positive senescent cells delays ageing-associated disorders. Nature 479, 232-236 (2011)). ULBP2, an MHCI-like ligand, has emerged as a cell surface protein associated with stress-induced SASP-positive fibroblasts and cancer cells (Sagiv, A. et al. NKG2D ligands mediate immunosurveillance of senescent cells. Aging 8, 328-344 (2016); Ruscetti, M. et al. NK cell-mediated cytotoxicity contributes to tumor control by a cytostatic drug combination. Science 362, 1416-1422 (2018); Muñoz, D. P. et al. Targetable mechanisms driving immunoevasion of persistent senescent cells link chemotherapy-resistant cancer to aging. JCI Insight 5, e124716, 124716 (2019)). ULBP2 or ULBP2/5/6 targeted drugs, in addition to eradicating cancer cells, have the potential to eliminate SASP-positive cells from tissues with the potential to improve tissue function which can prevent age-related disease and extend life span.

ULBP2 encompasses naturally occurring variants of ULBP2, including, for example, splice variants or allelic variants. ULBP2 includes, for example, human ULBP2 protein (UniProt ID: Q9BZM5), which is 246 amino acids in length.

In one aspect, the invention provides isolated antibodies that bind to ULBP2. In some instances the anti-ULBP2 antibody binds to a human ULBP2 polypeptide or a portion thereof. In some embodiments, the human ULBP2 polypeptide comprises the amino acid sequence of SEQ ID NO: 421.

ULBP5 (RAET1G) encompasses naturally occurring variants of ULBP5, including, for example, splice variants or allelic variants. ULBP5 includes, for example, human ULBP5 protein (UniProt ID: Q6H3X3), which is 334 amino acids in length.

In one aspect, the invention provides isolated antibodies that bind to ULBP5 (RAET1G). In some instances the anti-ULBP5 antibody binds to a human ULBP5 polypeptide or a portion thereof. In some embodiments, the human ULBP5 polypeptide comprises the amino acid sequence of SEQ ID NO: 422.

ULBP6 (RAET1L) encompasses naturally occurring variants of ULBP6, including, for example, splice variants or allelic variants. ULBP6 includes, for example, human ULBP6 protein (UniProt ID: Q5VY80), which is 246 amino acids in length.

In one aspect, the invention provides isolated antibodies that bind to ULBP6 (RAET1L). In some instances the anti-ULBP6 antibody binds to a human ULBP6 polypeptide or a portion thereof. In some embodiments, the human ULBP6 polypeptide comprises the amino acid sequence of SEQ ID NO: 423.

### Anti-ULBP2/5/6 Antibodies

Provided herein are antibodies that bind to anti-ULBP2/5/6. In some embodiments, alanine scanning mutagenesis may be performed on the “E12” anti-ULBP2/5/6 antibody to produce affinity modulated anti-ULBP2/5/6 antibodies. Also provided herein are antibodies that bind to anti-ULBP2. In some embodiments, alanine scanning mutagenesis may be performed on the “A06” anti-ULBP2 antibody to produce affinity modulated anti-ULBP2 antibodies.

In some embodiments, a anti-ULBP2/5/6 antibody of the disclosure comprises any one of the VH and VL sequences listed in Table 10. In Table 10, the underlined sequences are CDR sequence according to Kabat and the bolded sequences are CDR sequences according to Chothia.

In some embodiments, a anti-ULBP2 antibody of the disclosure comprises: a) a heavy chain variable region (VH) comprising a VH complementarity determining region 1 (VH_{CDR1}), a VH complementarity determining region 2 (VH_{CDR2}) and a VH complementarity determining region 3 (VH_{CDR3}); and b) a light chain variable region (VL) comprising a VL complementarity determining region 1 (VL_{CDR1}), a VL complementarity determining region 2 (VL_{CDR2}) and a VL complementarity determining region 3 (VL_{CDR3}). Tables 11 and 12 provide exemplary of CDR sequences of the anti-ULBP2 antibodies provided herein.

TABLE 10
Anti-ULBP2/5/6 Variable Heavy Chain and Variable Light Chain
Domains

| Ab | | Amino Acid Sequence | |
|---|---|---|---|
| ULBP2/ | VL-1 | EIVLTQSPATLSLSPGERATLSCRASQSIGTSIHWYQKKPGQAPR | SEQ ID |
| 5/6- | | LLIYFASESISGIPARFSGSGSGTDFTLTISSLEPEDFAVYYCQQ | NO: 1 |
| 01 | | SNSWPLYTFGGGTKVEIK | |
| | VH-1 | QVQLVQSGAEVKKPGASVKVSCKASGYTFTNYDINWVRDAPGQGL | SEQ ID |
| | | EWMGWIYPGDGSTYYNEKFKGRVTMTRDTSTSTVYMELSSLRSED | NO: 2 |
| | | TAVYYCARSGDYGLYYFDYWGQGTLVTVSS | |
| ULBP2/ | VL-2 | EIVLTQSPATLSLSPGERATLSCRASQSIGTSIHWYQDKPGQAPR | SEQ ID |
| 5/6- | | LLIYFASESISGIPARFSGSGSGTDFTLTISSLEPEDFAVYYCQQ | NO: 3 |
| 02 | | SNSWPLYTFGGGTKVEIK | |
| | VH-2 | QVQLVQSGAEVKKPGASVKVSCKASGYTFTNYDINWVRKAPGQGL | SEQ ID |
| | | EWMGWIYPGDGSTYYNEKFKGRVTMTRDTSTSTVYMELSSLRSED | NO: 4 |
| | | TAVYYCARSGDYGLYYFDYWGQGTLVTVSS | |
| E12 | VL-2 | EIVLTQSPATLSLSPGERATLSCRASQSIGTSIHWYQQKPGQAPR | SEQ ID |
| | | LLIYFASESISGIPARFSGSGSGTDFTLTISSLEPEDFAVYYCQQ | NO: 424 |
| | | SNSWPLYTFGGGTKVEIK | |
| | VH-2 | QVQLVQSGAEVKKPGASVKVSCKASGYTFTNYDINWVRQAPGQGL | SEQ ID |
| | | EWMGWIYPGDGSTYYNEKFKGRVTMTRDTSTSTVYMELSSLRSED | NO: 425 |
| | | TAVYYCARSGDYGLYYFDYWGQGTLVTVSS | |
| A06 | VL-2 | EIVMTQSPATLSLSPGERITLSCSASSSIHSNYLHWYQQKPGQAP | SEQ ID |
| | | RLLIYRTSNLASGIPARFSGSGSGTDFTLTISSLQPEDFAVYYCQ | NO: 426 |
| | | QGFSIPFTFGQGTKLEIK | |
| | VH-2 | EVQLVQSGAEVKKPGESLKISCKGSGYAFSTSWMNWVRQMPGKGL | SEQ ID |
| | | EWMGRIYPGDGDTNYNGNFKGQVTISADKSISTAYLQWSSLKASD | NO: 427 |
| | | TAMYYCARGGELRLEAMDSWGQGTLVTVSS | |
| ULBP2/ | VL-1 | EIVLTQSPATLSLSPGERATLSCRASQSIGTSIHWYQKKPGQAPR | SEQ ID |
| 5/6- | | LLIYFASESISGIPARFSGSGSGTDFTLTISSLEPEDFAVYYCQQ | NO: 1 |
| 03 | | SNSWPLYTFGGGTKVEIK | |
| | VH-3 | EVQLVQSGAEVKKPGASVKVSCKASGYTFTNYDINWVRDAPGQGL | SEQ ID |
| | | EWMGWIYPGDGSTYYNEKFKGRVTMTRDTSTSTVYMELSSLRSED | NO: 629 |
| | | TAVYYCARSGDYGLYYFDYWGQGTLVTVSS | |

TABLE 11
Anti-ULBP2/5/6 Heavy Chain CDRs

| | CDRH1 | CDRH2 | CDRH3 |
|---|---|---|---|
| ULBP2/5/6- | NYDIN (SEQ ID NO: 5) | WIYPGDGSTYYNEKFKG (SEQ | SGDYGLYYFDY |
| 01 | (Kabat) | ID NO: 7) (Kabat) | (SEQ ID NO: |
| | GYTFTNY (SEQ ID NO: | YPGDGS (SEQ ID NO: 8) | 9) |
| | 6) (Chothia) | (Chothia) | |
| ULBP2/5/6- | NYDIN (SEQ ID NO: 5) | WIYPGDGSTYYNEKFKG (SEQ | SGDYGLYYFDY |
| 02 | (Kabat) | ID NO: 7) (Kabat) | (SEQ ID NO: |
| | GYTFTNY (SEQ ID NO: | YPGDGS (SEQ ID NO: 8) | 9) |
| | 6) (Chothia) | (Chothia) | |
| E12 | NYDIN (SEQ ID NO: 5) | WIYPGDGSTYYNEKFKG (SEQ | SGDYGLYYFDY |
| | (Kabat) | ID NO: 7) (Kabat) | (SEQ ID NO: |
| | GYTFTNY (SEQ ID NO: | YPGDGS (SEQ ID NO: 8) | 9) |
| | 6) (Chothia) | (Chothia) | |
| A06 | TSWMN (SEQ ID NO: | RIYPGDGDTNYNGNFKG (SEQ | GGELRLEAMDS |
| | 428) (Kabat) | ID NO: 430) (Kabat) | (SEQ ID NO: |
| | GYAFSTS (SEQ ID NO: | YPGDGD (SEQ ID NO: | 432) |
| | 429) (Chothia) | 431) | |
| | | (Chothia) | |

TABLE 12
Anti-ULBP2/5/6 Light Chain CDRs

| | CDRL1 | CDRL2 | CDRL3 |
|---|---|---|---|
| ULBP2- | RASQSIGTSIH | FASESIS | QQSNSWPLYT |
| 01 | (SEQ ID NO: 10) | (SEQ ID NO: 11) | (SEQ ID NO: 12) |
| ULBP2- | RASQSIGTSIH | FASESIS | QQSNSWPLYT |
| 02 | (SEQ ID NO: 10) | (SEQ ID NO: 11) | (SEQ ID NO: 12) |
| E12 | RASQSIGTSIH | FASESIS | QQSNSWPLYT |
| | (SEQ ID NO: 10) | (SEQ ID NO: 11) | (SEQ ID NO: 12) |
| A06 | SASSSIHSNYLH | RTSNLAS | QQGFSIPFT |
| | (SEQ ID NO: 433) | (SEQ ID NO: 434) | (SEQ ID NO: 435) |

In some embodiments, the disclosure provides an antibody (e.g. including antibody fragments, such as single chain variable fragments (scFvs) which specifically bind to ULBP2, wherein the antibody comprises a) a heavy chain variable region (VH) comprising a i) a VH complementarity determining region 1 (VH_{CDR1}) comprising the amino acid sequence of SEQ ID NO: 5 or 6, ii) a VH complementarity determining region 2 (VH_{CDR2}) comprising the amino acid sequence of SEQ ID NO: 7 or 8, iii) a VH complementarity determining region 3 (VH_{CDR3}) comprising the amino acid sequence of SEQ ID NO: 9; and b) a light chain variable region (VL) comprising a i) a VL complementarity determining region 1 (VL_{CDR1}) comprising the amino acid sequence of SEQ ID NO: 10, ii) a VL complementarity determining region 2 (VL_{CDR2}) comprising the amino acid sequence of SEQ ID NO: 11, iii) a VL complementarity determining region 3 (VL_{CDR3}) comprising the amino acid sequence of SEQ ID NO: 12.

Exemplary anti-ULBP2 antibodies of the invention include ULBP2-01, ULBP2-02, E12, A06.

In some embodiments, the anti-ULBP2 antibody ULBP2-01 has a VH region comprising a VH_{CDR1} comprising the amino acid sequence of SEQ ID NO: 5, a VH_{CDR2} comprising the amino acid sequence of SEQ ID NO: 7, and a VH_{CDR3} comprising the amino acid sequence of SEQ ID NO: 9; and a VL region comprising a VL_{CDR1} comprising the amino acid sequence of SEQ ID NO: 10, a VL_{CDR2} comprising the amino acid sequence of SEQ ID NO: 11, and a VL_{CDR3} comprising the amino acid sequence of SEQ ID NO: 12.

In some embodiments, the anti-ULBP2 antibody ULBP2-01 has a VH region comprising the amino acid sequence shown in SEQ ID NO: 2 and a VL region comprising the amino acid sequence shown in SEQ ID NO: 1.

In some embodiments, the anti-ULBP2 antibody ULBP2-02 has a VH region comprising a VH_{CDR1} comprising the amino acid sequence of SEQ ID NO: 5, a VH_{CDR2} comprising the amino acid sequence of SEQ ID NO: 7, and a VH_{CDR3} comprising the amino acid sequence of SEQ ID NO: 9; and a VL region comprising a VL_{CDR1} comprising the amino acid sequence of SEQ ID NO: 10, a VL_{CDR2} comprising the amino acid sequence of SEQ ID NO: 11, and a VL_{CDR3} comprising the amino acid sequence of SEQ ID NO: 12.

In some embodiments, the anti-ULBP2 antibody ULBP2-02 has a VH region comprising the amino acid sequence shown in SEQ ID NO: 4 and a VL region comprising the amino acid sequence shown in SEQ ID NO: 3.

In some embodiments, the anti-ULBP2 antibody E12 has a VH region comprising a VH_{CDR1} comprising the amino acid sequence of SEQ ID NO: 5, a VH_{CDR2} comprising the amino acid sequence of SEQ ID NO: 7, and a VH_{CDR3} comprising the amino acid sequence of SEQ ID NO: 9; and a VL region comprising a VL_{CDR1} comprising the amino acid sequence of SEQ ID NO: 10, a VL_{CDR2} comprising the amino acid sequence of SEQ ID NO: 11, and a VL_{CDR3} comprising the amino acid sequence of SEQ ID NO: 12.

In some embodiments, the anti-ULBP2 antibody E12 has a VH region comprising the amino acid sequence shown in SEQ ID NO: 425 and a VL region comprising the amino acid sequence shown in SEQ ID NO: 424.

In some embodiments, the anti-ULBP2 antibody A06 has a VH region comprising a VH_{CDR1} comprising the amino acid sequence of SEQ ID NO: 428, a VH_{CDR2} comprising the amino acid sequence of SEQ ID NO: 430, and a VH_{CDR3} comprising the amino acid sequence of SEQ ID NO: 432; and a VL region comprising a VL_{CDR1} comprising the amino acid sequence of SEQ ID NO: 433, a VL_{CDR2} comprising the amino acid sequence of SEQ ID NO: 434, and a VL_{CDR3} comprising the amino acid sequence of SEQ ID NO: 435.

In some embodiments, the anti-ULBP2 antibody A06 has a VH region comprising the amino acid sequence shown in SEQ ID NO: 427 and a VL region comprising the amino acid sequence shown in SEQ ID NO: 426.

### Bispecific Anti-ULBP2/5/6 Antibodies

Provided herein are bispecific antibodies comprising a first antigen binding domain that binds a first antigen (e.g. cell surface antigen, CD3ε) and a second antigen binding domain that binds to a second antigen (e.g. ULBP2/5/6).

In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2).

In some embodiments, the bispecific antibody of the disclosure comprises a second antigen binding domain (e.g. binding to ULBP2/5/6) comprising any one of the VH2 and VL2 sequences listed in Table 10. In Table 10, the underlined sequences are CDR sequence according to Kabat and the bolded sequences are CDR sequences according to Chothia.

In some embodiments, the bispecific antibody of the disclosure comprises a second antigen binding domain (e.g. binding to ULBP2) comprising: a) a heavy chain variable region (VH2) comprising a VH complementarity determining region 1 (VH2_{CDR1}), a VH complementarity determining region 2 (VH2_{CDR2}) and a VH2 complementarity determining region 3 (VH2_{CDR3}); and b) a light chain variable region (VL2) comprising a VL complementarity determining region 1 (VL2_{CDR1}), a VL complementarity determining region 2 (VL2_{CDR2}) and a VL complementarity determining region 3 (VL2_{CDR3}). Tables 11 and 12 provide exemplary of CDR sequences of the anti-ULBP2 antibodies provided herein.

In some embodiments, the bispecific antibody comprises any one of the anti-ULBP2/5/6 antibodies of the disclosure. Exemplary anti-ULBP2/5/6 antibodies of the invention include ULBP2-01, ULBP2-02 and E12. In some embodiments, the bispecific antibody comprises any one of the anti-ULBP2 antibodies of the disclosure. Exemplary anti-ULBP2 antibodies of the invention include A06.

In some embodiments, the disclosure provides an isolated antibody (e.g. monospecific antibody or bispecific antibody) which specifically binds to ULPB2/5/6 and competes with any of the foregoing antibodies.

In some embodiments, the present invention provides an antibody (e.g. monospecific antibody or bispecific antibody) that binds to ULBP2/5/6 and competes with an antibody as described herein, including ULBP2-01, ULBP2-02, A06 and E12.

In some embodiments, the invention also provides CDR portions of antibodies to ULBP2/5/6 antibodies based on CDR contact regions. CDR contact regions are regions of an antibody that imbue specificity to the antibody for an antigen. In general, CDR contact regions include the residue positions in the CDRs and Vernier zones which are constrained in order to maintain proper loop structure for the antibody to bind a specific antigen. See, e.g., Makabe et al., J. Biol. Chem., 283:1156-1166, 2007. Determination of CDR contact regions is well within the skill of the art.

The binding affinity (K_{D}) of the ULBP2/5/6 antibody (e.g. monospecific antibody or bispecific antibody) as described herein to ULBP2/5/6 (such as human ULBP2 (e.g., (SEQ ID NO: 421), ULBP5 (SEQ ID NO: 422), ULBP6 (SEQ ID NO: 423)) can be about 0.001 to about 5000 nM.

In some embodiments, the binding affinity is about any of 5000 nM, 4500 nM, 4000 nM, 3500 nM, 3000 nM, 2500 nM, 2000 nM, 1789 nM, 1583 nM, 1540 nM, 1500 nM, 1490 nM, 1064 nM, 1000 nM, 933 nM, 894 nM, 750 nM, 705 nM, 678 nM, 532 nM, 500 nM, 494 nM, 400 nM, 349 nM, 340 nM, 353 nM, 300 nM, 250 nM, 244 nM, 231 nM, 225 nM, 207 nM, 200 nM, 186 nM, 172 nM, 136 nM, 113 nM, 104 nM, 101 nM, 100 nM, 90 nM, 83 nM, 79 nM, 74 nM, 54 nM, 50 nM, 45 nM, 42 nM, 40 nM, 35 nM, 32 nM, 30 nM, 25 nM, 24 nM, 22 nM, 20 nM, 19 nM, 18 nM, 17 nM, 16 nM, 15 nM, 12 nM, 10 nM, 9 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM, 0.1 nM, 0.01 nM, or 0.001 nM.

In some embodiments, the binding affinity is less than about any of 5000 nM, 4000 nM, 3000 nM, 2000 nM, 1000 nM, 900 nM, 800 nM, 250 nM, 200 nM, 100 nM, 50 nM, 30 nM, 20 nM, 10 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

In some embodiments, the disclosure provides a nucleic acid encoding any of the foregoing isolated anti-ULBP2/5/6 antibodies (e.g. monospecific antibody or bispecific antibody). In some embodiments, the disclosure provides a vector comprising such a nucleic acid. In some embodiments, the disclosure provides a host cell comprising such a nucleic acid.

Exemplary Bispecific Antibodies that Bind to CD3ε and ULBP2/5/6

Provided herein are bispecific antibodies comprising a first antigen binding domain that binds a first antigen (e.g. CD3ε) and a second antigen binding domain that binds to a second antigen (e.g. ULBP2/5/6).

In some embodiments the bispecific antibody has the following structure: a first heavy chain polypeptide (H1) comprising a variable region (VH1), and a constant region (CH1) having a constant region 1 domain (CH1_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1), and a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2). In some embodiments, the bispecific antibody of the disclosure comprises a first antigen binding domain (e.g. binding to CD3ε) comprising: a) a heavy chain variable region (VH1) comprising a VH complementarity determining region 1 (VH1_{CDR1}), a VH complementarity determining region 2 (VH1_{CDR2}) and a VH complementarity determining region 3 (VH1_{CDR3}); and b) a light chain variable region (VL) comprising a VL complementarity determining region 1 (VL1_{CDR1}), a VL complementarity determining region 2 (VL1_{CDR2}) and a VL complementarity determining region 3 (VL1_{CDR3}); and a second antigen binding domain (e.g. binding to ULBP2/5/6) comprising: a) a heavy chain variable region (VH2) comprising a VH complementarity determining region 1 (VH2_{CDR1}), a VH complementarity determining region 2 (VH2_{CDR2}) and a VH2 complementarity determining region 3 (VH2_{CDR3}); and b) a light chain variable region (VL2) comprising a VL complementarity determining region 1 (VL2_{CDR1}), a VL complementarity determining region 2 (VL2_{CDR2}) and a VL complementarity determining region 3 (VL2_{CDR3}). Tables 8 and 9 provide exemplary of CDR sequences of the anti-CD3ε antibodies provided herein. Tables 11 and 12 provide exemplary of CDR sequences of the anti-ULBP2/5/6 antibodies provided herein.

In some embodiments, the bispecific antibody of the disclosure comprises a first antigen binding domain (e.g. binding to CD3ε) comprising any one of the VH1 and VL1 sequences listed in Table 7 and a second antigen binding domain (e.g. binding to ULBP2/5/6) comprising any one of the VH2 and VL2 sequences listed in Table 10.

In some embodiments, the bispecific antibody of the disclosure comprises a first heavy chain polypeptide (H1) and a first light chain polypeptide (L1); and a second heavy chain polypeptide (H2) and a second light chain polypeptide (L2) comprising any one of the sequences listed in Table 13 and Table 14. The italicized sequences are the heavy chain variable regions and the light chain variable regions. The underlined sequences are CDRs according to Kabat and the bolded sequences are CDRs according to Chothia.

In some embodiments, the bispecific antibody of the disclosure provided herein comprises a H1 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 14.

In some embodiments, the bispecific antibody of the disclosure provided herein comprises a L1 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 14.

In some embodiments, the bispecific antibody of the disclosure provided herein comprises a H2 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 14.

In some embodiments, the bispecific antibody of the disclosure provided herein comprises a L2 that is at least 90%, at least 91%, at least 92%, at least 93%, at least 94%, at least 95%, at least 96%, at least 97%, at least 98%, at least 99% or at least 100% identical to the amino acid sequence of the sequences listed in Table 13 and Table 14.

In some embodiments, the H1 amino acid sequence is numbered in accordance with SEQ ID NO: 439. In some embodiments the L1 amino acid sequence is numbered in accordance with SEQ ID NO: 438. In some embodiments, the H2 amino acid sequence is numbered in accordance with SEQ ID NO: 437. In some embodiments, the L2 amino acid sequence is numbered in accordance with SEQ ID NO: 436.

TABLE 13
Exemplary Bispecific Antibodies that bind CD38 and ULBP2/5/6

| Antibody | Heavy/ | | SEQ ID |
|---|---|---|---|
| Name | Light Chain | Amino Acid Sequence | NO: |
| EIP | ULBP2/5/6 | EIVLTQSPATLSLSPGERATLSCRASQSIGTSIHWYQXKPGQAPRLLIYFASES | SEQ ID |
| bi- | light | ISGIPARFSGSGSGTDFTLTISSLEPEDFAVYYCQQSNSWPLYTFGGGTKVEIK | NO: 436 |
| specific | chain (L2) | RTVAAPXVXIFPPSDEQLKSGTASVVCLLNXFYPREAKVQWKVDNALQSGNSQE | |
| antibody | | XVTEQDSKDSTYSLSSTLXLSKADYEKHKVYACEVTHQGLSSPVTKSFNRGEX | |
| | ULBP2/5/6 | QVQLVQSGAEVKKPGASVKVSCKASGYTFTNYDINWVRXAPGQGLEWMGWIYPG | SEQ ID |
| | heavy | DGSTYYNEKFKGRVTMTRDTSTSTVYMELSSLRSEDTAVYYCARSGDYGLYYFD | NO: 437 |
| | chain (H2) | YWGQGTLVTVSSASTKGPSVFPLAPXSKXTXGGTAALGCLVXDYFPEPVTVSWN | |
| | | SGALTSGVHTXXAVLQSSGLYSLSSVVXVPSSSLGTQTYICNVNHKPSNTKVDK | |
| | | KVEPKSXDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQXKPGQAPRGLIGGTN | SEQ ID |
| | chain (L1) | KRAPGTPARFSGSLLGGKAALTLSGVQPEDEAEYYCALWYSNLWVFGGGTKLTV | NO: 438 |
| | | LGQPKANPTVTLXPPSSEELQANKAXLVCLIXDFYPGAVTVAWKADGSPVKAGV | |
| | | ETXKPSKQSNNKYAASSYLXLTPEQWKSHRSYSCQVTHEGSTVEKTVAPTEXS | |
| | CD3 heavy | EVQLLESGGGLVQPGGSLKLSCAASGFTFNTYAMNWVRXAPGKGLEWVARIRSK | SEQ ID |
| | chain (H1) | YNNYATYYADSVKDRFTISRDDSKNTAYLQMNNLKTEDTAVYYCVRHGNFGNSY | NO: 439 |
| | | VSWFAYWGQGTLVTVSSASTKGPSVFPXAPSSKSTSGGTAALGCXVXDYFPEPV | |
| | | TVSWNSGALTSGVHTXPAXLQSSGLYSLSSXVTVPSSSLGTQTYICNVNHKPSN | |
| | | TKVDKKVEPKSXDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0174 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 55 |
| | chain (L2) | RTVAAPSVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 56 |
| | chain (H2) | YWGQGTLVTVSSASTKGPSVFPLAPSSKSTSGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 57 |
| | | LGQPKANPTVTLCPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 58 |
| | | VSWFAYWGQGTLVTVSSASTKGPSVFPCAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0175 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 59 |
| | chain (L2) | RTVAAPSVCIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 60 |
| | chain (H2) | YWGQGTLVTVSSASTKGPSVFPLAPSSKCTSGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 61 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 62 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0187 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 63 |
| | chain (L2) | RTVAAPCVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 64 |
| | chain (H2) | YWGQGTLVTVSSASTKGPSVFPLAPSSKSTCGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 65 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 66 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0205 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 67 |
| | chain (L2) | RTVAAPSVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 68 |
| | chain (H2) | YWGQGTLVTVSSASTKGPSVFPLAPSSKSTSGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 69 |
| | | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 70 |
| | | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0206 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 71 |
| | chain (L2) | RTVAAPSVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 72 |
| | chain (H2) | YWGQGTLVTVSSASTKGPSVFPLAPSSKSTSGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 73 |
| | | LGQPKANPTVTLFPPSSEELQANKATLVCLIDDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 74 |
| | | VSWFAYWGQGTLVTVSSASTKGPSVFPLAPSSKSTSGGTAALGCLVKDYFPEPV | |
| | | TVSWNSGALTSGVHTFPACLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0207 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 75 |
| | chain (L2) | RTVAAPCVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 76 |
| | chain (H2) | YWGQGTLVTVSSASTKGPSVFPLAPCSKSTSGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 77 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 78 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0208 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 79 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 80 |
| | chain (H2) | YWGQGTLVTVSSASTKGPSVFPLAPSSKSTSGGTAALGCLVKDYFPEPVTVSWN | |
| | | SGALTSGVHTCPAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 81 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 82 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0294 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 83 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 84 |
| | chain (H2) | YWGQGTLVTVSSASTKGPSVFPLAPSSKSTSGGTAALGCLVKDYFPEPVTVSWN | |
| | | SGALTSGVHTFCAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 85 |
| | | LGQPKANPTVTLFPPSSEELQANKATLVCLIKDFYPGAVTVAWKADGSPVKAGV | |
| | | ETTKPSKQSNNKYAASSYLELTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 86 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSEVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| V295 | ULBP2/5/6 | EIVLTQSPATLSLSPGERATLSCRASQSIGTSIHWYQKKPGQAPRLLIYFASES | SEQ ID |
| EIP0295 | light | ISGIPARFSGSGSGTDFTLTISSLEPEDFAVYYCQQSNSWPLYTFGGGTKVEIK | NO: 87 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 88 |
| | chain (H2) | YWGQGTLVTVSSASTKGPSVFPLAPSSKSTSGGTAALGCLVKDYFPEPVTVSWN | |
| | | SGALTSGVHTFCAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 89 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 90 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0306 | ULBP2/5/6 | EIVLTQSPATLSLSPGERATLSCRASQSIGTSIHWYQKKPGQAPRLLIYFASES | SEQ ID |
| | | ISGIPARFSGSGSGTDFTLTISSLEPEDFAVYYCQQSNSWPLYTFGGGTKVEIK | |
| | light | RTVAAPSVFIFPPSDEQLKSGTASVVCLLNNFYPREAKVQWKVDNALQSGNSQE | NO: 91 |
| | chain (L2) | CVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 92 |
| | chain (H2) | YWGQGTLVTVSSASTKGPSVFPLAPSSKSTSGGTAALGCLVDDYFPEPVTVSWN | |
| | | SGALTSGVHTFCAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 93 |
| | | LGQPKANPTVTLFPPSSEELQANKATLVCLIKDFYPGAVTVAWKADGSPVKAGV | |
| | | ETTKPSKQSNNKYAASSYLELTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 94 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSEVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0307 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 95 |
| | chain (L2) | RTVAAPSVFIFPPSDEQLKSGTASVVCLLNNFYPREAKVQWKVDNALQSGNSQE | |
| | | CVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 96 |
| | chain (H2) | YWGQGTLVTVSSASTKGPSVFPLAPSSKSTSGGTAALGCLVDDYFPEPVTVSWN | |
| | | SGALTSGVHTFCAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 97 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 98 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0318 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 99 |
| | chain (L2) | RTVAAPSVFIFPPSDEQLKSGTASVVCLLNNFYPREAKVQWKVDNALQSGNSQE | |
| | | CVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 100 |
| | chain (H2) | YWGQGTLVTVSSASTKGPSVFPLAPSSKSTSGGTAALGCLVDDYFPEPVTVSWN | |
| | | SGALTSGVHTFCAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 101 |
| | | LGQPKANPTVTLFPPSSEELQANKADLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 102 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0340 | ULBP2/5/6 | EIVLTQSPATLSLSPGERATLSCRASQSIGTSIHWYQDKPGQAPRLLIYFASES | SEQ ID |
| | light | ISGIPARFSGSGSGTDFTLTISSLEPEDFAVYYCQQSNSWPLYTFGGGTKVEIK | NO: 103 |
| | chain (L2) | RTVAAPCVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKFNRGES | |
| | ULBP2/5/6 | QVQLVQSGAEVKKPGASVKVSCKASGYTFTNYDINWVRKAPGQGLEWMGWIYPG | SEQ ID |
| | heavy | DGSTYYNEKFKGRVTMTRDTSTSTVYMELSSLRSEDTAVYYCARSGDYGLYYFD | NO: 104 |
| | chain (H2) | YWGQGTLVTVSSASTKGPSVFPLAPSSKSTCGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQKKPGQAPRGLIGGTN | SEQ ID |
| | chain (L1) | KRAPGTPARFSGSLLGGKAALTLSGVQPEDEAEYYCALWYSNLWVFGGGTKLTV | NO: 105 |
| | | LGQPKANPTVTLFPPSSEELQANKATLVCLIKDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRDAPGKGLEWVARIRSK | SEQ ID |
| | chain (H1) | YNNYATYYADSVKDRFTISRDDSKNTAYLQMNNLKTEDTAVYYCVRHGNFGNSY | NO: 106 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSEVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0342 | ULBP2/5/6 | EIVLTQSPATLSLSPGERATLSCRASQSIGTSIHWYQDKPGQAPRLLIYFASES | SEQ ID |
| | light | ISGIPARFSGSGSGTDFTLTISSLEPEDFAVYYCQQSNSWPLYTFGGGTKVEIK | NO: 107 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRKAPGQGLEWMGWIYPG | SEQ ID |
| | heavy | DGSTYYNEKFKGRVTMTRDTSTSTVYMELSSLRSEDTAVYYCARSGDYGLYYFD | NO: 108 |
| | chain (H2) | YWGQGTLVTVSSASTKGPSVFPLAPSSKSTSGGTAALGCLVKDYFPEPVTVSWN | |
| | | SGALTSGVHTFCAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQKKPGQAPRGLIGGTN | SEQ ID |
| | chain (L1) | KRAPGTPARFSGSLLGGKAALTLSGVQPEDEAEYYCALWYSNLWVFGGGTKLTV | NO: 109 |
| | | LGQPKANPTVTLFPPSSEELQANKATLVCLIKDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRDAPGKGLEWVARIRSK | SEQ ID |
| | chain (H1) | YNNYATYYADSVKDRFTISRDDSKNTAYLQMNNLKTEDTAVYYCVRHGNFGNSY | NO: 110 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSEVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0354 | ULBP2/5/6 | EIVLTQSPATLSLSPGERATLSCRASQSIGTSIHWYQDKPGQAPRLLIYFASES | SEQ ID |
| | light | ISGIPARFSGSGSGTDFTLTISSLEPEDFAVYYCQQSNSWPLYTFGGGTKVEIK | NO: 111 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRKAPGQGLEWMGWIYPG | SEQ ID |
| | heavy | DGSTYYNEKFKGRVTMTRDTSTSTVYMELSSLRSEDTAVYYCARSGDYGLYYFD | NO: 112 |
| | chain (H2) | YWGQGTLVTVSSASTKGPSVFPLAPSSKSTSGGTAALGCLVKDYFPEPVTVSWN | |
| | | SGALTSGVHTFCAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQKKPGQAPRGLIGGTN | SEQ ID |
| | chain (L1) | KRAPGTPARFSGSLLGGKAALTLSGVQPEDEAEYYCALWYSNLWVFGGGTKLTV | NO: 113 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRDAPGKGLEWVARIRSK | SEQ ID |
| | chain (H1) | YNNYATYYADSVKDRFTISRDDSKNTAYLQMNNLKTEDTAVYYCVRHGNFGNSY | NO: 114 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0356 | ULBP2/5/6 | EIVLTQSPATLSLSPGERATLSCRASQSIGTSIHWYQDKPGQAPRLLIYFASES | SEQ ID |
| | light | ISGIPARFSGSGSGTDFTLTISSLEPEDFAVYYCQQSNSWPLYTFGGGTKVEIK | NO: 115 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRKAPGQGLEWMGWIYPG | SEQ ID |
| | heavy | DGSTYYNEKFKGRVTMTRDTSTSTVYMELSSLRSEDTAVYYCARSGDYGLYYFD | NO: 116 |
| | chain (H2) | YWGQGTLVTVSSASTKGPSVFPLAPSSKSTSGGTAALGCLVKDYFPEPVTVSWN | |
| | | SGALTSGVHTCPAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQKKPGQAPRGLIGGTN | SEQ ID |
| | chain (L1) | KRAPGTPARFSGSLLGGKAALTLSGVQPEDEAEYYCALWYSNLWVFGGGTKLTV | NO: 117 |
| | | LGQPKANPTVTLFPPSSEELQANKADLVCLIKDFYPGAVTVAWKADGSPVKAGV | |
| | | ETTKPSKQSNNKYAASSYLELTPEQWKSHRSYSCQVTHEGSTVEKTVAPTECS | |
| | CD3 heavy | EVQLLESGGGLVQPGGSLKLSCAASGFTFNTYAMNWVRDAPGKGLEWVARIRSK | SEQ ID |
| | chain (H1) | YNNYATYYADSVKDRFTISRDDSKNTAYLQMNNLKTEDTAVYYCVRHGNFGNSY | NO: 118 |
| | | VSWFAYWGQGTLVTVSSASTKGPSVFPLAPSSKSTSGGTAALGCSVKDYFPEPV | |
| | | TVSWNSGALTSGVHTFPAVLQSSGLYSLSSEVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| V367 | ULBP2/5/6 | EIVLTQSPATLSLSPGERATLSCRASQSIGTSIHWYQKKPGQAPRLLIYFASES | SEQ ID |
| EIP0367 | light | ISGIPARFSGSGSGTDFTLTISSLEPEDFAVYYCQQSNSWPLYTFGGGTKVEIK | NO: 119 |
| | chain (L2) | RTVAAPSVFIFPPSDEQLKSGTASVVCLLNNFYPREAKVQWKVDNALQSGNSQE | |
| | | CVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 120 |
| | chain (H2) | YWGQGTLVTVSSASTKGPSVFPLAPSSKSTSGGTAALGCLVDDYFPEPVTVSWN | |
| | | SGALTSGVHTCPAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 121 |
| | | LGQPKANPTVTLFPPSSEELQANKATLVCLIDDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 122 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSKVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0377 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 123 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 124 |
| | chain (H2) | YWGQGTLVTVSSASTKGPSVFPLAPSSKSTSGGTAALGCLVKDYFPEPVTVSWN | |
| | | SGALTSGVHTCPAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 125 |
| | | LGQPKANPTVTLFPPSSEELQANKAKLVCLISDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 126 |
| | | VSWFAYWGQGTLVTVSSASTKGPSVFPLAPSSKSTSGGTAALGCSVDDYFPEPV | |
| | | TVSWNSGALTSGVHTFPAVLQSSGLYSLSSVVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0404 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 127 |
| | chain (L2) | RTVAAPCVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 128 |
| | chain (H2) | YWGQGTLVTVSSASTKGPSVFPLAPSSKSTCGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQEKPGQAPRGLIGGTN | SEQ ID |
| | chain (L1) | KRAPGTPARFSGSLLGGKAALTLSGVQPEDEAEYYCALWYSNLWVFGGGTKLTV | NO: 129 |
| | | LGQPKANPTVTLFPPSSEELQANKADLVCLIKDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 130 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSEVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| V406 | ULBP2/5/6 | EIVLTQSPATLSLSPGERATLSCRASQSIGTSIHWYQKKPGQAPRLLIYFASES | SEQ ID |
| EIP0406 | light | ISGIPARFSGSGSGTDFTLTISSLEPEDFAVYYCQQSNSWPLYTFGGGTKVEIK | NO: 131 |
| | chain (L2) | RTVAAPCVFIFPPSDEQLKSGTASVVCLLNKFYPREAKVQWKVDNALQSGNSQE | |
| | | SVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 132 |
| | chain (H2) | YWGQGTLVTVSSASTKGPSVFPLAPSSKSTCGGTAALGCLVKDYFPEPVTVSWN | |
| | | SGALTSGVHTSPAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQEKPGQAPRGLIGGTN | SEQ ID |
| | chain (L1) | KRAPGTPARFSGSLLGGKAALTLSGVQPEDEAEYYCALWYSNLWVFGGGTKLTV | NO: 133 |
| | | LGQPKANPTVTLFPPSSEELQANKADLVCLIKDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 134 |
| | | VSWFAYWGQGTLVTVSSASTKGPSVFPLAPSSKSTSGGTAALGCLVKDYFPEPV | |
| | | TVSWNSGALTSGVHTFPAVLQSSGLYSLSSEVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0473 | ULBP2/5/6 | EIVLTQSPATLSLSPGERATLSCRASQSIGTSIHWYQDKPGQAPRLLIYFASES | SEQ ID |
| | light | ISGIPARFSGSGSGTDFTLTISSLEPEDFAVYYCQQSNSWPLYTFGGGTKVEIK | NO: 135 |
| | chain (L2) | RTVAAPSVFIFPPSDEQLKSGTASVVCLLNKFYPREAKVQWKVDNALQSGNSQE | |
| | | CVTEQDSKDSTYSLSSTLTLSKADYEKHKVYACEVTHQGLSSPVTKSFNRGES | |
| | ULBP2/5/6 | QVQLVQSGAEVKKPGASVKVSCKASGYTFTNYDINWVRKAPGQGLEWMGWIYPG | SEQ ID |
| | heavy | DGSTYYNEKFKGRVTMTRDTSTSTVYMELSSLRSEDTAVYYCARSGDYGLYYFD | NO: 136 |
| | chain (H2) | YWGQGTLVTVSSASTKGPSVFPLAPSSKSTSGGTAALGCLVKDYFPEPVTVSWN | |
| | | SGALTSGVHTFCAVLQSSGLYSLSSVVDVPSSSLGTQTYICNVNHKPSNTKVDK | |
| | | KVEPKSSDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 137 |
| | | LGQPKANPTVTLFPPSSEELQANKAKLVCLIKDFYPGAVTVAWKADGSPVKAGV | |
| | | ETTKPSKQSNNKYAASSYLSLTPEQWKSHRSYSCQVTHEGSTVEKTVAPTECS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 138 |
| | | VSWFAYWGQGTLVTVSSASTKGPSVFPLAPSSKSTSGGTAALGCLVDDYFPEPV | |
| | | TVSWNSGALTSGVHTFPAVLQSSGLYSLSSDVTVPSSSLGTQTYICNVNHKPSN | |
| | | TKVDKKVEPKSCDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0598 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 139 |
| | chain (L2) | RTVAAPSVFIFPPSDEQLKSGTASVVCLLNNFYPREAKVQWKVDNALQSGNSQE | |
| | | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 140 |
| | chain (H2) | YWGQGTLVTVSSASTKGPSVFPLAPSSKSTSGGTAALGCLVDDYFPEPVTVSWN | |
| | | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain (L1) | KRAPGTPARFSGSLLGGKAALTLSGVQPEDEAEYYCALWYSNLWVFGGGTKLTV | NO: 141 |
| | | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain (H1) | YNNYATYYADSVKDRFTISRDDSKNTAYLQMNNLKTEDTAVYYCVRHGNFGNSY | NO: 142 |
| | | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |

Exemplary, CD3ε x ULBP2/5/6 bispecific antibodies of the invention include EIP0174, EIP0175, EIP0187, EIP0205, EIP0206, EIP0207, EIP0208, EIP0294, EIP0295, EIP0306, EIP0307, EIP0318, EIP0340, EIP0342, EIP0354, EIP0356, EIP0367, EIP0377, EIP0404, EIP0406, EIP0473, and EIP0598.

Bispecific antibodies EIP0174, EIP0175, EIP0187, EIP0205, EIP0206, EIP0207, EIP0208, EIP0294, EIP0295, EIP0306, EIP0307, EIP0318, EIP0340, EIP0342, EIP0354, EIP0356, EIP0367, EIP0377, EIP0404, EIP0406, EIP0473, and EIP0598 comprise a first antigen binding domain that binds CD3ε which comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

Bispecific antibodies EIP0174, EIP0175, EIP0187, EIP0205, EIP0206, EIP0207, EIP0208, EIP0294, EIP0295, EIP0306, EIP0307, EIP0318, EIP0340, EIP0342, EIP0354, EIP0356, EIP0367, EIP0377, EIP0404, EIP0406, EIP0473, and EIP0598 comprise a second antigen binding domain that binds ULBP2/5/6 comprising a VH2, comprising VH2_{CDR1} having the amino acid sequence of SEQ ID NO: 5; a VH2_{CDR2} having the amino acid sequence of SEQ ID NO: 7; and a VH2_{CDR3} having the amino acid sequence of SEQ ID NO: 9; and a VL2, having VL2_{CDR1} having the amino acid sequence of SEQ ID NO: 10; a VL2_{CDR2} having the amino acid sequence of SEQ ID NO: 11; and a VL2_{CDR3} having the amino acid sequence of SEQ ID NO: 12.

In addition to the specific complementarity determining regions recited above, the bispecific antibody EIP0174 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acid at position 128 (EU numbering) of the CH1_{H1} is a C and the amino acid at position 118 (EU numbering) of the CL1 is a C; the amino acid at position 220 (EU numbering) of the H1H is a S and the amino acid at position 214 (EU numbering) of the CL1 is an S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0174 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0174 comprises a VH1 comprising the amino acid sequence of SEQ ID NO: 13, a VL1 comprising the amino acid sequence of SEQ ID NO: 22, a VH2 comprising the amino acid sequence of SEQ ID NO: 2, and a VL2 comprising the amino acid sequence of SEQ ID NO: 1.

In some embodiments, the bispecific antibody EIP0174 comprises a H1 comprising the amino acid sequence of SEQ ID NO: 58, a L1 comprising the amino acid sequence of SEQ ID NO: 57, a H2 comprising the amino acid sequence of SEQ ID NO: 56, and a L2 comprising the amino acid sequence of SEQ ID NO: 55.

In addition to the specific complementarity determining regions recited above, the bispecific antibody EIP0175 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0175 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 134 (EU numbering) of the CH2_{H1} is a C and the amino acid at position 116 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0175 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0175 comprises a H1 comprising the amino acid sequence of SEQ ID NO: 62, a H2 comprising the amino acid sequence of SEQ ID NO: 60, a L1 comprising the amino acid sequence of SEQ ID NO: 61, and a L2 comprising the amino acid sequence of SEQ ID NO: 59.

In addition to the specific complementarity determining regions recited above, the bispecific antibody EIP0187 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0187 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 136 (EU numbering) of the CH2_{H1} is a C and the amino acid at position 114 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436. In some embodiments, the bispecific antibody EIP0187 comprises a VH1

comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0187 comprises a H1 comprising the amino acid sequence of SEQ ID NO: 66, a H2 comprising the amino acid sequence of SEQ ID NO: 64, a L1 comprising the amino acid sequence of SEQ ID NO: 65, and a L2 comprising the amino acid sequence of SEQ ID NO: 63.

In addition to the specific complementarity determining regions recited above, the bispecific antibody EIP0205 comprises the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a K and the amino acid at position 131 (EU numbering) of the CL1 is a D; the amino acid at position 173 (EU numbering) of the CH1_{H1} is a C and the amino acid at position 162 (EU numbering) of the CL1 is a C; the amino acid at position 220 (EU numbering) of the H1H is a S and the amino acid at position 214 (EU numbering) of the CL1 is a S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0205 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0205 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0205 comprises a H1 comprising the amino acid sequence of SEQ ID NO: 70, a H2 comprising the amino acid sequence of SEQ ID NO: 68, a L1 comprising the amino acid sequence of SEQ ID NO: 69, and a L2 comprising the amino acid sequence of SEQ ID NO: 67.

In addition to the specific complementarity determining regions recited above, the bispecific antibody EIP0206 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acid at position 173 (EU numbering) of the CH1_{H1} is a C and the amino acid at position 162 (EU numbering) of the CL1 is a C; the amino acid at position 220 (EU numbering) in the H1H is a S and the amino acid at position 214 in CL1 is a S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0206 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 134 (EU numbering) of the CH2_{H1} is a C and the amino acid at position 116 (EU numbering) of the CL2 is a C; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0206 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0206 comprises a H1 comprising the amino acid sequence of SEQ ID NO: 74, a H2 comprising the amino acid sequence of SEQ ID NO: 72, a L1 comprising the amino acid sequence of SEQ ID NO: 73, and a L2 comprising the amino acid sequence of SEQ ID NO: 71.

In addition to the specific complementarity determining regions recited above, the bispecific antibody EIP0207 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0207 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 131 (EU numbering) of the CH2_{H1} is a C and the amino acid at position 114 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436. In some embodiments, the bispecific antibody EIP0207 comprises a VH1

comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0207 comprises a H1 comprising the amino acid sequence of SEQ ID NO: 78, a H2 comprising the amino acid sequence of SEQ ID NO: 76, a L1 comprising the amino acid sequence of SEQ ID NO: 77, and a L2 comprising the amino acid sequence of SEQ ID NO: 75.

In addition to the specific complementarity determining regions recited above, the bispecific antibody EIP0208 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0208 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 138 (EU numbering) of the CL2 is a K; the amino acid at position 170 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0208 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0208 comprises a H1 comprising the amino acid sequence of SEQ ID NO: 82, a H2 comprising the amino acid sequence of SEQ ID NO: 80, a L1 comprising the amino acid sequence of SEQ ID NO: 81, and a L2 comprising the amino acid sequence of SEQ ID NO: 79.

In addition to the specific complementarity determining regions recited above, the bispecific antibody EIP0294 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a E and the amino acid at position 137 (EU numbering) of the CL1 is a K and the amino acid at position 179 (EU numbering) of the CL1 is a E; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0294 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 138 (EU numbering) of the CL2 is a K; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0294 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0294 comprises a H1 comprising the amino acid sequence of SEQ ID NO: 86, a H2 comprising the amino acid sequence of SEQ ID NO: 84, a L1 comprising the amino acid sequence of SEQ ID NO: 85, and a L2 comprising the amino acid sequence of SEQ ID NO: 83.

In addition to the specific complementarity determining regions recited above, the bispecific antibody EIP0295 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0295 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 138 (EU numbering) of the CL2 is a K; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436. In some embodiments, the bispecific antibody EIP0295 comprises a VH1

comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0295 comprises a H1 comprising the amino acid sequence of SEQ ID NO: 90, a H2 comprising the amino acid sequence of SEQ ID NO: 88, a L1 comprising the amino acid sequence of SEQ ID NO: 89, and a L2 comprising the amino acid sequence of SEQ ID NO: 87.

In addition to the specific complementarity determining regions recited above, the bispecific antibody EIP0306 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a E and the amino acid at position 137 (EU numbering) of the CL1 is a K and the amino acid at position 179 (EU numbering) of the CL1 is a E; the amino acid at position 185 (EU numbering) of the CH1_{H1} is a E and the amino acid at position 179 (EU numbering) of the CL1 is a E; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0306 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0306 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0306 comprises a H1 comprising the amino acid sequence of SEQ ID NO: 94, a H2 comprising the amino acid sequence of SEQ ID NO: 92, a L1 comprising the amino acid sequence of SEQ ID NO: 93, and a L2 comprising the amino acid sequence of SEQ ID NO: 91.

In addition to the specific complementarity determining regions recited above, the bispecific antibody EIP0307 comprises the following amino acids substitutions in the H1 and L1: the amino acid at position 39 (Kabat numbering) of the VH1 is a K and the amino acid at position 38 (Kabat numbering) of the VL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0307 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 171 (EU numbering) of the CH21 is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0307 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0307 comprises a H1 comprising the amino acid sequence of SEQ ID NO: 98, a H2 comprising the amino acid sequence of SEQ ID NO: 96, a L1 comprising the amino acid sequence of SEQ ID NO: 97, and a L2 comprising the amino acid sequence of SEQ ID NO: 95.

In addition to the specific complementarity determining regions recited above, the bispecific antibody EIP0318 comprises the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a K and the amino acid at position 131 (EU numbering) of the CL1 is a D; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0318 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0318 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0318 comprises a H1 comprising the amino acid sequence of SEQ ID NO: 102, a H2 comprising the amino acid sequence of SEQ ID NO: 100, a L1 comprising the amino acid sequence of SEQ ID NO: 101, and a L2 comprising the amino acid sequence of SEQ ID NO: 99.

In addition to the specific complementarity determining regions recited above, the bispecific antibody EIP0340 comprises the following amino acids substitutions in the H1 and L1: the amino acid at position 39 (Kabat numbering) of the VH1 is a D and the amino acid at position 38 (Kabat numbering) of the VL1 is a K; the amino acid at position 185 (EU numbering) of the CH1_{H1} is a E and the amino acid at position 137 (EU numbering) of the CL1 is a K; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0340 comprises the following amino acids substitutions in the H2 and L2: the amino acid at position 39 (Kabat numbering) of the VH2 is a K and the amino acid at position 38 (Kabat numbering) of the VL2 is a D; the amino acid at position 187 (EU numbering) of the CH2_{H1} is a D and the amino acid at position 138 (EU numbering) of the CL2 is a K; the amino acid at position 136 (EU numbering) of the CH2_{H1} is a C and the amino acid at position 114 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0340 comprises a VH1 comprising the amino acid sequence of SEQ ID NO: 14, a VH2 comprising the amino acid sequence of SEQ ID NO: 4, a VL1 comprising the amino acid sequence of SEQ ID NO: 23, and a VL2 comprising the amino acid sequence of SEQ ID NO: 3.

In some embodiments, the bispecific antibody EIP0340 comprises a H1 comprising the amino acid sequence of SEQ ID NO: 106, a H2 comprising the amino acid sequence of SEQ ID NO: 104, a L1 comprising the amino acid sequence of SEQ ID NO: 105, and a L2 comprising the amino acid sequence of SEQ ID NO: 103.

In addition to the specific complementarity determining regions recited above, the bispecific antibody EIP0342 comprises the following amino acids substitutions in the H1 and L1: the amino acid at position 39 (Kabat numbering) of the VH1 is a D and the amino acid at position 38 (Kabat numbering) of the VL1 is a K; the amino acid at position 185 (EU numbering) of the CH1H is a E and the amino acid at position 137 (EU numbering) of the CL1 is a K; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0342 comprises the following amino acids substitutions in the H2 and L2: the amino acid at position 39 (Kabat numbering) of the VH2 is a K and the amino acid at position 38 (Kabat numbering) of the VL2 is a D; the amino acid at position 187 (EU numbering) of the CH2_{H1} is a D and the amino acid at position 138 (EU numbering) of the CL2 is a K; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0342 comprises a VH1 comprising the amino acid sequence of SEQ ID NO: 14, a VH2 comprising the amino acid sequence of SEQ ID NO: 4, a VL1 comprising the amino acid sequence of SEQ ID NO: 23, and a VL2 comprising the amino acid sequence of SEQ ID NO: 3.

In some embodiments, the bispecific antibody EIP0342 comprises a H1 comprising the amino acid sequence of SEQ ID NO: 110, a H2 comprising the amino acid sequence of SEQ ID NO: 108, a L1 comprising the amino acid sequence of SEQ ID NO: 109, and a L2 comprising the amino acid sequence of SEQ ID NO: 107.

In addition to the specific complementarity determining regions recited above, the bispecific antibody EIP0354 comprises the following amino acids substitutions in the H1 and L1: the amino acid at position 39 (Kabat numbering) of the VH1 is a D and the amino acid at position 38 (Kabat numbering) of the VL1 is a K; the amino acid at position 185 (EU numbering) of the CH1_{H1} is a K and the amino acid at position 137 (EU numbering) of the CL1 is a D; the amino acids at positions 234, 235, and 237 (EU numbering) in the H1H are an A the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0354 comprises the following amino acids substitutions in the H2 and L2: the amino acid at position 39 (Kabat numbering) of the VH2 is a K and the amino acid at position 38 (Kabat numbering) of the VL2 is a D; the amino acid at position 187 (EU numbering) of the CH2_{H1} is a D and the amino acid at position 138 (EU numbering) of the CL2 is a K; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0354 comprises a VH1 comprising the amino acid sequence of SEQ ID NO: 14, a VH2 comprising the amino acid sequence of SEQ ID NO: 4, a VL1 comprising the amino acid sequence of SEQ ID NO: 23, and a VL2 comprising the amino acid sequence of SEQ ID NO: 3.

In some embodiments, the bispecific antibody EIP0354 comprises a H1 comprising the amino acid sequence of SEQ ID NO: 114, a H2 comprising the amino acid sequence of SEQ ID NO: 112, a L1 comprising the amino acid sequence of SEQ ID NO: 113, and a L2 comprising the amino acid sequence of SEQ ID NO: 111.

In addition to the specific complementarity determining regions recited above, the bispecific antibody EIP0356 comprises the following amino acids substitutions in the H1 and L1: the amino acid at position 39 (Kabat numbering) of the VH1 is a D and the amino acid at position 38 (Kabat numbering) of the VL1 is a K; the amino acid at position 147 (EU numbering) of the CH1_{H1} is a K and the amino acid at position 131 (EU numbering) of the CL1 is a D; the amino acid at position 185 (EU numbering) of the CH1_{H1} is a E and the amino acid at position 137 (EU numbering) of the CL1 is a K; the amino acid at position 145 (EU numbering) of the CH1_{H1} is a S and the amino acid at position 180 (EU numbering) of the CL1 is a E; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0356 comprises the following amino acids substitutions in the H2 and L2: the amino acid at position 39 (Kabat numbering) of the VH2 is a K and the amino acid at position 38 (Kabat numbering) of the VL2 is a D; the amino acid at position 187 (EU numbering) of the CH21 is a D and the amino acid at position 138 (EU numbering) of the CL2 is a K; the amino acid at position 170 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0356 comprises a VH1 comprising the amino acid sequence of SEQ ID NO: 14, a VH2 comprising the amino acid sequence of SEQ ID NO: 4, a VL1 comprising the amino acid sequence of SEQ ID NO: 23, and a VL2 comprising the amino acid sequence of SEQ ID NO: 3.

In some embodiments, the bispecific antibody EIP0356 comprises a H1 comprising the amino acid sequence of SEQ ID NO: 118, a H2 comprising the amino acid sequence of SEQ ID NO: 116, a L1 comprising the amino acid sequence of SEQ ID NO: 117, and a L2 comprising the amino acid sequence of SEQ ID NO: 115.

In addition to the specific complementarity determining regions recited above, the bispecific antibody EIP0367 comprises the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a D and the amino acid at position 131 (EU numbering) of the CL1 is a K; the amino acid at position 185 (EU numbering) of the CH1_{H1} is a E and the amino acid at position 137 (EU numbering) of the CL1 is a D; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0367 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 137 (EU numbering) of the CL2 is a K and the amino acid at position 138 (EU numbering) of the CL2 is a R; the amino acid at position 170 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0367 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0367 comprises a H1 comprising the amino acid sequence of SEQ ID NO: 122, a H2 comprising the amino acid sequence of SEQ ID NO: 120, a L1 comprising the amino acid sequence of SEQ ID NO: 121, and a L2 comprising the amino acid sequence of SEQ ID NO: 119.

In addition to the specific complementarity determining regions recited above, the bispecific antibody EIP0377 comprises the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a D and the amino acid at position 145 (EU numbering) of the CH1_{H1} is a S and the amino acid at position 131 (EU numbering) of the CL1 is a K; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0377 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 138 (EU numbering) of the CL2 is a K; the amino acid at position 170 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0377 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0377 comprises a H1 comprising the amino acid sequence of SEQ ID NO: 126, a H2 comprising the amino acid sequence of SEQ ID NO: 124, a L1 comprising the amino acid sequence of SEQ ID NO: 125, and a L2 comprising the amino acid sequence of SEQ ID NO: 123.

In addition to the specific complementarity determining regions recited above, the bispecific antibody EIP0404 comprises the following amino acids substitutions in the H1 and L1: the amino acid at position 39 (Kabat numbering) of the VH1 is a D and the amino acid at position 38 (Kabat numbering) of the VL1 is a K; the amino acid at position 147 (EU numbering) of the CH1_{H1} is a K and the amino acid at position 131 (EU numbering) of the CL1 is a D; the amino acid at position 185 (EU numbering) of the CH1_{H1} is a E and the amino acid at position 137 (EU numbering) of the CL1 is a K; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0404 comprises the following amino acids substitutions in the H2 and L2: the amino acid at position 39 (Kabat numbering) of the VH2 is a K and the amino acid at position 38 (Kabat numbering) of the VL2 is a D; the amino acid at position 147 (EU numbering) of the CH2_{H1} is a D and the amino acid at position 180 (EU numbering) of the CL2 is a R; the amino acid at position 136 (EU numbering) of the CH2_{H1} is a C and the amino acid at position 114 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436. In some embodiments, the bispecific antibody EIP0404 comprises a VH1

comprising the amino acid sequence of SEQ ID NO: 13, a VH2 comprising the amino acid sequence of SEQ ID NO: 2, a VL1 comprising the amino acid sequence of SEQ ID NO: 24, and a VL2 comprising the amino acid sequence of SEQ ID NO: 1.

In some embodiments, the bispecific antibody EIP0404 comprises a H1 comprising the amino acid sequence of SEQ ID NO: 130, a H2 comprising the amino acid sequence of SEQ ID NO: 128, a L1 comprising the amino acid sequence of SEQ ID NO: 129, and a L2 comprising the amino acid sequence of SEQ ID NO: 127.

In addition to the specific complementarity determining regions recited above, the bispecific antibody EIP0406 comprises the following amino acids substitutions in the H1 and L1: the amino acid at position 39 (Kabat numbering) of the VH1 is a K and the amino acid at position 38 (Kabat numbering) of the VL1 is a E; the amino acid at position 147 (EU numbering) of the CH1_{H1} is a K and the amino acid at position 131 (EU numbering) of the CL1 is a D; the amino acid at position 185 (EU numbering) of the CH1_{H1} is a E and the amino acid at position 137 (EU numbering) of the CL1 is a K; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0406 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 138 (EU numbering) of the CL2 is a K; the amino acid at position 136 (EU numbering) of the CH2_{H1} is a C and the amino acid at position 114 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0406 comprises a VH1 comprising the amino acid sequence of SEQ ID NO: 13, a VH2 comprising the amino acid sequence of SEQ ID NO: 2, a VL1 comprising the amino acid sequence of SEQ ID NO: 24, and a VL2 comprising the amino acid sequence of SEQ ID NO: 1.

In some embodiments, the bispecific antibody EIP0406 comprises a H1 comprising the amino acid sequence of SEQ ID NO: 134, a H2 comprising the amino acid sequence of SEQ ID NO: 132, a L1 comprising the amino acid sequence of SEQ ID NO: 133, and a L2 comprising the amino acid sequence of SEQ ID NO: 131.

In addition to the specific complementarity determining regions recited above, the bispecific antibody EIP0473 comprises the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a D and the amino acid at position 131 (EU numbering) of the CL1 is a K; the amino acid at position 185 (EU numbering) of the CH1_{H1} is a D and the amino acid at position 137 (EU numbering) of the CL1 is a K; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0473 comprises the following amino acids substitutions in the H2 and L2: the amino acid at position 39 (Kabat numbering) of the VH2 is a D and the amino acid at position 38 (Kabat numbering) of the VL2 is a K; the amino acid at position 187 (EU numbering) of the CH2_{H1} is a D and the amino acid at position 138 (EU numbering) of the CL2 is a K; the amino acid at position 171 (EU numbering) of the CH2_{H1} is a C and the amino acid at position 162 (EU numbering) of the CL2 is a C; the amino acid at position 220 (EU numbering) in the H2H is a S and the amino acid at position 214 (EU numbering) of the CL2 is a S; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436. In some embodiments, the bispecific antibody EIP0473 comprises a VH1

comprising the amino acid sequence of SEQ ID NO: 13, a VH2 comprising the amino acid sequence of SEQ ID NO: 4, a VL1 comprising the amino acid sequence of SEQ ID NO: 22, and a VL2 comprising the amino acid sequence of SEQ ID NO: 3.

In some embodiments, the bispecific antibody EIP0473 comprises a H1 comprising the amino acid sequence of SEQ ID NO: 138, a H2 comprising the amino acid sequence of SEQ ID NO: 136, a L1 comprising the amino acid sequence of SEQ ID NO: 137, and a L2 comprising the amino acid sequence of SEQ ID NO: 135.

In addition to the specific complementarity determining regions recited above, the bispecific antibody EIP0598 comprises the following amino acids substitutions in the H1 and L1: the amino acid at position 39 (Kabat numbering) of the VH1 is a K and the amino acid at position 38 (Kabat numbering) of the VL1 is a D; the amino acid at position 170 (EU numbering) of the CH1_{H1} is a S and the amino acid at position 131 (EU numbering) of the CL1 is a D; the amino acid at position 173 (EU numbering) of the CH1_{H1} is a C and the amino acid at position 162 (EU numbering) of the CL1 is a C; the amino acid at position 220 (EU numbering) of the H1H is a S and the amino acid at position 214 (EU numbering) of the CL1 is an S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In addition to the specific complementarity determining regions recited above, the bispecific antibody EIP0598 comprises the following amino acids substitutions in the H2 and L2: the amino acid at position 39 (Kabat numbering) of the VH2 is a D and the amino acid at position 38 (Kabat numbering) of the VL2 is a K; the amino acid at position 147 (EU numbering) of the CH2_{H1} is a D and the amino acid at position 180 (EU numbering) of the CL2 is a R; the amino acids at positions 234, 235, and 237 (EU numbering) of the H2H are an A; the amino acid at position 354 (EU numbering) of the CH2_{H3} is a C; the amino acid at position 366 (EU numbering) of the CH2_{H3} is a W; and the amino acid at position 447 (EU numbering) of the CH2_{H3} is deleted, when numbered in accordance with H2 amino acid sequence of SEQ NO: 437 and L2 amino acid sequence of SEQ NO: 436.

In some embodiments, the bispecific antibody EIP0598 comprises a VH1 comprising the amino acid sequence of SEQ ID NO: 13, a VH2 comprising the amino acid sequence of SEQ ID NO: 2, a VL1 comprising the amino acid sequence of SEQ ID NO: 22, and a VL2 comprising the amino acid sequence of SEQ ID NO: 1.

In some embodiments, the bispecific antibody EIP0598 comprises a H1 comprising the amino acid sequence of SEQ ID NO: 142, a H2 comprising the amino acid sequence of SEQ ID NO: 140, a L1 comprising the amino acid sequence of SEQ ID NO: 141, and a L2 comprising the amino acid sequence of SEQ ID NO: 139.

Any one of the bispecific antibodies shown above in Table 13 can be further modified by substituting any one of the anti-CD3ε antigen binding regions with any one of the anti-CD3ε binding regions shown in Tables 7-9. For example, the anti-CD3ε antigen binding regions of bispecific antibody “EIP0205” can be substituted with any one of the anti-CD3ε binding regions shown in Tables 7-9 to produce the bispecific antibodies of the invention. Exemplary antibodies are shown in Table 14. The underlined sequences are CDR sequence according to Kabat and the bolded sequences are CDR sequences according to Chothia.

TABLE 14
Exemplary Bispecific Antibodies that bind to CD38 and ULBP2/5/6

| Antibody | Heavy/ | | SEQ ID |
|---|---|---|---|
| Name | Light Chain | Amino Acid Sequence | NO: |
| EIP0527 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 143 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 144 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWDSNLWVFGGGTKLTV | NO: 145 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGNSY | NO: 146 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0624 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 147 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 148 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 149 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGDSY | NO: 150 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0486 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 151 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 152 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGDTN | SEQ ID |
| | chain | KRAPGTPARFSGSLLGGKAALTLSGVQPEDEAEYYCALWYSNLWVFGGGTKLTV | NO: 153 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGNSY | NO: 154 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0626 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 155 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 156 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 157 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGDDY | NO: 158 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0483 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 159 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 160 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSNLWVFGGGTKLTV | NO: 161 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNDYAMNWVRKAPGKGLEWVARIRSK | SEQ ID |
| | chain | YNNYATYYADSVKDRFTISRDDSKNTAYLQMNNLKTEDTAVYYCVRHGNFGNSY | NO: 162 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0623 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 163 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 164 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 165 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVAKIRSK | SEQ ID |
| | chain | YNNYATYYADSVKDRFTISRDDSKNTAYLQMNNLKTEDTAVYYCVRHGNFGDSY | NO: 166 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0621 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 167 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 168 |
| | chain | YWGQGTLVTVSSASTKGPSVFPLAPSSKSTSGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 169 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGSFGNSY | NO: 170 |
| | (H1) | VSWFAYWGQGTLVTVSSASTKGPSVFPLAPSSKSTSGGTAALGCLVKDYFPEPV | |
| | (N96S) | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0625 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 171 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 172 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSNLWVFGGGTKLTV | NO: 173 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVAKIRSK | SEQ ID |
| | chain | YNNYATYYADSVKDRFTISRDDSKNTAYLQMNNLKTEDTAVYYCVRHGNFGDSY | NO: 174 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0622 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 175 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 176 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 177 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGTFGNSY | NO: 178 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0525 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 179 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 180 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSNLWVFGGGTKLTV | NO: 181 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMDWVRKAPGKGLEWVARIRSK | SEQ ID |
| | chain | YNNYATYYADSVKDRFTISRDDSKNTAYLQMNNLKTEDTAVYYCVRHGNFGNSY | NO: 182 |
| | (H1) | VSWFAYWGQGTLVTVSSASTKGPSVFPLAPSSKSTSGGTAALGCLVKDYFPEPV | |
| | | TVSWNSGALTSGVHTFPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0630 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 183 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 184 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWDSNLWVFGGGTKLTV | NO: 185 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGNSY | NO: 186 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0540 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 187 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 188 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 189 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGDSY | NO: 190 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0628 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 191 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 192 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGDTN | SEQ ID |
| | chain | KRAPGTPARFSGSLLGGKAALTLSGVQPEDEAEYYCALWYSNLWVFGGGTKLTV | NO: 193 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGNSY | NO: 194 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0542 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 195 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 196 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 197 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGNFGDDY | NO: 198 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0627 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 199 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 200 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSNLWVFGGGTKLTV | NO: 201 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNDYAMNWVRKAPGKGLEWVARIRSK | SEQ ID |
| | chain | YNNYATYYADSVKDRFTISRDDSKNTAYLQMNNLKTEDTAVYYCVRHGNFGNSY | NO: 202 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0515 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 203 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 204 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 205 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVAKIRSK | SEQ ID |
| | chain | YNNYATYYADSVKDRFTISRDDSKNTAYLQMNNLKTEDTAVYYCVRHGNFGDSY | NO: 206 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0477 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 207 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 208 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 209 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGSFGNSY | NO: 210 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0541 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 211 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 212 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSNLWVFGGGTKLTV | NO: 213 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVAKIRSK | SEQ ID |
| | chain | YNNYATYYADSVKDRFTISRDDSKNTAYLQMNNLKTEDTAVYYCVRHGNFGDSY | NO: 214 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0513 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 215 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 216 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSQLWVFGGGTKLTV | NO: 217 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMNWVRKAPGKGLEWVARIRSK | SEQ ID |
| | chain | YNNYATYYADSVKDRFTISRDDSKNTAYLQMNNLKTEDTAVYYCVRHGTFGNSY | NO: 218 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |
| EIP0629 | ULBP2/5/6 | EIVLTQSPATLSLSPGERATLSCRASQSIGTSIHWYQKKPGQAPRLLIYFASES | SEQ ID |
| | light | ISGIPARFSGSGSGTDFTLTISSLEPEDFAVYYCQQSNSWPLYTFGGGTKVEIK | NO: 219 |
| | chain | RTVAAPSVFIFPPSDEQLKSGTASVVCLLNNFYPREAKVQWKVDNALQSGNSQE | |
| | (L2) | SVTEQDSKDSTYSLSSTLRLSKADYEKHKVYACEVTHQGLSSPVTKSFNRGEC | |
| | ULBP2/5/6 | QVQLVQSGAEVKKPGASVKVSCKASGYTFTNYDINWVRDAPGQGLEWMGWIYPG | SEQ ID |
| | heavy | DGSTYYNEKFKGRVTMTRDTSTSTVYMELSSLRSEDTAVYYCARSGDYGLYYFD | NO: 220 |
| | chain | YWGQGTLVTVSSASTKGPSVFPLAPSSKSTSGGTAALGCLVDDYFPEPVTVSWN | |
| | (H2) | SGALTSGVHTFPAVLQSSGLYSLSSVVTVPSSSLGTQTYICNVNHKPSNTKVDK | |
| | | KVEPKSCDKTHTCPPCPAPEAAGAPSVFLFPPKPKDTLMISRTPEVTCVVVDVS | |
| | | HEDPEVKFNWYVDGVEVHNAKTKPREEQYNSTYRVVSVLTVLHQDWLNGKEYKC | |
| | | KVSNKALPAPIEKTISKAKGQPREPQVYTLPPCREEMTKNQVSLWCLVKGFYPS | |
| | | DIAVEWESNGQPENNYKTTPPVLDSDGSFFLYSKLTVDKSRWQQGNVFSCSVMH | |
| | | EALHNHYTQKSLSLSP | |
| | CD3 light | ELVVTQEPSLTVSPGGTVTLTCRSSTGAVTTSNYANWVQDKPGQAPRGLIGGTN | SEQ ID |
| | chain | KRAPGTPARFSGSLLGGKAALTLSGVQPEDEAEYYCALWYSNLWVFGGGTKLTV | NO: 221 |
| | (L1) | LGQPKANPTVTLFPPSSEELQANKADLVCLISDFYPGAVTVAWKADGSPVKAGV | |
| | | ETCKPSKQSNNKYAASSYLSLTPEQWKSHRSYSCQVTHEGSTVEKTVAPTESS | |
| | CD3 heavy | EVQLLESGGGLVQPGGSLKLSCAASGFTFNTYAMDWVRKAPGKGLEWVARIRSK | SEQ ID |
| | chain | YNNYATYYADSVKDRFTISRDDSKNTAYLQMNNLKTEDTAVYYCVRHGNFGNSY | NO: 222 |
| | (H1) | VSWFAYWGQGTLVTVSSASTKGPSVFPLAPSSKSTSGGTAALGCLVKDYFPEPV | |
| | | TVSWNSGALTSGVHTSPACLQSSGLYSLSSVVTVPSSSLGTQTYICNVNHKPSN | |
| | | TKVDKKVEPKSSDKTHTCPPCPAPEAAGAPSVFLFPPKPKDTLMISRTPEVTCV | |
| | | VVDVSHEDPEVKFNWYVDGVEVHNAKTKPREEQYNSTYRVVSVLTVLHQDWLNG | |
| | | KEYKCKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQGNVFS | |
| | | CSVMHEALHNHYTQKSLSLSP | |

In some embodiments, exemplary, CD3ε x ULBP2/5/6 bispecific antibodies of the invention include EIP0527, EIP0624, EIP0486, EIP0626, EIP0483, EIP0623, EIP0621, EIP0625, EIP0622, EIP0525.

Bispecific antibodies EIP0527, EIP0624, EIP0486, EIP0626, EIP0483, EIP0623, EIP0621, EIP0625, EIP0622, and EIP0525 comprise the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a K and the amino acid at position 131 (EU numbering) of the CL1 is a D; the amino acid at position 173 (EU numbering) of the CH1_{H1} is a C and the amino acid at position 162 (EU numbering) of the CL1 is a C; the amino acid at position 220 (EU numbering) of the H1H is a S and the amino acid at position 214 (EU numbering) of the CL1 is a S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

Bispecific antibodies EIP0527, EIP0624, EIP0486, EIP0626, EIP0483, EIP0623, EIP0621, EIP0625, EIP0622, and EIP0525 have a second antigen binding domain that binds ULBP2/5/6 comprising a VH2, comprising VH2_{CDR1} having the amino acid sequence of SEQ ID NO: 5; a VH2_{CDR2} having the amino acid sequence of SEQ ID NO: 7; and a VH2_{CDR3} having the amino acid sequence of SEQ ID NO: 9; and a VL2, comprising a VL2_{CDR1} having the amino acid sequence of SEQ ID NO: 10; a VL2_{CDR2} having the amino acid sequence of SEQ ID NO: 11; and a VL2_{CDR3} having the amino acid sequence of SEQ ID NO: 12.

Bispecific antibodies EIP0527, EIP0624, EIP0486, EIP0626, EIP0483, EIP0623, EIP0621, EIP0625, EIP0622, and EIP0525 comprise a VH2 comprising the amino acid sequence of SEQ ID NO: 2; and a VL2 comprising the amino acid sequence of SEQ ID NO: 1.

Bispecific antibodies EIP0527, EIP0624, EIP0486, EIP0626, EIP0483, EIP0623, EIP0621, EIP0625, EIP0622, and EIP0525 have a H2 comprising the amino acid sequence of SEQ ID NO: 68; and a L2 comprising the amino acid sequence of SEQ ID NO: 67.

In addition to the VH2, VL2, H2, L2 sequences and amino acid substitutions shown above, the bispecific antibody EIP0527 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 46.

In some embodiments, the bispecific antibody EIP0527 comprises a VH1 having the amino acid sequence of SEQ ID NO: 13 and a VL1 having the amino acid sequence of SEQ ID NO: 25. In some embodiments, the bispecific antibody EIP0527 comprises a H1 having the amino acid sequence of SEQ ID NO: 146 and a L1 having the amino acid sequence of SEQ ID NO: 145.

In addition to the VH2, VL2, H2, L2 sequences and amino acid substitutions shown above, the bispecific antibody EIP0624 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0624 comprises a VH1 having the amino acid sequence of SEQ ID NO: 15 and a VL1 having the amino acid sequence of SEQ ID NO: 26. In some embodiments, the bispecific antibody EIP0624 comprises a H1 having the amino acid sequence of SEQ ID NO: 150 and a L1 having the amino acid sequence of SEQ ID NO: 149.

In addition to the VH2, VL2, H2, L2 sequences and amino acid substitutions shown above, the bispecific antibody EIP0486 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 44; and a VL1_{CDR3} having the amino acid sequence of SEQ ID NO: 45.

In some embodiments, the bispecific antibody EIP0486 comprises a VH1 having the amino acid sequence of SEQ ID NO: 13 and a VL1 having the amino acid sequence of SEQ ID NO: 27. In some embodiments, the bispecific antibody EIP0486 comprises a H1 having the amino acid sequence of SEQ ID NO: 154 and a L1 having the amino acid sequence of SEQ ID NO: 153.

In addition to the VH2, VL2, H2, L2 sequences and amino acid substitutions shown above, the bispecific antibody EIP0626 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 39; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0626 comprises a VH1 having the amino acid sequence of SEQ ID NO: 16 and a VL1 having the amino acid sequence of SEQ ID NO: 26. In some embodiments, the bispecific antibody EIP0626 comprises a H1 having the amino acid sequence of SEQ ID NO: 158 and a L1 having the amino acid sequence of SEQ ID NO: 157.

In addition to the VH2, VL2, H2, L2 sequences and amino acid substitutions shown above, the bispecific antibody EIP0483 comprises a first antigen binding domain that binds CD3ε comprising a VH1, comprising a VH1_{CDR1} having the amino acid sequence of SEQ ID NO: 30; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0483 comprises a VH1 having the amino acid sequence of SEQ ID NO: 17 and a VL1 having the amino acid sequence of SEQ ID NO: 22. In some embodiments, the bispecific antibody EIP0483 comprises a H1 having the amino acid sequence of SEQ ID NO: 162 and a L1 having the amino acid sequence of SEQ ID NO: 161.

In addition to the VH2, VL2, H2, L2 sequences and amino acid substitutions shown above, the bispecific antibody EIP0623 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 35; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0623 comprises a VH1 having the amino acid sequence of SEQ ID NO: 18 and a VL1 having the amino acid sequence of SEQ ID NO: 26. In some embodiments, the bispecific antibody EIP0623 comprises a H1 having the amino acid sequence of SEQ ID NO: 166 and a L1 having the amino acid sequence of SEQ ID NO: 165.

In addition to the VH2, VL2, H2, L2 sequences and amino acid substitutions shown above, the bispecific antibody EIP0621 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 40; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0621 comprises a VH1 having the amino acid sequence of SEQ ID NO: 19 and a VL1 having the amino acid sequence of SEQ ID NO: 26. In some embodiments, the bispecific antibody EIP0621 comprises a H1 having the amino acid sequence of SEQ ID NO: 170 and a L1 having the amino acid sequence of SEQ ID NO: 169.

In addition to the VH2, VL2, H2, L2 sequences and amino acid substitutions shown above, the bispecific antibody EIP0625 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 35; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0625 comprises a VH1 having the amino acid sequence of SEQ ID NO: 18 and a VL1 having the amino acid sequence of SEQ ID NO: 22. In some embodiments, the bispecific antibody EIP0625 comprises a H1 having the amino acid sequence of SEQ ID NO: 174 and a L1 having the amino acid sequence of SEQ ID NO: 173.

In addition to the VH2, VL2, H2, L2 sequences and amino acid substitutions shown above, the bispecific antibody EIP0622 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 41; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0622 comprises a VH1 having the amino acid sequence of SEQ ID NO: 20 and a VL1 having the amino acid sequence of SEQ ID NO: 26. In some embodiments, the bispecific antibody EIP0622 comprises a H1 having the amino acid sequence of SEQ ID NO: 178 and a L1 having the amino acid sequence of SEQ ID NO: 177.

In addition to the VH2, VL2, H2, L2 sequences and amino acid substitutions shown above, the bispecific antibody EIP0525 comprises a first antigen binding domain that binds CD3ε comprising a VH1, comprising a VH1_{CDR1} having the amino acid sequence of SEQ ID NO: 31; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0525 comprises a VH1 having the amino acid sequence of SEQ ID NO: 21 and a VL1 having the amino acid sequence of SEQ ID NO: 22. In some embodiments, the bispecific antibody EIP0525 comprises a H1 having the amino acid sequence of SEQ ID NO: 182 and a L1 having the amino acid sequence of SEQ ID NO: 181.

In some embodiments, exemplary, CD3ε x ULBP2/5/6 bispecific antibodies of the invention include EIP0630, EIP0540, EIP0628, EIP0542, EIP0627, EIP0515, EIP0477, EIP0541, EIP0513, EIP0629.

Bispecific antibodies EIP0630, EIP0540, EIP0628, EIP0542, EIP0627, EIP0515, EIP0477, EIP0541, EIP0513, and EIP0629 have the following amino acids substitutions in the H1 and L1: the amino acid at position 39 (Kabat numbering) of the VH1 is a K and the amino acid at position 38 (Kabat numbering) of the VL1 is a D; the amino acid at position 170 (EU numbering) of the CH1_{H1} is a S and the amino acid at position 131 (EU numbering) of the CL1 is a D; the amino acid at position 173 (EU numbering) of the CH1_{H1} is a C and the amino acid at position 162 (EU numbering) of the CL1 is a C; the amino acid at position 220 (EU numbering) of the H1H is a S and the amino acid at position 214 (EU numbering) of the CL1 is an S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

Bispecific antibodies EIP0630, EIP0540, EIP0628, EIP0542, EIP0627, EIP0515, EIP0477, EIP0541, EIP0513, and EIP0629 have a second antigen binding domain that binds ULBP2/5/6 comprising a VH2, having VH2_{CDR1} having the amino acid sequence of SEQ ID NO: 5; a VH2_{CDR2} comprising the amino acid sequence of SEQ ID NO: 7; and a VH2_{CDR3} having the amino acid sequence of SEQ ID NO: 9; and a VL2, having VL2_{CDR1} having the amino acid sequence of SEQ ID NO: 10; a VL2_{CDR2} having the amino acid sequence of SEQ ID NO: 11; and a VL2_{CDR3} having the amino acid sequence of SEQ ID NO: 12.

Bispecific antibodies EIP0630, EIP0540, EIP0628, EIP0542, EIP0627, EIP0515, EIP0477, EIP0541, EIP0513, and EIP0629 have a VH2 having the amino acid sequence of SEQ ID NO: 2; and a VL2 having the amino acid sequence of SEQ ID NO: 1.

Bispecific antibodies EIP0630, EIP0540, EIP0628, EIP0542, EIP0627, EIP0515, EIP0477, EIP0541, EIP0513, and EIP0629 have a H2 having the amino acid sequence of SEQ ID NO: 140; and a L2 having the amino acid sequence of SEQ ID NO: 139.

In addition to the VH2, VL2, H2, L2 sequences and amino acid substitutions shown above, the bispecific antibody EIP0630 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 46.

In some embodiments, the bispecific antibody EIP0630 comprises a VH1 having the amino acid sequence of SEQ ID NO: 13 and a VL1 having the amino acid sequence of SEQ ID NO: 25. In some embodiments, the bispecific antibody EIP0630 comprises a H1 having the amino acid sequence of SEQ ID NO: 186 and a L1 having the amino acid sequence of SEQ ID NO: 185.

In addition to the VH2, VL2, H2, L2 sequences and amino acid substitutions shown above, the bispecific antibody EIP0540 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0540 comprises a VH1 having the amino acid sequence of SEQ ID NO: 15 and a VL1 having the amino acid sequence of SEQ ID NO: 26. In some embodiments, the bispecific antibody EIP0540 comprises a H1 having the amino acid sequence of SEQ ID NO: 190 and a L1 having the amino acid sequence of SEQ ID NO: 189.

In addition to the VH2, VL2, H2, L2 sequences and amino acid substitutions shown above, the bispecific antibody EIP0628 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 44; and a VL1_{CDR3} having the amino acid sequence of SEQ ID NO: 45.

In some embodiments, the bispecific antibody EIP0628 comprises a VH1 having the amino acid sequence of SEQ ID NO: 13 and a VL1 having the amino acid sequence of SEQ ID NO: 27. In some embodiments, the bispecific antibody EIP0628 comprises a H1 having the amino acid sequence of SEQ ID NO: 194 and a L1 having the amino acid sequence of SEQ ID NO: 193.

In addition to the VH2, VL2, H2, L2 sequences and amino acid substitutions shown above, the bispecific antibody EIP0542 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 39; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0542 comprises a VH1 having the amino acid sequence of SEQ ID NO: 16 and a VL1 having the amino acid sequence of SEQ ID NO: 26. In some embodiments, the bispecific antibody EIP0542 comprises a H1 having the amino acid sequence of SEQ ID NO: 198 and a L1 having the amino acid sequence of SEQ ID NO: 197.

In addition to the VH2, VL2, H2, L2 sequences and amino acid substitutions shown above, the bispecific antibody EIP0627 comprises a first antigen binding domain that binds CD3ε comprising a VH1, comprising a VH1_{CDR1} having the amino acid sequence of SEQ ID NO: 30; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0627 comprises a VH1 having the amino acid sequence of SEQ ID NO: 17 and a VL1 having the amino acid sequence of SEQ ID NO: 22. In some embodiments, the bispecific antibody EIP0627 comprises a H1 having the amino acid sequence of SEQ ID NO: 202 and a L1 having the amino acid sequence of SEQ ID NO: 201.

In addition to the VH2, VL2, H2, L2 sequences and amino acid substitutions shown above, the bispecific antibody EIP0515 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 35; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0515 comprises a VH1 having the amino acid sequence of SEQ ID NO: 18 and a VL1 having the amino acid sequence of SEQ ID NO: 26. In some embodiments, the bispecific antibody EIP0515 comprises a H1 having the amino acid sequence of SEQ ID NO: 206 and a L1 having the amino acid sequence of SEQ ID NO: 205.

In addition to the VH2, VL2, H2, L2 sequences and amino acid substitutions shown above, the bispecific antibody EIP0477 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 40; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0477 comprises a VH1 having the amino acid sequence of SEQ ID NO: 19 and a VL1 having the amino acid sequence of SEQ ID NO: 26. In some embodiments, the bispecific antibody EIP0477 comprises a H1 having the amino acid sequence of SEQ ID NO: 210 and a L1 having the amino acid sequence of SEQ ID NO: 209.

In addition to the VH2, VL2, H2, L2 sequences and amino acid substitutions shown above, the bispecific antibody EIP0541 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 35; and a VH1_{CDR3} having the amino acid sequence of SEQ ID NO: 38; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0541 comprises a VH1 having the amino acid sequence of SEQ ID NO: 18 and a VL1 having the amino acid sequence of SEQ ID NO: 22. In some embodiments, the bispecific antibody EIP0541 comprises a H1 having the amino acid sequence of SEQ ID NO: 214 and a L1 having the amino acid sequence of SEQ ID NO: 213.

In addition to the VH2, VL2, H2, L2 sequences and amino acid substitutions shown above, the bispecific antibody EIP0513 comprises a first antigen binding domain that binds CD3ε comprising a VH1, comprising a VH1_{CDR1} having the amino acid sequence of SEQ ID NO: 29; a VH1_{CDR2} having the amino acid sequence of SEQ ID NO: 34; and a VH1_{CDR3} having the amino acid sequence of SEQ ID NO: 41; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 47.

In some embodiments, the bispecific antibody EIP0513 comprises a VH1 having the amino acid sequence of SEQ ID NO: 20 and a VL1 having the amino acid sequence of SEQ ID NO: 26. In some embodiments, the bispecific antibody EIP0513 comprises a H1 having the amino acid sequence of SEQ ID NO: 218 and a L1 having the amino acid sequence of SEQ ID NO: 217.

In addition to the VH2, VL2, H2, L2 sequences and amino acid substitutions shown above, the bispecific antibody EIP0629 comprises a first antigen binding domain that binds CD3ε comprising a VH1, comprising a VH1_{CDR1} having the amino acid sequence of SEQ ID NO: 31; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0629 comprises a VH1 having the amino acid sequence of SEQ ID NO: 21 and a VL1 having the amino acid sequence of SEQ ID NO: 22. In some embodiments, the bispecific antibody EIP0629 comprises a H1 having the amino acid sequence of SEQ ID NO: 222 and a L1 having the amino acid sequence of SEQ ID NO: 221.

In some embodiments, an exemplary CD3ε x ULBP2/5/6 bispecific antibody of the invention includes EIP0820.

In some embodiments, bispecific antibody EIP0820 comprises the following amino acids substitutions in the H1 and L1: the amino acid at position 39 (Kabat numbering) of the VH1 is a K and the amino acid at position 38 (Kabat numbering) of the VL1 is a D; the amino acid at position 147 (EU numbering) of the CH1_{H1} is a K and the amino acid at position 131 (EU numbering) of the CL1 is a D; the amino acid at position 173 (EU numbering) of the CH1_{H1} is a C and the amino acid at position 162 (EU numbering) of the CL1 is a C; the amino acid at position 220 (EU numbering) of the H1H is a S and the amino acid at position 214 (EU numbering) of the CL1 is a S; the amino acids at positions 234, 235, and 237 (EU numbering) in the H1H are an A; the amino acid at position 349 (EU numbering) of the CH1_{H3} is a C; the amino acid at position 366 (EU numbering) of the CH1_{H3} is a S; the amino acid at position 368 (EU numbering) of the CH1_{H3} is a A; the amino acid at position 407 (EU numbering) of CH1_{H3} is a V; and the amino acid at position 447 (EU numbering) of the CH1_{H3} is deleted, when numbered in accordance with H1 amino acid sequence of SEQ NO: 439 and L1 amino acid sequence of SEQ NO: 438.

In some embodiments, bispecific antibody EIP0820 has a second antigen binding domain that binds ULBP2/5/6 comprising a VH2, comprising VH2_{CDR1} having the amino acid sequence of SEQ ID NO: 5; a VH2_{CDR2} having the amino acid sequence of SEQ ID NO: 7; and a VH2_{CDR3} having the amino acid sequence of SEQ ID NO: 9; and a VL2, comprising a VL2_{CDR1} having the amino acid sequence of SEQ ID NO: 10; a VL2_{CDR2} having the amino acid sequence of SEQ ID NO: 11; and a VL2_{CDR3} having the amino acid sequence of SEQ ID NO: 12.

In some embodiments, bispecific antibody EIP0820 comprises a VH2 comprising the amino acid sequence of SEQ ID NO: 2; and a VL2 comprising the amino acid sequence of SEQ ID NO: 1.

In some embodiments, bispecific antibody EIP0820 comprises a VH2 comprising the amino acid sequence of SEQ ID NO: 629; and a VL2 comprising the amino acid sequence of SEQ ID NO: 1.

In some embodiments, bispecific antibody EIP0820 comprises a H2 comprising the amino acid sequence of SEQ ID NO: 621; and a L2 comprising the amino acid sequence of SEQ ID NO: 67.

In addition to the VH2, VL2, H2, L2 sequences and amino acid substitutions shown above, the bispecific antibody EIP0820 comprises a first antigen binding domain that binds CD3ε comprising a VH1, comprising a VH1_{CDR1} having the amino acid sequence of SEQ ID NO: 30; a VH1_{CDR2} having the amino acid sequence of SEQ ID NO: 34; and a VH1_{CDR3} having the amino acid sequence of SEQ ID NO: 37; and a VL1, comprising a VL1_{CDR1} having the amino acid sequence of SEQ ID NO: 42; a VL1_{CDR2} having the amino acid sequence of SEQ ID NO: 43; and a VL1_{CDR3} having the amino acid sequence of SEQ ID NO: 45.

In some embodiments, the bispecific antibody EIP0820 comprises a VH1 having the amino acid sequence of SEQ ID NO: 17 and a VL1 having the amino acid sequence of SEQ ID NO: 22. In some embodiments, the bispecific antibody EIP0820 comprises a H1 having the amino acid sequence of SEQ ID NO: 622 and a L1 having the amino acid sequence of SEQ ID NO: 69.

In some embodiments, exemplary CD3ε x ULBP2/5/6 bispecific antibodies of the present disclosure comprise an amino acid at position 446 (EU numbering) and/or at position 447 (EU numbering) of the CH1_{H3} and/or of the CH2_{H3}. In some embodiments, wherein the exemplary bispecific antibodies comprise an amino acid at position 446 (EU numbering) of the CH1_{H3} and/or of the CH2_{H3}, the amino acid at position 446 (EU numbering) is a G. In some embodiments, wherein the exemplary bispecific antibodies comprise an amino acid at position 446 (EU numbering) of the CH1_{H3} and/or of the CH2_{H3}, the bispecific antibodies further comprise an amino acid at position 447 (EU numbering) of the CH1_{H3} and/or of the CH2_{H3}. In some embodiments, wherein the exemplary bispecific antibodies comprise an amino acid at position 447 (EU numbering) of the CH1_{H3} and/or of the CH2_{H3}, the amino acid at position 447 (EU numbering) is a K.

In some embodiments, exemplary CD3ε x ULBP2/5/6 bispecific antibodies of the present disclosure comprise an amino acid at position 446 (EU numbering) of the CH1_{H3} or CH2_{H3}. In some embodiments, the bispecific antibodies comprise an amino acid at position 446 (EU numbering) of the CH1_{H3} and CH2_{H3}. In some embodiments, the bispecific antibodies comprise an amino acid at position 446 (EU numbering) of the CH1_{H3}. In some embodiments, the bispecific antibodies comprise an amino acid at position 446 (EU numbering) of the CH2_{H3}.

In some embodiments, wherein the bispecific antibodies comprise an amino acid at position 446 (EU numbering) of the CH1_{H3} and/or CH2_{H3}, the amino acid at position 446 (EU numbering) is a G. In some embodiments, wherein the bispecific antibodies comprise an amino acid at position 446 (EU numbering) of the CH1_{H3}, the amino acid at position 446 (EU numbering) is a G. In some embodiments, wherein the bispecific antibodies comprise an amino acid at position 446 (EU numbering) of the CH2_{H3}, the amino acid at position 446 (EU numbering) is a G. In some embodiments, wherein the bispecific antibodies comprise an amino acid at position 446 (EU numbering) of the CH1_{H3} and CH2_{H3}, the amino acid at position 446 (EU numbering) of the CH1_{H3} and CH2_{H3} is a G.

In some embodiments, exemplary CD3ε x ULBP2/5/6 bispecific antibodies of the comprise an amino acid at position 447 (EU numbering) of the CH1_{H3} and/or of the CH2_{H3}. In some embodiments, the bispecific antibodies comprise an amino acid at position 447 (EU numbering) of the CH1_{H3} or of the CH2_{H3}. In some embodiments, the bispecific antibodies comprise an amino acid at position 447 (EU numbering) of the CH1_{H3} and of the CH2_{H3}. In some embodiments, the bispecific antibodies comprise an amino acid at position 447 (EU numbering) of the CH1_{H3}. In some embodiments, the bispecific antibodies comprise an amino acid at position 447 (EU numbering) of the CH2_{H3}.

In some embodiments, wherein CD3ε x ULBP2/5/6 bispecific antibodies comprise an amino acid at position 447 (EU numbering) of the CH1_{H3} and/or of the CH2_{H3}, the amino acid at position 447 (EU numbering) is a K. In some embodiments, wherein the bispecific antibodies comprise an amino acid at position 447 (EU numbering) of the CH1_{H3}, the amino acid at position 447 (EU numbering) is a K. In some embodiments, wherein the bispecific antibodies comprise an amino acid at position 447 (EU numbering) of the CH2_{H3}, the amino acid at position 447 (EU numbering) is a K.

### Fusion Peptides

Provided herein is an antibody (e.g. monospecific antibody or bispecific antibody) that has a fusion peptide fused to the N-terminus or the C-terminus of the first heavy chain polypeptide or the second heavy chain polypeptide.

Critical to the initial T cell response is the capacity for T cells to detect foreign and mutated proteins through their T cell receptor. This response, often referred to as signal 1 of T cell activation, occurs when the T cell receptor engages a cell that displays a foreign or mutated protein fragment or antigen in a specific protein complex called the Major Histocompatibility Complex I (MHCI). The activation of the T cell receptor is by itself both activating and auto-regulatory to T cells. Strong binding of the TCR to an MHCI complex creates chronic activation of the TCR. This form of signal is associated with T cells that are reactive to self-antigens. T cells are programed to inactivate when they experience this form activation. T cells with TCR that bind weaker, but sufficient for activation, experience acute signaling with the potential to remain active and differentiate into memory T cells. This is emerging as important consideration in the design the T cell therapeutics.

T cell cytokine activation, often referred to signal 3, is important in T cell transitions, either from non-dividing to a state of rapid cell division or from one phenotypic state to another. T cell cytokine receptors bind to cytokines that are produced by immune and non-immune cells and depending on the cytokine and the state of the T cell at the time of receiving the cytokine signal can induce cell proliferation, can sustain vitality, or can induce differentiation of T cells into a specialized cell state appropriate for sustained activation or inactivation following infection.

One example is the transition that naïve cells experience through cytokines which can induce naïve T cells to proliferate and promote T cell differentiation into memory T cells. Exemplary cytokines include but are not limited to IL-2, IL-7, IL-10, IL-12, IL-15, IL-18 and IL-21.

Costimulatory receptor activation, referred to as signal 2 provides a context specific cell-to-cell reinforcement of T activation. The most recognized form of costimulation occurs when T cells interact with activated antigen presenting cells through the T cell costimulatory receptor CD28 with CD80 and CD86 ligands found on APCs. These interactions can “prime” specific T cells armed with T cell receptors responsive to pathogen or cancer proteins.

Less appreciated is costimulation induced at the site of infection and malignancies. This includes costimulation that acts through CD2 and NKG2D receptors responsive to ligands like CD58 and UL16 binding proteins (e.g. ULBP2/5/6) that are induced in immune cells and epithelial cells upon viral infection. These signals provide not only reinforcement of T activation, but confirmation that the T cell's lethal effector activities are targeted with single cell accuracy. While many costimulatory receptors have been discovered, the importance of each receptor's specific context and the impact of concurrent signaling of multiple costimulatory receptors remains largely unknown and an area to greatly advance our understanding of T cell biology and creating possibilities for novel tumor-targeted T cell therapeutic development.

Costimulatory ligands include but are not limited to CD48, CD58, CD86, TNFSF9, OX40L, 4-1BBL, GITL, CD70, CD80, MR1, TNFSF4, ICOSL or ICOSLG.

CD58 is advantageous over other costimulatory ligands in that it is the primary costimulatory pathway available at the tumor site as tumor infiltrating T lymphocytes often lose expression of other costimulatory receptors like CD28, or due to the low immunogenicity of tumor cells, tumor cells do not sufficiently activate T cell, thus limiting the potential of inducible costimulatory receptors like 41BB.

As described previously, the anti-CD3ε antibodies of the disclosure induce varying levels of T cell receptor activation that confer alteration in T cell vitality and cytokine production. Accordingly, a fusion of the costimulatory ligand CD58 to the anti-CD3ε bispecific antibody provides integrated costimulatory T cell activation for optimal T cell activation.

In some embodiments, the bispecific antibody has a peptide fused to the N-terminus of the first heavy chain polypeptide (H1). In some embodiments, the bispecific antibody has a peptide fused to the C-terminus of the first heavy chain polypeptide (H1). In some embodiments, the bispecific antibody has a polypeptide fused to the N-terminus of the second heavy chain polypeptide (H2). In some embodiments, the bispecific antibody has a peptide fused to the C-terminus of the second heavy chain polypeptide (H2). Exemplary peptides include but are not limited to IL-2, IL-7, IL-10, IL-12, IL-15, IL-18, IL-21 or portions thereof. Exemplary peptides include but are not limited to CD48, CD58, CD86, TNFSF9, OX40L, 4-1BBL, GITL, CD70, CD80, MR1, TNFSF4, ICOSL, ICOSLG or portions thereof. Exemplary peptide sequences that are fused to the bispecific antibodies include but are not limited to those listed in Table 15.1.

TABLE 15.1
Exemplary Fusion Peptide Sequences

| Domain | Amino Acid Sequence | SEQ ID NO: |
|---|---|---|
| CD58-ECD | FSQQIYGVVYGNVTFHVPSNVPLKEVLWKKQKDKVAELENSE | SEQ ID NO: 49 |
| (CD58) | FRAFSSFKNRVYLDTVSGSLTIYNLTSSDEDEYEMESPNITD | |
| | TMKFFLYVLESLPSPILTCALINGSIEVQCMIPEHYNSHRGL | |
| | IMYSWDCPMEQCKRNSTSIYFKMENDLPQKIQCTLSNPLENT | |
| | TSSIILTTCIPSSGHSRHRY | |
| CD58- | FSQQIYGVVYGNVTFHVPSNVPLKEVLWKKQKDKVAELENSE | SEQ ID NO: 50 |
| variable | FRAFSSFKNRVYLDTVSGSLTIYNLTSSDEDEYEMESPNITD | |
| domain | TMKFFLYVLE | |
| (″CD58v*″) | | |
| IL-7 | DCDIEGKDGKQYESVLMVSIDQLLDSMKEIGSNCLNNEFNFF | SEQ ID NO: 51 |
| | KRHICDANKEGMFLFRAARKLRQFLKMNSTGDFDLHLLKVSE | |
| | GTTILLNCTGQVKGRKPAALGEAQPTKSLEENKSLKEQKKLN | |
| | DLCFLKRLLQEIKTCWNKILMGTKEH | |

In some embodiments, the CD3 x ULBP2/5/6 bispecific antibodies of the invention have a CD58 fusion peptide comprising any one of the CD58 sequences of Table 15.2. In some embodiments, the CD3 x ULBP2/5/6 bispecific antibodies of the invention have a CD58 fusion peptide comprising any one of SEQ ID NOs: 624-628.

TABLE 15.2
CD58 sequences

| | | | SEQ |
|---|---|---|---|
| Name | Description | Sequence | ID NO: |
| CD58- | Full length CD58, | MVAGSDAGRALGVLSVVCLLHCFGFIS | 624 |
| 1 | including signal | CFSQQIYGVVYGNVTFHVPSNVPLKEV | |
| | sequence | LWKKQKDKVAELENSEFRAFSSFKNR | |
| | | VYLDTVSGSLTIYNLTSSDEDEYEMES | |
| | | PNITDTMKFFLYVLESLPSPTLTCALTN | |
| | | GSIEVQCMIPEHYNSHRGLIMYSWDCP | |
| | | MEQCKRNSTSIYFKMENDLPQKIQCTL | |
| | | SNPLFNTTSSIILTTCIPSSGHSRHRYALI | |
| | | PIPLAVITTCIVLYMNGILKCDRKPDRT | |
| | | NSN | |
| CD58- | Extracellular | FSQQIYGVVYGNVTFHVPSNVPLKEVL | 625 |
| 2 | domain of CD58, | WKKQKDKVAELENSEFRAFSSFKNRV | |
| | corresponding to | YLDTVSGSLTIYNLTSSDEDEYEMESP | |
| | amino acids 29- | NITDTMKFFLYVLESLPSPTLTCALTNG | |
| | 215 of CD58 | SIEVQCMIPEHYNSHRGLIMYSWDCPM | |
| | (WT) | EQCKRNSTSIYFKMENDLPQKIQCTLS | |
| | | NPLFNTTSSIILTTCIPSSGHSRHR | |
| CD58- | Extracellular | BSQQIYGVJYGNVTFHVPSNOPLKEVL | 626 |
| 3 | domain of CD58, | WKKQKDKVAELENSEFRAFSSFKNRV | |
| | corresponding to | YLDTUSGSLTIYNLTSSDEDEYEMESP | |
| | amino acids 29- | NITDXMKFFLYVZESLPSPTLTCALTNG | |
| | 215 of CD58 | SIEVQCMIPEHYNSHRGLIMYSWDCPM | |
| | (with permitted | EQCKRNSTSIYFKMENDLPQKIQCTLS | |
| | substitutions) | NPLFNTTSSIILTTCIPSSGHSRHR | |
| | | B = F or S | |
| | | J = V or K | |
| | | O = V or Q | |
| | | U = V or K | |
| | | X = T or S | |
| | | Z = L or G | |
| CD58- | Amino acids 30- | SQQIYGVVYGNVTFHVPSNVPLKEVL | 627 |
| 4 | 123 (WT) | WKKQKDKVAELENSEFRA | |
| CD58- | Amino acids 30- | SQQIYGVJYGNVTFHVPSNOPLKEVLW | 628 |
| 5 | 123 (with | KKQKDKVAELENSEFRA | |
| | permitted | J = V or K | |
| | substitutions) | O = V or Q | |

In some embodiments the polypeptide is fused directly to the bispecific antibody. In some embodiments, the polypeptide is fused indirectly through a linker. In some embodiments, the bispecific antibody fused with a peptide comprises a linker sequence. Exemplary linker sequences include but are not limited to those listed in Table 16.1.

TABLE 16.1
Exemplary Linker Sequences

| Domain | Amino Acid Sequence | SEQ ID NO: |
|---|---|---|
| Linker-1 | GGGGS | SEQ ID NO: 52 |
| Linker-2 | GGGGSGGGGS | SEQ ID NO: 53 |
| Linker-3 | GGGGSGGGGSGGGGS | SEQ ID NO: 54 |

In some embodiments, the CD3 x ULBP2/5/6 bispecific antibodies of the invention have a CD58 fusion peptide (SEQ ID NO: 49) fused indirectly at the C-terminus of the first heavy chain polypeptide (H1) using linker-1 (SEQ ID NO: 52). In some embodiments, the CD3 x ULBP2/5/6 bispecific antibodies of the invention have a CD58v* fusion peptide (SEQ ID NO: 50) fused indirectly at the C-terminus of the first heavy chain polypeptide (H1) using linker-1 (SEQ ID NO: 52). In some embodiments, the CD3 x ULBP2/5/6 bispecific antibodies of the invention have a IL-7 fusion peptide (SEQ ID NO: 51) fused indirectly at the C-terminus of the first heavy chain polypeptide (H1) using linker-1 (SEQ ID NO: 52).

In some embodiments, the CD3 x ULBP2/5/6 bispecific antibodies of the invention have a CD58 fusion peptide (SEQ ID NO: 49) fused indirectly at the C-terminus of the first heavy chain polypeptide (H1) using linker-2 (SEQ ID NO: 53). In some embodiments, the CD3 x ULBP2/5/6 bispecific antibodies of the invention have a CD58v* fusion peptide (SEQ ID NO: 50) fused indirectly at the C-terminus of the first heavy chain polypeptide (H1) using linker-2 (SEQ ID NO: 53). In some embodiments, the CD3 x ULBP2/5/6 bispecific antibodies of the invention have a IL-7 fusion peptide (SEQ ID NO: 51) fused indirectly at the C-terminus of the first heavy chain polypeptide (H1) using linker-2 (SEQ ID NO: 53).

In some embodiments, the CD3 x ULBP2/5/6 bispecific antibodies of the invention have a CD58 fusion peptide (SEQ ID NO: 49) fused indirectly at the C-terminus of the first heavy chain polypeptide (H1) using linker-3 (SEQ ID NO: 54). In some embodiments, the CD3 x ULBP2/5/6 bispecific antibodies of the invention have a CD58v* fusion peptide (SEQ ID NO: 50) fused indirectly at the C-terminus of the first heavy chain polypeptide (H1) using linker-3 (SEQ ID NO: 54). In some embodiments, the CD3 x ULBP2/5/6 bispecific antibodies of the invention have a IL-7 fusion peptide (SEQ ID NO: 51) fused indirectly at the C-terminus of the first heavy chain polypeptide (H1) using linker-3 (SEQ ID NO: 54).

In some embodiments, the CD3 x ULBP2/5/6 bispecific antibodies of the invention have a hinge sequence comprising any one of the linker sequences of Table 16.2.

In some embodiments, the CD3 x ULBP2/5/6 bispecific antibodies of the invention have a linker sequence comprising any one of SEQ ID NOs: 530-552.

TABLE 16.2
Hinge Sequences

| Hinge | Hinge | | SEQ |
|---|---|---|---|
| Name | Description | Hinge Sequence | ID NO: |
| H1 | Human IgA1 | VPSTPPTPSPSTPPTPSPS | 530 |
| H2 | Human IgA2 | VPPPPP | 531 |
| H3 | Human IgD | ESPKAQASSVPTAQPQAEGSLAKATTAP | 532 |
| | | ATTRN | |
| | | TGRGGEEKKKEKEKEEQEERETKTP | |
| H4 | Human IgG1 | EPKSCDKTHTCPPCP | 533 |
| H5 | Human IgG2 | ERKCCVECPPCP | 534 |
| H6 | Human IgG3 | ELKTPLGDTTHTCPRCPEPKSCDTPPPCP | 535 |
| | | RCPE | |
| | | PKSCDTPPPCPRCPEPKSCDTPPPCPRCP | |
| H7 | Human IgG4 | ESKYGPPCPSCP | 536 |
| H8 | Human IgG4(P) | ESKYGPPCPPCP | 537 |
| H9 | Engineered v1 | CPPC | 538 |
| H10 | Engineered v2 | CPSC | 539 |
| H11 | Engineered v3 | CPRC | 540 |
| H12 | Engineered v4 | SPPC | 541 |
| H13 | Engineered v5 | CPPS | 542 |
| H14 | Engineered v6 | SPPS | 543 |
| H15 | Engineered v7 | DKTHTCAA | 544 |
| H16 | Engineered v8 | DKTHTCPPCPA | 545 |
| H17 | Engineered v9 | DKTHTCPPCPATCPPCPA | 546 |
| H18 | Engineered v10 | DKTHTCPPCPATCPPCPATCPPCPA | 547 |
| H19 | Engineered v11 | DKTHTCPPCPAGKPTLYNSLVMSDTAG | 548 |
| | | TCY | |
| H20 | Engineered v12 | DKTHTCPPCPAGKPTHVNVSVVMAEV | 549 |
| | | DGTCY | |
| H21 | Engineered v13 | DKTHTCCVECPPCPA | 550 |
| H22 | Engineered v14 | DKTHTCPRCPEPKSCDTPPPCPRCPA | 551 |
| H23 | Engineered v15 | DKTHTCPSCPA | 552 |

In some embodiments, the CD3 x ULBP2/5/6 bispecific antibodies of the invention have a linker sequence comprising any one of the linker sequences of Table 16.3.

In some embodiments, the CD3 x ULBP2/5/6 bispecific antibodies of the invention have a linker sequence comprising any one of SEQ ID NOs: 553-606.

TABLE 16.3
Linker Sequences

| Linker | | SEQ ID |
|---|---|---|
| Name | Linker Sequence | NO: |
| L1 | ADAAP | 553 |
| L2 | ADAAPTVSIFP | 554 |
| L3 | ADAAPTVSIFPP | 555 |
| L4 | AKTTAP | 556 |
| L5 | AKTTAPSVYPLAP | 557 |
| L6 | AKTTPKLEEGEFSEARV | 558 |
| L7 | AKTTPKLGG | 559 |
| L8 | AKTTPP | 560 |
| L9 | AKTTPPSVTPLAP | 561 |
| L10 | ASTKGP | 562 |
| L11 | ASTKGPSVFPLAP | 563 |
| L12 | ASTKGPSVFPLAPASTKGPSVFPLAP | 564 |
| L13 | EGKSSGSGSESKST | 565 |
| L14 | GEGESGEGESGEGES | 566 |
| L15 | GEGESGEGESGEGESGEGES | 567 |
| L16 | GEGGSGEGGSGEGGS | 568 |
| L17 | GENKVEYAPALMALS | 569 |
| L18 | GGEGSGGEGSGGEGS | 570 |
| L19 | GGGESGGEGSGEGGS | 571 |
| L20 | GGGESGGGESGGGES | 572 |
| L21 | (GGGGS)ₙ (also referred to as | 573 |
| | (G4S)ₙ), wherein n can be 1-10. | |
| L22 | GGGGSGGGGS | 574 |
| L23 | GGGGSGGGGSGGGGS | 575 |
| L24 | GGGGSGGGGSGGGGSGGGGS | 576 |
| L25 | GGGKSGGGKSGGGKS | 577 |
| L26 | GGGKSGGKGSGKGGS | 578 |
| L27 | GGKGSGGKGSGGKGS | 579 |
| L28 | GGSGG | 580 |
| L29 | GGSGGGGSG | 581 |
| L30 | GGSGGGGSGGGGS | 582 |
| L31 | GHEAAAVMQVQYPAS | 583 |
| L32 | GKGGSGKGGSGKGGS | 584 |
| L33 | GKGKSGKGKSGKGKS | 585 |
| L34 | GKGKSGKGKSGKGKSGKGKS | 586 |
| L35 | GKPGSGKPGSGKPGS | 587 |
| L36 | GKPGSGKPGSGKPGSGKPGS | 588 |
| L37 | GPAKELTPLKEAKVS | 589 |
| L38 | GSAGSAAGSGEF | 590 |
| L39 | IRPRAIGGSKPRVA | 591 |
| L40 | KESGSVSSEQLAQFRSLD | 592 |
| L41 | KTTPKLEEGEFSEAR | 593 |
| L42 | QPKAAP | 594 |
| L43 | QPKAAPSVTLFPP | 595 |
| L44 | RADAAAA(G4S)₄ | 596 |
| L45 | RADAAAAGGPGS | 597 |
| L46 | RADAAP | 598 |
| L47 | RADAAPTVS | 599 |
| L48 | SAKTTP | 600 |
| L49 | SAKTTPKLEEGEFSEARV | 601 |
| L50 | SAKTTPKLGG | 602 |
| L51 | STAGDTHLGGEDFD | 603 |
| L52 | TVAAP | 604 |
| L53 | TVAAPSVFIFPP | 605 |
| L54 | TVAAPSVFIFPPTVAAPSVFIFPP | 606 |

Exemplary CD3 x ULBP2/5/6 bispecific antibodies having a CD58, CD58v* and IL-7 fusion are shown in Table 17 and Table 18.

TABLE 17
Exemplary Bispecific Fusion Molecules

| EIP0205 | EIP0205- | EIP0205- | EIP0205- | EIP0598 | EIP0598- | EIP0377 | EIP0377- | EIP0187 | EIP0187- |
|---|---|---|---|---|---|---|---|---|---|
| Affinity | CD58 | CD58v* | IL-7 | Affinity | CD58 | Affinity | CD58 | Affinity | CD58 |
| variants | fusion | fusion | fusion | variants | fusion | variants | fusion | variants | fusion |
| EIP0205 | EIP0359 | EIP0360 | EIP0363 | EIP0598 | EIP0599 | EIP0377 | EIP0547 | EIP0187 | EIP0641 |
| EIP0527 | EIP0566 | | | EIP0630 | EIP0640 | EIP0694 | EIP0652 | EIP0712 | EIP0642 |
| EIP0624 | EIP0634 | | | EIP0540 | EIP0567 | EIP0695 | EIP0653 | EIP0713 | EIP0643 |
| EIP0486 | EIP0562 | | | EIP0628 | EIP0638 | EIP0696 | EIP0654 | EIP0714 | EIP0644 |
| EIP0626 | EIP0636 | | | EIP0542 | EIP0568 | EIP0697 | EIP0655 | EIP0715 | EIP0645 |
| EIP0483 | EIP0561 | | | EIP0627 | EIP0637 | EIP0698 | EIP0656 | EIP0716 | EIP0646 |
| EIP0623 | EIP0633 | | | EIP0515 | EIP0564 | EIP0699 | EIP0657 | EIP0717 | EIP0647 |
| EIP0621 | EIP0631 | | | EIP0477 | EIP0560 | EIP0700 | EIP0658 | EIP0718 | EIP0648 |
| EIP0625 | EIP0635 | | | EIP0541 | EIP0662 | | EIP0659 | | |
| EIP0622 | EIP0632 | | | EIP0513 | EIP0563 | | EIP0660 | | |
| EIP0525 | EIP0565 | | | EIP0629 | EIP0639 | | EIP0661 | | |

TABLE 18
Exemplary Bispecific Antibodies that bind to CD38 and ULBP2/5/6
with a C-terminus fusion peptide

| | | | SEQ ID |
|---|---|---|---|
| Antibody | Domain | Amino Acid Sequence | NO |
| EIP0359 | ULBP2 | EIVLTQSPATLSLSPGERATLSCRASQSIGTSIHWYQKKPGQAPRLLIYFAS | SEQ ID |
| (EIP0205- | light | ESISGIPARFSGSGSGTDFTLTISSLEPEDFAVYYCQQSNSWPLYTFGGGTK | NO: |
| linker2- | chain (L2) | VEIKRTVAAPSVFIFPPSDEQLKSGTASVVCLLNNFYPREAKVQWKVDNALQ | 223 |
| CD58) | | SGNSQESVTEQDSKDSTYSLSSTLRLSKADYEKHKVYACEVTHQGLSSPVTK | |
| | | SFNRGEC | |
| | ULBP2 | QVQLVQSGAEVKKPGASVKVSCKASGYTFTNYDINWVRDAPGQGLEWMGWIY | SEQ ID |
| | heavy | PGDGSTYYNEKFKGRVTMTRDTSTSTVYMELSSLRSEDTAVYYCARSGDYGL | NO: |
| | chain (H2) | YYFDYWGQGTLVTVSSASTKGPSVFPLAPSSKSTSGGTAALGCLVDDYFPEP | 224 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 225 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 226 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0566 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 239 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 240 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWDSNLWVFGGGT | NO: |
| | | KLTVLGQPKANPTVTLFPPSSEELQANKADLVCLISDFYPGAVTVAWKADGS | 241 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 242 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALIN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0634 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 243 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 244 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 245 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GDSYVSWFAYWGQGTLVTVSSASTKGPSVFPLAPSSKSTSGGTAALGCLVKD | 246 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0562 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 247 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 248 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGD | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 249 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 250 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0636 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 251 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 252 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 253 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GDDYVSWFAYWGQGTLVTVSSASTKGPSVFPLAPSSKSTSGGTAALGCLVKD | 254 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0561 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 255 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 256 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 257 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNDYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 258 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0820 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: 67 |
| ΔGK- | | VEIKRTVAAPSVFIFPPSDEQLKSGTASVVCLLNNFYPREAKVQWKVDNALQ | |
| linker2- | | SGNSQESVTEQDSKDSTYSLSSTLRLSKADYEKHKVYACEVTHQGLSSPVTK | |
| CD58) | | SFNRGEC | |
| | ULBP2 heavy | EVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 621 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: 69 |
| | | KLTVLGQPKANPTVTLFPPSSEELQANKADLVCLISDFYPGAVTVAWKADGS | |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNDYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 622 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGGGGSGGGGSFSQQI | |
| | | YGVVYGNVTFHVPSNVPLKEVLWKKQKDKVAELENSEFRAFSSFKNRVYLDT | |
| | | VSGSLTIYNLTSSDEDEYEMESPNITDTMKFFLYVLESLPSPTLTCALINGS | |
| | | IEVQCMIPEHYNSHRGLIMYSWDCPMEQCKRNSTSIYFKMENDLPQKIQCTL | |
| | | SNPLENTTSSIILTTCIPSSGHSRHRY | |
| EIP0633 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 259 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 260 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 261 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVAKIR | SEQ ID |
| | chain (H1) | SKYNNYATYYADSVKDRFTISRDDSKNTAYLQMNNLKTEDTAVYYCVRHGNF | NO: |
| | | GDSYVSWFAYWGQGTLVTVSSASTKGPSVFPLAPSSKSTSGGTAALGCLVKD | 262 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0631 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 263 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 264 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 265 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGSF | NO: |
| | | GNSYVSWFAYWGQGTLVTVSSASTKGPSVFPLAPSSKSTSGGTAALGCLVKD | 266 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0635 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 267 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 268 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 269 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVAKIR | SEQ ID |
| | chain (H1) | SKYNNYATYYADSVKDRFTISRDDSKNTAYLQMNNLKTEDTAVYYCVRHGNF | NO: |
| | | GDSYVSWFAYWGQGTLVTVSSASTKGPSVFPLAPSSKSTSGGTAALGCLVKD | 270 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0632 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 271 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 272 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 273 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGTF | NO: |
| | | GNSYVSWFAYWGQGTLVTVSSASTKGPSVFPLAPSSKSTSGGTAALGCLVKD | 274 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0565 | ULBP2 light | EIVLTQSPATLSLSPGERATLSCRASQSIGTSIHWYQKKPGQAPRLLIYFAS | SEQ ID |
| (EIP0205- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 275 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 276 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 277 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMDWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 278 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0599 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 279 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 280 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 281 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 282 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0640 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 283 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 284 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWDSNLWVFGGGT | NO: |
| | | KLTVLGQPKANPTVTLFPPSSEELQANKADLVCLISDFYPGAVTVAWKADGS | 285 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 286 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0567 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 287 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 288 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 289 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GDSYVSWFAYWGQGTLVTVSSASTKGPSVFPLAPSSKSTSGGTAALGCLVKD | 290 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0638 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 291 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 292 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGD | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 293 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 294 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0568 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 295 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 296 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 297 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GDDYVSWFAYWGQGTLVTVSSASTKGPSVFPLAPSSKSTSGGTAALGCLVKD | 298 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0637 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 299 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 300 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 301 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNDYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 302 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0564 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 303 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 304 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 305 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVAKIR | SEQ ID |
| | chain (H1) | SKYNNYATYYADSVKDRFTISRDDSKNTAYLQMNNLKTEDTAVYYCVRHGNF | NO: |
| | | GDSYVSWFAYWGQGTLVTVSSASTKGPSVFPLAPSSKSTSGGTAALGCLVKD | 306 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0560 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 307 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 308 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 309 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGSF | NO: |
| | | GNSYVSWFAYWGQGTLVTVSSASTKGPSVFPLAPSSKSTSGGTAALGCLVKD | 310 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0662 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 311 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 312 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 313 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVAKIR | SEQ ID |
| | chain (H1) | SKYNNYATYYADSVKDRFTISRDDSKNTAYLQMNNLKTEDTAVYYCVRHGNF | NO: |
| | | GDSYVSWFAYWGQGTLVTVSSASTKGPSVFPLAPSSKSTSGGTAALGCLVKD | 314 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0563 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 315 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 316 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSQLWVFGGGT | NO: |
| | | KLTVLGQPKANPTVTLFPPSSEELQANKADLVCLISDFYPGAVTVAWKADGS | 317 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGTF | NO: |
| | | GNSYVSWFAYWGQGTLVTVSSASTKGPSVFPLAPSSKSTSGGTAALGCLVKD | 318 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0639 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 319 |
| CD58) | | SGNSQESVTEQDSKDSTYSLSSTLRLSKADYEKHKVYACEVTHQGLSSPVTK | |
| | | SFNRGEC | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 320 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 321 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMDWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 322 |
| | | YFPEPVTVSWNSGALTSGVHTSPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0547 | ULBP2 light | EIVLTQSPATLSLSPGERATLSCRASQSIGTSIHWYQKKPGQAPRLLIYFAS | SEQ ID |
| (EIP0598- | chain (L2) | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNKFYPREAKVQWKVDNALQ | 375 |
| CD58) | | SGNSQECVTEQDSKDSTYSLSSTLTLSKADYEKHKVYACEVTHQGLSSPVTK | |
| | | SFNRGES | |
| | ULBP2 heavy | QVQLVQSGAEVKKPGASVKVSCKASGYTFTNYDINWVRDAPGQGLEWMGWIY | SEQ ID |
| | chain (H2) | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVKDYFPEP | 376 |
| | | VTVSWNSGALTSGVHTCPAVLQSSGLYSLSSVVDVPSSSLGTQTYICNVNHK | |
| | | PSNTKVDKKVEPKSSDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSPG | |
| | CD3 light | ELVVTQEPSLTVSPGGTVTLTCRSSTGAVTTSNYANWVQDKPGQAPRGLIGG | SEQ ID |
| | chain (L1) | TNKRAPGTPARFSGSLLGGKAALTLSGVQPEDEAEYYCALWYSNLWVFGGGT | NO: |
| | | KLTVLGQPKANPTVTLFPPSSEELQANKAKLVCLISDFYPGAVTVAWKADGS | 377 |
| | | PVKAGVETTKPSKQSNNKYAASSYLSLTPEQWKSHRSYSCQVTHEGSTVEKT | |
| | | VAPTECS | |
| | CD3 heavy | EVQLLESGGGLVQPGGSLKLSCAASGFTFNTYAMNWVRKAPGKGLEWVARIR | SEQ ID |
| | chain (H1) | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCSVDD | 378 |
| | | YFPEPVTVSWNSGALTSGVHTFPAVLQSSGLYSLSSVVTVPSSSLGTQTYIC | |
| | | NVNHKPSNTKVDKKVEPKSCDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLENTTSSIILTTCIPSSGHSRHRY | |
| EIP0641 | TAA light | EIVLTQSPATLSLSPGERATLSCRASQSIGTSIHWYQKKPGQAPRLLIYFAS | SEQ ID |
| (EIP0187- | chain | ESISGIPARFSGSGSGTDFTLTISSLEPEDFAVYYCQQSNSWPLYTFGGGTK | NO: |
| linker2- | | VEIKRTVAAPCVFIFPPSDEQLKSGTASVVCLLNNFYPREAKVQWKVDNALQ | 442 |
| CD58) | | SGNSQESVTEQDSKDSTYSLSSTLRLSKADYEKHKVYACEVTHQGLSSPVTK | |
| | | SFNRGES | |
| | TAA heavy | QVQLVQSGAEVKKPGASVKVSCKASGYTFTNYDINWVRDAPGQGLEWMGWIY | SEQ ID |
| | chain | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTCGGTAALGCLVDDYFPEP | 443 |
| | | VTVSWNSGALTSGVHTFPAVLQSSGLYSLSSVVTVPSSSLGTQTYICNVNHK | |
| | | PSNTKVDKKVEPKSSDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSPG | |
| | CD3 light | ELVVTQEPSLTVSPGGTVTLTCRSSTGAVTTSNYANWVQDKPGQAPRGLIGG | SEQ ID |
| | chain | TNKRAPGTPARFSGSLLGGKAALTLSGVQPEDEAEYYCALWYSNLWVFGGGT | NO: |
| | | KLTVLGQPKANPTVTLFPPSSEELQANKATLVCLIDDFYPGAVTVAWKADGS | 444 |
| | | PVKAGVETTKPSKQSNNKYAASSYLSLTPEQWKSHRSYSCQVTHEGSTVEKT | |
| | | VAPTECS | |
| | CD3 heavy | EVQLLESGGGLVQPGGSLKLSCAASGFTFNTYAMNWVRKAPGKGLEWVARIR | SEQ ID |
| | chain | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 445 |
| | | YFPEPVTVSWNSGALTSGVHTFPAVLQSSGLYSLSSKVTVPSSSLGTQTYIC | |
| | | NVNHKPSNTKVDKKVEPKSCDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLESLPSPTLTCALTN | |
| | | GSIEVQCMIPEHYNSHRGLIMYSWDCPMEQCKRNSTSIYFKMENDLPQKIQC | |
| | | TLSNPLFNTTSSIILTTCIPSSGHSRHRY | |
| EIP0360 | TAA light | EIVLTQSPATLSLSPGERATLSCRASQSIGTSIHWYQKKPGQAPRLLIYFAS | SEQ ID |
| (EIP0598- | chain | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 227 |
| CD58v*) | | SGNSQESVTEQDSKDSTYSLSSTLRLSKADYEKHKVYACEVTHQGLSSPVTK | |
| | | SFNRGEC | |
| | TAA heavy | QVQLVQSGAEVKKPGASVKVSCKASGYTFTNYDINWVRDAPGQGLEWMGWIY | SEQ ID |
| | chain | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 228 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 229 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 230 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSFSQ | |
| | | QIYGVVYGNVTFHVPSNVPLKEVLWKKQKDKVAELENSEFRAFSSFKNRVYL | |
| | | DTVSGSLTIYNLTSSDEDEYEMESPNITDTMKFFLYVLE | |
| EIP0363 | TAA light | EIVLTQSPATLSLSPGERATLSCRASQSIGTSIHWYQKKPGQAPRLLIYFAS | SEQ ID |
| (EIP0205- | chain | ESISGIPARFSGSGSGTDFTLTISSLEPEDFAVYYCQQSNSWPLYTFGGGTK | NO: |
| linker2- | | VEIKRTVAAPSVFIFPPSDEQLKSGTASVVCLLNNFYPREAKVQWKVDNALQ | 323 |
| IL-7) | | SGNSQESVTEQDSKDSTYSLSSTLRLSKADYEKHKVYACEVTHQGLSSPVTK | |
| | | SFNRGEC | |
| | TAA heavy | QVQLVQSGAEVKKPGASVKVSCKASGYTFTNYDINWVRDAPGQGLEWMGWIY | SEQ ID |
| | chain | PGDGSTYYNEKFKGRVTMTRDTSTSTVYMELSSLRSEDTAVYYCARSGDYGL | NO: |
| | | YYFDYWGQGTLVTVSSASTKGPSVFPLAPSSKSTSGGTAALGCLVDDYFPEP | 324 |
| | | VTVSWNSGALTSGVHTFPAVLQSSGLYSLSSVVTVPSSSLGTQTYICNVNHK | |
| | | PSNTKVDKKVEPKSCDKTHTCPPCPAPEAAGAPSVFLFPPKPKDTLMISRTP | |
| | | EVTCVVVDVSHEDPEVKFNWYVDGVEVHNAKTKPREEQYNSTYRVVSVLTVL | |
| | | HQDWLNGKEYKCKVSNKALPAPIEKTISKAKGQPREPQVYTLPPCREEMTKN | |
| | | QVSLWCLVKGFYPSDIAVEWESNGQPENNYKTTPPVLDSDGSFFLYSKLTVD | |
| | | KSRWQQGNVFSCSVMHEALHNHYTQKSLSLSP | |
| | CD3 light | ELVVTQEPSLTVSPGGTVTLTCRSSTGAVTTSNYANWVQDKPGQAPRGLIGG | SEQ ID |
| | chain | TNKRAPGTPARFSGSLLGGKAALTLSGVQPEDEAEYYCALWYSNLWVFGGGT | NO: |
| | | KLTVLGQPKANPTVTLFPPSSEELQANKADLVCLISDFYPGAVTVAWKADGS | 325 |
| | | PVKAGVETCKPSKQSNNKYAASSYLSLTPEQWKSHRSYSCQVTHEGSTVEKT | |
| | | VAPTESS | |
| | CD3 heavy | EVQLLESGGGLVQPGGSLKLSCAASGFTFNTYAMNWVRKAPGKGLEWVARIR | SEQ ID |
| | chain | SKYNNYATYYADSVKDRFTISRDDSKNTAYLQMNNLKTEDTAVYYCVRHGNF | NO: |
| | | GNSYVSWFAYWGQGTLVTVSSASTKGPSVFPLAPSSKSTSGGTAALGCLVKD | 326 |
| | | YFPEPVTVSWNSGALTSGVHTFPACLQSSGLYSLSSVVTVPSSSLGTQTYIC | |
| | | NVNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVVS | |
| | | VLTVLHQDWLNGKEYKCKVSNKALPAPIEKTISKAKGQPREPQVCTLPPSRE | |
| | | EMTKNQVSLSCAVKGFYPSDIAVEWESNGQPENNYKTTPPVLDSDGSFFLVS | |
| | | KLTVDKSRWQQGNVFSCSVMHEALHNHYTQKSLSLSPGKGGGGSGGGGSDCD | |
| | | IEGKDGKQYESVLMVSIDQLLDSMKEIGSNCLNNEFNFFKRHICDANKEGMF | |
| | | LFRAARKLRQFLKMNSTGDFDLHLLKVSEGTTILLNCTGQVKGRKPAALGEA | |
| | | QPTKSLEENKSLKEQKKLNDLCFLKRLLQEIKTCWNKILMGTKEH | |

TABLE 19
Exemplary Bispecific Antibodies that bind to CD38 and a Second
Antigen

| | | | |
|---|---|---|---|
| EIP0373 | TAA light | DILLTQSPVILSVSPGERVSFSCRASQSIGTNIHWYQKRINGSPRLLI | SEQ ID |
| (V373) | chain (L2) | KYASESISGIPSRFSGSGSGTDFTLSINSVESEDIADYYCQQNNNWPT | NO: 450 |
| EGFRxCD3- | (Cetuximab, | TFGAGTKLELKRTVAAPSVFIFPPSDEQLKSGTASVVCLLNNFYPREA | |
| 01 | WO1996040210) | KVQWKVDNALQSGNSQESVTEQDSKDSTYSLSSTLRLSKADYEKHKVY | |
| | | ACEVTHQGLSSPVTKSFNRGEC | |
| | TAA heavy | QVQLKQSGPGLVQPSQSLSITCTVSGFSLTNYGVHWVRDSPGKGLEWL | SEQ ID |
| | chain (H2) | GVIWSGGNTDYNTPFTSRLSINKDNSKSQVFFKMNSLQSNDTAIYYCA | NO: 451 |
| | (Cetuximab, | RALTYYDYEFAYWGQGTLVTVSAASTKGPSVFPLAPSSKSTSGGTAAL | |
| | WO1996040210) | GCLVDDYFPEPVTVSWNSGALTSGVHTFPAVLQSSGLYSLSSVVTVPS | |
| | | SSLGTQTYICNVNHKPSNTKVDKKVEPKSCDKTHTCPPCPAPEAAGAP | |
| | | SVFLFPPKPKDTLMISRTPEVTCVVVDVSHEDPEVKFNWYVDGVEVHN | |
| | | AKTKPREEQYNSTYRVVSVLTVLHQDWLNGKEYKCKVSNKALPAPIEK | |
| | | TISKAKGQPREPQVYTLPPCREEMTKNQVSLWCLVKGFYPSDIAVEWE | |
| | | SNGQPENNYKTTPPVLDSDGSFFLYSKLTVDKSRWQQGNVFSCSVMHE | |
| | | ALHNHYTQKSLSLSPG | |
| | CD3 light | ELVVTQEPSLTVSPGGTVTLTCRSSTGAVTTSNYANWVQDKPGQAPRG | SEQ ID452 |
| | chain (L1) | LIGGTNKRAPGTPARFSGSLLGGKAALTLSGVQPEDEAEYYCALWYSN | NO: |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 453 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPG | |
| EIP0535 | TAA light | DILLTQSPVILSVSPGERVSFSCRASQSIGTNIHWYQKRINGSPRLLI | SEQ ID |
| (V535) | chain (L2) | KYASESISGIPSRFSGSGSGTDFTLSINSVESEDIADYYCQQNNNWPT | NO: 454 |
| EGFRxCD3- | (Cetuximab, | TFGAGTKLELKRTVAAPSVFIFPPSDEQLKSGTASVVCLLNNFYPREA | |
| 01xCD58 | WO1996040210) | KVQWKVDNALQSGNSQESVTEQDSKDSTYSLSSTLRLSKADYEKHKVY | |
| | | ACEVTHQGLSSPVTKSFNRGEC | |
| | TAA heavy | QVQLKQSGPGLVQPSQSLSITCTVSGFSLTNYGVHWVRDSPGKGLEWL | SEQ ID |
| | chain (H2) | GVIWSGGNTDYNTPFTSRLSINKDNSKSQVFFKMNSLQSNDTAIYYCA | NO: 455 |
| | (Cetuximab, | RALTYYDYEFAYWGQGTLVTVSAASTKGPSVFPLAPSSKSTSGGTAAL | |
| | WO1996040210) | GCLVDDYFPEPVTVSWNSGALTSGVHTFPAVLQSSGLYSLSSVVTVPS | |
| | | SSLGTQTYICNVNHKPSNTKVDKKVEPKSCDKTHTCPPCPAPEAAGAP | |
| | | SVFLFPPKPKDTLMISRTPEVTCVVVDVSHEDPEVKFNWYVDGVEVHN | |
| | | AKTKPREEQYNSTYRVVSVLTVLHQDWLNGKEYKCKVSNKALPAPIEK | |
| | | TISKAKGQPREPQVYTLPPCREEMTKNQVSLWCLVKGFYPSDIAVEWE | |
| | | SNGQPENNYKTTPPVLDSDGSFFLYSKLTVDKSRWQQGNVFSCSVMHE | |
| | | ALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 456 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 457 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPGKGGGGSGGGGSGGGGSFSQQIYGVVY | |
| | | GNVTFHVPSNVPLKEVLWKKQKDKVAELENSEFRAFSSFKNRVYLDTV | |
| | | SGSLTIYNLTSSDEDEYEMESPNITDTMKFFLYVLESLPSPTLTCALT | |
| | | NGSIEVQCMIPEHYNSHRGLIMYSWDCPMEQCKRNSTSIYFKMENDLP | |
| | | QKIQCTLSNPLFNTTSSIILTTCIPSSGHSRHRY | |
| EIP0506 | TAA light | DIQMTQSPSSLSASVGDRVTITCSASQDISNYLNWYQKKPGKAPKLLI | SEQ ID |
| (V506) | chain (L2) | YYTSNLHSGVPSRFSGSGSGTDFTLTISSLQPEDFATYYCQQYRKLPW | NO: 458 |
| BCMAxCD3- | Belantamab, (U.S. | TFGQGTKLEIKRTVAAPSVFIFPPSDEQLKSGTASVVCLLNNFYPREA | |
| 01 | 20140105915) | KVQWKVDNALQSGNSQESVTEQDSKDSTYSLSSTLRLSKADYEKHKVY | |
| | | ACEVTHQGLSSPVTKSFNRGEC | |
| | TAA heavy | QVQLVQSGAEVKKPGSSVKVSCKASGGTFSNYWMHWVRDAPGQGLEWM | SEQ ID |
| | chain (H2) | GATYRGHSDTYYNQKFKGRVTITADKSTSTAYMELSSLRSEDTAVYYC | NO: 459 |
| | Belantamab, (U.S. | ARGAIYDGYDVLDNWGQGTLVTVSSASTKGPSVFPLAPSSKSTSGGTA | |
| | 20140105915) | ALGCLVDDYFPEPVTVSWNSGALTSGVHTFPAVLQSSGLYSLSSVVTV | |
| | | PSSSLGTQTYICNVNHKPSNTKVDKKVEPKSCDKTHTCPPCPAPEAAG | |
| | | APSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPI | |
| | | EKTISKAKGQPREPQVYTLPPCREEMTKNQVSLWCLVKGFYPSDIAVE | |
| | | WESNGQPENNYKTTPPVLDSDGSFFLYSKLTVDKSRWQQGNVFSCSVM | |
| | | HEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 460 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 461 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPG | |
| EIP0534 | TAA light | DIQMTQSPSSLSASVGDRVTITCSASQDISNYLNWYQKKPGKAPKLLI | SEQ ID |
| (V534) | chain (L2) | YYTSNLHSGVPSRFSGSGSGTDFTLTISSLQPEDFATYYCQQYRKLPW | NO: 462 |
| BCMAxCD3- | Belantamab, (U.S. | TFGQGTKLEIKRTVAAPSVFIFPPSDEQLKSGTASVVCLLNNFYPREA | |
| 01xCD58 | 20140105915) | KVQWKVDNALQSGNSQESVTEQDSKDSTYSLSSTLRLSKADYEKHKVY | |
| | | ACEVTHQGLSSPVTKSFNRGEC | |
| | TAA heavy | QVQLVQSGAEVKKPGSSVKVSCKASGGTFSNYWMHWVRDAPGQGLEWM | SEQ ID |
| | chain (H2) | GATYRGHSDTYYNQKFKGRVTITADKSTSTAYMELSSLRSEDTAVYYC | NO: 463 |
| | Belantamab, (U.S. | ARGAIYDGYDVLDNWGQGTLVTVSSASTKGPSVFPLAPSSKSTSGGTA | |
| | 20140105915) | ALGCLVDDYFPEPVTVSWNSGALTSGVHTFPAVLQSSGLYSLSSVVTV | |
| | | PSSSLGTQTYICNVNHKPSNTKVDKKVEPKSCDKTHTCPPCPAPEAAG | |
| | | APSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPI | |
| | | EKTISKAKGQPREPQVYTLPPCREEMTKNQVSLWCLVKGFYPSDIAVE | |
| | | WESNGQPENNYKTTPPVLDSDGSFFLYSKLTVDKSRWQQGNVFSCSVM | |
| | | HEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 464 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 465 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPGKGGGGSGGGGSGGGGSFSQQIYGVVY | |
| | | GNVTFHVPSNVPLKEVLWKKQKDKVAELENSEFRAFSSFKNRVYLDTV | |
| | | SGSLTIYNLTSSDEDEYEMESPNITDTMKFFLYVLESLPSPTLTCALT | |
| | | NGSIEVQCMIPEHYNSHRGLIMYSWDCPMEQCKRNSTSIYFKMENDLP | |
| | | QKIQCTLSNPLFNTTSSIILTTCIPSSGHSRHRY | |
| EIP0702 | TAA light | — | SEQ ID |
| (V702) | chain (L2) | | NO: 466 |
| TNB-383B | TAA heavy | EVQLLESGGGLVQPGGSLRLSCAASGFTVSSYGMSWVRQAPGKGPEWV | SEQ ID |
| | chain (H2) | SGIRGSDGSTYYADSVKGRFTISRDNSKNTLYLQMNSLRAEDTAVYYC | NO: 467 |
| | TNB-383B, | AKQGENDGPFDHRGQGTLVTVSSGGGGSEVQLLESGGGLVQPGGSLRL | |
| | (US20210403587 | SCAASGFTVSSYGMSWVRQAPGKGPEWVSGIRGSDGSTYYADSVKGRF | |
| | A1) | TISRDNSKNTLYLQMNSLRAEDTAVYYCAKQGENDGPFDHRGQGTLVT | |
| | | VSSESKYGPPCPPCPAPEAAGGPSVFLFPPKPKDTLMISRTPEVTCVV | |
| | | VDVSQEDPEVQFNWYVDGVEVHNAKTKPREEQFNSTYRVVSVLTVLHQ | |
| | | DWLNGKEYKCKVSNKGLPSSIEKTISKAKGQPREPQVYTLPPSQEEMT | |
| | | KNQVSLSCAVKGFYPSDIAVEWESNGQPENNYKTTPPVLDSDGSFFLV | |
| | | SRLTVDKSRWQEGNVFSCSVMHEALHNHYTQKSLSLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 468 |
| | TNB-383B, | TFGQGTKVEIKRTVAAPSVFIFPPSDEQLKSGTASVVCLLNNFYPREA | |
| | (US20210403587 | KVQWKVDNALQSGNSQESVTEQDSKDSTYSLSSTLTLSKADYEKHKVY | |
| | A1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 469 |
| | TNB-383B, | AKDSRGYGDYRLGGAYWGQGTLVTVSSASTKGPSVFPLAPCSRSTSES | |
| | (US20210403587 | TAALGCLVKDYFPEPVTVSWNSGALTSGVHTFPAVLQSSGLYSLSSVV | |
| | A1) | TVPSSSLGTKTYTCNVDHKPSNTKVDKRVESKYGPPCPPCPAPEAAGG | |
| | | PSVFLFPPKPKDTLMISRTPEVTCVVVDVSQEDPEVQFNWYVDGVEVH | |
| | | NAKTKPREEQFNSTYRVVSVLTVLHQDWLNGKEYKCKVSNKGLPSSIE | |
| | | KTISKAKGQPREPQVYTLPPSQEEMTKNQVSLWCLVKGFYPSDIAVEW | |
| | | ESNGQPENNYKTTPPVLDSDGSFFLYSRLTVDKSRWQEGNVFSCSVMH | |
| | | EALHNHYTQKSLSLSLG | |
| EIP0703 | TAA light | | SEQ ID |
| (V703) | chain (L2) | | NO: 470 |
| TNB-383B | TAA heavy | EVQLLESGGGLVQPGGSLRLSCAASGFTVSSYGMSWVRQAPGKGPEWV | SEQ ID |
| x CD58 | chain (H2) | SGIRGSDGSTYYADSVKGRFTISRDNSKNTLYLQMNSLRAEDTAVYYC | NO: 471 |
| | TNB-383B, | AKQGENDGPFDHRGQGTLVTVSSGGGGSEVQLLESGGGLVQPGGSLRL | |
| | (US20210403587 | SCAASGFTVSSYGMSWVRQAPGKGPEWVSGIRGSDGSTYYADSVKGRF | |
| | A1) | TISRDNSKNTLYLQMNSLRAEDTAVYYCAKQGENDGPFDHRGQGTLVT | |
| | | VSSESKYGPPCPPCPAPEAAGGPSVFLFPPKPKDTLMISRTPEVTCVV | |
| | | VDVSQEDPEVQFNWYVDGVEVHNAKTKPREEQFNSTYRVVSVLTVLHQ | |
| | | DWLNGKEYKCKVSNKGLPSSIEKTISKAKGQPREPQVYTLPPSQEEMT | |
| | | KNQVSLSCAVKGFYPSDIAVEWESNGQPENNYKTTPPVLDSDGSFFLV | |
| | | SRLTVDKSRWQEGNVFSCSVMHEALHNHYTQKSLSLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 472 |
| | TNB-383B, | TFGQGTKVEIKRTVAAPSVFIFPPSDEQLKSGTASVVCLLNNFYPREA | |
| | (US20210403587 | KVQWKVDNALQSGNSQESVTEQDSKDSTYSLSSTLTLSKADYEKHKVY | |
| | A1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 473 |
| | TNB-383B, | AKDSRGYGDYRLGGAYWGQGTLVTVSSASTKGPSVFPLAPCSRSTSES | |
| | (US20210403587 | TAALGCLVKDYFPEPVTVSWNSGALTSGVHTFPAVLQSSGLYSLSSVV | |
| | A1) | TVPSSSLGTKTYTCNVDHKPSNTKVDKRVESKYGPPCPPCPAPEAAGG | |
| | | PSVFLFPPKPKDTLMISRTPEVTCVVVDVSQEDPEVQFNWYVDGVEVH | |
| | | NAKTKPREEQFNSTYRVVSVLTVLHQDWLNGKEYKCKVSNKGLPSSIE | |
| | | KTISKAKGQPREPQVYTLPPSQEEMTKNQVSLWCLVKGFYPSDIAVEW | |
| | | ESNGQPENNYKTTPPVLDSDGSFFLYSRLTVDKSRWQEGNVFSCSVMH | |
| | | EALHNHYTQKSLSLSLGGGGSGGGGSFSQQIYGVVYGNVTFHVPSNVP | |
| | | LKEVLWKKQKDKVAELENSEFRAFSSFKNRVYLDTVSGSLTIYNLTSS | |
| | | DEDEYEMESPNITDTMKFFLYVLESLPSPTLTCALINGSIEVQCMIPE | |
| | | HYNSHRGLIMYSWDCPMEQCKRNSTSIYFKMENDLPQKIQCTLSNPLF | |
| | | NTTSSIILTTCIPSSGHSRHRY | |
| EIP0765 | TAA light | | SEQ ID |
| (V765) | chain (L2) | | NO: 474 |
| TNB-383B | TAA heavy | EVQLLESGGGLVQPGGSLRLSCAASGFTVSSYGMSWVRQAPGKGPEWV | SEQ ID |
| disulfide | chain (H2) | SGIRGSDGSTYYADSVKGRFTISRDNSKNTLYLQMNSLRAEDTAVYYC | NO: 475 |
| stabilized | TNB-383B, | AKQGENDGPFDHRGQGTLVTVSSGGGGSEVQLLESGGGLVQPGGSLRL | |
| | (US20210403587 | SCAASGFTVSSYGMSWVRQAPGKGPEWVSGIRGSDGSTYYADSVKGRF | |
| | A1) | TISRDNSKNTLYLQMNSLRAEDTAVYYCAKQGENDGPFDHRGQGTLVT | |
| | | VSSESKYGPPCPPCPAPEAAGGPSVFLFPPKPKDTLMISRTPEVTCVV | |
| | | VDVSQEDPEVQFNWYVDGVEVHNAKTKPREEQFNSTYRVVSVLTVLHQ | |
| | | DWLNGKEYKCKVSNKGLPSSIEKTISKAKGQPREPQVCTLPPSQEEMT | |
| | | KNQVSLSCAVKGFYPSDIAVEWESNGQPENNYKTTPPVLDSDGSFFLV | |
| | | SRLTVDKSRWQEGNVFSCSVMHEALHNHYTQKSLSLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 476 |
| | TNB-383B, | TFGQGTKVEIKRTVAAPSVFIFPPSDEQLKSGTASVVCLLNNFYPREA | |
| | (US20210403587 | KVQWKVDNALQSGNSQESVTEQDSKDSTYSLSSTLTLSKADYEKHKVY | |
| | A1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 477 |
| | TNB-383B, | AKDSRGYGDYRLGGAYWGQGTLVTVSSASTKGPSVFPLAPCSRSTSES | |
| | (US20210403587 | TAALGCLVKDYFPEPVTVSWNSGALTSGVHTFPAVLQSSGLYSLSSVV | |
| | A1) | TVPSSSLGTKTYTCNVDHKPSNTKVDKRVESKYGPPCPPCPAPEAAGG | |
| | | PSVFLFPPKPKDTLMISRTPEVTCVVVDVSQEDPEVQFNWYVDGVEVH | |
| | | NAKTKPREEQFNSTYRVVSVLTVLHQDWLNGKEYKCKVSNKGLPSSIE | |
| | | KTISKAKGQPREPQVYTLPPCQEEMTKNQVSLWCLVKGFYPSDIAVEW | |
| | | ESNGQPENNYKTTPPVLDSDGSFFLYSRLTVDKSRWQEGNVFSCSVMH | |
| | | EALHNHYTQKSLSLSLG | |
| V766 | TAA light | | SEQ ID |
| (EIP0766) | chain (L2) | | NO: 478 |
| TNB-383B | TAA heavy | EVQLLESGGGLVQPGGSLRLSCAASGFTVSSYGMSWVRQAPGKGPEWV | SEQ ID |
| disulfide | chain (H2) | SGIRGSDGSTYYADSVKGRFTISRDNSKNTLYLQMNSLRAEDTAVYYC | NO: 478 |
| stabilized | TNB-383B, | AKQGENDGPFDHRGQGTLVTVSSGGGGSEVQLLESGGGLVQPGGSLRL | |
| x CD58 | (US20210403587 | SCAASGFTVSSYGMSWVRQAPGKGPEWVSGIRGSDGSTYYADSVKGRF | |
| | A1) | TISRDNSKNTLYLQMNSLRAEDTAVYYCAKQGENDGPFDHRGQGTLVT | |
| | | VSSESKYGPPCPPCPAPEAAGGPSVFLFPPKPKDTLMISRTPEVTCVV | |
| | | VDVSQEDPEVQFNWYVDGVEVHNAKTKPREEQFNSTYRVVSVLTVLHQ | |
| | | DWLNGKEYKCKVSNKGLPSSIEKTISKAKGQPREPQVCTLPPSQEEMT | |
| | | KNQVSLSCAVKGFYPSDIAVEWESNGQPENNYKTTPPVLDSDGSFFLV | |
| | | SRLTVDKSRWQEGNVFSCSVMHEALHNHYTQKSLSLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 480 |
| | TNB-383B, | TFGQGTKVEIKRTVAAPSVFIFPPSDEQLKSGTASVVCLLNNFYPREA | |
| | (US20210403587 | KVQWKVDNALQSGNSQESVTEQDSKDSTYSLSSTLTLSKADYEKHKVY | |
| | A1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 481 |
| | TNB-383B, | AKDSRGYGDYRLGGAYWGQGTLVTVSSASTKGPSVFPLAPCSRSTSES | |
| | (US20210403587 | TAALGCLVKDYFPEPVTVSWNSGALTSGVHTFPAVLQSSGLYSLSSVV | |
| | A1) | TVPSSSLGTKTYTCNVDHKPSNTKVDKRVVESKYGPPCPPCPAPEAAG | |
| | | GPSVFLFPPKPKDTLMISRTPEVTCVVVDVSQEDPEVQFNWYVDGVEV | |
| | | HNAKTKPREEQFNSTYRVVSVLTVLHQDWLNGKEYKCKVSNKGLPSSI | |
| | | EKTISKAKGQPREPQVYTLPPCQEEMTKNQVSLWCLVKGFYPSDIAVE | |
| | | WESNGQPENNYKTTPPVLDSDGSFFLYSRLTVDKSRWQEGNVFSCSVM | |
| | | HEALHNHYTQKSLSLSLGGGGSGGGGSFSQQIYGVVYGNVTFHVPSNV | |
| | | PLKEVLWKKQKDKVAELENSEFRAFSSFKNRVYLDTVSGSLTIYNLTS | |
| | | SDEDEYEMESPNITDTMKFFLYVLESLPSPTLTCALINGSIEVQCMIP | |
| | | EHYNSHRGLIMYSWDCPMEQCKRNSTSIYFKMENDLPQKIQCTLSNPL | |
| | | FNTTSSIILTTCIPSSGHSRHRY | |
| EIP0990 | TAA light | | SEQ ID |
| (V990) | chain (L2) | | NO: 482 |
| TNB-486 | TAA heavy | EVQLVESGGGLVQPGGSLRLSCAASGFSFSDFWMSWVRQAPGKGLEWV | SEQ ID |
| | chain (H2) | ATISQAGSEKDYVDSVKGRFTISRDNAKKSLYLQMNSLRAEDTAVYYC | NO: 483 |
| | TNB-486, | ASGVYSFDYRGQGTLVTVSSESKYGPPCPPCPAPEAAGGPSVFLFPPK | |
| | (US20210332133 | PKDTLMISRTPEVTCVVVDVSQEDPEVQFNWYVDGVEVHNAKTKPREE | |
| | A1) | QFNSTYRVVSVLTVLHQDWLNGKEYKCKVSNKGLPSSIEKTISKAKGQ | |
| | | PREPQVCTLPPSQEEMTKNQVSLSCAVKGFYPSDIAVEWESNGQPENN | |
| | | YKTTPPVLDSDGSFFLVSRLTVDKSRWQEGNVFSCSVMHEALHNHYTQ | |
| | | KSLSLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 484 |
| | TNB-486, | TFGQGTKVEIKRTVAAPSVFIFPPSDEQLKSGTASVVCLLNNFYPREA | |
| | (US20210332133 | KVQWKVDNALQSGNSQESVTEQDSKDSTYSLSSTLTLSKADYEKHKVY | |
| | A1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 485 |
| | TNB-486, | AKDSRGYGDYRLGGAYWGQGTLVTVSSASTKGPSVFPLAPCSRSTSES | |
| | (US20210332133 | TAALGCLVKDYFPEPVTVSWNSGALTSGVHTFPAVLQSSGLYSLSSVV | |
| | A1) | TVPSSSLGTKTYTCNVDHKPSNTKVDKRVESKYGPPCPPCPAPEAAGG | |
| | | PSVFLFPPKPKDTLMISRTPEVTCVVVDVSQEDPEVQFNWYVDGVEVH | |
| | | NAKTKPREEQFNSTYRVVSVLTVLHQDWLNGKEYKCKVSNKGLPSSIE | |
| | | KTISKAKGQPREPQVYTLPPCQEEMTKNQVSLWCLVKGFYPSDIAVEW | |
| | | ESNGQPENNYKTTPPVLDSDGSFFLYSRLTVDKSRWQEGNVFSCSVMH | |
| | | EALHNHYTQKSLSLSLG | |
| EIP0991 | TAA light | | SEQ ID |
| (V991) | chain (L2) | | NO: 486 |
| TNB-486 x | TAA heavy | EVQLVESGGGLVQPGGSLRLSCAASGFSFSDFWMSWVRQAPGKGLEWV | SEQ ID |
| CD58 | chain (H2) | ATISQAGSEKDYVDSVKGRFTISRDNAKKSLYLQMNSLRAEDTAVYYC | NO: 487 |
| | TNB-486, | ASGVYSFDYRGQGTLVTVSSESKYGPPCPPCPAPEAAGGPSVFLFPPK | |
| | (US20210332133 | PKDTLMISRTPEVTCVVVDVSQEDPEVQFNWYVDGVEVHNAKTKPREE | |
| | A1) | QFNSTYRVVSVLTVLHQDWLNGKEYKCKVSNKGLPSSIEKTISKAKGQ | |
| | | PREPQVCTLPPSQEEMTKNQVSLSCAVKGFYPSDIAVEWESNGQPENN | |
| | | YKTTPPVLDSDGSFFLVSRLTVDKSRWQEGNVFSCSVMHEALHNHYTQ | |
| | | KSLSLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 488 |
| | TNB-486, | TFGQGTKVEIKRTVAAPSVFIFPPSDEQLKSGTASVVCLLNNFYPREA | |
| | (US20210332133 | KVQWKVDNALQSGNSQESVTEQDSKDSTYSLSSTLTLSKADYEKHKVY | |
| | A1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 489 |
| | TNB-486, | AKDSRGYGDYRLGGAYWGQGTLVTVSSASTKGPSVFPLAPCSRSTSES | |
| | (US20210332133 | TAALGCLVKDYFPEPVTVSWNSGALTSGVHTFPAVLQSSGLYSLSSVV | |
| | A1) | TVPSSSLGTKTYTCNVDHKPSNTKVDKRVVESKYGPPCPPCPAPEAAG | |
| | | GPSVFLFPPKPKDTLMISRTPEVTCVVVDVSQEDPEVQFNWYVDGVEV | |
| | | HNAKTKPREEQFNSTYRVVSVLTVLHQDWLNGKEYKCKVSNKGLPSSI | |
| | | EKTISKAKGQPREPQVYTLPPCQEEMTKNQVSLWCLVKGFYPSDIAVE | |
| | | WESNGQPENNYKTTPPVLDSDGSFFLYSRLTVDKSRWQEGNVFSCSVM | |
| | | HEALHNHYTQKSLSLSLGGGGSGGGGSFSQQIYGVVYGNVTFHVPSNV | |
| | | PLKEVLWKKQKDKVAELENSEFRAFSSFKNRVYLDTVSGSLTIYNLTS | |
| | | SDEDEYEMESPNITDTMKFFLYVLESLPSPTLTCALINGSIEVQCMIP | |
| | | EHYNSHRGLIMYSWDCPMEQCKRNSTSIYFKMENDLPQKIQCTLSNPL | |
| | | FNTTSSIILTTCIPSSGHSRHRY | |
| EIP0992 | TAA light | | SEQ ID |
| (V992) | chain (L2) | | NO: 490 |
| TNB-585 | TAA heavy | QLQLQESGPGLVKPSETLSLTCTVSGGSISSSNYFWGWIRQSPGKGLE | SEQ ID |
| | chain (H2) | WIGSIDYSGYTYYNPSLKSRVTISVDTSKNQFSLKLSSVTAADTAVYN | NO: 491 |
| | TNB-585, | CARHKAATADFDYRGQGTLVTVSSGGGGSGGGGSQVQLVESGGGVVQP | |
| | (WO2021222578A | GRSLRLSCAASGFSFSRYGMHWVRQAPGKGLEGVAVIWYDGSNRYYAD | |
| | 1) | SVKGRFTISRDNSKNTLYLQMNSLRAEDTAVYYCAREPRIGYYYESSG | |
| | | YYSLDYRGQGTLVTVSSESKYGPPCPPCPAPEAAGGPSVFLFPPKPKD | |
| | | TLMISRTPEVTCVVVDVSQEDPEVQFNWYVDGVEVHNAKTKPREEQFN | |
| | | STYRVVSVLTVLHQDWLNGKEYKCKVSNKGLPSSIEKTISKAKGQPRE | |
| | | PQVCTLPPSQEEMTKNQVSLSCAVKGFYPSDIAVEWESNGQPENNYKT | |
| | | TPPVLDSDGSFFLVSRLTVDKSRWQEGNVFSCSVMHEALHNHYTQKSL | |
| | | SLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 492 |
| | TNB-585, | TFGQGTKVEIKRTVAAPSVFIFPPSDEQLKSGTASVVCLLNNFYPREA | |
| | (WO2021222578A | KVQWKVDNALQSGNSQESVTEQDSKDSTYSLSSTLTLSKADYEKHKVY | |
| | 1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 493 |
| | TNB-585, | AKDSRGYGDYRLGGAYWGQGTLVTVSSASTKGPSVFPLAPCSRSTSES | |
| | (WO2021222578A | TAALGCLVKDYFPEPVTVSWNSGALTSGVHTFPAVLQSSGLYSLSSVV | |
| | 1) | TVPSSSLGTKTYTCNVDHKPSNTKVDKRVESKYGPPCPPCPAPEAAGG | |
| | | PSVFLFPPKPKDTLMISRTPEVTCVVVDVSQEDPEVQFNWYVDGVEVH | |
| | | NAKTKPREEQFNSTYRVVSVLTVLHQDWLNGKEYKCKVSNKGLPSSIE | |
| | | KTISKAKGQPREPQVYTLPPCQEEMTKNQVSLWCLVKGFYPSDIAVEW | |
| | | ESNGQPENNYKTTPPVLDSDGSFFLYSRLTVDKSRWQEGNVFSCSVMH | |
| | | EALHNHYTQKSLSLSLG | |
| EIP0993 | TAA light | | SEQ ID |
| (V993) | chain (L2) | | NO: 494 |
| TNB-585 x | TAA heavy | QLQLQESGPGLVKPSETLSLTCTVSGGSISSSNYFWGWIRQSPGKGLE | SEQ ID |
| CD58 | chain (H2) | WIGSIDYSGYTYYNPSLKSRVTISVDTSKNQFSLKLSSVTAADTAVYN | NO: 495 |
| | TNB-585, | CARHKAATADFDYRGQGTLVTVSSGGGGSGGGGSQVQLVESGGGVVQP | |
| | (WO2021222578A | GRSLRLSCAASGFSFSRYGMHWVRQAPGKGLEGVAVIWYDGSNRYYAD | |
| | 1) | SVKGRFTISRDNSKNTLYLQMNSLRAEDTAVYYCAREPRIGYYYESSG | |
| | | YYSLDYRGQGTLVTVSSESKYGPPCPPCPAPEAAGGPSVFLFPPKPKD | |
| | | TLMISRTPEVTCVVVDVSQEDPEVQFNWYVDGVEVHNAKTKPREEQFN | |
| | | STYRVVSVLTVLHQDWLNGKEYKCKVSNKGLPSSIEKTISKAKGQPRE | |
| | | PQVCTLPPSQEEMTKNQVSLSCAVKGFYPSDIAVEWESNGQPENNYKT | |
| | | TPPVLDSDGSFFLVSRLTVDKSRWQEGNVFSCSVMHEALHNHYTQKSL | |
| | | SLSLG | |
| | CD3 light | EIVMTQSPATLSVSPGERATLSCRASQSVSSNLAWYQQKPGQAPRLLI | SEQ ID |
| | chain (L1) | YGASTRATGIPARFSGSGSGTEFTLTISSLQSEDFAVYYCQQYNNWPW | NO: 496 |
| | TNB-585, | TFGQGTKVEIKRTVAAPSVFIFPPSDEQLKSGTASVVCLLNNFYPREA | |
| | (WO2021222578A | KVQWKVDNALQSGNSQESVTEQDSKDSTYSLSSTLTLSKADYEKHKVY | |
| | 1) | ACEVTHQGLSSPVTKSFNRGEC | |
| | CD3 heavy | EVQLVESGGGLVQPGRSLRLSCAASGFTFDDYAMHWVRQAPGKGLEWV | SEQ ID |
| | chain (H1) | SGISWNSGSIGYADSVKGRFTISRDNAKNSLYLQMNSLRAEDTALYYC | NO: 497 |
| | TNB-585, | AKDSRGYGDYRLGGAYWGQGTLVTVSSASTKGPSVFPLAPCSRSTSES | |
| | (WO2021222578A | TAALGCLVKDYFPEPVTVSWNSGALTSGVHTFPAVLQSSGLYSLSSVV | |
| | 1) | TVPSSSLGTKTYTCNVDHKPSNTKVDKRVVESKYGPPCPPCPAPEAAG | |
| | | GPSVFLFPPKPKDTLMISRTPEVTCVVVDVSQEDPEVQFNWYVDGVEV | |
| | | HNAKTKPREEQFNSTYRVVSVLTVLHQDWLNGKEYKCKVSNKGLPSSI | |
| | | EKTISKAKGQPREPQVYTLPPCQEEMTKNQVSLWCLVKGFYPSDIAVE | |
| | | WESNGQPENNYKTTPPVLDSDGSFFLYSRLTVDKSRWQEGNVFSCSVM | |
| | | HEALHNHYTQKSLSLSLGGGGSGGGGSFSQQIYGVVYGNVTFHVPSNV | |
| | | PLKEVLWKKQKDKVAELENSEFRAFSSFKNRVYLDTVSGSLTIYNLTS | |
| | | SDEDEYEMESPNITDTMKFFLYVLESLPSPTLTCALINGSIEVQCMIP | |
| | | EHYNSHRGLIMYSWDCPMEQCKRNSTSIYFKMENDLPQKIQCTLSNPL | |
| | | FNTTSSIILTTCIPSSGHSRHRY | |
| EIP0869 | TAA light | EIVLTQSPATLSLSPGERATLSCSASSSVSYMHWYQKKPGQAPRLLIY | SEQ ID |
| (V869) | chain (L2) | DTSKLASGIPARFSGSGSGTDFTLTISSLEPEDVAVYYCFQGSVYPFT | NO: 498 |
| CD19 | Denintumab, | FGQGTKLEIKRTVAAPSVFIFPPSDEQLKSGTASVVCLLNNFYPREAK | |
| (Denin) | (US20120294853A | VQWKVDNALQSGNSQESVTEQDSKDSTYSLSSTLRLSKADYEKHKVYA | |
| x CD3-01 | 1) | CEVTHQGLSSPVTKSFNRGEC | |
| | TAA heavy | QVQLQESGPGLVKPSQTLSLTCTVSGGSISTSGMGVGWIRDHPGKGLE | SEQ ID |
| | chain (H2) | WIGHIWWDDDKRYNPALKSRVTISVDTSKNQFSLKLSSVTAADTAVYY | NO: 499 |
| | Denintumab, | CARMELWSYYFDYWGQGTLVTVSSASTKGPSVFPLAPSSKSTSGGTAA | |
| | (US20120294853A | LGCLVDDYFPEPVTVSWNSGALTSGVHTFPAVLQSSGLYSLSSVVTVP | |
| | 1) | SSSLGTQTYICNVNHKPSNTKVDKKVEPKSCDKTHTCPPCPAPEAAGA | |
| | | PSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPIE | |
| | | KTISKAKGQPREPQVYTLPPCREEMTKNQVSLWCLVKGFYPSDIAVEW | |
| | | ESNGQPENNYKTTPPVLDSDGSFFLYSKLTVDKSRWQQGNVFSCSVMH | |
| | | EALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 500 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 501 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPG | |
| EIP0870 | TAA light | EIVLTQSPATLSLSPGERATLSCSASSSVSYMHWYQKKPGQAPRLLIY | SEQ ID |
| (V870) | chain (L2) | DTSKLASGIPARFSGSGSGTDFTLTISSLEPEDVAVYYCFQGSVYPFT | NO: 502 |
| CD19 | Denintumab, | FGQGTKLEIKRTVAAPSVFIFPPSDEQLKSGTASVVCLLNNFYPREAK | |
| (Denin) x | (US20120294853A | VQWKVDNALQSGNSQESVTEQDSKDSTYSLSSTLRLSKADYEKHKVYA | |
| CD3- | 1) | CEVTHQGLSSPVTKSFNRGEC | |
| 01 x CD58 | TAA heavy | QVQLQESGPGLVKPSQTLSLTCTVSGGSISTSGMGVGWIRDHPGKGLE | SEQ ID |
| | chain (H2) | WIGHIWWDDDKRYNPALKSRVTISVDTSKNQFSLKLSSVTAADTAVYY | NO: 503 |
| | Denintumab, | CARMELWSYYFDYWGQGTLVTVSSASTKGPSVFPLAPSSKSTSGGTAA | |
| | (US20120294853A | LGCLVDDYFPEPVTVSWNSGALTSGVHTFPAVLQSSGLYSLSSVVTVP | |
| | 1) | SSSLGTQTYICNVNHKPSNTKVDKKVEPKSCDKTHTCPPCPAPEAAGA | |
| | | PSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPIE | |
| | | KTISKAKGQPREPQVYTLPPCREEMTKNQVSLWCLVKGFYPSDIAVEW | |
| | | ESNGQPENNYKTTPPVLDSDGSFFLYSKLTVDKSRWQQGNVFSCSVMH | |
| | | EALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 504 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 505 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPGGGGSGGGGSGGGGSFSQQIYGVVYGN | |
| | | VTFHVPSNVPLKEVLWKKQKDKVAELENSEFRAFSSFKNRVYLDTVSG | |
| | | SLTIYNLTSSDEDEYEMESPNITDTMKFFLYVLESLPSPTLTCALING | |
| | | SIEVQCMIPEHYNSHRGLIMYSWDCPMEQCKRNSTSIYFKMENDLPQK | |
| | | IQCTLSNPLFNTTSSIILTTCIPSSGHSRHRY | |
| EIP0871 | TAA light | DIVMTQSPATLSLSPGERATLSCRSSKSLQNVNGNTYLYWFQKKPGQS | SEQ ID |
| (V871) | chain (L2) | PQLLIYRMSNLNSGVPDRFSGSGSGTEFTLTISSLEPEDFAVYYCMQH | NO: 506 |
| CD19 | Obexelimab, (U.S. | LEYPITFGAGTKLEIKRTVAAPSVFIFPPSDEQLKSGTASVVCLLNNF | |
| (Obex) x | 20120082664) | YPREAKVQWKVDNALQSGNSQESVTEQDSKDSTYSLSSTLRLSKADYE | |
| CD3-01 | | KHKVYACEVTHQGLSSPVTKSFNRGEC | |
| | TAA heavy | EVQLVESGGGLVKPGGSLKLSCAASGYTFTSYVMHWVRDAPGKGLEWI | SEQ ID |
| | chain (H2) | GYINPYNDGTKYNEKFQGRVTISSDKSISTAYMELSSLRSEDTAMYYC | NO: 507 |
| | Obexelimab, (U.S. | ARGTYYYGTRVFDYWGQGTLVTVSSASTKGPSVFPLAPSSKSTSGGTA | |
| | 20120082664) | ALGCLVDDYFPEPVTVSWNSGALTSGVHTFPAVLQSSGLYSLSSVVTV | |
| | | PSSSLGTQTYICNVNHKPSNTKVDKKVEPKSCDKTHTCPPCPAPEAAG | |
| | | APSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPI | |
| | | EKTISKAKGQPREPQVYTLPPCREEMTKNQVSLWCLVKGFYPSDIAVE | |
| | | WESNGQPENNYKTTPPVLDSDGSFFLYSKLTVDKSRWQQGNVFSCSVM | |
| | | HEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 508 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 509 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPG | |
| EIP0872 | TAA light | DIVMTQSPATLSLSPGERATLSCRSSKSLQNVNGNTYLYWFQKKPGQS | SEQ ID |
| (V872) | chain (L2) | PQLLIYRMSNLNSGVPDRFSGSGSGTEFTLTISSLEPEDFAVYYCMQH | NO: 510 |
| CD19 | Obexelimab, (U.S. | LEYPITFGAGTKLEIKRTVAAPSVFIFPPSDEQLKSGTASVVCLLNNF | |
| (Obex) x | 20120082664) | YPREAKVQWKVDNALQSGNSQESVTEQDSKDSTYSLSSTLRLSKADYE | |
| CD3-01 x | | KHKVYACEVTHQGLSSPVTKSFNRGEC | |
| CD58 | TAA heavy | EVQLVESGGGLVKPGGSLKLSCAASGYTFTSYVMHWVRDAPGKGLEWI | SEQ ID |
| | chain (H2) | GYINPYNDGTKYNEKFQGRVTISSDKSISTAYMELSSLRSEDTAMYYC | NO: 511 |
| | Obexelimab, (U.S. | ARGTYYYGTRVFDYWGQGTLVTVSSASTKGPSVFPLAPSSKSTSGGTA | |
| | 20120082664) | ALGCLVDDYFPEPVTVSWNSGALTSGVHTFPAVLQSSGLYSLSSVVTV | |
| | | PSSSLGTQTYICNVNHKPSNTKVDKKVEPKSCDKTHTCPPCPAPEAAG | |
| | | APSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPI | |
| | | EKTISKAKGQPREPQVYTLPPCREEMTKNQVSLWCLVKGFYPSDIAVE | |
| | | WESNGQPENNYKTTPPVLDSDGSFFLYSKLTVDKSRWQQGNVFSCSVM | |
| | | HEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 512 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTENTYAMNWVRKAPGKGLEWV | SEQ ID |
| | chain (H1) | ARIRSKYNNYATYYADSVKDRFTISRDDSKNTAYLQMNNLKTEDTAVY | NO: 513 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPGGGGSGGGGSGGGGSFSQQIYGVVYGN | |
| | | VTFHVPSNVPLKEVLWKKQKDKVAELENSEFRAFSSFKNRVYLDTVSG | |
| | | SLTIYNLTSSDEDEYEMESPNITDTMKFFLYVLESLPSPTLTCALING | |
| | | SIEVQCMIPEHYNSHRGLIMYSWDCPMEQCKRNSTSIYFKMENDLPQK | |
| | | IQCTLSNPLFNTTSSIILTTCIPSSGHSRHRY | |
| EIP0546 | TAA light | EIVLTQSPGTLSLSPGERATFSCRSSHSIRSRRVAWYQKKPGQAPRLV | SEQ ID |
| (V546) | chain (L2) | IHGVSNRASGISDRFSGSGSGTDFTLTITRVEPEDFALYYCQVYGASS | NO: 514 |
| B12 x | B12 ab | YTFGQGTKLERKRTVAAPSVFIFPPSDEQLKSGTASVVCLLNNFYPRE | |
| CD3-01 x | (US20130251726 | AKVQWKVDNALQSGNSQESVTEQDSKDSTYSLSSTLRLSKADYEKHKV | |
| CD58 | A1) | YACEVTHQGLSSPVTKSFNRGEC | |
| | TAA heavy | QVQLVQSGAEVKKPGASVKVSCQASGYRFSNFVIHWVRDAPGQRQEWM | SEQ ID |
| | chain (H2) | GWINPYNGNKEFSAKFQDRVTFTADTSANTAYMELRSLRSADTAVYYC | NO: 515 |
| | B12 ab | ARVGPYSWDDSPQDNYYMDVWGKGTTVIVSSASTKGPSVFPLAPSSKS | |
| | (US20130251726 | TSGGTAALGCLVDDYFPEPVTVSWNSGALTSGVHTFPAVLQSSGLYSL | |
| | A1) | SSVVTVPSSSLGTQTYICNVNHKPSNTKVDKKVEPKSCDKTHTCPPCP | |
| | | APEAAGAPSVFLFPPKPKDTLMISRTPEVTCVVVDVSHEDPEVKENWY | |
| | | VDGVEVHNAKTKPREEQYNSTYRVVSVLTVLHQDWLNGKEYKCKVSNK | |
| | | ALPAPIEKTISKAKGQPREPQVYTLPPCREEMTKNQVSLWCLVKGFYP | |
| | | SDIAVEWESNGQPENNYKTTPPVLDSDGSFFLYSKLTVDKSRWQQGNV | |
| | | FSCSVMHEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 516 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 517 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKENWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPG | |
| EIP0607 | TAA light | EIVLTQSPGTLSLSPGERATFSCRSSHSIRSRRVAWYQKKPGQAPRLV | SEQ ID |
| (V607) | chain (L2) | IHGVSNRASGISDRFSGSGSGTDFTLTITRVEPEDFALYYCQVYGASS | NO: 518 |
| B12 x | B12 ab | YTFGQGTKLERKRTVAAPSVFIFPPSDEQLKSGTASVVCLLNNFYPRE | |
| CD3-A5 x | (US20130251726 | AKVQWKVDNALQSGNSQESVTEQDSKDSTYSLSSTLRLSKADYEKHKV | |
| CD58 | A1) | YACEVTHQGLSSPVTKSFNRGEC | |
| | TAA heavy | QVQLVQSGAEVKKPGASVKVSCQASGYRFSNFVIHWVRDAPGQRQEWM | SEQ ID |
| | chain (H2) | GWINPYNGNKEFSAKFQDRVTFTADTSANTAYMELRSLRSADTAVYYC | NO: 519 |
| | B12 ab | ARVGPYSWDDSPQDNYYMDVWGKGTTVIVSSASTKGPSVFPLAPSSKS | |
| | (US20130251726 | TSGGTAALGCLVDDYFPEPVTVSWNSGALTSGVHTFPAVLQSSGLYSL | |
| | A1) | SSVVTVPSSSLGTQTYICNVNHKPSNTKVDKKVEPKSCDKTHTCPPCP | |
| | | APEAAGAPSVFLFPPKPKDTLMISRTPEVTCVVVDVSHEDPEVKENWY | |
| | | VDGVEVHNAKTKPREEQYNSTYRVVSVLTVLHQDWLNGKEYKCKVSNK | |
| | | ALPAPIEKTISKAKGQPREPQVYTLPPCREEMTKNQVSLWCLVKGFYP | |
| | | SDIAVEWESNGQPENNYKTTPPVLDSDGSFFLYSKLTVDKSRWQQGNV | |
| | | FSCSVMHEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSQ | NO: 520 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTFNTYAMNWVRKAPGKGLEWV | SEQ ID |
| | chain (H1) | ARIRSKYNNYATYYADSVKDRFTISRDDSKNTAYLQMNNLKTEDTAVY | NO: 521 |
| | | YCVRHGNFGDDYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPGGGGSGGGGSGGGGSFSQQIYGVVYGN | |
| | | VTFHVPSNVPLKEVLWKKQKDKVAELENSEFRAFSSFKNRVYLDTVSG | |
| | | SLTIYNLTSSDEDEYEMESPNITDTMKFFLYVLESLPSPTLTCALING | |
| | | SIEVQCMIPEHYNSHRGLIMYSWDCPMEQCKRNSTSIYFKMENDLPQK | |
| | | IQCTLSNPLFNTTSSIILTTCIPSSGHSRHRY | |
| EIP0614 | TAA light | EIVLTQSPGTLSLSPGERATFSCRSSHSIRSRRVAWYQKKPGQAPRLV | SEQ ID |
| (V614) | chain (L2) | IHGVSNRASGISDRFSGSGSGTDFTLTITRVEPEDFALYYCQVYGASS | NO: 522 |
| B12 x | B12 ab | YTFGQGTKLERKRTVAAPSVFIFPPSDEQLKSGTASVVCLLNNFYPRE | |
| CD3-A6 x | (US20130251726 | AKVQWKVDNALQSGNSQESVTEQDSKDSTYSLSSTLRLSKADYEKHKV | |
| CD58 | A1) | YACEVTHQGLSSPVTKSFNRGEC | |
| | TAA heavy | QVQLVQSGAEVKKPGASVKVSCQASGYRFSNFVIHWVRDAPGQRQEWM | SEQ ID |
| | chain (H2) | GWINPYNGNKEFSAKFQDRVTFTADTSANTAYMELRSLRSADTAVYYC | NO: 523 |
| | B12 ab | ARVGPYSWDDSPQDNYYMDVWGKGTTVIVSSASTKGPSVFPLAPSSKS | |
| | (US20130251726 | TSGGTAALGCLVDDYFPEPVTVSWNSGALTSGVHTFPAVLQSSGLYSL | |
| | A1) | SSVVTVPSSSLGTQTYICNVNHKPSNTKVDKKVEPKSCDKTHTCPPCP | |
| | | APEAAGAPSVFLFPPKPKDTLMISRTPEVTCVVVDVSHEDPEVKFNWY | |
| | | VDGVEVHNAKTKPREEQYNSTYRVVSVLTVLHQDWLNGKEYKCKVSNK | |
| | | ALPAPIEKTISKAKGQPREPQVYTLPPCREEMTKNQVSLWCLVKGFYP | |
| | | SDIAVEWESNGQPENNYKTTPPVLDSDGSFFLYSKLTVDKSRWQQGNV | |
| | | FSCSVMHEALHNHYTQKSLSLSPG | |
| | CD3 light | ELVVTQEPSLTVSPGGTVTLTCRSSTGAVTTSNYANWVQDKPGQAPRG | SEQ ID |
| | chain (L1) | LIGGTNKRAPGTPARFSGSLLGGKAALTLSGVQPEDEAEYYCALWYSN | NO: 524 |
| | | LWVFGGGTKLTVLGQPKANPTVTLFPPSSEELQANKADLVCLISDFYP | |
| | | GAVTVAWKADGSPVKAGVETCKPSKQSNNKYAASSYLSLTPEQWKSHR | |
| | | SYSCQVTHEGSTVEKTVAPTESS | |
| | CD3 heavy | EVQLLESGGGLVQPGGSLKLSCAASGFTENDYAMNWVRKAPGKGLEWV | SEQ ID |
| | chain (H1) | ARIRSKYNNYATYYADSVKDRFTISRDDSKNTAYLQMNNLKTEDTAVY | NO: 526 |
| | | YCVRHGNFGNSYVSWFAYWGQGTLVTVSSASTKGPSVFPLAPSSKSTS | |
| | | GGTAALGCLVKDYFPEPVTVSWNSGALTSGVHTFPACLQSSGLYSLSS | |
| | | VVTVPSSSLGTQTYICNVNHKPSNTKVDKKVEPKSSDKTHTCPPCPAP | |
| | | EAAGAPSVFLFPPKPKDTLMISRTPEVTCVVVDVSHEDPEVKFNWYVD | |
| | | GVEVHNAKTKPREEQYNSTYRVVSVLTVLHQDWLNGKEYKCKVSNKAL | |
| | | PAPIEKTISKAKGQPREPQVCTLPPSREEMTKNQVSLSCAVKGFYPSD | |
| | | IAVEWESNGQPENNYKTTPPVLDSDGSFFLVSKLTVDKSRWQQGNVFS | |
| | | CSVMHEALHNHYTQKSLSLSPGGGGSGGGGSGGGGSFSQQIYGVVYGN | |
| | | VTFHVPSNVPLKEVLWKKQKDKVAELENSEFRAFSSFKNRVYLDTVSG | |
| | | SLTIYNLTSSDEDEYEMESPNITDTMKFFLYVLESLPSPTLTCALING | |
| | | SIEVQCMIPEHYNSHRGLIMYSWDCPMEQCKRNSTSIYFKMENDLPQK | |
| | | IQCTLSNPLFNTTSSIILTTCIPSSGHSRHRY | |

(Italics-Variable region, Italics and underline-Kabat CDR definition, Italics and bold-Chothia CDR definition, Underline-CD58 fusion, Underline-cysteine mutation)

In some embodiments, the bispecific antibody EIP0373 comprises a L2 comprising the amino acid sequence of SEQ ID NO: 450, a H2 comprising the amino acid sequence of SEQ ID NO: 451, a L1 comprising the amino acid sequence of SEQ ID NO: 452, and a H1 comprising the amino acid sequence of SEQ ID NO: 453.

In some embodiments, the bispecific antibody EIP0535 comprises a L2 comprising the amino acid sequence of SEQ ID NO: 454, a H2 comprising the amino acid sequence of SEQ ID NO: 455, a L1 comprising the amino acid sequence of SEQ ID NO: 456, and a H1 comprising the amino acid sequence of SEQ ID NO: 457.

In some embodiments, the bispecific antibody EIP0506 comprises a L2 comprising the amino acid sequence of SEQ ID NO: 458, a H2 comprising the amino acid sequence of SEQ ID NO: 459, a L1 comprising the amino acid sequence of SEQ ID NO: 460, and a H1 comprising the amino acid sequence of SEQ ID NO: 461.

In some embodiments, the bispecific antibody EIP0534 comprises a L2 comprising the amino acid sequence of SEQ ID NO: 462, a H2 comprising the amino acid sequence of SEQ ID NO: 463, a L1 comprising the amino acid sequence of SEQ ID NO: 464, and a H1 comprising the amino acid sequence of SEQ ID NO: 465.

In some embodiments, the bispecific antibody EIP0702 comprises a L2 comprising the amino acid sequence of SEQ ID NO: 466, a H2 comprising the amino acid sequence of SEQ ID NO: 467, a L1 comprising the amino acid sequence of SEQ ID NO: 468, and a H1 comprising the amino acid sequence of SEQ ID NO: 469.

In some embodiments, the bispecific antibody EIP0703 comprises a L2 comprising the amino acid sequence of SEQ ID NO: 470, a H2 comprising the amino acid sequence of SEQ ID NO: 471, a L1 comprising the amino acid sequence of SEQ ID NO: 472, and a H1 comprising the amino acid sequence of SEQ ID NO: 473.

In some embodiments, the bispecific antibody EIP0765 comprises a L2 comprising the amino acid sequence of SEQ ID NO: 474, a H2 comprising the amino acid sequence of SEQ ID NO: 475, a L1 comprising the amino acid sequence of SEQ ID NO: 476, and a H1 comprising the amino acid sequence of SEQ ID NO: 477.

In some embodiments, the bispecific antibody EIP0766 comprises a L2 comprising the amino acid sequence of SEQ ID NO: 478, a H2 comprising the amino acid sequence of SEQ ID NO: 479, a L1 comprising the amino acid sequence of SEQ ID NO: 480, and a H1 comprising the amino acid sequence of SEQ ID NO: 481.

In some embodiments, the bispecific antibody EIP0990 comprises a L2 comprising the amino acid sequence of SEQ ID NO: 482, a H2 comprising the amino acid sequence of SEQ ID NO: 483, a L1 comprising the amino acid sequence of SEQ ID NO: 484, and a H1 comprising the amino acid sequence of SEQ ID NO: 485.

In some embodiments, the bispecific antibody EIP0991 comprises a L2 comprising the amino acid sequence of SEQ ID NO: 486, a H2 comprising the amino acid sequence of SEQ ID NO: 487, a L1 comprising the amino acid sequence of SEQ ID NO: 488, and a H1 comprising the amino acid sequence of SEQ ID NO: 489.

In some embodiments, the bispecific antibody EIP0991 comprises a L2 comprising the amino acid sequence of SEQ ID NO: 486, a H2 comprising the amino acid sequence of SEQ ID NO: 487, a L1 comprising the amino acid sequence of SEQ ID NO: 488, and a H1 comprising the amino acid sequence of SEQ ID NO: 489.

In some embodiments, the bispecific antibody EIP0992 comprises a L2 comprising the amino acid sequence of SEQ ID NO: 490, a H2 comprising the amino acid sequence of SEQ ID NO: 491, a L1 comprising the amino acid sequence of SEQ ID NO: 492, and a H1 comprising the amino acid sequence of SEQ ID NO: 493.

In some embodiments, the bispecific antibody EIP0993 comprises a L2 comprising the amino acid sequence of SEQ ID NO: 494, a H2 comprising the amino acid sequence of SEQ ID NO: 495, a L1 comprising the amino acid sequence of SEQ ID NO: 496, and a H1 comprising the amino acid sequence of SEQ ID NO: 497.

In some embodiments, the bispecific antibody EIP0869 comprises a L2 comprising the amino acid sequence of SEQ ID NO: 498, a H2 comprising the amino acid sequence of SEQ ID NO: 499, a L1 comprising the amino acid sequence of SEQ ID NO: 500, and a H1 comprising the amino acid sequence of SEQ ID NO: 501.

In some embodiments, the bispecific antibody EIP0870 comprises a L2 comprising the amino acid sequence of SEQ ID NO: 502, a H2 comprising the amino acid sequence of SEQ ID NO: 503, a L1 comprising the amino acid sequence of SEQ ID NO: 504, and a H1 comprising the amino acid sequence of SEQ ID NO: 505.

In some embodiments, the bispecific antibody EIP0871 comprises a L2 comprising the amino acid sequence of SEQ ID NO: 506, a H2 comprising the amino acid sequence of SEQ ID NO: 507, a L1 comprising the amino acid sequence of SEQ ID NO: 508, and a H1 comprising the amino acid sequence of SEQ ID NO: 509.

In some embodiments, the bispecific antibody EIP0872 comprises a L2 comprising the amino acid sequence of SEQ ID NO: 510, a H2 comprising the amino acid sequence of SEQ ID NO: 511, a L1 comprising the amino acid sequence of SEQ ID NO: 512, and a H1 comprising the amino acid sequence of SEQ ID NO: 513.

In some embodiments, the bispecific antibody EIP0546 comprises a L2 comprising the amino acid sequence of SEQ ID NO: 514, a H2 comprising the amino acid sequence of SEQ ID NO: 515, a L1 comprising the amino acid sequence of SEQ ID NO: 516, and a H1 comprising the amino acid sequence of SEQ ID NO: 517.

In some embodiments, the bispecific antibody EIP0607 comprises a L2 comprising the amino acid sequence of SEQ ID NO: 518, a H2 comprising the amino acid sequence of SEQ ID NO: 519, a L1 comprising the amino acid sequence of SEQ ID NO: 520, and a H1 comprising the amino acid sequence of SEQ ID NO: 521.

In some embodiments, the bispecific antibody EIP0614 comprises a L2 comprising the amino acid sequence of SEQ ID NO: 522, a H2 comprising the amino acid sequence of SEQ ID NO: 523, a L1 comprising the amino acid sequence of SEQ ID NO: 524, and a H1 comprising the amino acid sequence of SEQ ID NO: 525.

TABLE 20
Bispecific Antibodies of the Present Disclosure

| | | | | | | | | Light Chain | |
|---|---|---|---|---|---|---|---|---|---|
| | | | | | | | | Pairing | |
| | | | | | | | | Mutation Set | CD3 |
| | | | | | | | | (A/B/C) (400 | binder |
| CD3 | ULBP2 | EIP | V | EV | EV104 | EVOLVE | EVOLVE104 | series?) | (affinity) |
| | E12 | | | | EV101.1 | | | | |
| | A06 | | | | EV101.2 | | | | |
| CD3-01 | E12 | EIP0174 | | | | | | A | Strongest |
| (SP34) | | | | | | | | | |
| CD3-01 | E12 | EIP0175 | | | | | | B | Strongest |
| (SP34) | | | | | | | | | |
| CD3-01 | E12 | EIP0187 | | | | | | C | Strongest |
| (SP34) | | | | | | | | | |
| CD3-01 | E12 | EIP0205 | V555 | VCD3.1/Bis | EV104.1:CD3.1/ | BISP | BISP | D | Strongest |
| (SP34) | | | | CD3.1 | EV104.1:CD3.1- | CD3.1 | EV104.1- | | |
| | | | | | ELCP | | CD3.1 | | |
| CD3-01 | E12 | EIP0206 | | | | | | E | Strongest |
| (SP34) | | | | | | | | | |
| CD3-01 | E12 | EIP0207 | | | | | | F | Strongest |
| (SP34) | | | | | | | | | |
| CD3-01 | E12 | EIP0208 | V208 | | | | | G | Strongest |
| (SP34) | | | | | | | | | |
| CD3-01 | E12 | EIP0294 | | | | | | H | Strongest |
| (SP34) | | | | | | | | | |
| CD3-01 | E12 | EIP0295 | | | | | | I | Strongest |
| (SP34) | | | | | | | | | |
| CD3-01 | E12 | EIP0306 | | | | | | J | Strongest |
| (SP34) | | | | | | | | | |
| CD3-01 | E12 | EIP0307 | | | | | | K | Strongest |
| (SP34) | | | | | | | | | |
| CD3-01 | E12 | EIP0318 | | | | | | L | Strongest |
| (SP34) | | | | | | | | | |
| CD3-01 | E12 | EIP0342 | | | | | | M | Strongest |
| (SP34) | | | | | | | | | |
| CD3-01 | E12 | EIP0354 | | | | | | N | Strongest |
| (SP34) | | | | | | | | | |
| CD3-01 | E12 | EIP0356 | | | | | | O | Strongest |
| (SP34) | | | | | | | | | |
| CD3-01 | E12 | EIP0377 | | | | | | P | Strongest |
| (SP34) | | | | | | | | | |
| CD3-01 | E12 | EIP0598 | | | | | | Q | Strongest |
| (SP34) | | | | | | | | | |
| CD3-01 | E12 | EIP0112 | | | EV104.1- | | | CrossMabControl | Strongest |
| (SP34) | | | | | CD3- | | | | |
| | | | | | CrossMab/ | | | | |
| | | | | | EV104.1- | | | | |
| | | | | | CD3.1- | | | | |
| | | | | | CrossMab | | | | |
| CD3-01 | E12 | EIP0340 | | | | | | 340 | Strongest |
| (SP34) | | | | | | | | | |
| CD3-01 | E12 | | V354 | | | | | N | Strongest |
| (SP34) | | | | | | | | | |
| CD3-01 | E12 | EIP0356 | | | | | | O | Strongest |
| (SP34) | | | | | | | | | |
| CD3-01 | E12 | EIP0367 | | | | | | 367 | Strongest |
| (SP34) | | | | | | | | | |
| CD3-01 | E12 | EIP0404 | | | | | | 404 | Strongest |
| (SP34) | | | | | | | | | |
| CD3-01 | E12 | EIP0406 | V406 | | | | | 406 | Strongest |
| (SP34) | | | | | | | | | |
| CD3-01 | E12 | EIP0473 | | | | | | 473 | Strongest |
| (SP34) | | | | | | | | | |
| CD3-A1 | E12 | EIP0527 | | | | | | D | Strongest |
| CD3-A4 | E12 | EIP0624 | | | | | | D | Strongest |
| CD3-A2 | E12 | EIP0486 | V486 | VCD3.2/Bis | | | | D | Strong |
| | | | | CD3.2 | | | | | |
| CD3-A5 | E12 | EIP0626 | | | | | | D | Strong |
| CD3-A6 | E12 | EIP0483 | V483 | VCD3.5/Bis | EV104.1:CD3.5 | | | D | Moderate |
| | | | | CD3.2 | | | | | |
| CD3-A8 | E12 | EIP0623 | | | | | | D | Moderate |
| CD3-A9 | E12 | EIP0621 | | | | | | D | Weak |
| CD3-A7 | E12 | EIP0625 | | | | | | D | Weak |
| CD3-A10 | E12 | EIP0622 | | | | | | D | Very weak |
| CD3-A11 | E12 | EIP0525 | | | | | | D | Very Weak |
| CD3-A1 | E12 | EIP0630 | | | | | | Q | Strongest |
| CD3-A4 | E12 | EIP0540 | | | | | | Q | Strongest |
| CD3-A2 | E12 | EIP0628 | | | | | | Q | Strong |
| CD3-A5 | E12 | EIP0542 | V542 | VCD3.3/Bis | EV104.1:CD3.4 | | | Q | Strong |
| | | | | CD3.3 | | | | | |
| CD3-A6 | E12 | EIP0627 | | | | | | Q | Moderate |
| CD3-A8 | E12 | EIP0515 | V515 | VCD3.6/Bis | EV104.1:CD3.6 | | | Q | Moderate |
| | | | | CD3.6 | | | | | |
| CD3-A9 | E12 | EIP0477 | V477 | VCD3.7 | EV104.1:CD3.7 | | | Q | Weak |
| CD3-A7 | E12 | EIP0541 | | | EV104.1:CD3.8 | | | Q | Weak |
| CD3-A10 | E12 | EIP0513 | | | EV104.1:CD3.9/ | | | Q | Very weak |
| | | | | | EV104.2:CD3.8 | | | | |
| CD3-A11 | E12 | EIP0629 | | | | | | Q | Very Weak |
| CD3-01 | E12 | EIP0359 | V557 | EVCD3.1/ | EV104.1:CD3.1- | EVOLVECD3.1 | EVOLVE104.1- | D | Strongest |
| | | | | EV557 | COSTIM | | CD3.1 | | |
| CD3-A1 | E12 | EIP0566 | | | EV104.1:CD3.12- | | | D | Strongest |
| | | | | | COSTIM | | | | |
| CD3-A4 | E12 | EIP0634 | | | EV104.1:CD3.3- | | | D | Strongest |
| | | | | | COSTIM | | | | |
| CD3-A2 | E12 | EIP0562 | | EVCD3.2/ | EV104.1:CD3.2- | EVOLVECD3.2 | EVOLVE104.1- | D | Strong |
| | | | | EV562/ | COSTIM | | CD3.2 | | |
| | | | | EVCD3.3 | | | | | |
| CD3-A5 | E12 | EIP0636 | | | | | | D | Strong |
| CD3-A6 | E12 | EIP0561 | V561 | EVCD3.5/ | EV104.1:CD3.5- | EVOLVECD3.5 | EVOLVE104.1- | D | Moderate |
| | | | | EV561 | COSTIM | | CD3.5/ | | |
| | | | | | | | EVOLVE-104/ | | |
| | | | | | | | EVOLVE104 | | |
| CD3-A8 | E12 | EIP0633 | | | | | | D | Moderate |
| CD3-A9 | E12 | EIP0631 | | | | | | D | Weak |
| CD3-A7 | E12 | EIP0635 | | | EV104.1:CD3.8- | | | D | Weak |
| | | | | | COSTIM | | | | |
| CD3-A10 | E12 | EIP0632 | | | | | | D | Very weak |
| CD3-A11 | E12 | EIP0565 | | | | | | D | Very Weak |
| CD3-01 | E12 | EIP0599 | | | | | | Q | Strongest |
| CD3-A1 | E12 | EIP0640 | | | | | | Q | Strongest |
| CD3-A4 | E12 | EIP0567 | | | | | | Q | Strongest |
| CD3-A2 | E12 | EIP0638 | | | | | | Q | Strong |
| CD3-A5 | E12 | EIP0568 | | EVCD3.3/ | EV104.1:CD3.4- | EVOLVECD3.3 | EVOLVE104.1- | Q | Strong |
| | | | | EV568/ | COSTIM | | CD3.3 | | |
| | | | | EVCD3.4 | | | | | |
| CD3-A6 | E12 | EIP0637 | | | | | | Q | Moderate |
| CD3-A8 | E12 | EIP0564 | | EVCD3.6/ | EV104.1:CD3.6- | EVOLVECD3.6 | | Q | Moderate |
| | | | | EV564/ | COSTIM | | | | |
| | | | | EVCD3.7 | | | | | |
| CD3-A9 | E12 | EIP0560 | | EVCD3.7/ | EV104.1:CD3.7- | EVOLVECD3.7 | EVOLVE104.1- | Q | Weak |
| | | | | EV560/ | COSTIM | | CD3.7 | | |
| | | | | EVCD3.2 | | | | | |
| CD3-A7 | E12 | EIP0662 | | | | | | Q | Weak |
| CD3-A10 | E12 | EIP0563 | | | EV104.1:CD3.9- | | | Q | Very weak |
| | | | | | COSTIM | | | | |
| CD3-A11 | E12 | EIP0639 | | | EV104.1:CD3.10- | | | Q | Very Weak |
| | | | | | COSTIM | | | | |
| CD3-01 | E12 | EIP0360 | | | EV104.1:CD3.1- | | | D | Strongest |
| | | | | | COSTIMV | | | | |
| CD3-01 | E12 | EIP0363 | | | | | | D | Strongest |
| CD3-A1 | E12 | EIP0694 | | | | | | P | Strongest |
| CD3-A4 | E12 | EIP0695 | | | | | | P | Strongest |
| CD3-A2 | E12 | EIP0696 | | | | | | P | Strong |
| CD3-A5 | E12 | EIP0697 | | | | | | P | Strong |
| CD3-A6 | E12 | EIP0698 | | | | | | P | Moderate |
| CD3-A8 | E12 | EIP0699 | | | | | | P | Moderate |
| CD3-A9 | E12 | EIP0700 | | | | | | P | Weak |
| CD3-01 | E12 | EIP0547 | | | | | | P | Strongest |
| CD3-A1 | E12 | EIP0652 | | | | | | P | Strongest |
| CD3-A4 | E12 | EIP0653 | | | | | | P | Strongest |
| CD3-A2 | E12 | EIP0654 | | | | | | P | Strong |
| CD3-A5 | E12 | EIP0655 | | | | | | P | Strong |
| CD3-A6 | E12 | EIP0656 | | | | | | P | Moderate |
| CD3-A8 | E12 | EIP0657 | | | | | | P | Moderate |
| CD3-A9 | E12 | EIP0658 | | | | | | P | Weak |
| CD3-A7 | E12 | EIP0659 | | | | | | P | Weak |
| CD3-A10 | E12 | EIP0660 | | | | | | P | Very weak |
| CD3-A1 | E12 | EIP0712 | | | | | | C | Strongest |
| CD3-A4 | E12 | EIP0713 | | | | | | C | Strongest |
| CD3-A2 | E12 | EIP0714 | | | | | | C | Strong |
| CD3-A5 | E12 | EIP0715 | | | | | | C | Strong |
| CD3-A6 | E12 | EIP0716 | | | | | | C | Moderate |
| CD3-A8 | E12 | EIP0717 | | | | | | C | Moderate |
| CD3-A9 | E12 | EIP0718 | | | | | | C | Weak |
| CD3-01 | E12 | EIP0641 | | | | | | C | Strongest |
| CD3-A1 | E12 | EIP0642 | | | | | | C | Strongest |
| CD3-A4 | E12 | EIP0643 | | | | | | C | Strongest |
| CD3-A2 | E12 | EIP0644 | | | | | | C | Strong |
| CD3-A5 | E12 | EIP0645 | | | | | | C | Strong |
| CD3-A6 | E12 | EIP0646 | | | | | | C | Moderate |
| CD3-A8 | E12 | EIP0647 | | | | | | C | Moderate |
| CD3-A9 | E12 | EIP0648 | | | | | | C | Weak |
| CD3-01 | E12 | EIP0205 | | | | | | D | Strongest |
| (SP34) | | | | | | | | | |
| CD3-01 | A06 | EIP0504 | | | EV104.2:CD3.1/ | | | D | Strongest |
| (SP34) | | | | | EV104.2- | | | | |
| | | | | | CD3 ELCP | | | | |
| CD3-01 | | EIP0373 | V373 | | EGFR-CD3 | | | D | Strongest |
| (SP34) | | | | | ELCP | | | | |
| CD3-01 | | EIP0506 | V506 | | BCMA-CD3- | | | D | Strongest |
| (SP34) | | | | | ELCP | | | | |
| | E12 | EIP0491 | V491 | | | | | D | Very weak |
| | E12 | EIP0577 | V577 | | EV104.1:CD3.9 | | | D | Very weak |
| | | EIPO120 | V120 | | | | | | |

TABLE 21
Exemplary Bispecific Antibodies of the Disclosure

| Antigen | Antibody Name/Description | CD3 Arm | EIP |
|---|---|---|---|
| EGFR | Cetuximab-CD3-01 | CD3-01 (SP34) | EIP0373 |
| BCMA | Belantamab-CD3-01 | CD3-01 (SP34) | EIP0506 |
| EGFR | Cetuximab × CD3-01 × CD58 | CD3-01 (SP34) | EIP0535 |
| BCMA | Belantamab × CD3-01 × CD58 | CD3-01 (SP34) | EIP0534 |
| BCMA | TNB-383B | | EIP0702 |
| BCMA | TNB-383B × CD58 | | EIP0703 |
| BCMA | TNB-383B - disulfide stabilized | | EIP0765 |
| BCMA | TNB-383B × CD58 - disulfide | | EIP0766 |
| | stabilized | | |
| CD19 | TNB-486 | | EIP0990 |
| CD19 | TNB-486 × CD58 | | EIP0991 |
| PSMA | TNB-585 | | EIP0992 |
| PSMA | TNB-585 × CD58 | | EIP0993 |
| CD19 | Denintumab × CD3 | CD3-01 (SP34) | EIP0869 |
| CD19 | Denintumab × CD3 × CD58 | CD3-01 (SP34) | EIP0870 |
| CD19 | Obexelimab × CD3 | CD3-01 (SP34) | EIP0871 |
| CD19 | Obexelimab × CD3 × CD58 | CD3-01 (SP34) | EIP0872 |
| gp120 | B12 × CD3 | CD3-01 (SP34) | EIP0546 |
| gp120 | B12 × CD3-A5 × CD58 | CD3-A5 | EIP0607 |
| gp120 | B12 × CD3-A6 × CD58 | CD3-A6 | EIP0614 |

### Methods of Production

Various procedures known within the art may be used for the production of polyclonal or monoclonal antibodies directed against a given target, such as, for example, ULBP2/5/6, a disease associated antigen or other target, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

In some embodiments, the antibodies of the invention are monoclonal antibodies. Monoclonal antibodies are generated, for example, by using the procedures set forth in the Examples provided herein. Antibodies are also generated, e.g., by immunizing BALB/c mice with combinations of cell transfectants expressing high levels of a given target on their surface. Hybridomas resulting from myeloma/B cell fusions are then screened for reactivity to the selected target.

Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Monoclonal antibodies of the invention include humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization is performed, e.g., by following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies also comprise, e.g., residues which are found neither in the recipient antibody nor in the imported CDR or framework sequences. In general, the humanized antibody includes substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also includes at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Fully human antibodies are antibody molecules in which the entire sequence of both the light chain and the heavy chain, including the CDRs, arise from human genes. Such antibodies are termed “human antibodies”, or “fully human antibodies” herein. Monoclonal antibodies can be prepared by using trioma technique; the human B-cell hybridoma technique (see Kozbor, et al., 1983 Immunol Today 4:72); and the EBV hybridoma technique to produce monoclonal antibodies (see Cole, et al., 1985 In: MONOCLONAL ANTIBODIES AND CANCER THERAPY, Alan R. Liss, Inc., pp. 77-96). Monoclonal antibodies may be utilized and may be produced by using human hybridomas (see Cote, et al., 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. An example of such a nonhuman animal is a mouse termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds specifically to the relevant epitope with high affinity are disclosed in PCT publication WO 99/53049.

The antibody can be expressed by a vector containing a DNA segment encoding the single chain antibody described above.

These can include vectors, liposomes, naked DNA, adjuvant-assisted DNA. gene gun, catheters, etc. Vectors include chemical conjugates such as described in WO 93/64701, which has targeting moiety (e.g., a ligand to a cellular surface receptor), and a nucleic acid binding moiety (e.g., polylysine), viral vector (e.g., a DNA or RNA viral vector), fusion proteins such as described in PCT/US 95/02140 (WO 95/22618) which is a fusion protein containing a target moiety (e.g., an antibody specific for a target cell) and a nucleic acid binding moiety (e.g., a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell's cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g., infection, transfection, transduction or transformation. Examples of modes of gene transfer include e.g., naked DNA, (Ca)₂(PO₄)₃ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g., adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

Bispecific antibodies are antibodies that have binding specificities for at least two different antigens. In the present case, one of the binding specificities is for a first target such as CD3ε or any fragment thereof. The second binding target is a disease associated antigen such as ULBP2/5/6 or any fragment thereof.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Bispecific and/or monovalent antibodies of the invention can be made using any of a variety of art-recognized techniques, including those disclosed in co-pending application WO 2012/023053, filed Aug. 16, 2011, the contents of which are hereby incorporated by reference in their entirety. The methods described in WO 2012/023053 generate bispecific antibodies that are identical in structure to a human immunoglobulin. This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However, it is also possible to obtain bispecific antibodies of the invention by fusing a Kappa light chain variable domain to a constant Lambda domain for a first specificity and fusing a Lambda light chain variable domain to a constant Kappa domain for the second specificity. The bispecific antibodies described in WO 2012/023053 are referred to as IgGκλ antibodies or “κλ bodies,” a new fully human bispecific IgG format. This κλ-body format allows the affinity purification of a bispecific antibody that is undistinguishable from a standard IgG molecule with characteristics that are undistinguishable from a standard monoclonal antibody and, therefore, favorable as compared to previous formats.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof. (See, e.g., Antibodies: A Laboratory Manual, Harlow E, and Lane D, 1988, Cold Spring Harbor Laboratory Press, Cold Spring Harbor, NY, incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4:72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”). Alternatively, a library can be prepared in yeast as protein or peptide fusions to a cell wall protein on the surface of yeast cells and encoding DNA sequences contained within the yeast cells (i.e. “yeast display library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Although not strictly impossible, the serendipitous identification of different antibodies having the same heavy chain variable domain but directed against different antigens is highly unlikely. Indeed, in most cases the heavy chain contributes largely to the antigen binding surface and is also the most variable in sequence. In particular the CDR3 on the heavy chain is the most diverse CDR in sequence, length and structure. Thus, two antibodies specific for different antigens will almost invariably carry different heavy chain variable domains.

The methods disclosed in co-pending application WO 2012/023053 overcomes this limitation and greatly facilitates the isolation of antibodies having the same heavy chain variable domain by the use of antibody libraries in which the heavy chain variable domain is the same for all the library members and thus the diversity is confined to the light chain variable domain. Such libraries are described, for example, in co-pending applications WO 2010/135558 and WO 2011/084255, each of which is hereby incorporated by reference in its entirety. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. The bispecific antibodies of the invention can be of different Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies of the invention. (see for example Strohl, WR Curr Opin Biotechnol 2009 (6):685-91; U.S. Pat. No. 6,528,624; PCT/US2009/0191199 filed Jan. 9, 2009). The methods of the invention can also be used to generate bispecific antibodies and antibody mixtures in a F(ab′)2 format that lacks the Fc portion.

The common heavy chain and two different light chains are co-expressed into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%. However, it is likely that different light chains are expressed at different levels and/or do not assemble with the same efficiency. Therefore, a means to modulate the relative expression of the different polypeptides is used to compensate for their intrinsic expression characteristics or different propensities to assemble with the common heavy chain. This modulation can be achieved via promoter strength, the use of internal ribosome entry sites (IRES) featuring different efficiencies or other types of regulatory elements that can act at transcriptional or translational levels as well as acting on mRNA stability. Different promoters of different strength could include CMV (Immediate-early Cytomegalovirus virus promoter); EF1-1α (Human elongation factor 1α-subunit promoter); Ubc (Human ubiquitin C promoter); SV40 (Simian virus 40 promoter). Different IRES have also been described from mammalian and viral origin. (See e.g., Hellen C U and Sarnow P. Genes Dev 2001 15:1593-612). These IRES can greatly differ in their length and ribosome recruiting efficiency. Furthermore, it is possible to further tune the activity by introducing multiple copies of an IRES (Stephen et al. 2000 Proc Natl Acad Sci USA 97:1536-1541). The modulation of the expression can also be achieved by multiple sequential transfections of cells to increase the copy number of individual genes expressing one or the other light chain and thus modify their relative expressions. The Examples provided herein demonstrate that controlling the relative expression of the different chains is critical for maximizing the assembly and overall yield of the bispecific antibody.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

Other suitable purification methods include those disclosed in co-pending application PCT/IB2012/003028, filed on Oct. 19, 2012, published as WO2013/088259, the contents of which are hereby incorporated by reference in their entirety.

In other embodiments of producing bispecific antibodies, antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface includes at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148 (5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate antibodies are also within the scope of the present invention. Heteroconjugate antibodies are composed of two covalently joined antibodies. Such antibodies have, for example, been proposed to target immune system cells to unwanted cells (see U.S. Pat. No. 4,676,980), and for treatment of HIV infection (see WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in treating cancer and/or other diseases and disorders associated with aberrant ULBP2/5/6 expression and/or activity. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176:1191-1195 (1992) and Shopes, J. Immunol., 148:2918-2922 (1992)). Alternatively, an antibody can be engineered that has dual Fc regions and can thereby have enhanced complement lysis and ADCC capabilities. (See Stevenson et al., Anti-Cancer Drug Design, 3:219-230 (1989)).

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257:286-288 (1982) via a disulfide-interchange reaction.

### Methods of Use

Any of the anti-CD3ε antibodies of the invention (e.g., bispecific anti-CD3 antibodies of the invention that bind to CD3ε, and a second biological molecule, e.g., a disease associated antigen), may be used in therapeutic methods.

In one aspect, an anti-CD3 antibody (e.g. bispecific anti-CD3 and ULBP2 antibody) for use as a medicament is provided. In further aspects, an anti-CD3 antibody for use in treating or delaying progression of a cell proliferative disorder (e.g., cancer, e.g., esophageal cancer or an adenocarcinoma) or an autoimmune disorder (e.g., arthritis) is provided. In some aspects, an anti-CD3 antibody for use in treating or delaying aged related diseases is provided.

In certain embodiments, an anti-CD3ε antibody for use in a method of treatment is provided. In certain embodiments, the invention provides an anti-CD3ε antibody for use in a method of treating an individual having a cell proliferative disorder or an autoimmune disorder comprising administering to the individual an effective amount of the anti-CD3ε antibody. In one such embodiment, the method further comprises administering to the individual an effective amount of at least one additional therapeutic agent, for example, as described below. In further embodiments, the invention provides an anti-CD3ε antibody for use in enhancing immune function in an individual having a cell proliferative disorder or an autoimmune disorder. In certain embodiments, the invention provides an anti-CD3ε antibody for use in a method of enhancing immune function in an individual having a cell proliferative disorder or an autoimmune disorder comprising administering to the individual an effective of the anti-CD3ε antibody to activate effector cells (e.g., T cells, e.g., CD8+ and/or CD4+ T cells), expand (increase) an effector cell population, reduce a target cell (e.g., a cell expressing a second biological molecule recognized by an anti-CD3ε antibody of the invention, such as a bispecific anti-CD3ε and ULBP2/5/6 antibody of the invention) population, and/or kill a target cell (e.g., target tumor cell). An “individual” according to any of the above embodiments may be a human.

In a further aspect, the invention provides for the use of an anti-anti-CD3ε antibody (e.g. bispecific anti-anti-CD3ε and ULBP2/5/6 antibody) in the manufacture or preparation of a medicament. In one embodiment, the medicament is for treatment of a cell proliferative disorder (e.g., cancer, e.g., esophageal cancer or an adenocarcinoma) or an autoimmune disorder (e.g., arthritis). In a further embodiment, the medicament is for use in a method of treating a cell proliferative disorder or an autoimmune disorder comprising administering to an individual having a cell proliferative disorder or an autoimmune disorder an effective amount of the medicament. In one such embodiment, the method further comprises administering to the individual an effective amount of at least one additional therapeutic agent, for example, as described below. In a further embodiment, the medicament is for activating effector cells (e.g., T cells, e.g., CD8+ and/or CD4+ T cells), expanding (increasing) an effector cell population, reducing a target cell (e.g., a cell expressing a second biological molecule recognized by an anti-CD3 antibody of the invention, such as a bispecific TDB antibody of the invention) population, and/or killing target cells (e.g., target tumor cells) in the individual. In a further embodiment, the medicament is for use in a method of enhancing immune function in an individual having a cell proliferative disorder or an autoimmune disorder comprising administering to the individual an amount effective of the medicament to activate effector cells (e.g., T cells, e.g., CD8+ and/or CD4+ T cells), expand (increase) an effector cell population, reduce a target cell (e.g., a cell expressing a second biological molecule recognized by an anti-CD3 antibody of the invention, such as a bispecific anti-CD3 and ULBP2 antibody of the invention) population, and/or kill a target cell (e.g., target tumor cell). An “individual” according to any of the above embodiments may be a human.

In a further aspect, the invention provides a method for treating a cell proliferative disorder (e.g., cancer, e.g., esophageal cancer or an adenocarcinoma) or an autoimmune disorder (e.g., arthritis). In one embodiment, the method comprises administering to an individual having such a cell proliferative disorder or an autoimmune disorder an effective amount of an anti-anti-CD3ε antibody (e.g. bispecific anti-CD3ε and ULBP2/5/6 antibody). In one such embodiment, the method further comprises administering to the individual an effective amount of at least one additional therapeutic agent, for example, as described below. An “individual” according to any of the above embodiments may be a human. In a further aspect, the invention provides a method for enhancing immune function in an individual having a cell proliferative disorder or an autoimmune disorder in an individual having a cell proliferative disorder or an autoimmune disorder. In one embodiment, the method comprises administering to the individual an effective amount of an anti-CD3ε antibody to activate effector cells (e.g., T cells, e.g., CD8+ and/or CD4+ T cells), expand (increase) an effector cell population, reduce a target cell (e.g., a cell expressing a second biological molecule recognized by an anti-CD3ε antibody of the invention, such as a e.g. bispecific anti-CD3ε and ULBP2/5/6 antibody of the invention) population, and/or kill a target cell (e.g., target tumor cell). In one embodiment, an “individual” is a human.

In a further aspect, the invention provides a method for treating urothelial cancer, esophageal cancer, stomach cancer, small intestine cancer, large intestine cancer, colorectal cancer, or an adenocarcinoma (e.g., colorectal adenocarcinoma, gastric adenocarcinoma, or pancreatic adenocarcinoma), which may be metastatic adenocarcinoma (e.g., metastatic colorectal adenocarcinoma, metastatic gastricadenocarcinoma, or metastatic pancreatic adenocarcinoma), by administering an effective amount of an anti-CD3ε antibody of the invention, such as a e.g. bispecific anti-CD3ε and ULBP2/5/6 antibody.

In other embodiments, the bispecific anti-CD3ε antibody is coadministered (concurrently, as a single or multiple compositions (e.g., formulations)) with one or more additional therapeutic agents. In other embodiments, the bispecific anti-CD3ε antibody is administered before one or more additional therapeutic agents. In other embodiments, the bispecific anti-CD3ε antibody is administered after one or more additional therapeutic agents. Exemplary additional therapeutic agents include but are not limited to CDK4/6 inhibitors (e.g. Palbociclib (Ibrance®)), anti-PD1 antibodies (e.g. Nivolumab, Pembrolizumab and Cemiplimab), FOLFOX (oxaliplatin (ELOXATIN™) combined with 5-fluorouracil and leucovorin), capecitabine (XELODA®), 5-fluorouracil (5-FU), CapeOx (XELOX; capecitabine with oxaliplatin), leucovorin (folinic acid), bevacizumab (AVASTIN®), cetuximab (ERBITUX®), panitumumab (VECTIBIX®), regorafenib (STIVARGA®), irinotecan (CPT-11; CAMPTOSAR®), and FLOX (5-fluorouracil with oxaliplatin).

In another aspect, the invention provides a method for treating a hematological cancer, such as a B cell cancer (for example, mature B-cell lymphoma) by administering an effective amount of an anti-CD3ε antibody of the invention, such as a bispecific TDB antibody of the invention, such as an anti-B cell targeting TDB, such as a CD20-TDB having an anti-CD3ε arm and an anti-CD20 arm. In a further aspect of the embodiment, the mature B-cell lymphoma is a Non-Hodgkin's Lymphoma (NHL). In a further aspect of the embodiment, the NHL is selected from the group comprising: germinal-center B-cell-like (GCB) DLBCL, activated B-cell like (ABC) DLBCL, follicular lymphoma (FL), mantle cell lymphoma (CL), acute myeloid leukemia (AML), chronic lymphoid leukemia (CLL), marginal zone lymphoma (ZL), small lymphocytic leukemia (SLL), lymphoplasmacytic lymphoma (LL), Waldenstrom macroglobulinemia (W), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, μ Heavy chain disease, Plasma cell myeloma, Solitary plasmacytoma of bone, [Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma. In a preferred embodiment of the invention, the method comprises treating a cancer comprising germinal-center B-cell like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), or Burkitt's lymphoma (BL).

In a further aspect, the invention provides pharmaceutical formulations comprising any of the anti-CD3ε antibodies provided herein, e.g., for use in any of the above therapeutic methods. In one embodiment, a pharmaceutical formulation comprises any of the anti-CD3ε antibodies provided herein and a pharmaceutically acceptable carrier. In another embodiment, a pharmaceutical formulation comprises any of the anti-CD3ε antibodies provided herein and at least one additional therapeutic agent, for example, as described herein.

Antibodies of the invention can be used either alone or in combination with other agents in a therapy. For instance, an antibody of the invention may be co-administered with at least one additional therapeutic agent. In certain embodiments, an additional therapeutic agent is a chemotherapeutic agent, growth inhibitory agent, cytotoxic agent, agent used in radiation therapy, anti-angiogenesis agent, apoptotic agent, anti-tubulin agent, or other agent, such as a epidermal growth factor receptor (EGFR) antagonist (e.g., a tyrosine kinase inhibitor), HER1/EGFR inhibitor (e.g., erlotinib (Tarceva™), platelet derived growth factor inhibitor (e.g., Gleevec™ (Imatinib Mesylate)), a COX-2 inhibitor (e.g., celecoxib), interferon, cytokine, antibody other than the anti-CD3 antibody of the invention, such as an antibody that bind to one or more of the following targets ErbB2, ErbB3, ErbB4, PDGFR-beta, BlyS, APRIL, BCMA VEGF, or VEGF receptor(s), TRAIL/Apo2, PD-1 (e.g. Nivolumab, Pembrolizumab, Cemiplimab), PD-L1 (Atezolizumab, Avelumab, Durvalumab), PD-L2, or another bioactive or organic chemical agent. In some embodiments, the invention provides a method wherein the additional therapeutic agent is a glucocorticoid. In one embodiment, the glucocorticoid is dexamethasone.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-CD3ε antibody and administration of an additional therapeutic agent occur within about one month, or within about one, two or three weeks, or within about one, two, three, four, five, or six days, of each other. Anti-CD3ε antibodies of the invention (e.g., bispecific anti-CD3ε antibodies of the invention that bind to CD3ε and a second biological molecule, e.g., a disease associated antigen) can also be used in combination with radiation therapy.

An antibody of the invention (and/or any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, the antibody is administered by subcutaneous administration. In some embodiments, an anti-CD3ε antibody administered by subcutaneous injection exhibits a less toxic response in a patient than the same anti-CD3ε antibody administered by intravenous injection. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

As a general proposition, the therapeutically effective amount of the anti-CD3ε antibody (e.g. bispecific anti-CD3ε and ULBP2/5/6 antibody) administered to human will be in the range of about 0.01 to about 100 mg/kg of patient body weight whether by one or more administrations. In some embodiments, the antibody used is about 0.01 to about 45 mg/kg, about 0.01 to about 40 mg kg, about 0.01 to about 35 mg/kg, about 0.01 to about 30 mg/kg, about 0.01 to about 25 mg/kg, about 0.01 to about 20 mg/kg, about 0.01 to about 15 mg kg, about 0.01 to about 10 mg/kg, about 0.01 to about 5 mg/kg, or about 0.01 to about 1 mg/kg administered daily, for example. In one embodiment, an anti-CD3ε antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg kg, or 10 mg/kg (or any combination thereof) may be administered to the patient. Such doses may be administered intermittently, for example, every week or every three weeks (e.g., such that the patient receives from about two to about twenty, or, for example, about six doses of the anti-CD3ε antibody). An initial higher loading dose, followed by one or more lower doses may be administered. The progress of this therapy is easily monitored by conventional techniques and assays.

In some embodiments, the methods may further comprise an additional therapy. The additional therapy may be radiation therapy, surgery, chemotherapy, gene therapy, DMA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be a separate administration of one or more of the therapeutic agents described above.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include an antibody of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as CD3ε, ULBP2/5/6, or a combination thereof (or a fragment thereof), may be used in methods known within the art relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the invention can be used to isolate a particular target using standard techniques, such as immunoaffinity, chromatography or immunoprecipitation. Antibodies of the invention (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90:7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., F_{ab}, SCFv, or F_{(ab)2}) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Pharmaceutical Compositions

The antibodies of the invention (also referred to herein as “active compounds”), and derivatives, fragments, analogs and homologs thereof, can be incorporated into pharmaceutical compositions suitable for administration. Such compositions typically comprise the antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, F_{ab}, F_{ab′} and F_{(ab′)2} fragments, scFvs, and an Fab expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, IgG4 and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen binding region” or “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” Various methods are known in the art for numbering the amino acids sequences of antibodies and identification of the complementary determining regions. For example, the Kabat numbering system (See Kabat, E. A., et al., Sequences of Protein of immunological interest, Fifth Edition, US Department of Health and Human Services, US Government Printing Office (1991)) or the IMGT numbering system (See IMGT®, the international ImMunoGeneTics information System®. Available online: http://www.imgt.org/). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (See Lefranc. M. P. et al., Biomolecules, 2014 December; 4 (4), 1102-1139). IMGT tools and databases run against IMGT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (See Lefranc. M. P. et al., Biomolecules, 2014 December; 4 (4), 1102-1139).

The term “hypervariable region” or “variable region” refers to the amino acid residues of an antibody that are typically responsible for antigen-binding. The hypervariable region generally comprises amino acid residues from a “complementarity determining region” or “CDR” (e.g., around about residues 24-34 (LI), 50-56 (L2) and 89-97 (L3) in the V_{L}, and around about 31-35 (HI), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (LI), 50-56 (L2) and 89-97 (L3) in the V_{L}, and 26-32 (HI), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop” VCDR (e.g., residues 27-38 (LI), 56-65 (L2) and 105-120 (L3) in the V_{L}, and 27-38 (HI), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally, the antibody has symmetrical insertions at one or more of the following points 28, 36 (LI), 63, 74-75 (L2) and 123 (L3) in the V_{L}, and 28, 36 (HI), 63, 74-75 (H2) and 123 (H3) in the VH when numbered in accordance with AHo; Honneger, A. and Plunkthun, A. J. Mol. Biol. 309:657-670 (2001)).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is the to specifically bind an antigen when the dissociation constant is ≤1 μM; e.g., ≤100 nM, preferably ≤10 nM and more preferably ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is the to specifically bind to its target, when the equilibrium binding constant (K_{d}) is ≤1 μM, e.g., ≤ 100 nM, preferably ≤10 nM, and more preferably ≤1 nM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules, and nucleic acid molecules encoding the light chain immunoglobulin molecules described herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules, and the light chain immunoglobulin molecules described herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to affect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

As used herein, the twenty conventional amino acids and their abbreviations follow conventional usage. See Immunology-A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹ In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

### EXAMPLES

### Example 1: Materials and Methods for Examples 2-8

### Proteins

Amino acids 26-216 of cynomolgus ULBP2 (NCBI Reference Sequence: XP_005552169.1) was fused to hIgG1 Fc and expressed in Expi293 HEK cells. Human ULBP2-Fc, ULBP2-His, ULBP5-His, ULBP6-His and NKG2D-Fc was purchased from R&D Systems. Biotin labeled human CD3 epsilon/delta and cynomolgus CD3 epsilon/delta were also purchased from Acro Biosystems.

### Bio-Layer Interferometry

Bio-layer interferometry (BLI) was performed on the Octet RED384 (Sartorius). ULBP2 antibodies were diluted to 5 μg/mL in kinetics buffer (PBS+0.02% Tween20, 0.1% BSA, 0.05% sodium azide) followed by loading to AHC biosensors (Sartorius) for 120 seconds. For association, sensors were transferred to wells containing ULBP2, ULBP5 and ULBP6 at the following concentrations (250, 125, 62.5 and 31.25 nM) for 300 seconds. Subsequently, dissociation was measured over 600 seconds. The kinetic and affinity constants are determined by fitting the data to a 1:1 binding model using the Octet analysis software.

### Surface Plasmon Resonance

Surface plasmon resonance (SPR) was performed on the Biacore 8K+ (Cytiva). Biotin labelled proteins were captured using the Sensor chip SA (Cytiva). Antibodies were diluted in the running buffer (10 mM HEPES, 150 mM NaCl, 0.05% v/v Surfactant P20, pH 7.4) to appropriate concentrations and injected to each channel at a flow rate of 30 μL/min for the multi-cycle kinetics/affinity analysis. The association contact and dissociation time are 60-120 seconds and 120-420 seconds respectively. The chip surface is regenerated by injecting 10 mM glycine, pH 1.5, at a flow rate of 30 μL/min for 60 seconds. The kinetic and affinity constants are determined by fitting the data to a 1:1 binding model using the Biacore insight evaluation software.

### NKG2D Competition ELISA

Maxisorp plates (Nunc) were coated with 5 μg/mL of ULBP2-His in PBS. Following 1 hour blocking with PBS containing 5% BSA, wells were incubated with 10 nM of biotin labeled NKG2D-Fc and increasing concentration of A06 or E12 antibodies (0.0003, 0.0011, 0.0046, 0.0183, 0.0732, 0.29, 1.17, 4.69, 18.8, 75, 300 nM) for an additional 1 hour. Wells were then washed and incubated with streptavidin horse radish peroxidase conjugate for another 1 hour. Chemiluminscence substrate (ThermoFisher) was added and luminescence was measured using EnSight reader (Perkin Elmer).

### In Vitro Repeat Stimulation of T Cells Using Biologics Conjugated to Dynabeads

T cells were isolated from healthy PBMC by negative selection using EasySep Human T cell CD3+ Isolation kit (STEMCELL) according to the manufacturer's instructions and resuspended in RPMI 1640 GlutaMAX Media plus 10% FBS. For each round of stimulation, isolated T cells (1×10⁶ cells/ml) were incubated with 10 nM of protein conjugated to Dynabeads (Thermofisher) at 37° C. for 2 to 3 days. At the end of each round of stimulation, the cells were passaged by removing the dynabeads using magnetic separation and cultured with new cell culture media and fresh dynabeads every 2 to 3 days. The cells were also collected at the end of each round for cell counting and surface and intracellular marker expression was determined by flow staining.

### T Cell Isolation and Activation

T-cells were isolated from healthy PBMCs donors using StemCell T-cell isolation kit. OpTmizer media was supplemented with 2% human AB serum, 1× penicillin/streptomycin, 4 mM glutaMAX, 2 mM glutamine. Cells were incubated at 37° C. in 5% CO2 in the supplemented OpTmizer media at 5×10⁵ cells/mL and treated with Dynabeads coated with CD3 and CD28 antibodies for 48 hours. Following removal of Dynabeads, T cells were incubated with IL-7 (2.5 ng/ml) and IL-15 (2.5 ng/ml) in supplemented OpTmizer media for an additional 6 days. The supplemented media was replaced with freshly thawed cytokines (IL7 and IL15) every 48 hours. On day of adoptive transfer T cell number and viability were measured using hemocytometer.

Tumor Studies with Co-Engraftment of Human T Cells

Five million CORL-105-GFP-luc cells and 1 million activated T cells were implanted subcutaneously were implanted subcutaneously into flank of NSG mice (NOD.Cg-Prkdcscid Il2rgtm1Wjl/SzJ, Jackson Laboratory). Antibodies were dosed 4 mg/kg biweekly starting at day 0 for 3 weeks. Tumor growth was monitored using electronic calipers and volume were calculated according to the formula: π/6×(length×width²).

### Pharmacokinetics of EIP0561 in Cynomolgus Monkey

Three cynomolgus monkeys were infused intravenously with EIP0561 for each of the following dose cohorts: 0.25, 1.0 and 4.0 mg/kg. Blood samples were collected at 0.03 0.5, 2, 8, 24, 48, 72 and 168 hours following infusion using tubes containing anticoagulant, K2EDTA, and centrifuged at 2500 rpm for 10 minutes at 4° C. The plasma was then divided into aliquots and stored at ≤60° C.

The levels of EIP0561 in cynomolgus plasma was determined using sandwich ELISA. Biotin conjugated ULBP2 (4.5 μg/mL) in blocking buffer (3% BSA in PBS) was incubated with streptavidin-coated plates (100 μL per well) for 16 hours at 4° C. Plasma samples were thawed on ice and centrifuged at 21,000×g for 5 minutes at 4° C. Serial dilutions of samples at each time point were prepared in blocking buffer while for the standard curve, four-fold serial dilutions of EIP0561 were made in blocking buffer plus 2% normal cynomolgus monkey serum in duplicate. ELISA plates were incubated overnight at 4° C. Assay plates were washed three times with PBST, mouse-α-human IgG-HRP (clone G18-145) detection antibody (diluted 1:500 in blocking buffer) was added to each well, and the plates were incubated for 1 hour at room temperature. After washing, chemiluminescent peroxidase substrate was added to the microplates, incubated 5 minutes at room temperature, and luminescence was measured on an EnSight plate reader.

The plasma concentration (Cp) of EIP0561 in each sample was plotted as a function of time. Non-compartmental analysis was performed with IQnca software package (by IntiQuan Version: 1.3.0 with compliance mode OFF) in R version 4.2.1 (2022-06-23 ucrt). IQnca default configurations were used except where specified and include the following: NCA model was defined by profile type: “single dose” and administration type: “IV bolus”, since IV infusion times were not provided. Under these conditions, any missing pre-dose imputation for AUC calculation are back-extrapolated according to the manufacturer's criteria. (https://iqnca.intiquan.com/). AUC calculation method was Linear Trapezoidal with Linear Interpolation. Lambda Z (λz) method was best fit for λz, Log regression and required a minimum of three points. No weighting was used in λz calculation.

### Yeast Surface Display

Plasmid DNA encoding the following: (1) human ULBP2 (26-216), human ULBP2 with the substitution R106L, alanine variants of human ULBP2 (26-216) containing single alanine substitutions or cynomolgus ULBP2 (26-216), (2) V5 epitope tag, and (3) Aga2 protein, all under galactose inducible promoter were transformed into competent EBY-100 yeast (ATCC) and plated on CM glucose media minus tryptophan and grown 30 □C for 2-4 days. Single colony transformants were then transferred into 2 mL deep well plates with CM glucose media minus tryptophan and grown at 30° C. overnight with shaking. Cells were pelleted and transferred to CM galactose media minus tryptophan and grown overnight at 20 DC overnight to induce expression and surface display of ULBP2.

For epitope mapping, 48 ULBP2 alanine variants were incubated with biotin labelled A06 and E12 (1 nM) and mouse anti-V5 antibody for 1 hour at room temperature. Cells were then washed using PBS containing 0.1% BSA and 0.01% Tween and incubated on ice with streptavidin-PE conjugate and mouse anti-V5 antibody for Alexa Fluor® 488 goat anti-mouse IgG antibody (ThermoFisher) and Alexa Fluor™ 647 streptavidin (ThermoFisher) for 30 minutes on ice. Cells were washed and analyzed using BD FACSymphony A3 flow cytometer (BD Biosciences).

### Molecular Modeling and Structural Bioinformatics

Antigen human ULBP2 structure was generated using “Build homology model” tool in, BioLuminate, Schrödinger, LLC, New York, NY, 2021. For this, human ULBP2 sequence (SEQ ID NO: 421) was used. Crystal structure of human NKG2D in complex with ULBP6 (PDB ID: 4S0U) was utilized as template for building human ULBP2 structure. Generated model was refined using “protein preparation workflow” in Bioluminate by performing a restrained minimization using OPLS_2005 forcefield. Human ULBP2 and ULBP6 (SEQ ID: 423) share 96% sequence identity.

Structure of human ULBP2-NKG2D complex was modeled using the crystal structure of human NKG2D in complex with ULBP6 (PDB ID: 4S0U). For this, the predicted structure of human ULBP2 was aligned over ULBP6 in ULBP6-NKG2D complex (PBD ID: 4S0U) using Pymol (The PyMOL Molecular Graphics System, Version 2.0 Schrödinger, LLC). Now, the structure of ULBP6 was removed from the complex and human ULBP2 was extracted with NKG2D as a complex.

Antibody structures for E12 and A06 were generated using “Antibody structure prediction” tool in, BioLuminate, Schrödinger, LLC, New York, NY, 2021. For modeling E12 antibody, the structure of human agonist antibody KMTR2 (PDB ID: 3X3F) was used for building both heavy and light chain of E12. Similarly for modeling A06 antibody, structure of Fab4AB007 (PDB ID: 5MVZ) was used. Generated models were refined using “protein preparation workflow” in Bioluminate by performing a restrained minimization using OPLS_2005 forcefield.

### Example 2: Characterization of Humanized ULBP2 Antibodies

### Humanized ULBP2 Antibodies

The binding kinetics of humanized ULBP2 antibodies A06 and E12 to human and cynomolgus ULBP2 was determined using surface plasmon resonance (SPR) for which the kinetic constants are summarized in Table 23 below.

TABLE 23
Association, dissociation, and equilibrium constants
for antibody binding to human and cynomolgus ULBP2

| Anti- | | ka | kd | KD |
|---|---|---|---|---|
| body | Antigen | (1/Ms) | (1/s) | n(M) |
| E12 | Human ULBP2 | 9.57E+05 | 2.11E−04 | 0.22 |
| | Cynomolgus ULBP2 | 3.41E+06 | 1.82E−03 | 0.534 |
| A06 | Human ULBP2 | 1.32E+06 | 2.14E−04 | 0.162 |
| | Cynomolgus ULBP2 | No binding | No binding | No binding |

The binding kinetics of humanized ULBP2 antibodies A06 and E12 to human ULBP2, ULBP5 and ULBP6 was determined using Bio-layer interferometry (BLI) for which the dissociation constant are summarized in Table 24 below.

TABLE 24
Equilibrium dissociation constants for antibody
binding to human ULBP2, ULBP5 and ULBP6

K_{D} (nM)

| Protein | E12 | A06 |
|---|---|---|
| ULBP2 | 0.198 | 0.532 |
| ULBP5 | 0.345 | No binding |
| ULBP6 | 0.456 | No binding |

The characteristic feature of A06 is lack of binding to ULBP5, ULBP6 and cynomolgus ULBP2 which all share a common amino acid leucine at position 106. In contrast arginine is found at position 106 in ULBP2. In Table 25 below, A06 is shown to bind to yeast displaying ULBP2 but not ULBP2 R106L nor cynomolgus ULBP2. In contrast, E12 binds to all three proteins displayed on yeast. This data suggests that R106 is a critical residue of the A06 epitope.

TABLE 25
Mean fluorescent intensities (MFI) of ULBP2 antibodies binding
to ULBP2 displaying yeast. MFI of unstained control is 385.

| | MFI | | |
|---|---|---|---|
| | | Human ULBP2 | Cynomolgus |
| Antibody | Human ULBP2 | R106L | ULBP2 |
| A06 | 8026 | 828 | 551 |
| E12 | 5182 | 9164 | 11682 |

Competition with NKG2D

ULBP2 antibodies were evaluated for their ability to compete with NKG2D binding to ULBP2. As shown in Table 26, A06 inhibited binding of NKG2D-Fc (10 nM) to plate bound ULBP2 with an IC50 of 2.26 nM whereas E12 had no effect.

TABLE 26
Half maximal inhibitory concentration of ULBP2
antibodies for NKG2D binding to ULBP2

| Antibody | IC50 (nM) - NKG2D binding to ULBP2 |
|---|---|
| A06 | 2.26 |
| E12 | No inhibition |

### ULBP2 Alanine Scanning Using Yeast Display

The following list of ULBP2 surface residues were mutated to alanine for interrogating A06 and E12 antibody binding (Table 27).

TABLE 27
Surface residues of ULBP2 for alanine substitution

| Ab Mutant # | Residue |
|---|---|
| 1 | THR38 |
| 2 | ILE40 |
| 3 | PRO41 |
| 4 | LYS42 |
| 5 | PHE43 |
| 6 | ARG44 |
| 7 | PRO45 |
| 8 | PRO47 |
| 9 | ARG48 |
| 10 | VAL52 |
| 11 | GLN53 |
| 12 | THR60 |
| 13 | HIE63 |
| 14 | ASP65 |
| 15 | Cys66 |
| 16 | ASN68 |
| 17 | LYS69 |
| 18 | Thr70 |
| 19 | THR72 |
| 20 | PRO73 |
| 21 | PRO92 |
| 22 | VAL93 |
| 23 | ARG95 |
| 24 | GLU96 |
| 25 | ASP99 |
| 26 | THR102 |
| 27 | GLU103 |
| 28 | GLN104 |
| 29 | LEU105 |
| 30 | ARG106 |
| 31 | ASP107 |
| 32 | ILE108 |
| 33 | GLN109 |
| 34 | LEU110 |
| 35 | GLU111 |
| 36 | THR114 |
| 37 | PRO115 |
| 38 | LYS116 |
| 39 | GLU117 |
| 40 | PRO118 |
| 41 | LEU119 |
| 42 | THR120 |
| 43 | SER142 |
| 44 | ASP144 |
| 45 | GLU170 |
| 46 | LYS171 |
| 47 | GLU173 |
| 48 | ASP175 |

Forty-eight individual alanine substitution mutants of ULBP2 were generated and displayed on surface of yeast and incubated with A06 and E12 antibody prior to being analyzed on the flow cytometer. Using FlowJo v10 software, the binding of A06 and E12 to each ULBP2 mutant was observed by the percentage of cells falling into 3 defined regions of the scatter plot: gate 1-corresponds to antibody binding to wild-type ULBP2, gate 2-represents intermediate binding to ULBP2, gate 3-represents weak to no binding to ULBP2. The frequency of cells falling into the 3 distinct gates for each ULBP2 mutant for A06 and E12 are shown in Table 28 and Table 29 respectively.

TABLE 28
Frequency of total cells for A06 (1 nM) binding to ULBP2 mutants

| ULBP2 | Gate 1 \| Freq. of | Gate 2 \| Freq. of | Gate 3 \| Freq. of |
|---|---|---|---|
| mutant | Total | Total | Total |
| P41A | 0.052 | 0.32 | 2.61 |
| L110A | 0.065 | 0.56 | 21.9 |
| E103A | 0.085 | 0.29 | 1.15 |
| D107A | 0.095 | 1.07 | 18.6 |
| T102A | 0.13 | 0.28 | 1.79 |
| R106A | 0.15 | 0.36 | 1.25 |
| P45A | 0.16 | 3.15 | 20.6 |
| D175A | 0.17 | 12.8 | 41.7 |
| R44A | 0.19 | 0.8 | 1.58 |
| P118A | 0.21 | 2.78 | 34.9 |
| F43A | 0.22 | 10.8 | 40.9 |
| D65A | 0.27 | 12.5 | 45.3 |
| I108A | 0.42 | 38.9 | 24.5 |
| K42A | 3.31 | 54.9 | 12.3 |
| E117A | 10.7 | 55.4 | 4.61 |
| R48A | 13.3 | 56.8 | 4.03 |
| E111A | 18.5 | 56.1 | 0.87 |
| D99A | 22.9 | 35.2 | 8.32 |
| E170A | 29.2 | 37.8 | 0.19 |
| Q109A | 32.4 | 35.5 | 0.2 |
| L119A | 35.2 | 31.3 | 0.88 |
| L105A | 37.6 | 32.2 | 0.31 |
| P47A | 49.7 | 27.1 | 0.22 |
| E173A | 51.2 | 5.64 | 0.067 |
| K171A | 55.2 | 12 | 0.042 |
| T114A | 56 | 16.1 | 0.15 |
| E96A | 57 | 4.54 | 0.042 |
| P115A | 64.5 | 0.35 | 0.00694 |
| Q104A | 67.2 | 3.82 | 0.025 |

TABLE 29
Frequency of total cells for E12 (1 nM) binding to ULBP2 mutants

| ULBP2 | Gate 1 \| Freq. of | Gate 2 \| Freq. of | Gate 3 \| Freq. of |
|---|---|---|---|
| mutant | Total | Total | Total |
| I40A | 0.05 | 0.29 | 1.05 |
| P41A | 0.098 | 0.41 | 4.73 |
| C66A | 0.23 | 0.73 | 1.99 |
| F43A | 0.23 | 2.48 | 19.9 |
| D65A | 0.25 | 6.71 | 30 |
| T60A | 0.28 | 11.1 | 37.2 |
| R48A | 0.29 | 0.59 | 1.17 |
| V52A | 0.52 | 24 | 39.4 |
| D144A | 0.72 | 34.7 | 24.7 |
| P118A | 0.92 | 35 | 19.4 |
| P45A | 2.24 | 44 | 22.2 |
| H63A | 2.95 | 42.1 | 21.4 |
| Q53A | 5.08 | 50.2 | 15.7 |
| T38A | 8.62 | 38.5 | 3.95 |
| K69A | 33.3 | 40.1 | 2.95 |
| E117A | 37.3 | 18.9 | 0.37 |
| T72A | 41.6 | 31.4 | 0.84 |
| P92A | 44 | 24.3 | 0.59 |
| P73A | 44.4 | 29.1 | 1.29 |
| P47A | 48.2 | 25.2 | 1.15 |
| K42A | 50.7 | 22 | 0.62 |
| R95A | 54.7 | 14.6 | 0.15 |
| T120A | 59.1 | 3.37 | 0.019 |
| R44A | 60.6 | 5.16 | 0.068 |
| K116A | 63.7 | 0.63 | 0.00926 |
| P115A | 65.6 | 0.12 | 0.019 |
| S142A | 66.9 | 0.42 | 0.018 |
| V93A | 67.2 | 0.055 | 0.005 |
| T70A | 73.8 | 2.12 | 0.038 |
| N68A | 75.7 | 0.69 | 0.03 |
| Wild type | 55.7 | 9.03 | 0.054 |
| E12 | | | |
| No | 0.15 | 0.014 | 0 |
| antibody | | | |

### Structural Modeling of A06 and E12 Binding to ULBP2

A homology model of NKG2D in complex with ULBP2 was created using the X-ray crystallographic structure of NKG2D/ULBP6 complex (PDB: 4S0U). Subsequently, the fragment variable regions (Fv) of A06 and E12 antibodies were docked on the surface of ULBP2 individually using protein-protein docking module in Bioluminate (Schrodinger). During docking, A06/E12 were marked as antibody and non-CDR regions were masked, such that the attractive potential in the non-CDR regions was removed. For addition restraints ULBP2 positions identified from alanine scanning for A06 and E12 were specified for attractive potential. For E12, poses were ranked based on having max H-bond interaction, CDR binding and no overlap with NKG2D. Similarly, A06 poses were screened for max H-bond interaction, CDR binding, and overlap with NKG2D. The highest ranked pose for A06-ULBP2 and E12-ULBP2 are shown in FIGS. 34A-34C.

### Epitope Analysis of A06 and E12

Using the predicted complexes of A06-ULBP2 and E12-ULBP2, the epitopes of both E12 and A06 was further refined and extended beyond what was observed using alanine scanning. The interacting residues for ULBP2 and A06, and for ULBP2 and E12 are listed in Table 30 and Table 31 respectively.

TABLE 30
Molecular interactions of ULBP2 and A06 antibody

| | | | | | | | Set1 | Set2 |
|---|---|---|---|---|---|---|---|---|
| ULBP2 | A06 | Distance | Specific | | #Salt | Surface | Buried | Buried |
| residues | residues | (A) | Interactions | #HB | Bridges | Complementarity | SASA (%) | SASA (%) |
| A:Val | L:Tyr | 3.9 | | 0 | 0 | 0.43 | 100 | 92.7 |
| 178 | 49 | | | | | | | |
| A:Val | L:Asn | 2 | 1x hb to | 1 | 0 | 0.69 | 58.9 | 100 |
| 177 | 53 | | L:Asn 53 | | | | | |
| A:Val | L:Ser | 2.9 | | 0 | 0 | 0.4 | 58.9 | 55.8 |
| 177 | 52 | | | | | | | |
| A:Val | L:Arg | 3 | | 0 | 0 | 0.59 | 58.9 | 86.3 |
| 177 | 50 | | | | | | | |
| A:Asp | L:Asn | 2.5 | | 0 | 0 | 0.65 | 100 | 100 |
| 175 | 53 | | | | | | | |
| A:Asp | L:Tyr | 1.7 | 1x hb to | 1 | 0 | 0.63 | 100 | 92.7 |
| 175 | 49 | | L:Tyr 49 | | | | | |
| A:Asp | L:Ser | 3.8 | | 0 | 0 | 0.36 | 100 | 55.8 |
| 175 | 52 | | | | | | | |
| A:Asp | L:Leu | 3.4 | | 0 | 0 | 0.48 | 100 | 65 |
| 175 | 54 | | | | | | | |
| A:Lys | L:Leu | 2.4 | | 0 | 0 | 0.52 | 78.8 | 65 |
| 171 | 54 | | | | | | | |
| A:Lys | L:Ala | 2.9 | | 0 | 0 | 0.75 | 78.8 | 74 |
| 171 | 55 | | | | | | | |
| A:Lys | L:Ser | 2.9 | | 0 | 0 | 0.68 | 78.8 | 74.6 |
| 171 | 56 | | | | | | | |
| A:Lys | L:Tyr | 2.1 | 1x hb to | 1 | 0 | 0.67 | 78.8 | 92.7 |
| 171 | 49 | | L:Tyr 49 | | | | | |
| A:Gln | L:Ser | 2.3 | | 0 | 0 | 0.63 | 40.1 | 74.6 |
| 109 | 56 | | | | | | | |
| A:Gln | L:Gly | 2.2 | | 0 | 0 | 0.68 | 40.1 | 38.6 |
| 109 | 57 | | | | | | | |
| A:Ile | L:Ser | 2.8 | | 0 | 0 | 0.42 | 10 | 74.6 |
| 108 | 56 | | | | | | | |
| A:Asp | L:Ser | 1.8 | 2x hb to | 2 | 0 | 0.76 | 82.6 | 74.6 |
| 107 | 56 | | L:Ser 56 | | | | | |
| A:Asp | L:Ala | 2.6 | | 0 | 0 | 0.82 | 82.6 | 74 |
| 107 | 55 | | | | | | | |
| A:Asp | L:Tyr | 3.1 | | 0 | 0 | 0.75 | 82.6 | 92.7 |
| 107 | 49 | | | | | | | |
| A:Arg | H:Arg | 2.7 | 1x hb to | 1 | 0 | 0.41 | 78.2 | 83.7 |
| 106 | 94 | | H:Arg 94 | | | | | |
| A:Arg | H:Glu | 2.1 | 2x hb, 1x | 2 | 1 | 0.59 | 78.2 | 99.6 |
| 106 | 97 | | salt bridge | | | | | |
| | | | to H:Glu 97 | | | | | |
| A:Arg | H:Gly | 2.1 | 1x hb to | 1 | 0 | 0.59 | 78.2 | 64.2 |
| 106 | 96 | | H:Gly 96 | | | | | |
| A:Arg | H:Trp | 3.3 | | 0 | 0 | 0.46 | 78.2 | 42.5 |
| 106 | 33 | | | | | | | |
| A:Arg | H:Asp | 3.7 | | 0 | 0 | 0.42 | 78.2 | 50.5 |
| 106 | 101 | | | | | | | |
| A:Arg | H:Gly | 3.8 | | 0 | 0 | 0.34 | 78.2 | 96.4 |
| 106 | 95 | | | | | | | |
| A:Arg | H:Thr | 4 | | 0 | 0 | 0.58 | 78.2 | 86.9 |
| 106 | 31 | | | | | | | |
| A:Gln | L:Tyr | 1.8 | | 0 | 0 | 0.69 | 100 | 92.7 |
| 104 | 49 | | | | | | | |
| A:Gln | L:Asn | 3.2 | | 0 | 0 | 0.71 | 100 | 100 |
| 104 | 53 | | | | | | | |
| A:Glu | L:Tyr | 2.6 | | 0 | 0 | 0.61 | 82.1 | 92.7 |
| 103 | 49 | | | | | | | |
| A:Glu | L:Arg | 2 | 1x hb, 1x | 1 | 1 | 0.53 | 82.1 | 86.3 |
| 103 | 50 | | salt bridge | | | | | |
| | | | to L:Arg 50 | | | | | |
| A:Glu | L:Asn | 3.7 | | 0 | 0 | 0.63 | 82.1 | 100 |
| 103 | 53 | | | | | | | |
| A:Glu | H:Glu | 3.7 | | 0 | 0 | 0.59 | 82.1 | 99.6 |
| 103 | 97 | | | | | | | |
| A:Thr | H:Glu | 2.6 | | 0 | 0 | 0.59 | 100 | 99.6 |
| 102 | 97 | | | | | | | |
| A:Ile | L:Arg | 2.7 | | 0 | 0 | 0.59 | 73.4 | 86.3 |
| 100 | 50 | | | | | | | |
| A:Asp | H:Glu | 2.8 | | 0 | 0 | 0.54 | 60.8 | 99.6 |
| 99 | 97 | | | | | | | |
| A:Asp | L:Arg | 3.3 | | 0 | 0 | 0.48 | 60.8 | 86.3 |
| 99 | 50 | | | | | | | |
| A:Asp | H:Leu | 3.7 | | 0 | 0 | 0 | 60.8 | 16.3 |
| 99 | 98 | | | | | | | |
| A:Pro | H:Thr | 2.8 | | 0 | 0 | 0.62 | 33.3 | 86.9 |
| 45 | 31 | | | | | | | |
| A:Pro | H:Tyr | 2.7 | | 0 | 0 | 0.41 | 33.3 | 59.7 |
| 45 | 52 | | | | | | | |
| A:Arg | H:Thr | 2.1 | | 0 | 0 | 0.63 | 78.7 | 86.9 |
| 44 | 31 | | | | | | | |
| A:Arg | H:Glu | 2 | 1x hb to | 1 | 0 | 0.64 | 78.7 | 99.6 |
| 44 | 97 | | H:Glu 97 | | | | | |
| A:Arg | H:Trp | 2.2 | | 0 | 0 | 0.51 | 78.7 | 42.5 |
| 44 | 33 | | | | | | | |
| A:Arg | H:Leu | 2.9 | | 0 | 0 | 0 | 78.7 | 16.3 |
| 44 | 98 | | | | | | | |
| A:Phe | H:Thr | 2.4 | | 0 | 0 | 0 | 15 | 86.9 |
| 43 | 31 | | | | | | | |
| A:Lys | H:Ala | 2.3 | | 0 | 0 | 0.78 | 73 | 51.2 |
| 42 | 28 | | | | | | | |
| A:Lys | H:Thr | 2.3 | | 0 | 0 | 0.69 | 73 | 86.9 |
| 42 | 31 | | | | | | | |
| A:Lys | H:Tyr | 2.1 | | 0 | 0 | 0.73 | 73 | 50.4 |
| 42 | 27 | | | | | | | |
| A:Lys | H:Gly | 2.2 | 1x hb to | 1 | 0 | 0.63 | 73 | 30.6 |
| 42 | 26 | | H:Gly 26 | | | | | |
| A:Pro | H:Thr | 3.7 | | 0 | 0 | 0 | 40.6 | 86.9 |
| 41 | 31 | | | | | | | |

#HB: Number of hydrogen bonds; Surface complementarity: Surface complementarity between the Set 1 and Set 2 residues in the row (ref: 2); Set1 Buried SASA: Fraction of the solvent-accessible surface area of the Set1 residue that is buried by the interaction with the Set2 residue; Set2 Buried SASA: Fraction of the solvent-accessible surface area of the Set2 residue that is buried by the interaction with the Set1 residue.

TABLE 31
Molecular interactions of ULBP2 and E12 antibody

| | | | | | | | Set1 | Set2 |
|---|---|---|---|---|---|---|---|---|
| ULBP2 | E12 | Distance | Specific | | #Salt | Surface | Buried | Buried |
| residues | residues | (A) | Interactions | #HB | Bridges | Complementarity | SASA (%) | SASA (%) |
| A:Pro | L:Ser | 2.1 | | 0 | 0 | 0.43 | 89.2 | 100 |
| 118 | 32 | | | | | | | |
| A:Pro | L:Phe | 2 | | 0 | 0 | 0.61 | 89.2 | 82 |
| 118 | 50 | | | | | | | |
| A:Glu | L:Thr | 2.1 | 2x hb to | 2 | 0 | 0.75 | 57.6 | 76 |
| 117 | 31 | | L:Thr 31 | | | | | |
| A:Glu | L:Ser | 3.4 | | 0 | 0 | 0.57 | 57.6 | 100 |
| 117 | 32 | | | | | | | |
| A:Glu | L:Phe | 2.5 | | 0 | 0 | 0.75 | 57.6 | 82 |
| 117 | 50 | | | | | | | |
| A:Glu | L:Gly | 2.1 | | 0 | 0 | 0.7 | 57.6 | 63.3 |
| 117 | 30 | | | | | | | |
| A:Glu | L:Asn | 3.9 | | 0 | 0 | 0.52 | 57.6 | 58.8 |
| 117 | 92 | | | | | | | |
| A:Lys | L:Thr | 2.2 | | 0 | 0 | 0.81 | 65.2 | 76 |
| 116 | 31 | | | | | | | |
| A:Lys | L:Phe | 2.5 | | 0 | 0 | 0.82 | 65.2 | 82 |
| 116 | 50 | | | | | | | |
| A:Lys | L:Ser | 2.2 | | 0 | 0 | 0.81 | 65.2 | 20.4 |
| 116 | 52 | | | | | | | |
| A:Lys | L:Glu | 1.9 | 1x hb, 1x salt | 1 | 0 | 0.83 | 65.2 | 29 |
| 116 | 53 | | bridge to | | | | | |
| | | | L:Glu 53 | | | | | |
| A:Lys | L:Ala | 4 | | 0 | 0 | 0 | 65.2 | 65 |
| 116 | 51 | | | | | | | |
| A:Thr | H:Asn | 3.4 | | 0 | 0 | 0.42 | 57.8 | 92.7 |
| 72 | 31 | | | | | | | |
| A:Thr | H:Thr | 3.2 | | 0 | 0 | 0.46 | 57.8 | 14.5 |
| 72 | 28 | | | | | | | |
| A:Thr | H:Thr | 3.8 | | 0 | 0 | 0.48 | 57.8 | 63.5 |
| 72 | 30 | | | | | | | |
| A:Lys | H:Asp | 1.8 | 1x hb, 1x salt | 1 | 0 | 0.76 | 27.1 | 44.4 |
| 69 | 54 | | bridge to | | | | | |
| | | | H:Asp 54 | | | | | |
| A:Lys | H:Tyr | 3.6 | | 0 | 0 | 0.66 | 27.1 | 87.1 |
| 69 | 52 | | | | | | | |
| A:Lys | H:Gly | 3.9 | | 0 | 0 | 0.65 | 27.1 | 44.2 |
| 69 | 53 | | | | | | | |
| A:Asn | H:Tyr | 2.8 | | 0 | 0 | 0.52 | 84.2 | 87.1 |
| 68 | 52 | | | | | | | |
| A:Asn | H:Thr | 2 | 1x hb to | 1 | 0 | 0.5 | 84.2 | 63.5 |
| 68 | 30 | | H:Thr 30 | | | | | |
| A:Asn | H:Asn | 3 | | 0 | 0 | 0.45 | 84.2 | 92.7 |
| 68 | 31 | | | | | | | |
| A:Asn | H:Gly | 3.7 | | 0 | 0 | 0.51 | 84.2 | 44.2 |
| 68 | 53 | | | | | | | |
| A:Gly | H:Tyr | 2.2 | | 0 | 0 | 0.7 | 76.8 | 87.1 |
| 67 | 52 | | | | | | | |
| A:Gly | H:Asp | 3.3 | | 0 | 0 | 0.8 | 76.8 | 44.4 |
| 67 | 54 | | | | | | | |
| A:Asp | H:Asn | 1.8 | 1x hb to | 1 | 0 | 0.54 | 100 | 92.7 |
| 65 | 31 | | H:Asn 31 | | | | | |
| A:Asp | H:Tyr | 1.8 | 1x hb to | 1 | 0 | 0.69 | 100 | 89.9 |
| 65 | 98 | | H:Tyr 98 | | | | | |
| A:His | H:Tyr | 2.7 | | 0 | 0 | 0.74 | 34.2 | 89.9 |
| 63 | 98 | | | | | | | |
| A:His | H:Asn | 3.1 | | 0 | 0 | 0.59 | 34.2 | 92.7 |
| 63 | 31 | | | | | | | |
| A:Ala | H:Tyr | 2.5 | | 0 | 0 | 0.75 | 82.9 | 89.9 |
| 51 | 98 | | | | | | | |
| A:Arg | H:Tyr | 1.8 | | 0 | 0 | 0.68 | 99.4 | 89.9 |
| 48 | 98 | | | | | | | |
| A:Arg | L:Trp | 2.1 | | 0 | 0 | 0.74 | 99.4 | 51.9 |
| 48 | 94 | | | | | | | |
| A:Arg | H:Gly | 2.3 | 1x hb to | 1 | 0 | 0.66 | 99.4 | 96.5 |
| 48 | 99 | | H:Gly 99 | | | | | |
| A:Arg | H:Asp | 2.7 | | 0 | 0 | 0.57 | 99.4 | 99.9 |
| 48 | 33 | | | | | | | |
| A:Arg | L:Ser | 3.3 | | 0 | 0 | 0.51 | 99.4 | 100 |
| 48 | 91 | | | | | | | |
| A:Arg | H:Trp | 2.3 | | 0 | 0 | 0.64 | 99.4 | 100 |
| 48 | 50 | | | | | | | |
| A:Arg | H:Ser | 3.3 | | 0 | 0 | 0.57 | 99.4 | 0 |
| 48 | 95 | | | | | | | |
| A:Arg | H:Tyr | 2.9 | | 0 | 0 | 0.43 | 99.4 | 100 |
| 48 | 100B | | | | | | | |
| A:Arg | L:Phe | 3.4 | | 0 | 0 | 0.73 | 99.4 | 82 |
| 48 | 50 | | | | | | | |
| A:Arg | L:Tyr | 3.9 | | 0 | 0 | 0.51 | 99.4 | 0 |
| 48 | 96 | | | | | | | |
| A:Pro | H:Tyr | 3 | | 0 | 0 | 0.63 | 99.7 | 89.9 |
| 47 | 98 | | | | | | | |
| A:Pro | H:Tyr | 2.5 | | 0 | 0 | 0.56 | 99.7 | 87.1 |
| 47 | 52 | | | | | | | |
| A:Pro | H:Asp | 2.5 | | 0 | 0 | 0.52 | 99.7 | 99.9 |
| 47 | 33 | | | | | | | |
| A:Pro | H:Asn | 4 | | 0 | 0 | 0.49 | 99.7 | 92.7 |
| 47 | 31 | | | | | | | |
| A:Phe | L:Trp | 2.6 | | 0 | 0 | 0.64 | 74.2 | 51.9 |
| 43 | 94 | | | | | | | |
| A:Phe | L:Ser | 3.2 | | 0 | 0 | 0.4 | 74.2 | 100 |
| 43 | 91 | | | | | | | |
| A:Phe | L:Phe | 3.8 | | 0 | 0 | 0.62 | 74.2 | 82 |
| 43 | 50 | | | | | | | |
| A:Lys | L:Asn | 2.1 | 1x hb to | 1 | 0 | 0.41 | 31.2 | 58.8 |
| 42 | 92 | | L:Asn 92 | | | | | |
| A:Lys | L:Ser | 3.9 | | 0 | 0 | 0.33 | 31.2 | 9.5 |
| 42 | 93 | | | | | | | |
| A:Ile | H:Tyr | 3.8 | | 0 | 0 | 0.5 | 90.9 | 89.9 |
| 40 | 98 | | | | | | | |

#HB: Number of hydrogen bonds; Surface complementarity: Surface complementarity between the Set 1 and Set 2 residues in the row (ref: 2); Set1 Buried SASA: Fraction of the solvent-accessible surface area of the Set1 residue that is buried by the interaction with the Set2 residue; Set2 Buried SASA: Fraction of the solvent-accessible surface area of the Set2 residue that is buried by the interaction with the Set1 residue.

### Example 3: Design and Expression of Bispecific Antibodies with Heavy Chain and Light Chain Heterodimerization

### Molecular Modeling and Structure Guided Design of Bispecific Antibodies

Challenges for bispecific IgG-like antibody format designs are (i) efficient heavy chain heterodimerization and, (ii) efficient light chain heterodimerization for correct cognate pairing with good specificity. Thus, bispecific or multispecific antibodies were designed to include (i) knob-into-hole mutations to promote efficient heavy chain heterodimerization and (ii) charged pairing (between VH1 and VL1 interface and/or VH2 and VL2 interface, and CHI_{H1} and CL1 interface and/or CH2_{H1} and CL2 interface) and disulfide stabilization mutations (between CH1_{H1} and CL1 interface and/or CH2_{H1} and CL2 interface), which together promote correct cognate pairing of heavy chain and light chains. Correct pairing of heavy and light chains is advantageous for efficient large scale antibody production and purification.

Bioluminate modeling suite and Pymol molecular graphics system from Schrodinger were used to perform all molecular modeling works. A high resolution crystal structure of Trastuzumab (PDB ID: 1N8Z) was used as a backbone template for homology modeling of VH2-CH2_{H1}/VL2-CL2ₖₐₚₚₐ antigen binding fragment (Fab) of the disease-associated antigen (DAA) binding portion of the antibody, and a high resolution crystal structure of Pertuzumab (PDB ID: 4LLU) was used as a backbone template for homology modeling of VH1-CH1_{H1}/VL1-CL1_{lambda} (Fab) of the CD3 binding portion of the antibody.

The evaluation of impact of individual mutation sets for charged pairs or knob into hole mutations on the Fab stability and on the respective affinity of each paratope was carried out with the BioLuminate Residue Scanning Tool. Residue pairs for disulfide repositioning were identified using PyMol molecular graphics system through structure guided design and validated by homology modeling or Bioluminate Cys-Cys engineering tool. Exemplary charged pairs and disulfide repositioning mutations are shown in Tables 1-6. A schematic depiction of the antibody designs are shown in FIG. 1A-D. This structural design of the antibody provides improved specificity and complete cognate pairing, which is advantageous for functional antibody expression and purification.

### Antibody Expression and Purification

All individual prioritized mutation sets were experimentally tested for expression in transient Expi293 cells. DNA sequences corresponding to antibody heavy and light chains were synthesized in pDT5 vector (ATUM, Newark CA). Plasmid DNA (1 μg/ml) were transfected into Expi293 cells (ThermoFisher) according to manufacturer's protocols. Cells were grown in flasks with rotation (125 rpm) at 37° C. with 8% CO2. Five days post-transfection, the conditioned media was harvested from the cells by centrifugation (3000×g) for 30 minutes and filtered using 0.2 μm filter. Antibodies were then purified using an AKTA Avant chromatography system (Cytiva) and a tandem purification method using HiTrap Protein A and HiLoad Superdex 200 columns (Cytiva). Antibodies were stored in PBS, pH 7.22 at 4 degrees Celsius following purification and prior to analysis.

### Biophysical Characterization

Analytical size exclusion chromatography (aSEC) was performed using AdvanceBio 1.9 μm SEC column (Agilent) equipped on a 1260 Infinity II Bioinert HPLC system for the determination of aggregates and other higher and low molecular weight mass species. Antibody samples were run with a linear gradient using 1×PBS (pH7.2) at a flow rate of 0.3 mL/min for 10 min with Ultraviolet (UV) absorbance monitoring at 280 nm. The eluted protein was quantified by UV absorbance and integration of peak areas. BEH SEC protein standard mix (Waters) served as a standard. Hydrophobic interaction chromatography (HIC) was performed using AdvanceBio HIC (4.6×100 mm). Antibody samples were mixed 1:1 with buffer A (1.8 M ammonium sulfate in 0.1 M Na-phosphate buffer pH 7.2) and run with a linear gradient using mobile phase A and B (0.1 M Na-phosphate buffer pH7.2) over 20 min at a flow rate of 0.4 mL/min with UV absorbance monitoring at 280 nm. The thermal stability of the antibodies was determined by differential scanning calorimetry (DSC) using Nano DSC calorimeter (TA Instrument).

For DSC, samples were prepared in 1×PBS in 1 mg/mL concentration, loaded into sample cell and scanned at 1° C./min increment from 25 to 95° C. Data was analyzed using NanoAnalyzer program subtracting PBS buffer background from each individual scan. For total protein purity analysis the NuPAGE Pre-Cast gel system (Invitrogen) was used according to the manufacturer's instruction. In particular, 4-12% NuPAGE Novex Bis-TRIS Pre-Cast gels (pH 6.4) and a NuPAGE MES (reduced gels, with NuPAGE Antioxidant running buffer additive) or MOPS (non-reduced gels) running buffer was used. Half of the sample was combined with NuPAGE Sample Reducing Agent or left unreduced, respectively, and heated for 10 min at 70° C. Subsequently, 5-20 μL were applied to a 4-12% NuPAGE Bis-Tris NUPAGE (Invitrogen) (with MOPS buffer for nonreduced NUPAGE and MES buffer with NuPAGE Antioxidant running buffer additive (Invitrogen) for reduced NUPAGE) and stained with Coomassie Blue.

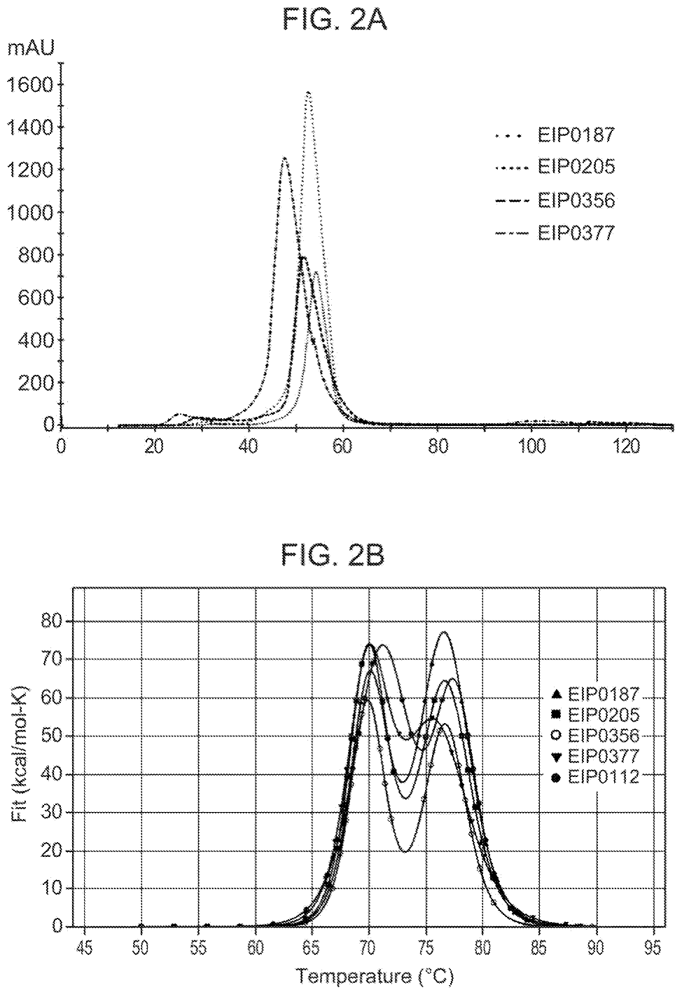

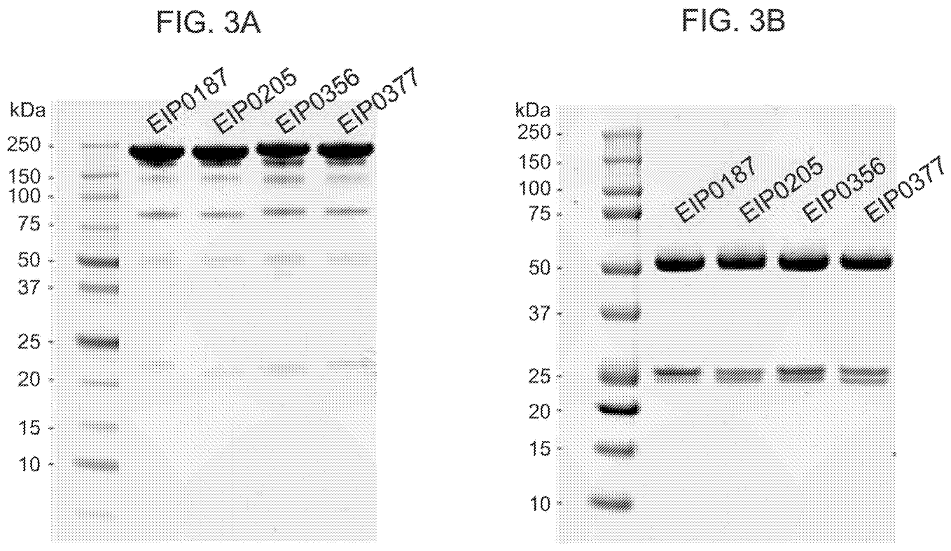

From initial screening a selected sets of mutations with favorable biophysical properties were combined and engineered into heavy chain and light chain sequences and advanced for further testing in bispecific human IgG1 format. Selected permutation of different sets of mutation containing at least one disulfide repositioning mutation pair (in either DAA Fab arm or CD3 Fab arm), one set of charge pairing mutations in the VH1/VL1 or VH2/VL2 sequences and at least one set of charge pairing mutation in the CH1_{H1}/CL1 or CH2_{H1}/CL2 sequences, were expressed, purified, and experimentally tested in the full IgG1 bispecific format and characterized. Preparative size exclusion chromatogram of representative bispecific variants (EIP0187, EIP0205, EIP0356 and EIP0377), obtained from tandem protein A-size exclusion column purification using AKTA system, are shown in FIG. 2A. The thermal stability of light chain pairing mutations were assessed using differential scanning calorimetry (DSC). As shown in FIG. 2B, significant differences were not observed in the melting of the Fab region (second transition) for EIP0187, EIP0205, EIP0356 and EIP0377 compared to EIP0112 (CrossMab), a bispecific using the benchmark light chain pairing technology, CrossMab, described by Schaefer et. al. [PNAS 2011, 108 (27) 11187-11192]). A majority of the mutational combinations led to significant decreases in expression of bispecific proteins and were not considered further. Integrity and purity of the bispecific proteins with good expression were analyzed by aSEC and NuPAGE gel electrophoresis (intact and reduced) (FIG. 3). Together, this demonstrates that bispecific antibodies have high thermal stability, high protein integrity and efficient and accurate assembly of light chain and heavy chain components.

### Enzyme Linked Immunosorbent Assay (ELISA)

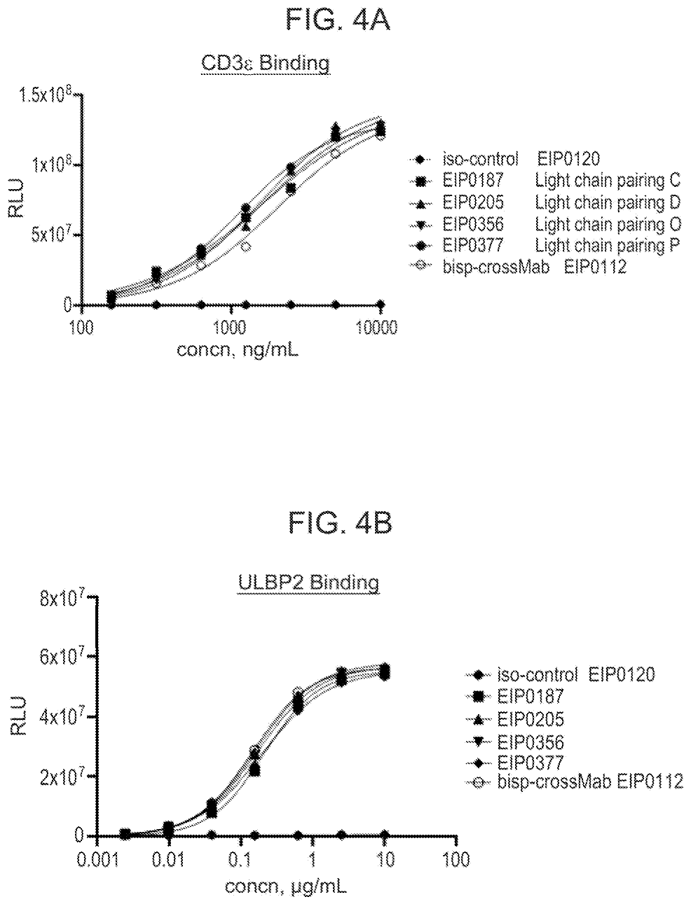

The bispecific variants without any undesired mass (HMMS or LMMS) on aSEC and on NuPAGE gel electrophoresis were advanced for further analysis by sandwich ELISA for binding to their corresponding antigens. Maxisorp plates (Nunc) were coated with proteins at a concentration of 1 μg/ml in bicarbonate buffer pH 9.2 or PBS, pH 7.22 for 16 hours at 4° C. All subsequent steps were performed at room temperature. The plates were then blocked with blocking buffer (PBS, pH 7.2, 1% BSA) for 1 hour. The plates were then washed using PBS pH 7.2, 0.1% Tween. Bispecific antibodies were diluted in blocking buffer at a 1 in 3 dilution starting at 100 nM and incubated with blocked wells for 1 hour. Plates were then washed and incubated with anti-human HRP conjugate at a 1 in 500 dilution. Following a final wash, the luminescence substrate was added according to manufacturer's instructions (SeraCare). Luminescence was measured using Ensight plate reader (Perkin Elmer). For comparison, a purified a benchmark bispecific antibody using therapeutically validated bispecific platform was tested. Bispecific antibodies were functional and able to engage both the disease associated antigen (i.e. ULBP2) and CD3 antigens simultaneously (FIGS. 4A-4B).

### Mass Spectrometry

The selected bispecific variants with desired biophysical properties were further characterized by mass spectrometry to determine intact antibody mass, and reduced masses of heavy chains and light chains, and the specificity of assembly of light chains with corresponding heavy chains was assessed.

The intact mass of the purified fusion proteins and antibody chains was confirmed by Xevo G2-XS QTof Quadrupole Time-of-Flight Mass Spectrometry coupled to a Acquity UHPLC system (Waters) equipped with a Protein BEH C4 (300 Å 1.7 μm) column. Antibody samples were deglycosylated first using rapid PNGase F enzyme (New England Biolabs) in reducing and non-reducing conditions following supplier's protocols. Reaction mixture was diluted to 1:10 in 50% Acetonitrile containing 0.1% Formic acid and 2 μL of which was injected into LC-MS. The total ion chromatogram and m/z data of the proteins were acquired with gradient run of 10 to 70 mL HPLC grade acetonitrile over 12 min. Mass of the protein samples was deconvoluted from total ion chromatogram using BYOS software from Protein Metrics.

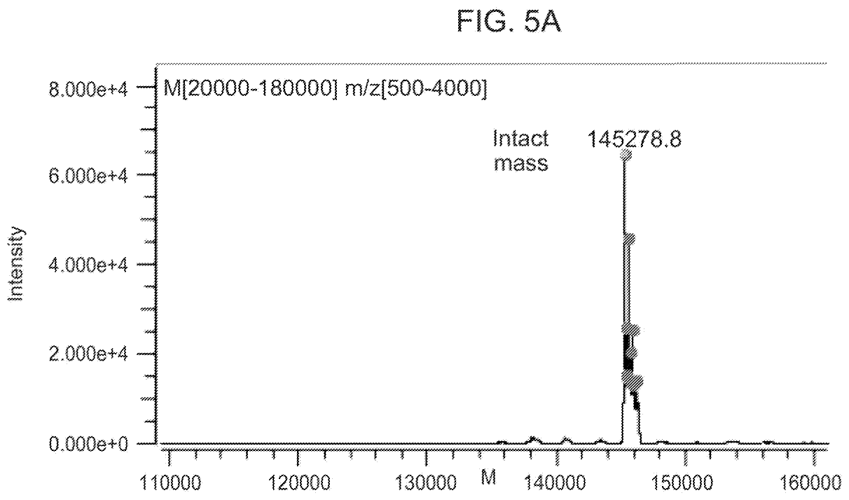

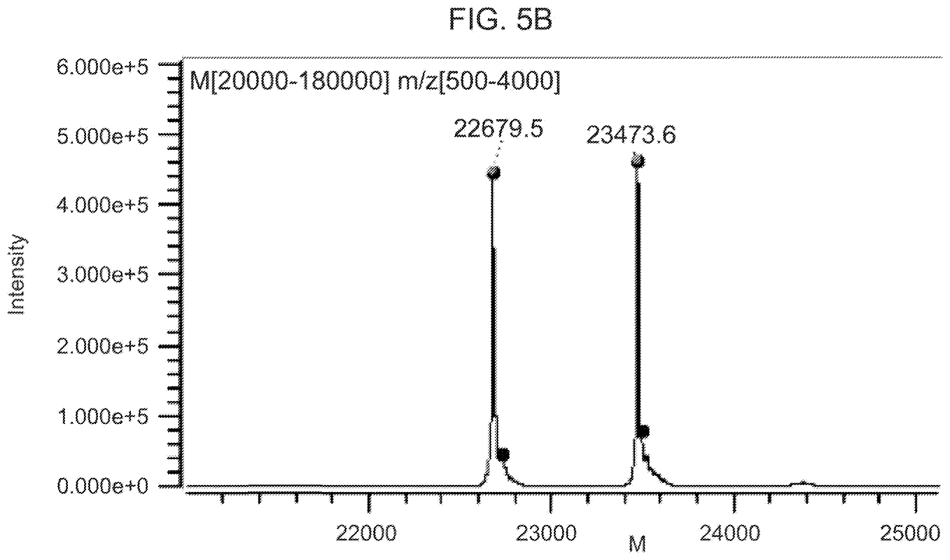

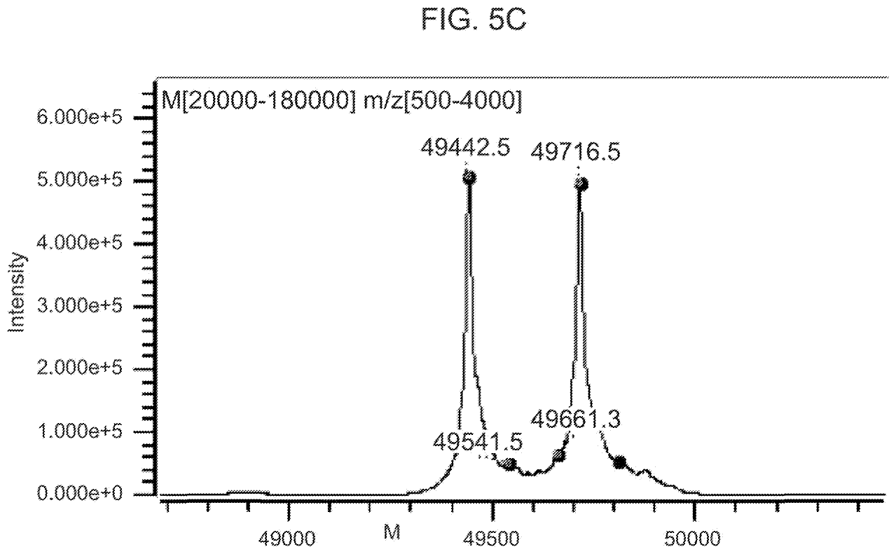

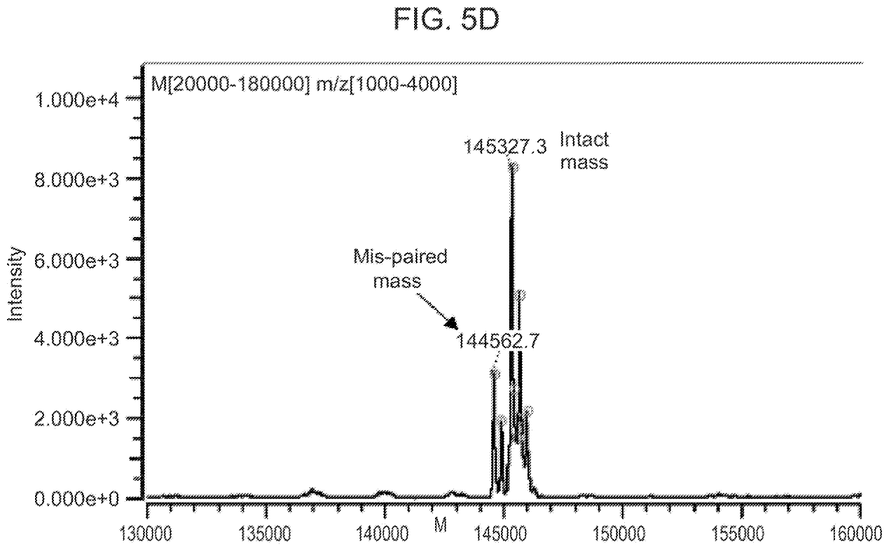

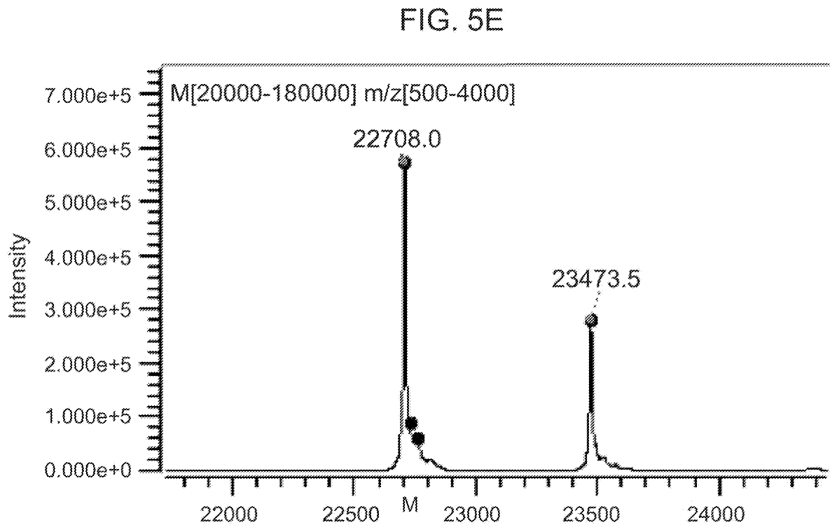

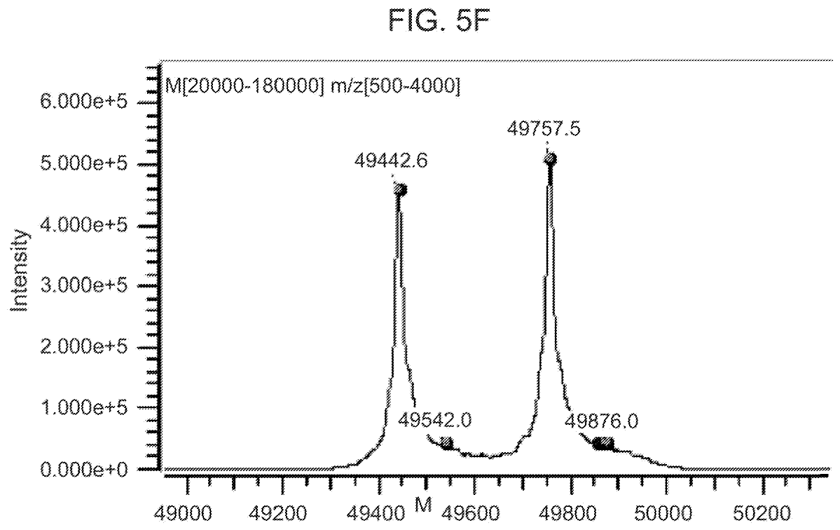

Deconvoluted intact mass of one of the representative bispecific variant EIP0205 is shown in FIG. 5A. The expected sizes for corresponding heavy chain masses (FIG. 5B) and light chain masses (FIG. 5C) were observed. No species or masses that corresponds to mispairing of the light chains with non-cognate heavy chains were observed. Relative intensity ratio of 1:1 of two heavy chains and two light chains suggests that the bispecific antibody is fully intact with near 100% cognate pairing. Certain bispecific antibodies which failed to achieve complete cognate pairing, the intact masses for mis-paired LC and HC were easily observable from deconvoluted ion chromatogram shown in FIG. 5D, as ‘mis-paired mass species’. The deconvoluted masses of reduced heavy chains and light chains are shown in FIGS. 5E-5F.

Together, the biophysical and antigen binding results indicate that the compensatory charge pairing mutations (designed within VH1 and VL1 interface and/or VH2 and VL2 interface, and CH1_{H1} and CL1 interface and/or CH2_{H1} and CL2 interface) and disulfide stabilized mutations (designed within CH1_{H1} and CL1 interface and/or CH2_{H1} and CL2 interface), resulted in intact bispecific antibodies with the correct conformation and high thermal stability.

### Example 4: Characterization of ULBP2-&CD3 Bispecific Antibodies with Mutations that Promote Heavy Chain and Light Chain Heterodimerization

Using the methods described above, αULBP2-αCD3 bispecific antibodies (e.g. EIP0174, EIP0175, EIP0187, EIP0205, EIP0206, EIP0207, EIP0208, EIP0294, EIP0295, EIP0306, EIP0307, EIP0318, EIP0342, EIP0344, EIP0356, EIP0377, EIP0598) were constructed with sixteen different mutation sets (A-P). Each bispecific antibody has identical CDRs that bind to ULBP2 (derived from “E12” antibody) and identical CDRs that bind to CD3 (derived from “SP34” antibody), but differed in (i) knob-into-hole mutations to promote efficient heavy chain heterodimerization and (ii) charged pairing (VH1 and VL1 interface and/or VH2 and VL2 interface, and CH1_{H1} and CL1 interface and/or CH2_{H1} and CL2 interface) and disulfide stabilization mutations (between CH1_{H1} and CL1 interface and/or CH2_{H1} and CL2 interface), which together promote correct cognate pairing of heavy chain and light chains.

The charged mutation sets (A-P) were evaluated for expression titers, percentage POI by analytical size exclusion chromatography, binding to ULBP2 and CD3 by sandwich ELISA, functional T cell activity and cognate light chain pairing by mass spectrometry.

TABLE 32
Characterization of αULBP2-αCD3 Bispecific Antibodies

| Light chain | | Expression | | | | T cell | |
|---|---|---|---|---|---|---|---|
| pairing | Protein | titer | POI | ULBP2 | CD3 | cytolytic | Cognate |
| mutation set | identifier | (mg/L) | (%) | binding | binding | activity | Pairing |
| A | EIP0174 | 92 | 90% | +++ | +++ | +++ | ~83% |
| B | EIP0175 | 96 | 93% | +++ | +++ | +++ | ~77% |
| C | EIP0187 | 165 | 97% | +++ | +++ | +++ | 100% (*UMS~20%) |
| D | EIP0205 | 70 | 97% | +++ | +++ | +++ | 100% |
| E | EIP0206 | 112 | 95% | +++ | +++ | +++ | ~82% |
| F | EIP0207 | 115 | 95% | +++ | +++ | +++ | ~73% |
| G | EIP0208 | 122 | 94% | +++ | +++ | +++ | 100%, (*UMS~8%) |
| H | EIP0294 | 95 | 90% | +++ | +++ | +++ | ~88% |
| I | EIP0295 | 120 | 92% | +++ | +++ | +++ | ~88% |
| J | EIP0306 | 95 | 94% | +++ | +++ | +++ | 65% |
| K | EIP0307 | 90 | 94% | +++ | +++ | ++ | 79% |
| L | EIP0318 | 70 | 97% | +++ | +++ | +++ | 74% |
| M | EIP0342 | 110 | 87% | +++ | +++ | +++ | 72% |
| N | EIP0354 | 100 | 89% | +++ | +++ | +++ | 83% |
| O | EIP0356 | 105 | 96% | +++ | +++ | +++ | 100% |
| P | EIP0377 | 115 | 96% | +++ | +++ | +++ | 100% |
| Q | EIP0598 | 135 | 98% | +++ | +++ | +++ | 100% |
| Crossmab | EIP0112 | 170 | 88% | +++ | +++ | +++ | — |
| (control) | | | | | | | |

*UMS—unknown mass species

### T-Cell Mediated Cytotoxicity Assay

T cells were isolated from healthy human PBMCs donors using StemCell T-cell isolation kit and resuspended in RPMI was supplemented with 10% FBS, 1× penicillin/streptomycin. To activate T cells, Dynabeads coated with αCD3 and αCD28 antibodies were added to the T cells at a ratio of 25 μL beads per million cells and incubated at 37° C. in 5% CO2 for 48 hours. Following removal of Dynabeads, T cells were incubated with IL-7 (10 ng/ml) and IL-15 (10 ng/ml) in supplemented in media for an additional 7 days. The supplemented media was replaced with freshly thawed cytokines (IL7 and IL15) every 48 hours. Human tumor cells were seeded at 10,000 cells per well in 96-well tissue culture plates (Perkin Elmer) and incubated for 24 hours at 37° C. with 5% CO2. The following day, naïve or activated human T cells (50-100,000) or PBMCs (250-300,000) were added to tumor cells in the presence or absence of antibodies and incubated for up to 3-7 days. Cytolysis of Green Fluorescent Protein engineered MDA-MB-231 and HCT116 (Genecopoeia) cells were visualized using fluorescent plate reader (Ensight, Perkin Elmer). Cytolysis of CORL105 and SiHa cells were measured using the LDH-Glo cytotoxicity assay (Promega).

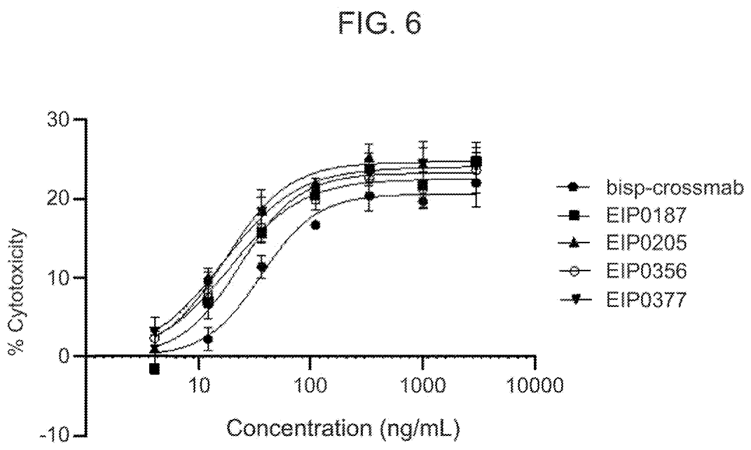

The bispecific variants with complete cognate pairing were then tested for their functional cytolytic activity using T cell or PBMC and tumor cell co-culture cytotoxicity assay with different E:T ratio. All the variants tested showed dose dependent cytolytic activity in different tumor cell model with varying degree (low to high) of target density, FIG. 6. Overall, biophysical and functional characterization results demonstrated that the bispecific variants EIP0187, EIP0205, EIP0356, EIP0377, and EIP0598 provided fully functional biologically active bispecific antibodies with high specificity and thermal stability.

### Example 5: Characterization αULBP2-αBCMA and αULBP2-αEGFR Bispecific Antibodies With Mutations That Promote Heavy Chain And Light Chain Heterodimerization

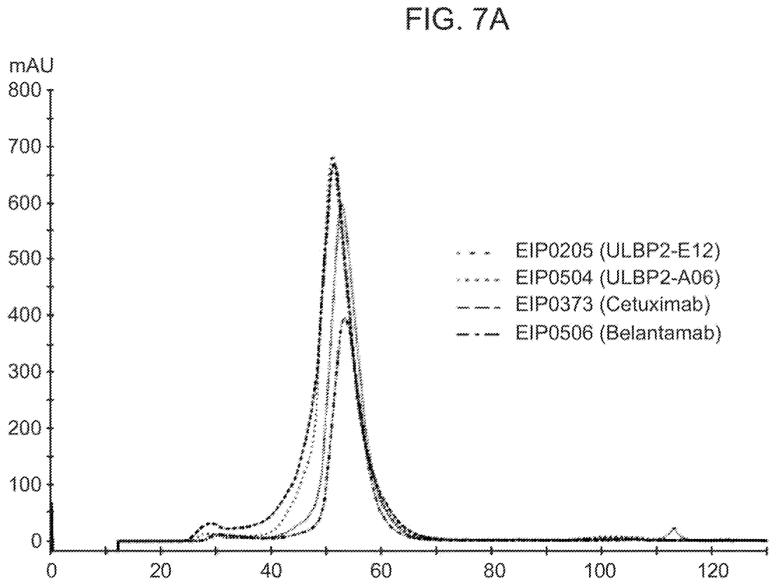

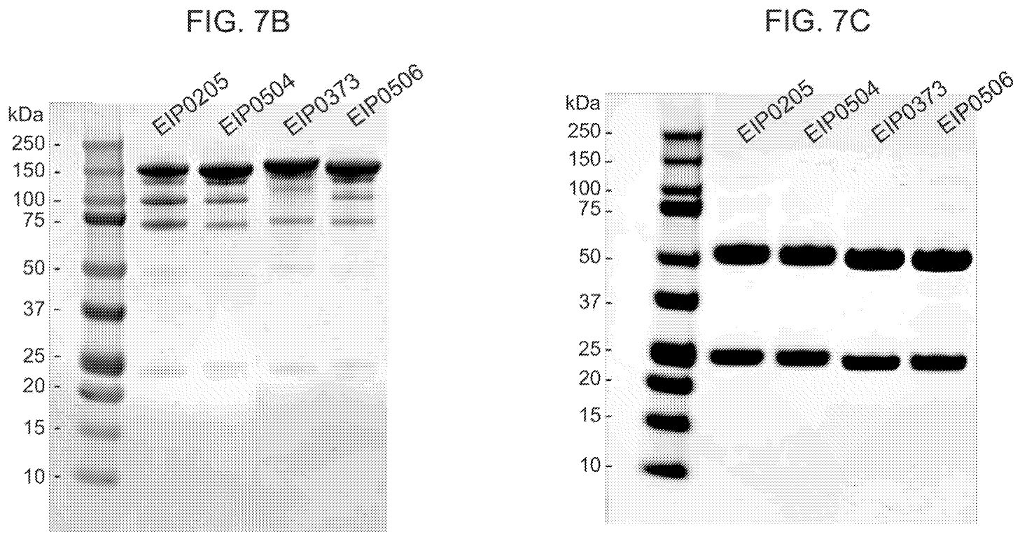

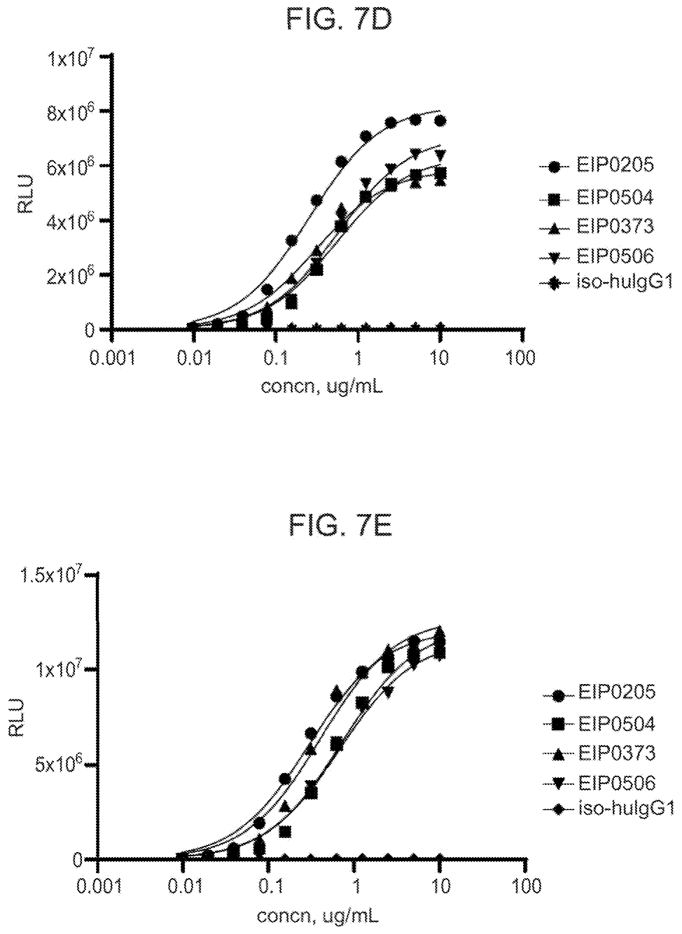

Further, the mutations sets from EIP0187, EIP0205, EIP0356, EIP0377, and EIP0598 were used to engineer asymmetric bispecifics using other therapeutically validated antibodies against different disease-associated antigen targets such as EGFR (Cetuximab) and BCMA (Belantamab) described in PCT application WO1996040210 and US application US20140105915, respectively, each of which is incorporated herein by reference. The purified bispecific antibodies were tested for their target binding, thoroughly characterized by mass spectrometry for specificity and correct LC and HC pairings, and functionally evaluated in retargeted T cell cytotoxicity assay. The biophysical and functional characterization data summarized in FIGS. 7A-7E and Table 33 suggest that the bispecific variants engineered with different target arms achieved 100% specificity and complete cognate LC/HC pairing with fully functional asymmetric bispecific antibody. This indicates that these novel combinations of mutation sets (compensatory charge pairing and disulfide stabilized mutations) could be applied universally to engineer asymmetric bispecific or multispecific antibodies.

TABLE 33
Characteristics of bispecific antibodies utilizing light chain pairing mutation set D.

| | | | | | T cell | | |
|---|---|---|---|---|---|---|---|
| | Protein | POI | ULBP2 | CD3 | cytolytic | Tₘ | Cognate |
| Bispecifics | identifier | (%) | binding | binding | activity | (DSC) | Pairing |
| ULBP2(E12)-SP34 | EIP0205 | 96% | +++ | +++ | +++ | 71/77 | 100% |
| ULBP2(A06)-SP34 | EIP0504 | 92% | +++ | +++ | +++ | 69/76 | 100% |
| EGFR(Cetuximab)-SP34 | EIP0373 | 96% | +++ | +++ | +++ | 70/78 | 100% |
| BCMA(Belantamab)-SP34 | EIP0506 | 98% | +++ | +++ | +++ | 69/77 | 100% |

### Example 6: Affinity Modulation of Anti-CD3 Antibodies

### Kinetic Binding Analysis of CD3 Affinity Variant Antibodies

The binding kinetics of ULBP2xCD3 bispecific CD58 fusions containing CD3 variants to human and cynomolgus CD3 epsilon/delta heterodimer was determined using surface plasmon resonance (SPR) for which the kinetic constants are summarized in Table 34.

TABLE 34
Association, dissociation, and equilibrium constants for binding
to human and cynomolgus CD3 epsilon/delta heterodimer

| | | ka | kd | K_{D} |
|---|---|---|---|---|
| CD3 variant | Antigen | (1/Ms) | (1/s) | (nM) |
| CD3-01 | Human CD3e/d | 1.76E+06 | 8.19E−03 | 4.7 |
| | Cyno CD3e/d | 1.60E+06 | 5.15E−03 | 3.2 |
| CD3-A2 | Human CD3e/d | 2.57E+05 | 2.63E−02 | 102 |
| | Cyno CD3e/d | 2.56E+05 | 2.27E−02 | 89 |
| CD3-A5 | Human CD3e/d | 6.92E+04 | 2.45E−02 | 354 |
| | Cyno CD3e/d | 9.17E+04 | 3.37E−02 | 368 |
| CD3-A6 | Human CD3e/d | 6.40E+05 | 4.79E−02 | 75 |
| | Cyno CD3e/d | 5.58E+05 | 4.18E−02 | 75 |
| CD3-A8 | Human CD3e/d | 4.32E+05 | 6.35E−02 | 147 |
| | Cyno CD3e/d | 4.48E+05 | 6.14E−02 | 137 |
| CD3-A9 | Human CD3e/d | 6.54E+05 | 1.01E−01 | 155 |
| | Cyno CD3e/d | 3.38E+05 | 7.18E−02 | 212 |

The hybridoma antibody derived from the SP34 clone was described to recognize both human and cynomolgus CD3 epsilon (Yoshino, N., Ami, Y., Terao, K., Tashiro, F. & Honda, M. Upgrading of flow cytometric analysis for absolute counts, cytokines and other antigenic molecules of cynomolgus monkeys (Macaca fascicularis) by using anti-human cross-reactive antibodies. Exp Anim 49, 97-110 (2000); Conrad, M. L., Davis, W. C. & Koop, B. F. TCR and CD3 antibody cross-reactivity in 44 species. Cytometry A 71, 925-933 (2007)). The humanized SP34 antibody was described in patent WO2007042261A2 (Micromet) and a homology model was created using molecular modeling software (Schrodinger). To understand the structural requirements of CD3 binding, alanine scanning was performed across all six CDRs of the humanized SP34 antibody to determine positions that were important to binding of human CD3 epsilon. Briefly, mutants of the humanized SP34 antibody in which a single CDR position was substituted with alanine were displayed as scFv on the surface of yeast and binding of recombinant human CD3 epsilon was assessed using flow cytometry.

Several strategies were used to identify SP34 mutants with a range of affinities to CD3 epsilon. In one strategy, alanine mutants with observed reductions in CD3 affinity were selected from the alanine scanning analysis described above. In a second strategy, alanine mutants with reduced CD3 affinity were interrogated using molecular modeling in order to replace alanine substitution with alternative amino acids in order to better modulate the degree of affinity reduction. In a third strategy, aspartate was substituted for alanine at select positions as it has been described that charged amino acids at certain CDR positions can reduce the propensity of antibodies to aggregation (Dudgeon, K. et al. General strategy for the generation of human antibody variable domains with increased aggregation resistance. Proc Natl Acad Sci USA 109, 10879-10884 (2012)). The list of SP34 mutants with reduced CD3 affinity is shown in Table 35.

TABLE 35
Bispecific Antibodies with SP34 having CDR mutations in the Variable Heavy and Variable Light Chain Domains

| | | | | | | Hu | Cyno | NFAT | NFAT | |
|---|---|---|---|---|---|---|---|---|---|---|
| | | | | | | CD3 | CD3 | activation | activation | |
| | | | | Variable | Variable | epsilon | epsilon | using | using | |
| | | | | heavy | light | binding | binding | SiHa | HCT116 | CD3 |
| Mutant* | Mutant* | Mutant | Mutant | chain | chain | (EC50, nM) | (EC50, nM) | (EC50, pM) | (EC50, pM) | Activity |
| EIP0205 | EIP0598 | EIP0377 | EIP0187 | SP34 | SP34 | 1.54 | 1.35 | 36 | 69 | Strongest |
| EIP0527 | EIP0630 | EIP0694 | EIP0712 | None | Y92D | 1.08 | 0.94 | 31 | | Strongest |
| EIP0624 | EIP0540 | EIP0695 | EIP0713 | N100D | N94Q | 2.01 | 1.60 | 38 | 103 | Strongest |
| EIP0486 | EIP0628 | EIP0696 | EIP0714 | None | G50D | 50.17 | 33.86 | 95 | 314 | Strong |
| EIP0626 | EIP0542 | EIP0697 | EIP0715 | N100D/ | N94Q | 15.25 | 12.36 | 110 | 348 | Strong |
| | | | | S100aD | | | | | | |
| EIP0483 | EIP0627 | EIP0698 | EIP0716 | T31D | None | 24.76 | 13.67 | 196 | 584 | Moderate |
| EIP0623 | EIP0515 | EIP0699 | EIP0717 | R50K/ | N94Q | 24.10 | 15.63 | 242 | 524 | Moderate |
| | | | | N100D | | | | | | |
| EIP0621 | EIP0477 | EIP0700 | EIP0718 | N97S | N94Q | 14.93 | 13.31 | 426 | 488 | Weak |
| EIP0625 | EIP0541 | | | R50K/ | None | 36.34 | 30.33 | 597 | 952 | Weak |
| | | | | N100D | | | | | | |
| EIP0622 | EIP0513 | | | N97T | N94Q | 18.01 | 15.15 | 2330 | Not | Very |
| | | | | | | | | | determined | weak |
| EIP0525 | EIP0629 | | | N35D | None | 819.4 | Not | | | Very |
| | | | | | | | determined | | | weak |

*Mutants for which human and cynomolgus CD3 binding and NFAT activation for SiHa and HCT116 cell lines are reported is indicated in bold.

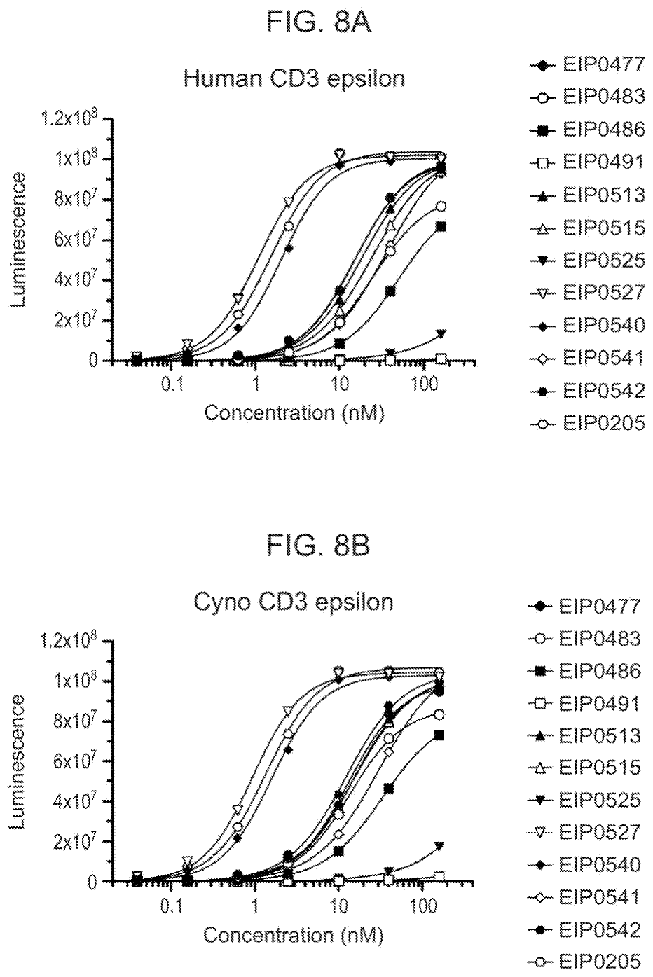

Bispecific antibodies were produced with SP34 affinity mutants and the ULBP2 antibody E12 using light chain pairing set D and evaluated for monovalent binding to human and cynomolgus CD3 epsilon by ELISA (FIG. 8A-8B). Three bispecific SP34 mutants, EIP0527, EIP0540 and EIP542, retained similar strong affinity to the bispecific having a SP34 arm without mutations (EIP0205). The majority of the bispecific SP34 mutants, EIP0477, EIP0483, EIP0486, EIP0491, EIP0513, EIP0515, and EIP0541 tested were of moderate affinity (EC50=10-50 nM) and one mutant, EIP0525, was observed to be of weak affinity (EC50>100 nM). In addition, the overall binding and the rank of affinities for the bispecific SP34 mutants did not change when assessed against cynomolgus CD3 epsilon, indicating that the mutations did not alter the cross reactivity and/or the binding between human and cynomolgus protein.

### Nuclear Factor of Activator of T Cells (NFAT) Luciferase Reporter Assay

Bispecific SP34 affinity mutants were evaluated for NFAT activation using the NFAT luciferase reporter Jurkat cell line in the presence of two cancer cell lines expressing high (SiHa) and low (HCT116) levels of ULBP2. Engagement of T cell antigen receptor (TCR)/CD3 complex in T cells leads to intracellular signaling events and the transcriptional activation of the Nuclear Factor of Activated T cells (NFAT) pathway. Human tumor cells were seeded at 40,000 cells per well in 96-well tissue culture plates (Perkin Elmer) and incubated for 24 hours at 37° C. with 5% CO2. Subsequently, 100,000 NFAT Luciferase Reporter Jurkat cells (Signosis) were added to tumor cells in the presence or absence of bispecific antibodies and incubated for 5 hours at 37° C. Upon addition of Bio-Glo reagent (Promega), luminescence was measured using luminescent plate reader (Ensight, Perkin Elmer).

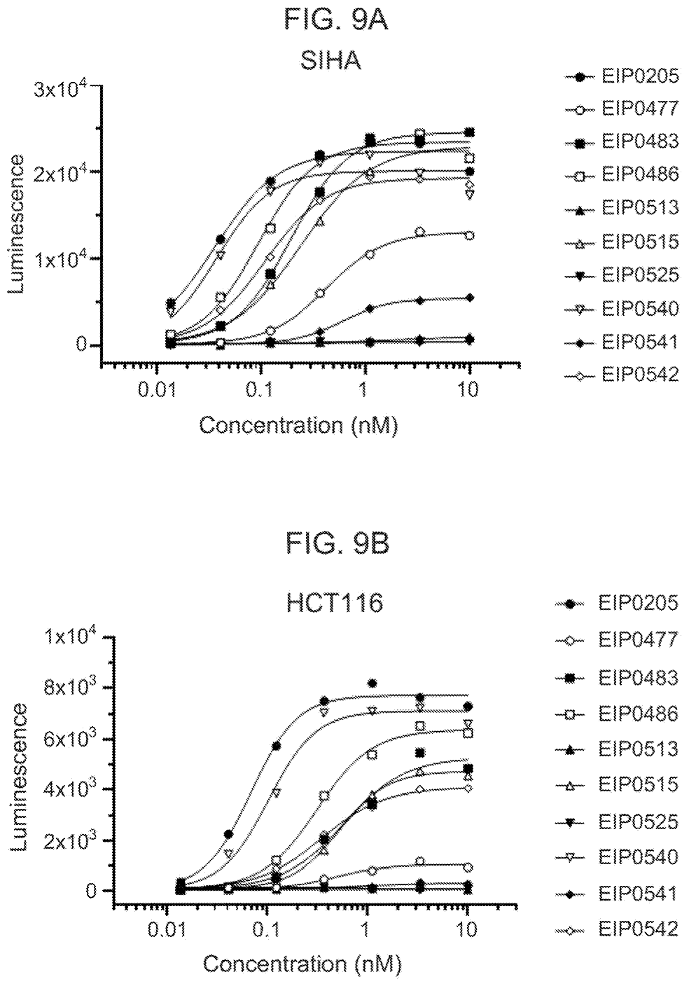

The degree of NFAT activation as measured by luciferase (FIG. 9A-9B) revealed a much broader range of activity among the bispecific SP34 mutants than indicated by the CD3 epsilon binding ELISA. Whereas EIP0477 and EIP0541 were observed to be moderate affinity by ELISA (14.93 and 36.34 nM EC50 respectively), the NFAT luciferase reporter assay revealed that these two mutants had significant reductions in NFAT response in the high ULBP2 cell line, SiHa (FIG. 9A) and little to no activity in the low ULBP2 cell line, HCT116 (FIG. 9B). In contrast, other SP34 mutants, EIP0483, EIP0486, EIP0515, and EIP0542, displayed the expected moderate NFAT response but with a broader range of differences between the mutants than observed by ELISA.

The greatest range of values between the CD3 variants were observed using NFAT Jurkat reporter and SiHa cell line (Table 35). Thus, this assay was used to bin the CD3 variants into the following categories of CD3 activity: strongest, EC50<50 pM; strong, EC50=50-150 pM; moderate, EC50=150-300 pM; weak, EC50=301-1000 pM; very weak, EC50>1000 pM.

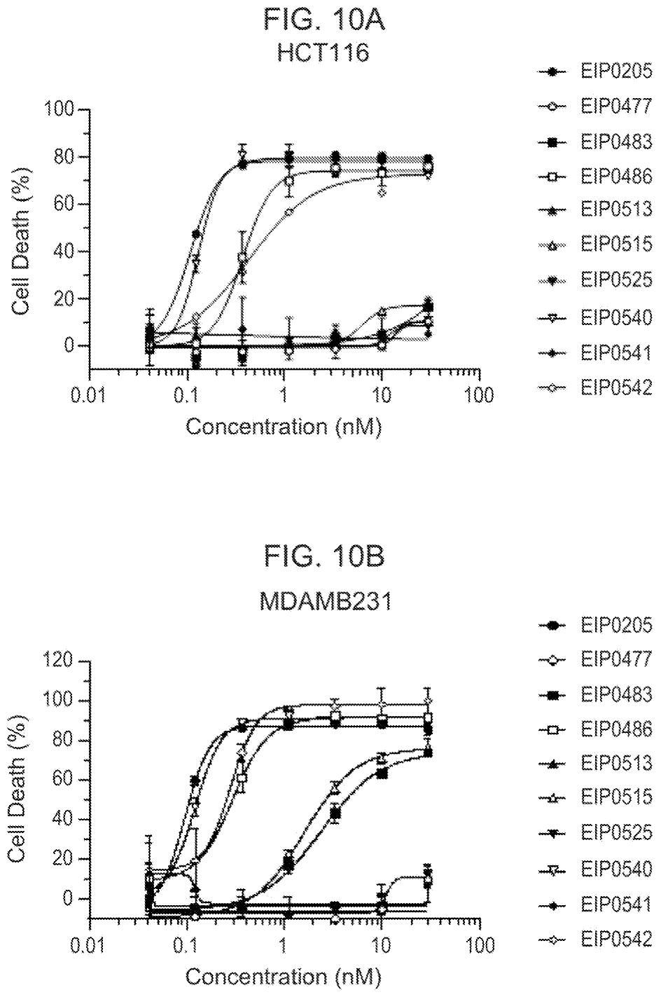

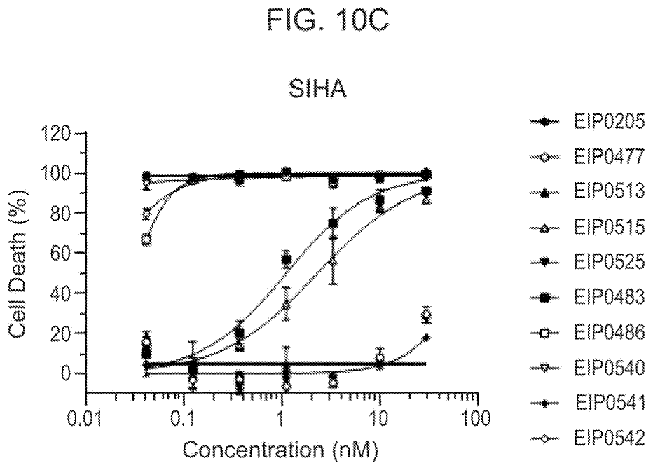

Bispecific SP34 affinity mutants were evaluated for their ability to induce cytolysis of cancer cells lines with varying levels of ULBP2 expression (SiHa>MDA-MB-231>HCT116) in the presence of activated T cells. Consistent with the NFAT luciferase reporter assay, a broad range of reduced in cytolytic activity for bispecific SP34 mutants across the three cell lines was observed (FIG. 10A-10C). The rank order of cytolytic activity across the mutants was consistent with the order of NFAT activation observed previously. For each individual bispecific, cytolytic activity was also directly correlated with the levels of ULBP2 on the tumor cells. For example, the EC50 for EIP0483 among the 3 cell lines are: SiHa, 1.11 nM; MDA-MB-23, 2.93 nM; and HCT116, 71.21 nM.

### T-Cell Secreted Cytokines

T cell secreted cytokines (IFNγ, IL-2, and TNFα) from the SiHa cytotoxicity assay were also measured. Human tumor cells were seeded at 10,000 cells per well in 96-well tissue culture plates (Perkin Elmer) and incubated for 24 hours at 37° C. with 5% CO₂. Naïve or activated human T cells (50-100,000) or PBMCs (250-300,000) were added to tumor cells in the presence or absence of antibodies and incubated for an additional 24-48 hours. Conditioned media was then transferred to separate 96-well plate and centrifuged (1000×g) for 5 min to pellet cells. Aliquots of conditioned media were removed for cytokine measurements using interferon gamma (IFNγ), interleukin 2 (IL-2) and Tumor necrosis factor alpha (TNFα) ELISA kits (Biolegend).

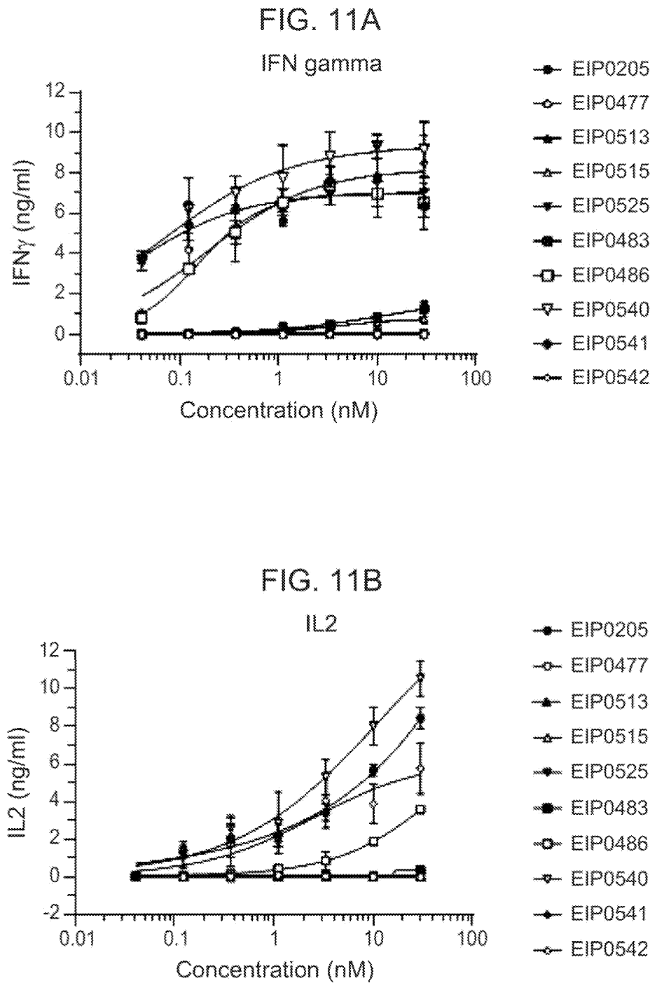

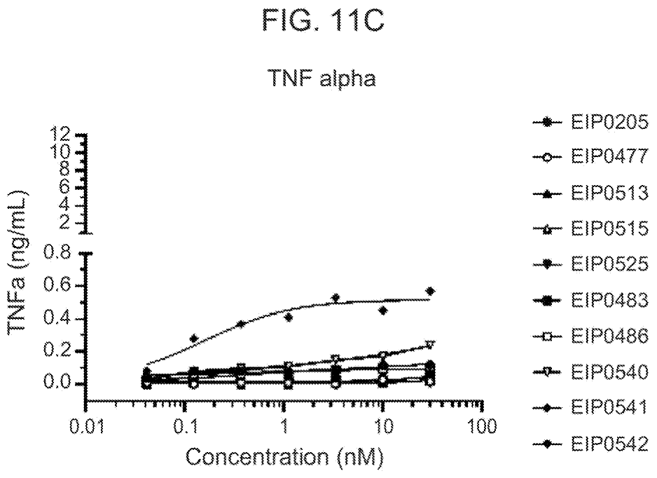

Three SP34 affinity mutants (EIP0486, EIP0540 and EIP0542) induced similar levels of IFNγ release as the bispecific SP34 wild-type (EIP0205) while the remaining mutants produced very little cytokine (FIG. 11A-11C). The same three SP34 affinity mutants also induced IL-2 though at levels different than the bispecific SP34 wild-type. Overall, TNFα levels were very low though EIP0542 did produce the highest level among all the bispecifics tested.

### Example 7-Production and Biophysical Characterization of αULBP2-αCD3 Bispecific Antibodies with Fusion Peptides

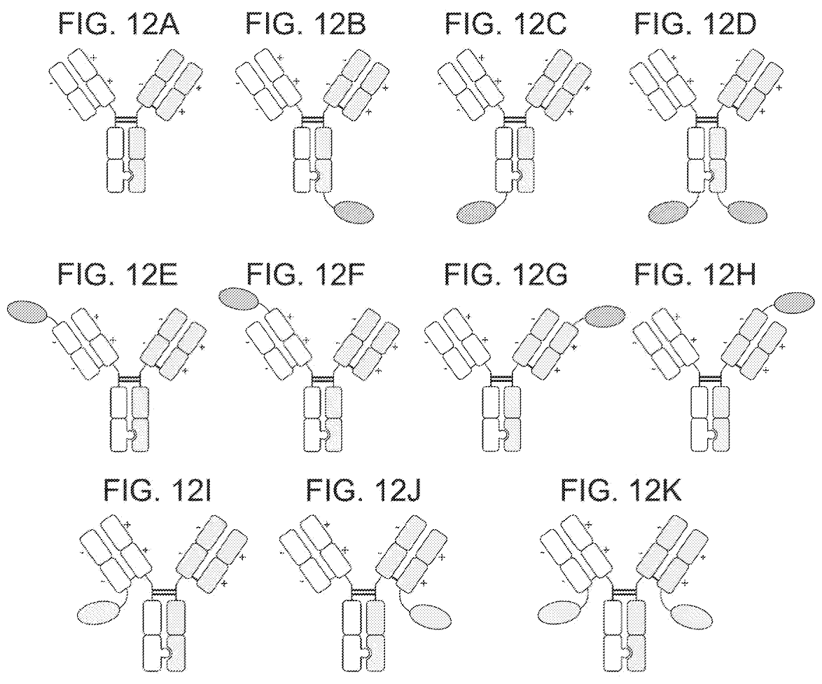

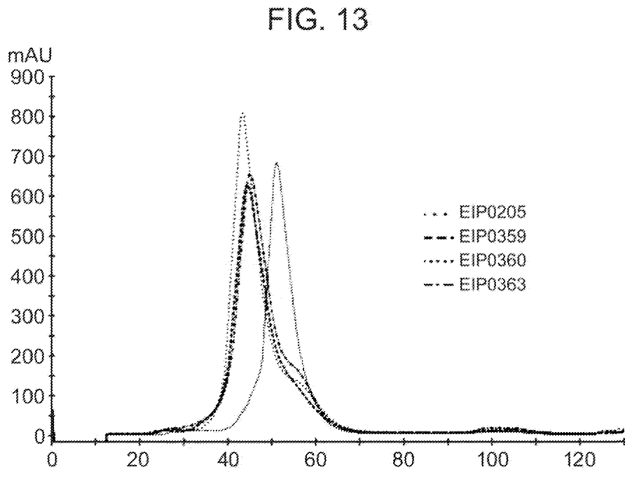

The crystal structure of human CD58 in complex with human CD2 (PDB 1QA9) confirmed that the interface occurs through amino terminal or IgV-like domain of CD58 (CD58v) which is one of two domain in the extracellular region (the other CD58 domain is IgC2-like; https://doi.org/10.3389/fimmu.2018.01204) and the amino-terminal domain of CD2 (Sung H, Ferlay J, Siegel R L, Laversanne M, Soerjomataram I, Jemal A, Bray F. Global Cancer Statistics 2020: GLOBOCAN Estimates of Incidence and Mortality Worldwide for 36 Cancers in 185 Countries. CA Cancer J Clin. 2021 May; 71(3):209-249. doi: 10.3322/caac.21660. Epub 2021 Feb. 4. PMID: 33538338; Wang, J. et al. Structure of a Heterophilic Adhesion Complex between the Human CD2 and CD58 (LFA-3) Counterreceptors. Cell 97, 791-803 (1999)). Given that the amino-terminus of CD58 is distal to the CD2 binding interface, CD58 or CD58v may be functional as both amino and carboxyl terminal fusions to all four antibody chains of the bispecific antibody allowing several formats possible (FIG. 12A-12K). Both amino and carboxyl terminal IL-7 fusions (WO2020236655A1 and WO2005063820A2) and could also follow the similar formats as CD58.

Bispecific fusion was generated by fusing the full-length extracellular domain of CD58 (UNIPROTKB: P19256, amino acids: 29-216), CD58v (amino acids: 29-122), IL7 (UNIPROTKB: P13232, amino acids: 26-177) using glycine serine linker to the carboxyl-terminus of the hole heavy chain of the bispecific consisting of ULBP2 antibody E12 and CD3 antibody SP34 (EIP0205). Bispecifics consisting of the ULBP2 antibody E12 and SP34 affinity mutants were fused with CD58 at the carboxyl-terminus of the hole heavy chain to create a series of bispecific CD58 fusions. Listed in Table 17 are bispecific CD58 fusions and their cognate bispecific antibodies.

FIG. 13 shows the size exclusion chromatograms of bispecific αULBP2-αCD3 (EIP0205) and bispecific fusions: αULBP2-αCD3-CD58 (EIP0359), αULBP2-αCD3-CD58v (EIP0360), and αULBP2-αCD3-IL7 (EIP0363) following elution from protein A.

Given that expression culture volume was equivalent for all four antibodies, the calculated area under the curve (AUC) is representative of antibody yields, thus fusions with CD58 and CD58v and IL-7 were not detrimental to the expression compared to the bispecific αULBP2-αCD3. Despite the emergence of lower molecular weight species as evidenced by the shoulder to the right of the peak of interest (POI) for all three bispecific fusions, the overall shape of the POI is consistent with that of monoclonal antibodies and suitable for drug development.

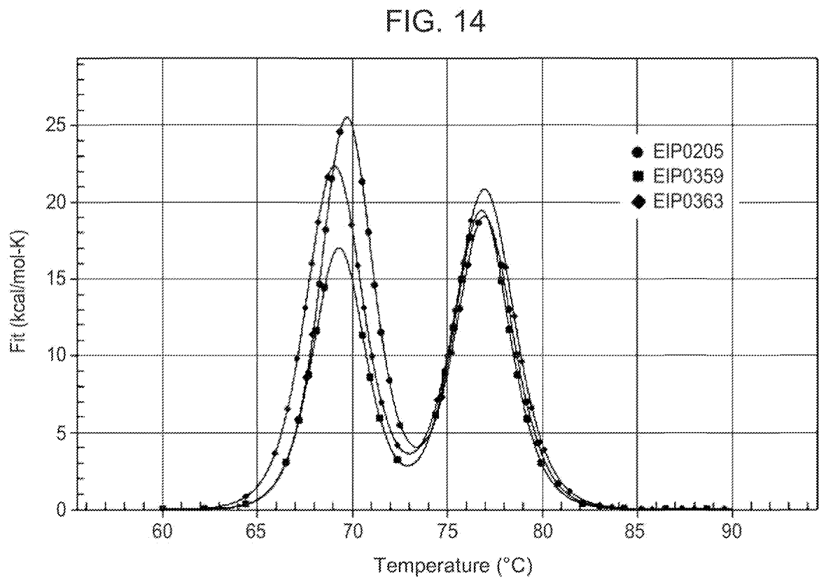

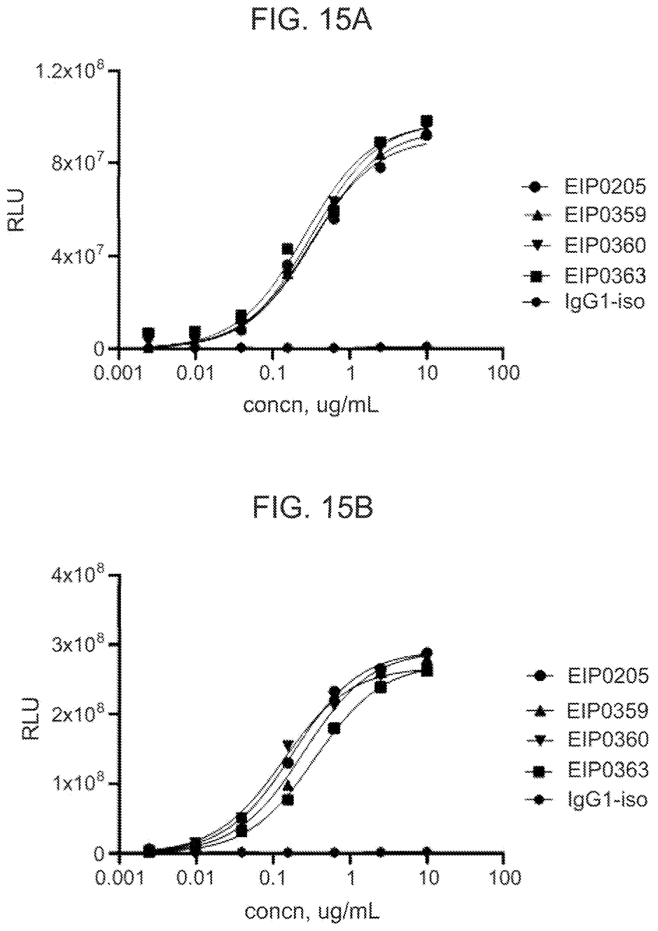

Differential scanning calorimetry (DSC) was used to determine whether the addition of CD58 and IL-7 would alter the transition temperature of the bispecific antibody. As shown in FIG. 14, two major transitions for antibodies produced using knob-into-hole (KIH) technology. The engineered KIH mutations in the CH1_{H3} or CH2_{H3} resulted in a reduced melting temperature that now overlaps with the CH2 domain (70° C.), while the second transition represents melting of Fab region (75° C.). The addition of CD58 or IL-7 to the C-terminus of the bispecific antibody had a minimal effect reducing the transition temperature of CH1_{H2} or CH2_{H2} or CH1_{H3} or CH2_{H3} by only 1° C. while the Fab was unaltered. Finally, the fusion of CD58, CD58v and IL-7 to the carboxyl terminus of the hole heavy chain of the bispecific did not alter binding to ULBP2 or CD3 by the αULBP2-αCD3 bispecifics as evaluated by sandwich ELISA (FIG. 15A-15B).

### Example 8-In Vitro Functional Characterization of Bispecific Antibodies with Fusion Peptides

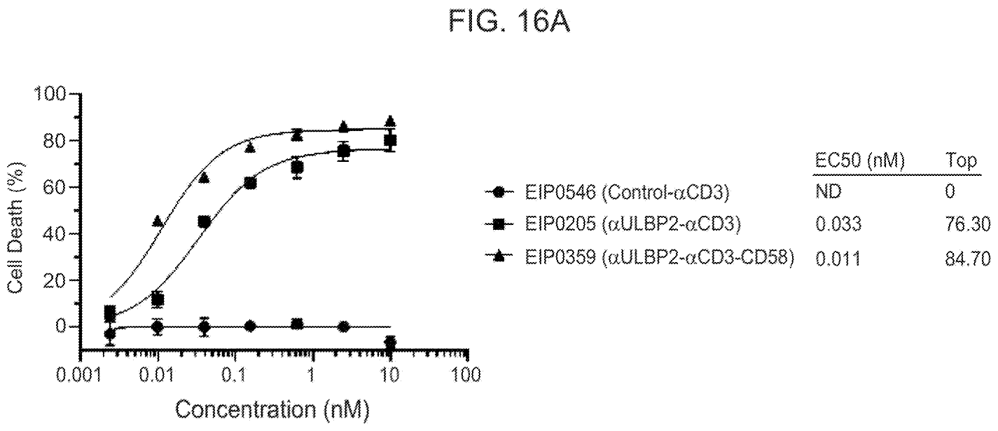

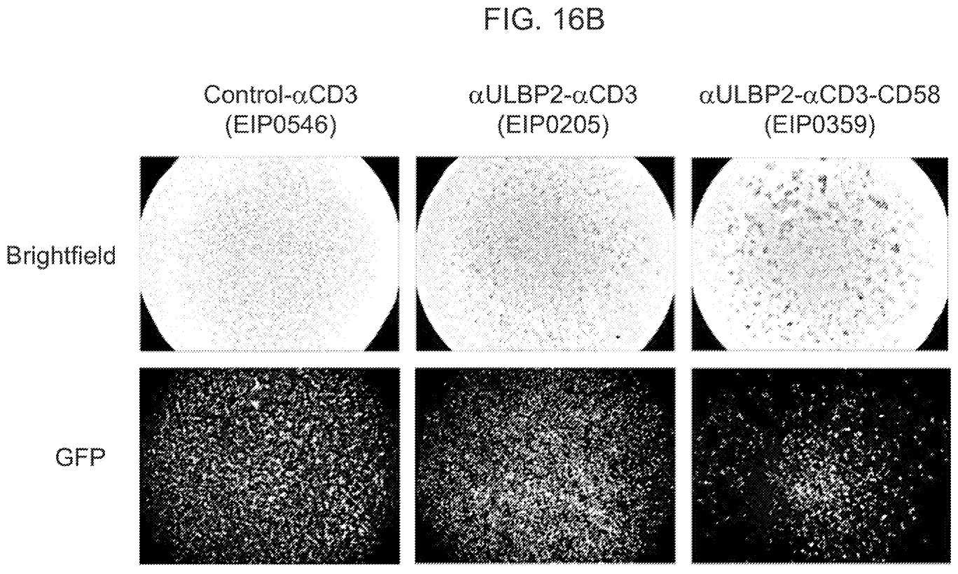

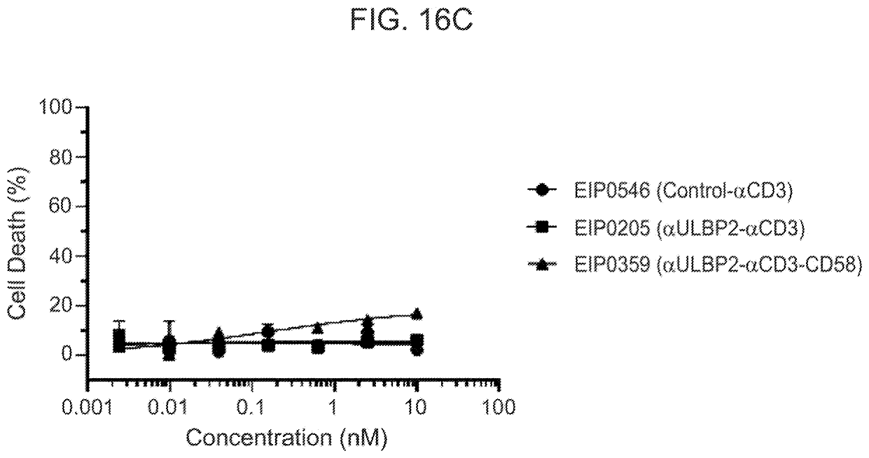

The activity of bispecific fusion, αULBP2-αCD3-CD58 (EIP0359), was compared to the bispecific, αULBP2-αCD3 bispecific (EIP0205), in a cytotoxicity assay with naïve T cells co-cultured with MDA-MB-231 GFP/luciferase cells. After 7-day incubation, the percentage of live GFP labeled tumor cells were determined using fluorescent imaging. As shown in FIG. 16A, αULBP2-αCD3-CD58 showed greater cytolytic activity than αULBP2-αCD3 as indicated by EC50 (3-fold lower concentration) and maximum cell death (9% increase). The difference in fluorescent signal of MDA-MB-231 GFP cells between αULBP2-αCD3, and αULBP2-αCD3-CD58 at a concentration of 625 pM as shown by fluorescent cell images (FIG. 16B). The activity for both αULBP2-αCD3 and αULBP2-αCD3-CD58 is dependent on target expression as ULBP2 deficient MDA-MB-231 cells (generated through CRISPR-Cas9 targeted inactivation of the ULBP2 gene) were largely resistant to cytolysis (FIG. 16C). In addition, no cytotoxicity was observed using Control-αCD3 bispecific (EIP0546) for both parental and ULBP2-deficient MDA-MB-231 cells.

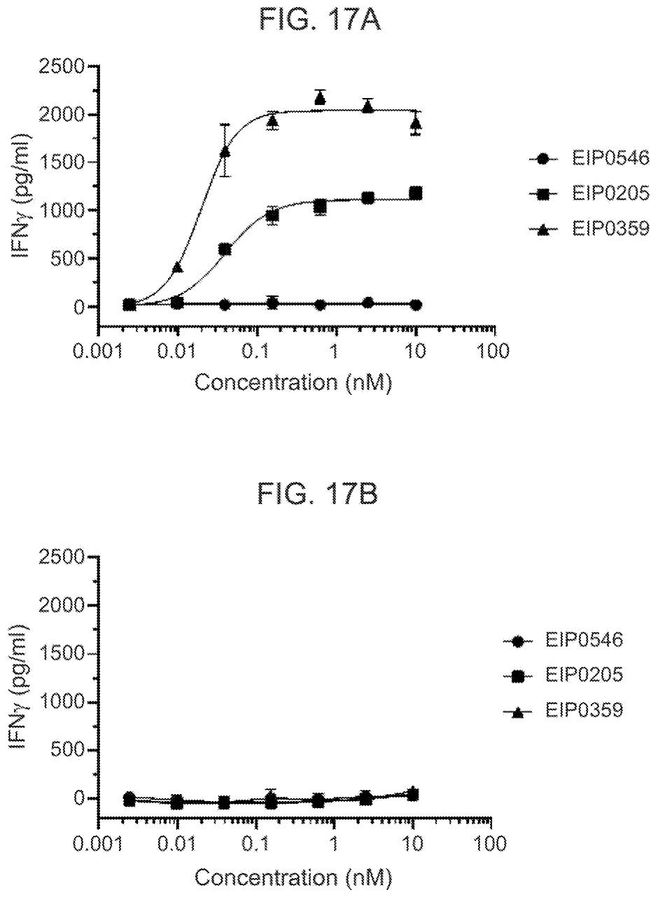

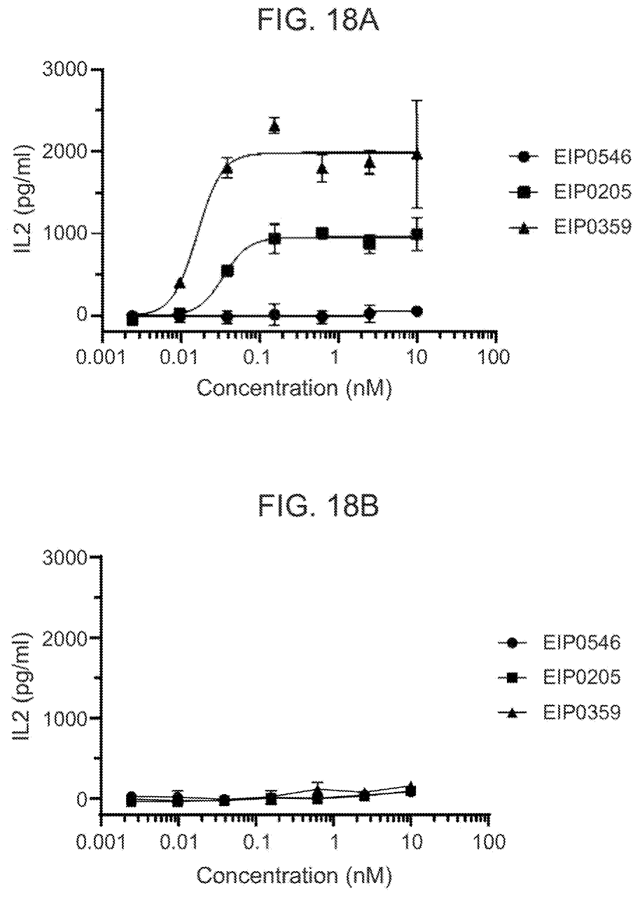

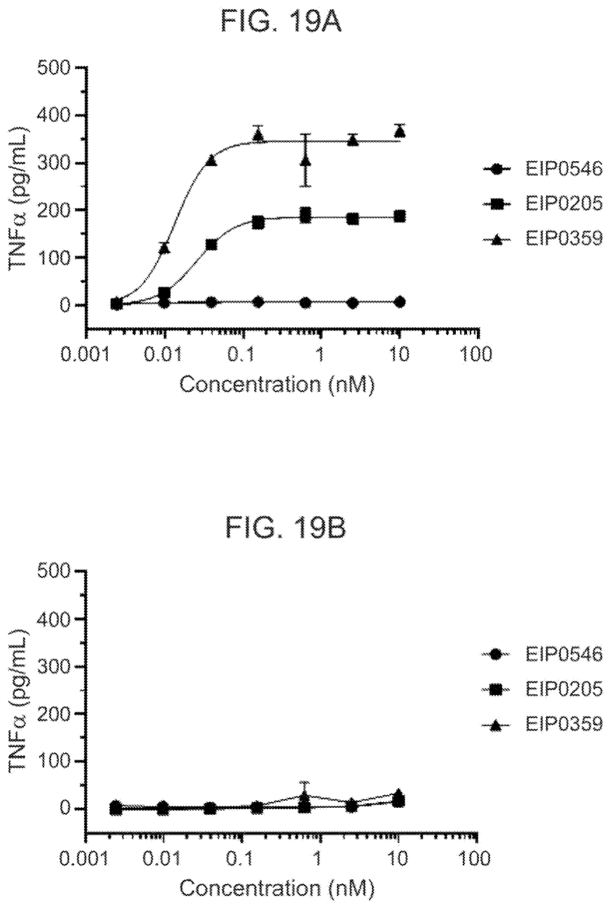

The release of the T cell cytokines IFNγ, IL-2 and TNFα were also measured from the media of naïve T cells co-cultured with MDA-MB-231 GFP/luciferase cells in the presence of bispecific αULBP2-αCD3 (EIP0205) and the bispecific fusion, αULBP2-αCD3-CD58 (EIP0359) after 24 hours. An increase of 79% was observed for IFNγ levels (FIG. 17A), an increase of 104% was observed for IL-2 (FIG. 18A) and an increase of 89% was observed for TNFα (FIG. 19A) with αULBP2-αCD3-CD58 compared to αULBP2-αCD3 bispecific as measured by the area under the curve (AUC) over the entire dose range. Consistent with the lack of cytotoxicity, the absence of ULBP2 on MDA-MB-231 cells resulted in no measurable levels for all three cytokines (FIGS. 17B, 18B, and 19B). The Control-αCD3 bispecific (EIP0546) did not induce cytokines from both parental and ULBP2-deficient MDA-MB-231 cells.

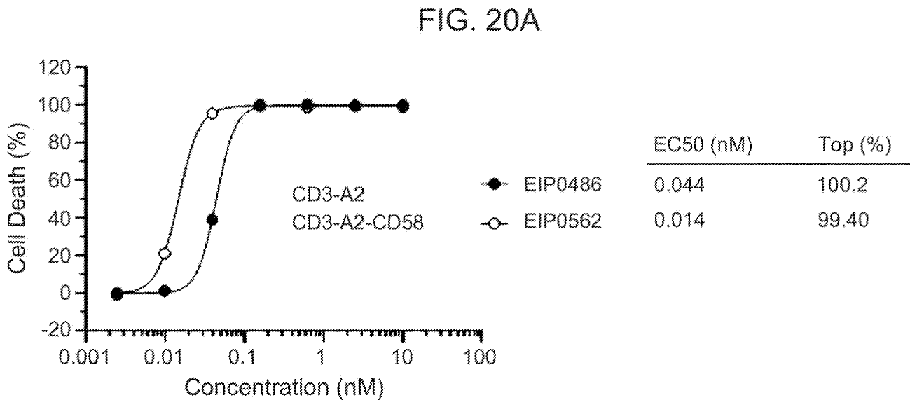

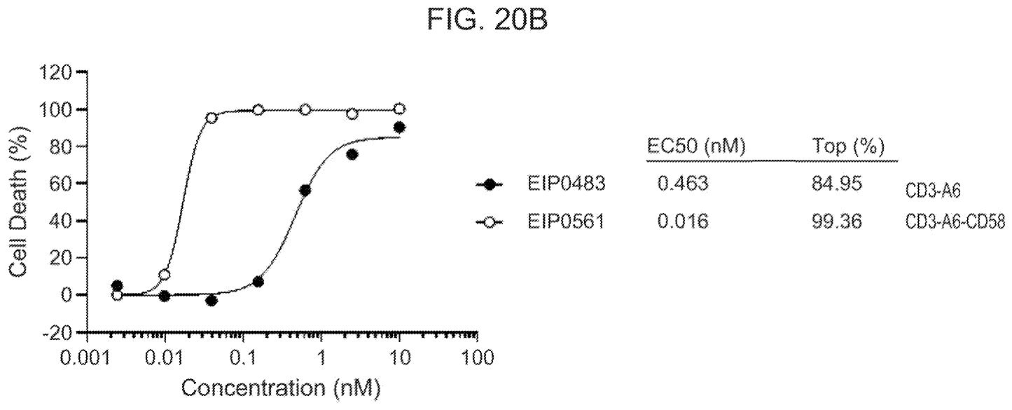

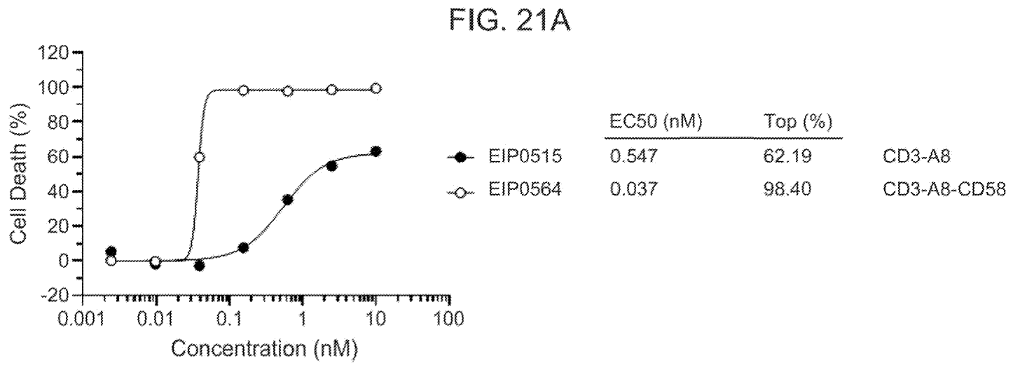

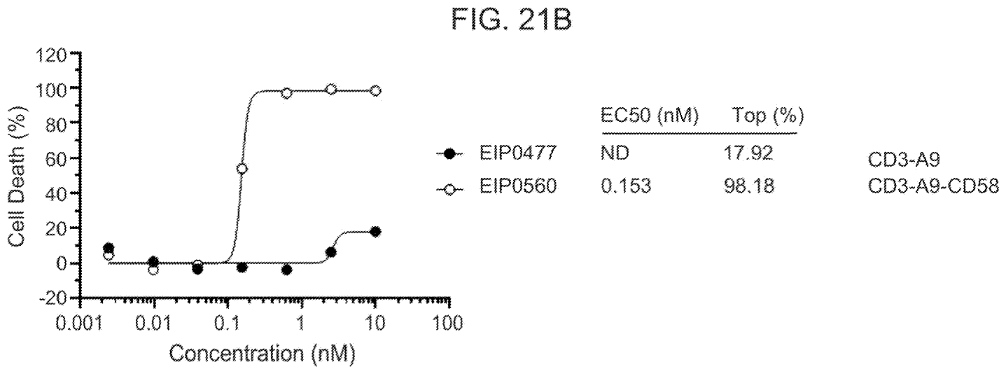

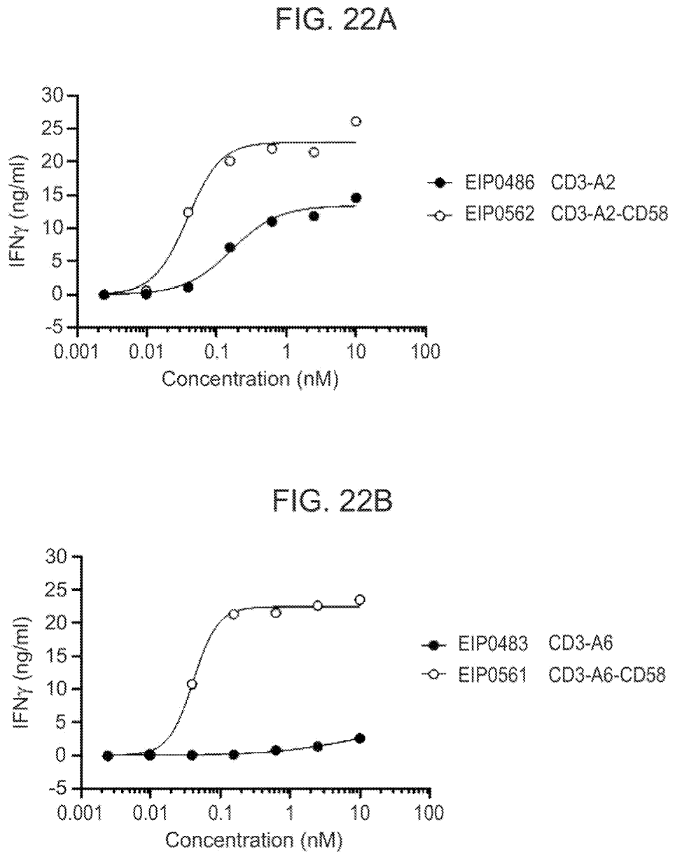

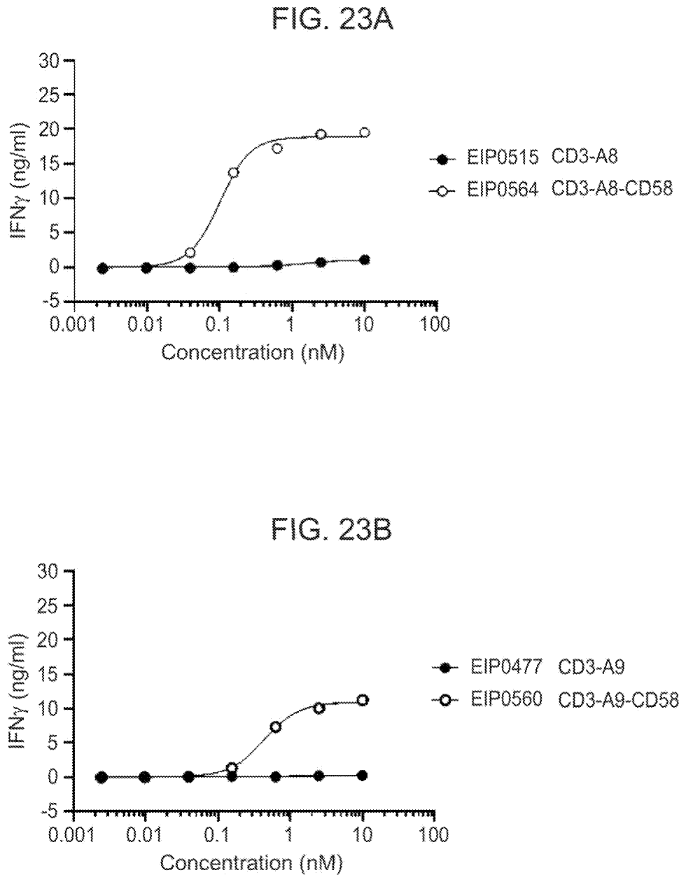

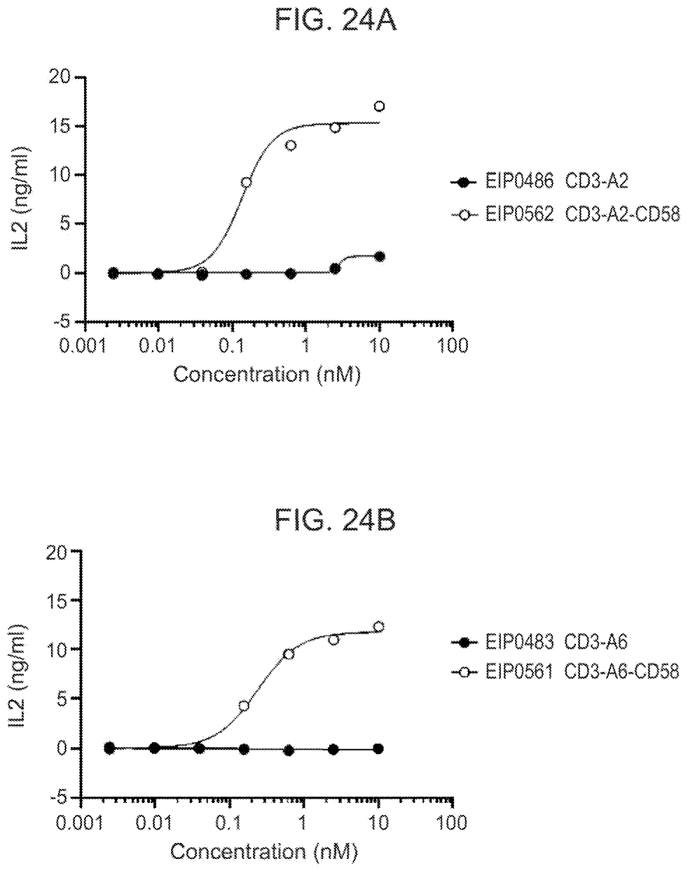

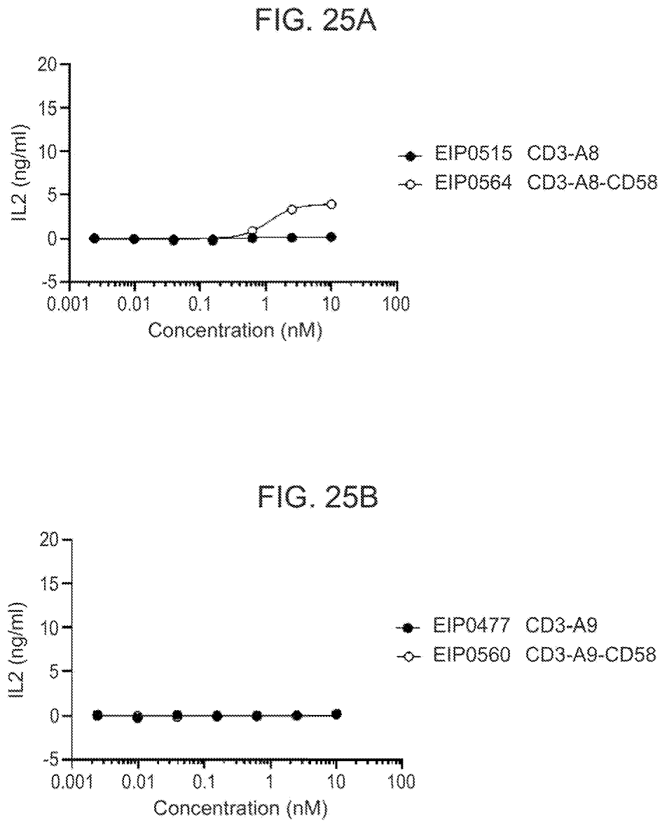

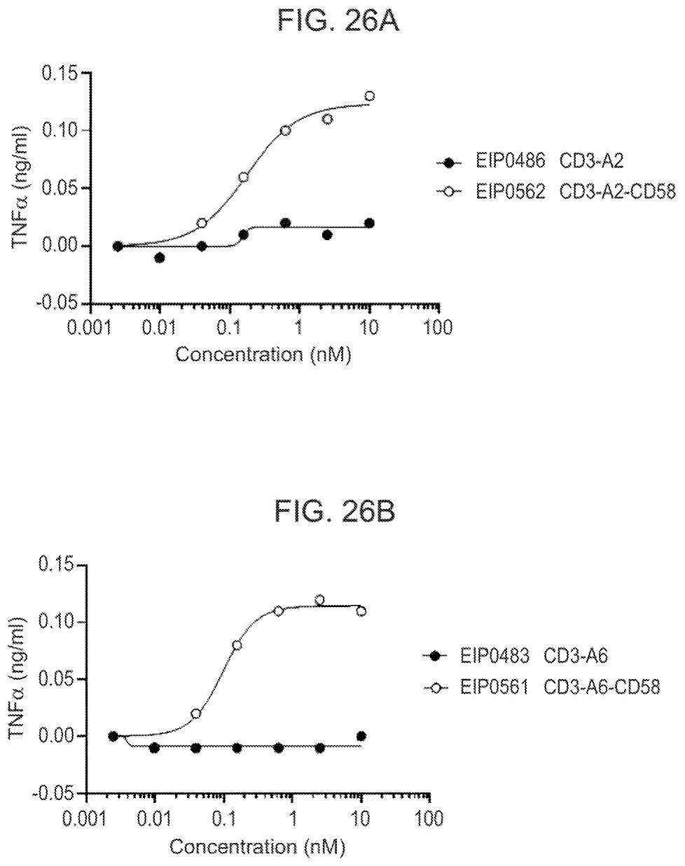

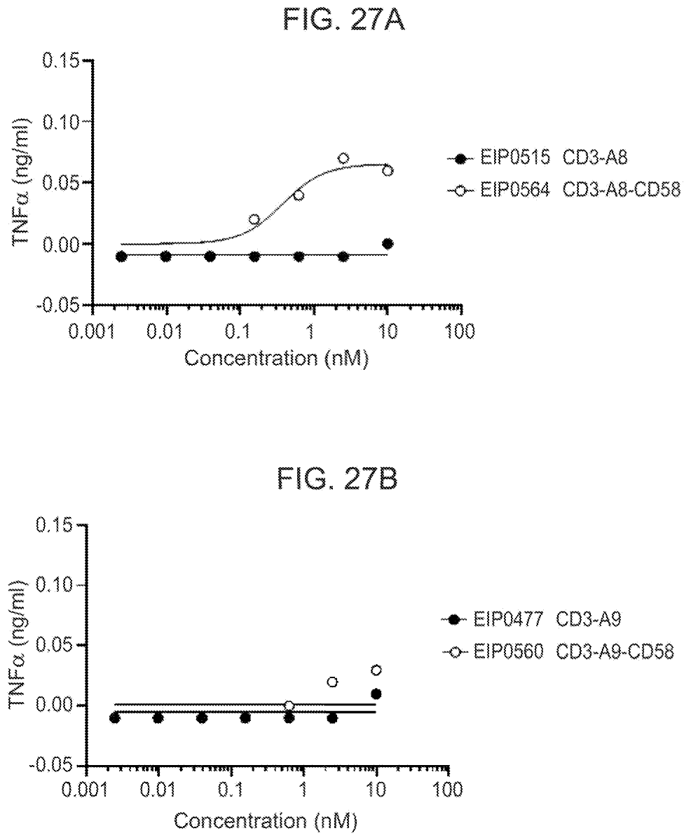

Bispecific SP34 affinity mutants and their cognate CD58 fusions were evaluated for cytotoxicity (FIGS. 20 and 21) and for cytokine release (FIGS. 22-27) using in a co-culture assay with SiHa cells and activated T cells. Significant improvements in cytotoxicity were observed as measured both in EC50 and in maximum cell death when CD58 was fused to bispecifics with lower CD3 affinity. In general, the improvement in cytolytic activity of the bispecific CD58 fusion compared to the bispecific was inversely related to the CD3 affinity of the bispecific. For example, the lowest CD3 affinity bispecific, EIP0477, induces minimal cell death only at the highest concentration of antibody with undetermined EC50 (FIGS. 20 and 21), whereas the equivalent bispecific CD58 fusion (EIP0560) induced 98% cell death with an EC50 of 0.15 nM. Conversely, for a high CD3 affinity bispecific, EIP0486, the fusion of CD58 (EIP0562) improved cytotoxicity modestly lowering the EC50 concentration by 3-fold (FIG. 20). Similar trends were observed for bispecific CD58 fusions and their cognate bispecifics for IFNγ release (FIGS. 22 and 23), IL2 (FIGS. 24 and 25), and TNFα (FIGS. 26 and 27). Again, the gain of cytokine release for the bispecific CD58 fusion compared to the bispecific was inversely related to the CD3 affinity of the bispecific. By creating a series of bispecific CD58 fusions with different CD3 affinities, the therapeutic window can be modulated, as defined by balance of cytotoxicity and cytokine release, to suit a variety of tumor antigens with different expression patterns both in distribution and in absolute receptor number.

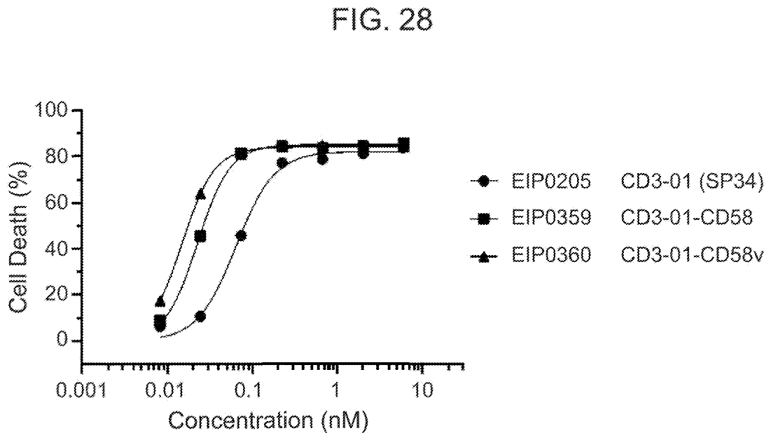

Bispecific fusion with CD58v, αULBP2-αCD3-CD58v (EIP0360) show similar improvements in cytolytic activity as the αULBP2-&CD3-CD58 (EIP0359) compared to the bispecific αULBP2-αCD3 (EIP0205) as determined by the cytolysis of MDA-MB-231 GFP cells using human PBMCs (FIG. 28).

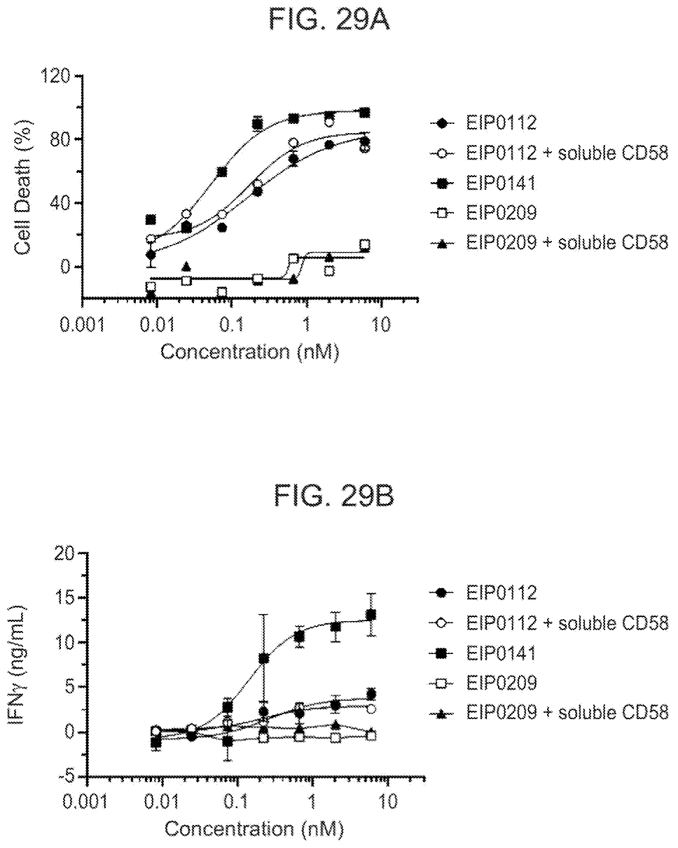

The improved activity of bispecific fusion, αULBP2-αCD3-CD58 (EIP0141) is dependent on the physical linkage between CD58 and the bispecific ULBP2-CD3 bispecific. As shown in FIGS. 29A and B, combining equimolar amounts of CD58-Fc protein and the bispecific, αULBP-αCD3 (EIP0121) did not produce the equivalent amount of cytolysis or IFNγ release as the integrated bispecific fusion (EIP0141).

To further understand the impact of CD58 costimulation, both the bispecific fusion, αULBP2-&CD3-CD58 (EIP0141), and the bispecific, αULBP2-xCD3 (EIP0112), were assessed in a cytotoxicity assay using CD8 T cells in different states of exhaustion. To achieve this, naïve CD8 T cells were first subjected to increasing rounds of stimulation with each round consisting of a 2-day incubation with Dynabeads coupled to anti-CD3 and anti-CD28 antibodies. As shown by fluorescent imaging of live tumor cells, the αULBP2-αCD3-CD58 was able to induce cytolysis of tumor cells even with exhausted T cells from round 5, while the activity of ULBP2-CD3 peaked with early exhausted T cells from round 3 (FIG. 30). The Control-αCD3 bispecific (EIP0209) did not induce cytolysis from both naïve or exhausted T cells.

Next, intracellular and cell surface markers of T cell activation and exhaustion in CD8 T cells from co-cultures of human PBMCs with MDA-MB-231 cells in the presence of bispecific, αULBP2-αCD3 (EIP0205) and the bispecific fusion, αULBP2-&CD3-CD58 (EIP0359) was assessed. The following T cell markers: intracellular IL2, intracellular IFNγ, CD25, CD69, granzyme B (GRZMB), CD2, PD-1, CD3ε, and TIM3 were measured using flow cytometry after 72-hour incubation. The MFI for each marker was normalized for the ULBP2-CD3 bispecific and represented in a radar plot (FIG. 31). Compared to the αULBP2-αCD3, αULBP2-αCD3-CD8, produced a distinct phenotype that is consistent with greater T cell activation as observed by the increased levels of IFNγ, CD25 and GZMB. In addition, T cells appeared to be less exhausted when treated with αULBP2-αCD3-CD58 bispecific fusion as evidenced by the reduction in CD3ε and no increase seen for TIM3 compared to αULBP2-αCD3. As a negative control, the Control-αCD3 (EIP0209) which does not bind to any known target protein on MDA-MB-231 cells, did not induce expression in any of the markers. This is consistent with the mechanism of CD3 bispecifics which require the presence of the target for T cell activation to occur.

### T Cell Proliferation Induced by CD3 Affinity Variant Antibodies

Differences in CD8+ T cell proliferation induced by bispecifics versus bispecific CD58 fusions across the different CD3 affinities were observed using a T cell stimulation assay. Freshly isolated naïve T cells were repeatedly exposed to Dynabeads coupled bispecifics and bispecfic CD58 fusions for successive rounds of 48-72 hour stimulation. Table 36 shows the CD8 T cell counts following each round of stimulation. As shown in Table 36, none of the bispecifics induce significant T cell proliferation beyond round 2 whereas all bispecific CD58 fusions continued to stimulate proliferation up to round 7.

TABLE 36
CD8+ T cell counts for T cell stimulation assay

| | | Stimulation Round | | | | | | |
|---|---|---|---|---|---|---|---|---|
| Protein | Description | 1 | 2 | 3 | 4 | 5 | 6 | 7 |
| EIP0205 | ULBP2 × CD3-01 | 4760 | 2365 | 325 | 153 | 48 | 243 | 16 |
| EIP0359 | ULBP2 × CD3-01 × CD58 | 4349 | 2061 | 2471 | 12135 | 9845 | 2200 | 691 |
| EIP0486 | ULBP2 × CD3-A2 | 4472 | 3352 | 828 | 364 | 140 | 277 | 40 |
| EIP0562 | ULBP2 × CD3-A2 × CD58 | 4419 | 3472 | 9064 | 34219 | 51012 | 36560 | 28691 |
| EIP0542 | ULBP2 × CD3-A5 | 4256 | 2997 | 877 | 476 | 144 | 253 | 3 |
| EIP0568 | ULBP2 × CD3-A5 × CD58 | 4200 | 2728 | 5851 | 37421 | 50951 | 24992 | 14739 |
| EIP0483 | ULBP2 × CD3-A6 | 3776 | 2957 | 1344 | 717 | 175 | 197 | 45 |
| EIP0561 | ULBP2 × CD3-A6 × CD58 | 4112 | 5896 | 31232 | 44848 | 42539 | 78784 | 131187 |
| EIP0515 | ULBP2 × CD3-A8 | 3619 | 3195 | 2233 | 4295 | 2067 | 189 | 83 |
| EIP0564 | ULBP2 × CD3-A8 × CD58 | 3581 | 5392 | 44791 | 66724 | 118255 | 36536 | 25947 |
| EIP0477 | ULBP2 × CD3-A9 | 3333 | 3725 | 1893 | 1095 | 260 | 256 | 67 |
| EIP0560 | ULBP2 × CD3-A9 × CD58 | 3016 | 4120 | 44688 | 101615 | 90000 | 67461 | 168363 |
| SP34 | CD3-01 | 1320 | 1648 | 56 | 15 | 5 | 80 | 5 |
| OKT3 | CD3 | 1013 | 2603 | 29 | 3 | 7 | 24 | 0 |

Tumor Studies with Co-Engrafted Human T Cells

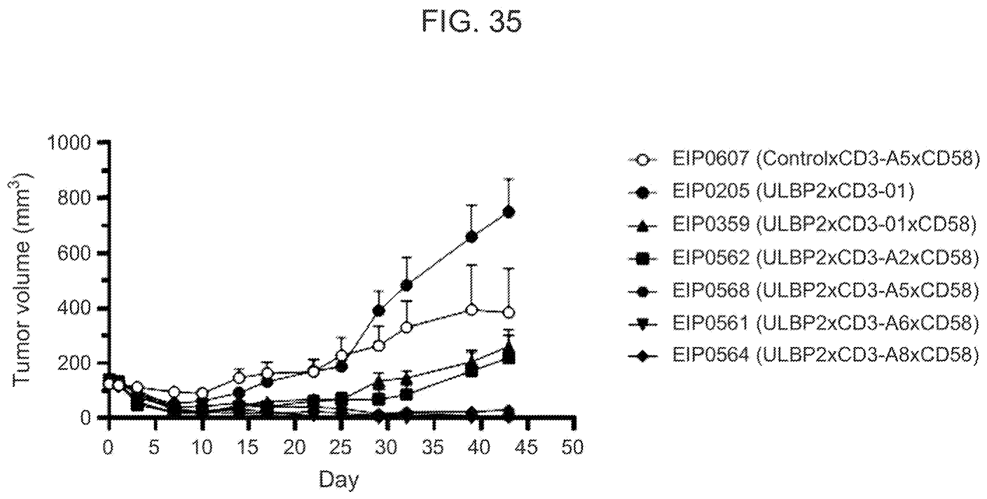

The activity of bispecific CD58 fusions with various CD3 affinities were evaluated in mouse xenograft study with human T cells and CORL-105 tumor cells co-engrafted simultaneously into flank of NSG mice. Mice were then dosed intravenously with bispecific CD58 fusions (EIP0359, EIP0562, EIP0568, EIP0561 and EIP0564), bispecific (EIP0205) and non-targeting control (EIP0607) at 4 mg/kg biweekly for 3 weeks (FIG. 35A). The administration of three bispecific CD58 fusions, EIP0568, EIP0561, and EIP0564, prevented the growth of tumors. Two bispecific CD58 fusions, EIP0359 and EIP0562, were moderate in their ability to control tumor growth while the non-targeting control was the least effective among all bispecific CD58 fusions in inhibiting tumor growth. However, the bispecific lacking CD58 fusion appeared had little effect on tumor growth of all molecules tested.

### Fusion of CD58 Improves T Cell Redirected Killing Using Multiple Tumor Targeting Antibodies

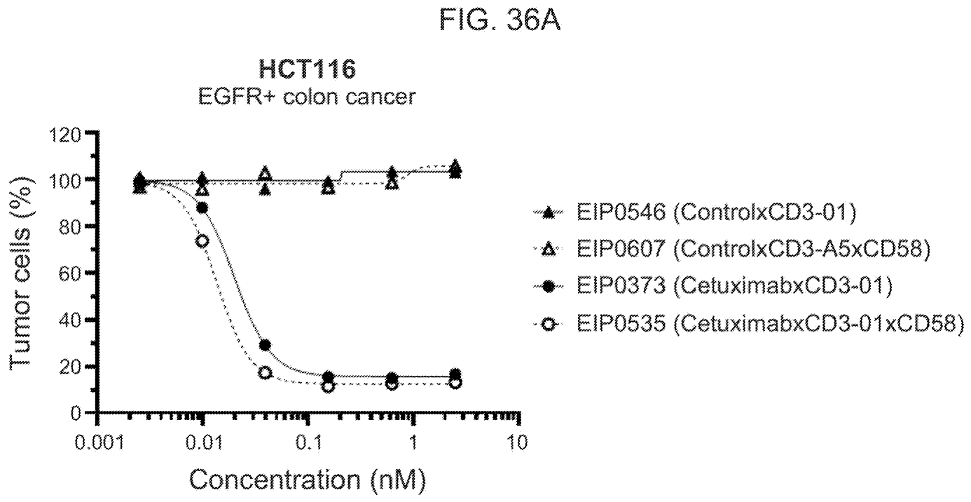

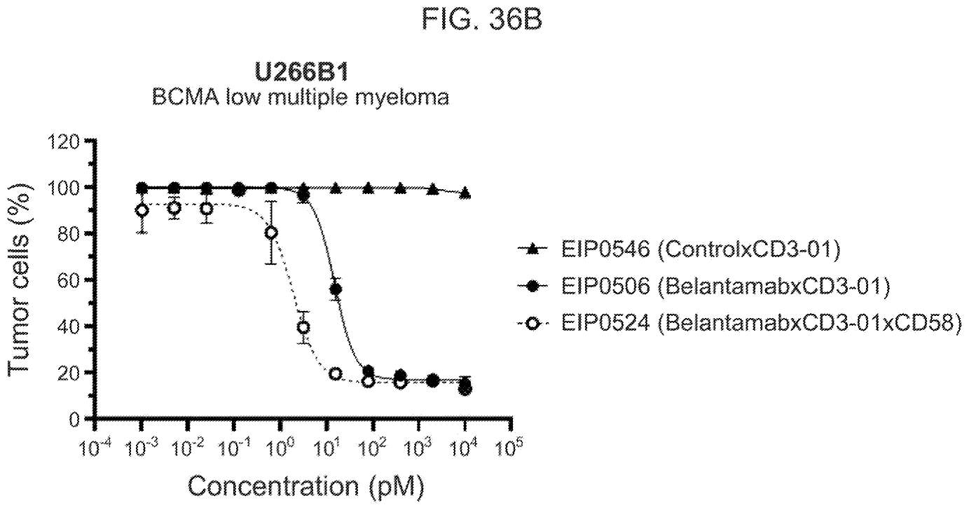

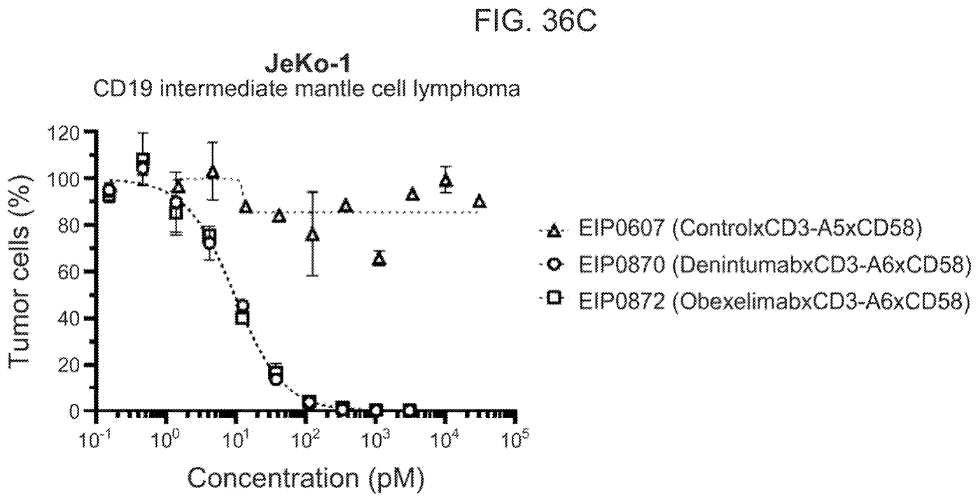

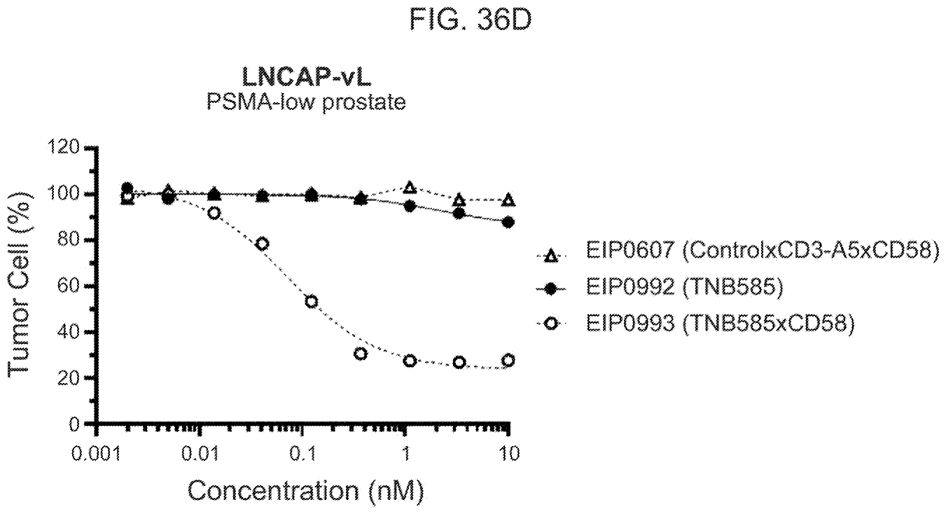

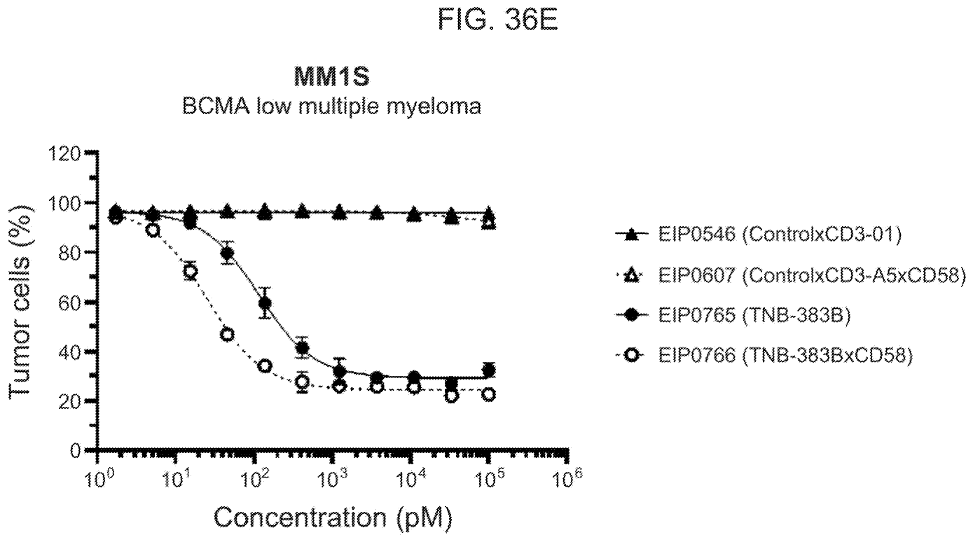

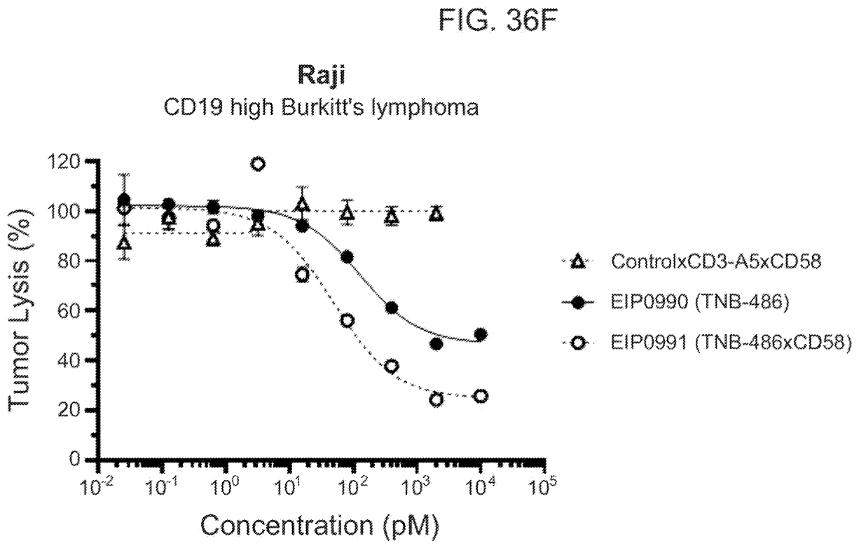

CD58 fusion to asymmetric CD3 bispecifics using therapeutically validated antibodies cetuximab (EGFR), belantamab (BCMA), denintumab (CD19) and obexelimab (CD19) was demonstrated to lead to improvements in tumor cell cytotoxicity, as shown in FIGS. 36A-36C. In FIG. 36A, HCT116 colon carcinoma cells were co-cultured with activated T cells in the presence of EIP0373 (CetuximabxCD3-01), EIP0535 (CetuximabxCD3-01xCD58), EIP0546 (ControlxCD3-01) and EIP0607 (ControlxCD3-A5xCD58). In FIG. 36B, U266B1 multiple myeloma cells were co-cultured with activated T cells in the presence of EIP0506 (BelantamabxCD3-01), EIP0524 (BelantamabxCD3-01xCD58), and EIP0546 (ControlxCD3-01). In FIG. 36C, JeKo-1 mantle lymphoma cells were co-cultured with activated T cells in the presence of EIP0870 (DenintumabxCD3-A6xCD58), EIP0872 (ObexelimabxCD3-A5xCD58), and EIP0607 (ControlxCD3-A5xCD58).

In all 3 cell lines, the bispecific CD58 fusions showed improved tumor cell cytotoxicity compared to the bispecifics lacking the CD58 fusion. As expected, the non-targeted control bispecific and bispecific CD58 fusion did not show any activity.

The modularity of CD58 can also lead to improvements tumor cell cytotoxicity when added to pre-existing CD3 bispecifics, as shown in FIGS. 36D-36F. TNB-383B (BCMA), TNB-486 (CD19), and TNB-585 are CD3 bispecifics in various stages of clinical development. In FIG. 36D, PSMA negative LNCAP prostate cancer cells (generated by CRISPR-Cas9 inactivation of PSMA gene) were transduced using PSMA encoding lentivirus to create a PSMA low LNCAP cell line. This cell line was co-cultured with activated T cells in the presence of EIP0993 (TNB-585xCD8), EIP0992 (TNB-585), and EIP0607 (ControlxCD3-A5xCD58). In FIG. 36E, MM1S multiple myeloma cells were co-cultured with activated T cells in the presence of EIP0765 (TNB-383B), EIP0766 (TNB-383BxCD58), EIP0546 (ControlxCD3-01), and EIP0607 (ControlxCD3-A5xCD58). In FIG. 36F, Raji lymphoma cells were co-cultured with activated T cells in the presence of EIP0990 (TNB-486), EIP0991 (TNB-486xCD58), and EIP0607 (ControlxCD3-A5xCD58).

In all three models, the fusion of CD58 to clinical CD3 bispecifics lead to significant improvements in cytotoxicity compared to bispecific itself. The superiority of the bispecific CD58 fusion was most significant in cell models with lower levels of target antigen. As expected, the non-targeted bispecific and non-targeted bispecific CD58 fusion did not show any activity.

### Engineered Disulfide Improves Expression and Increases Homogeneity of Antibodies

An engineered disulfide through cysteine substitutions at Y370 and S375 (Kabat numbering) in the CH2 regions of TNB-383B sequence (Publication No.

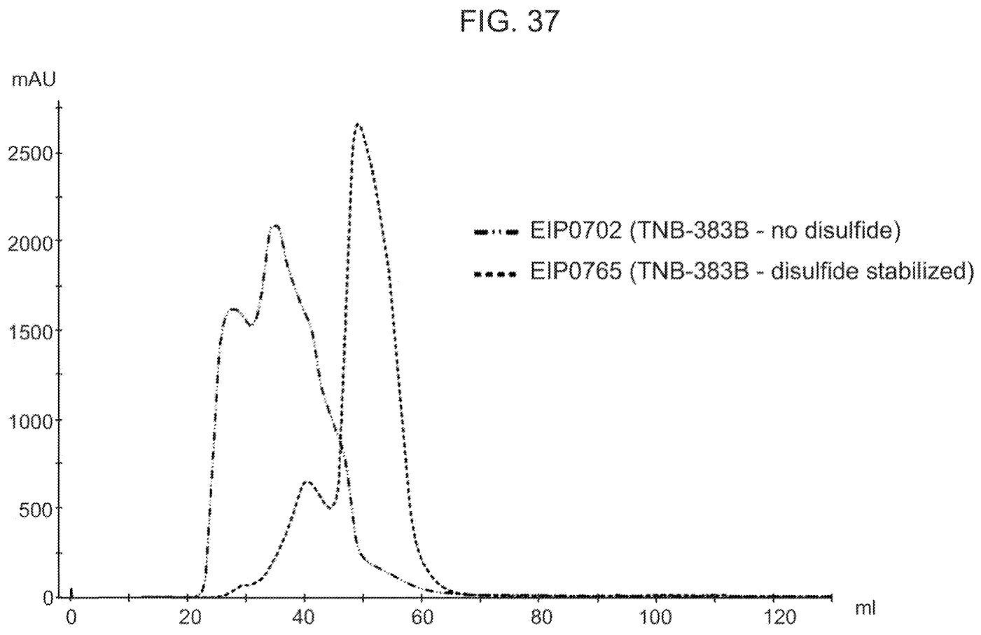

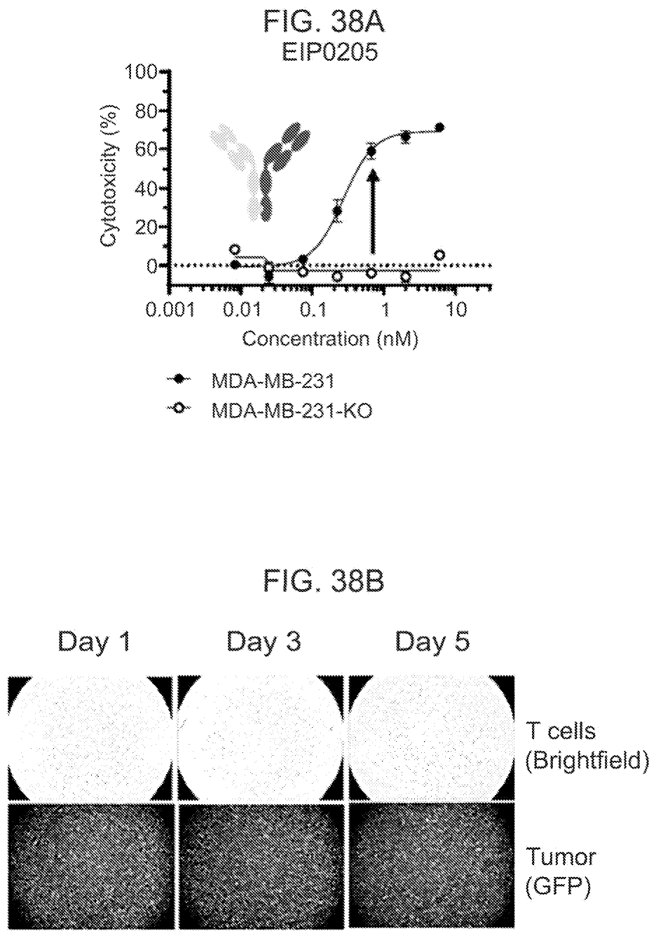

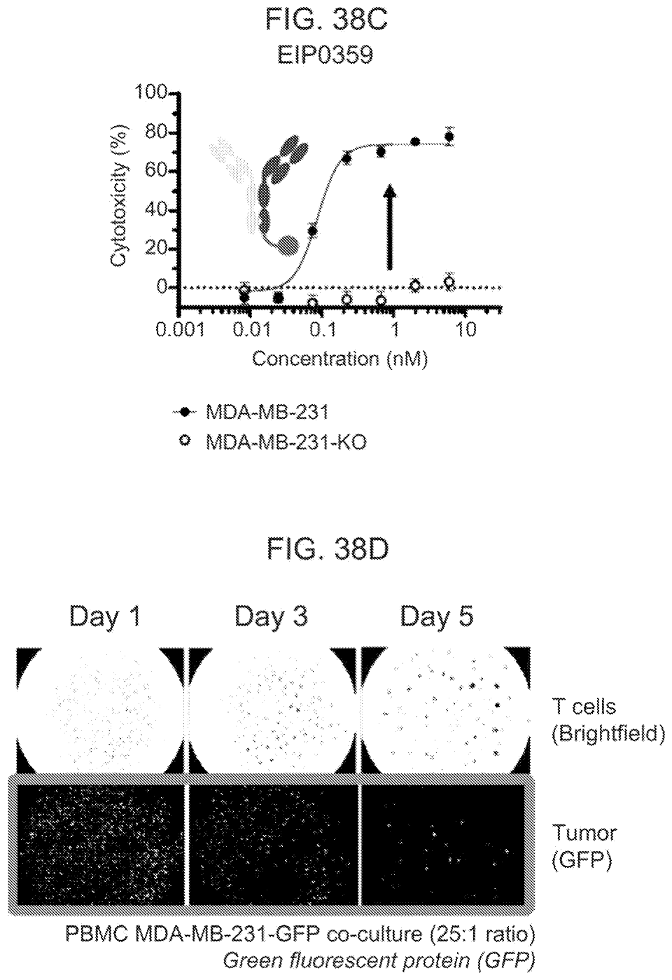

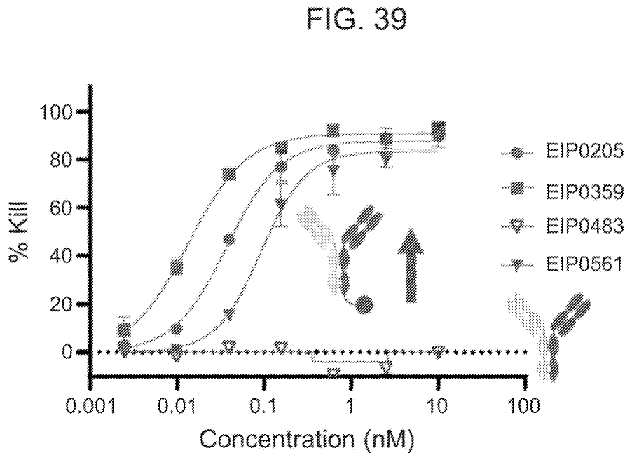

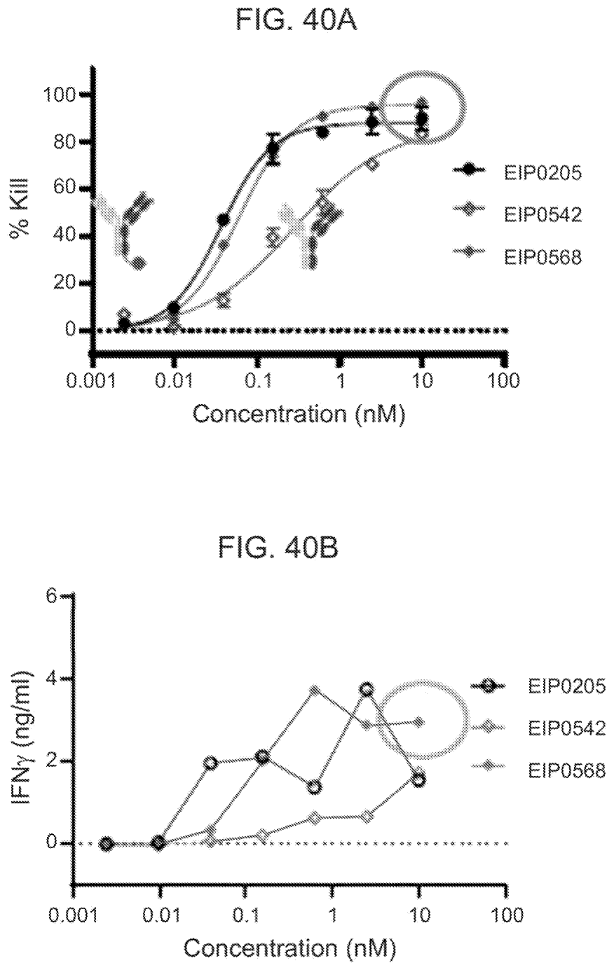

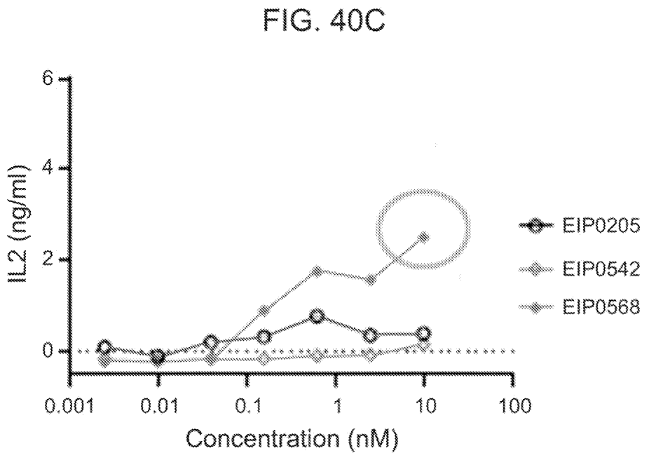

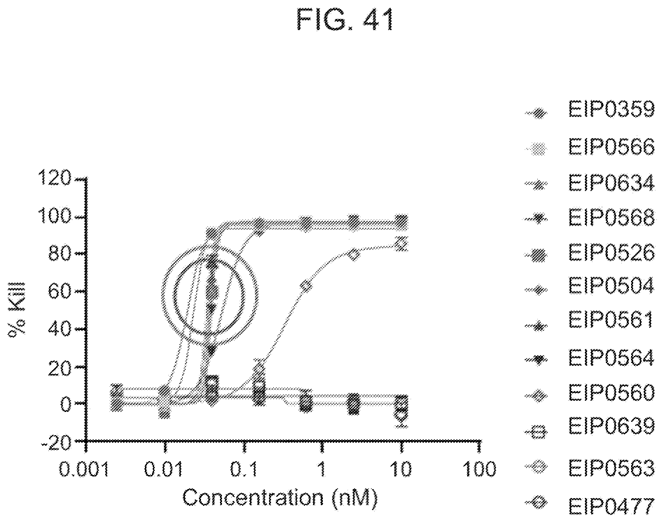

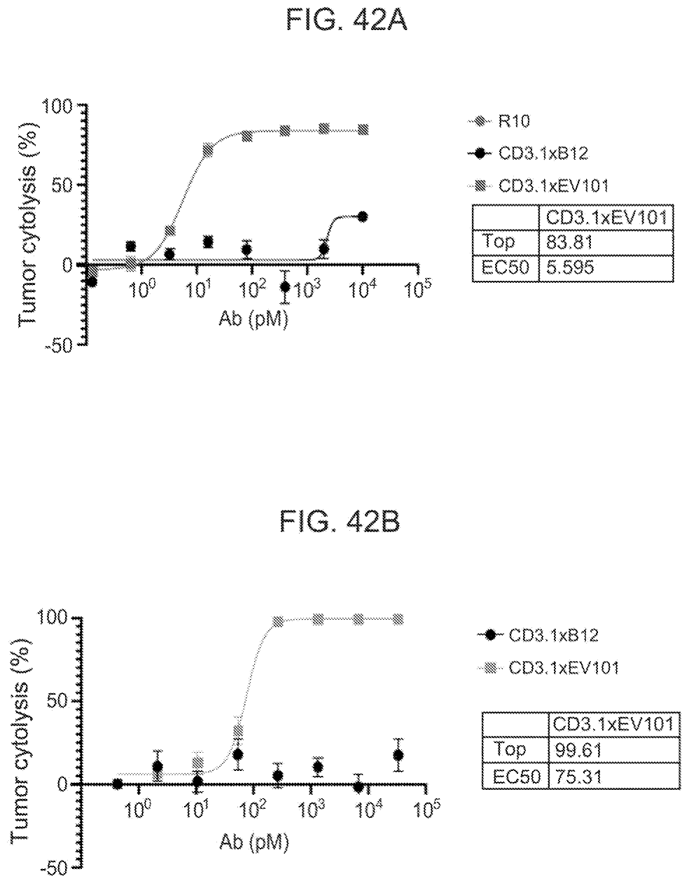

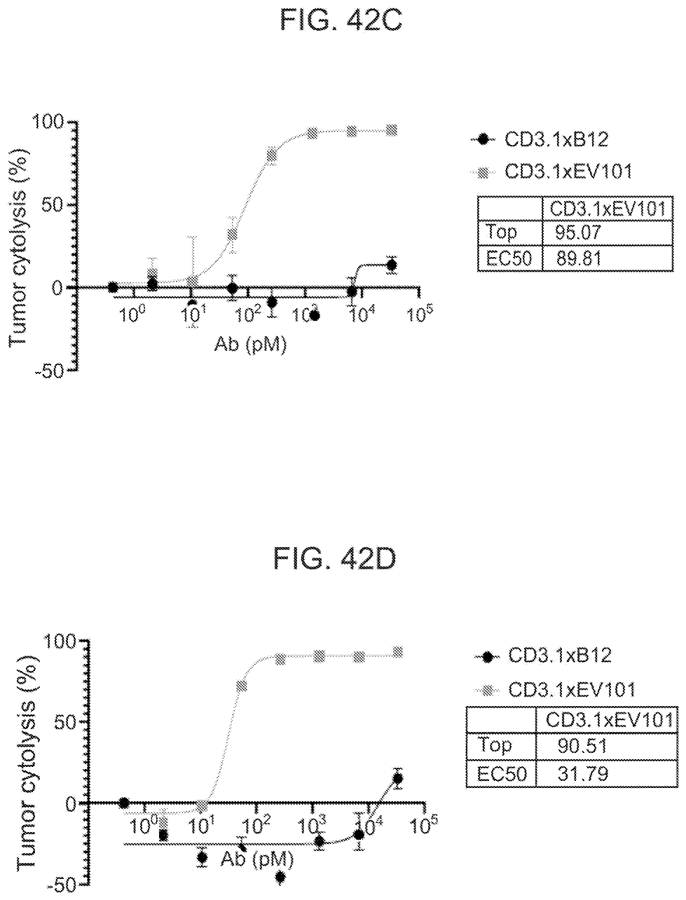

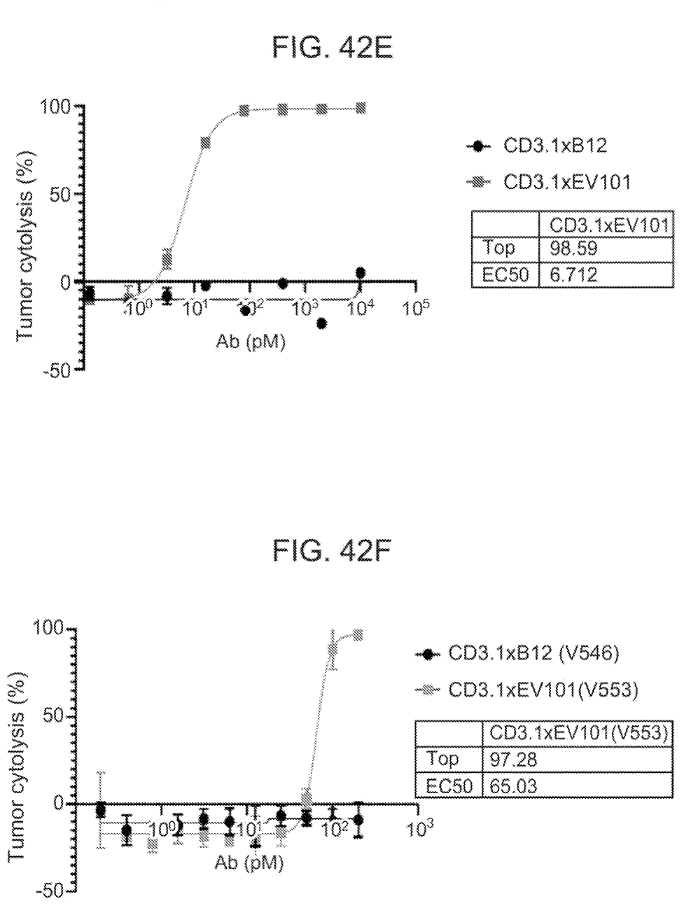

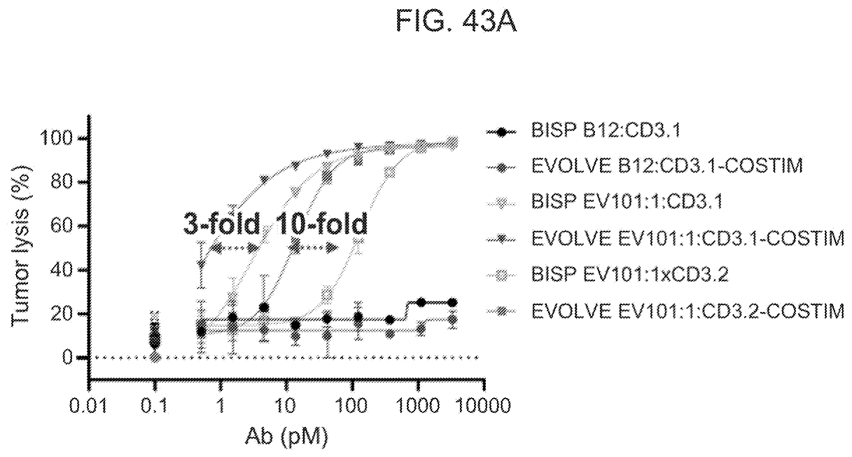

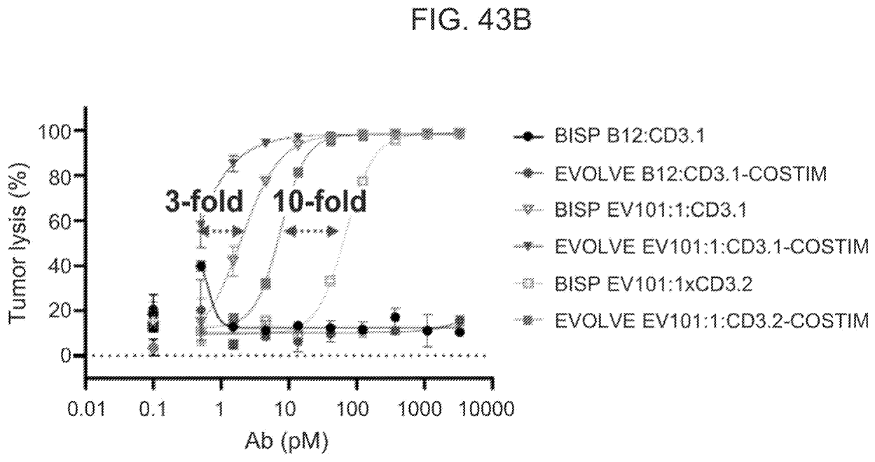

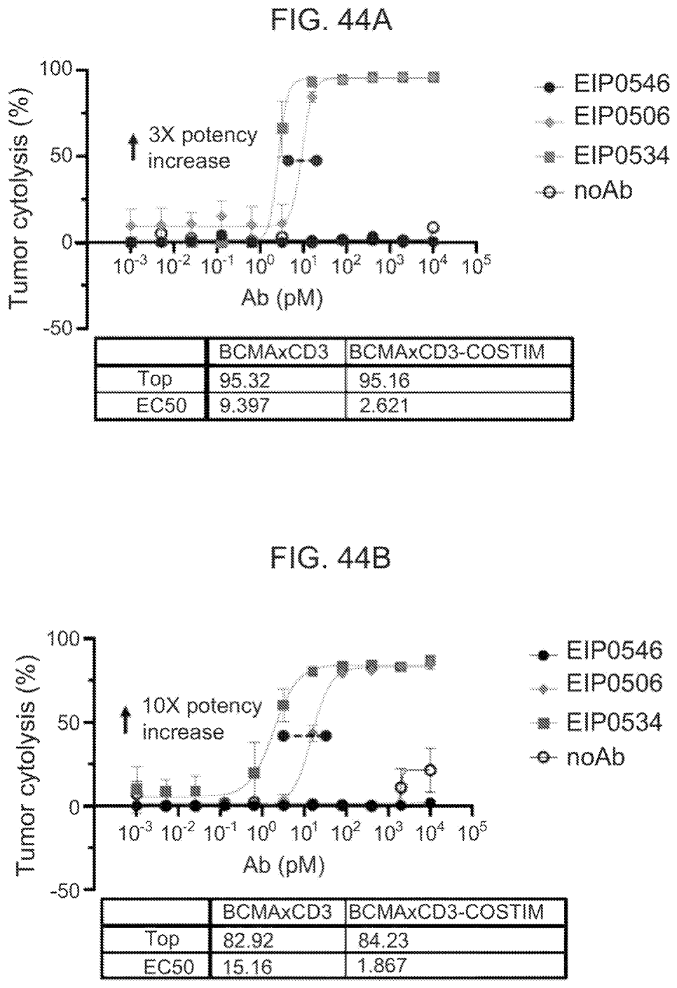

US20210403587A1) can improve expression titers and increased homogeneity of the antibody post protein A purification as observed by size exclusion chromatography, as shown in FIG. 37.

TABLE 37
Additional Antibodies of the Present Disclosure

| | | | |
|---|---|---|---|
| V120 | B12 | EIVLTQSPGTLSLSPGERATFSCRSSHSIRSRRVAWYQHKPGQAPRLVIHG | SEQ ID |
| (EIP0120) | light | VSNRASGISDRFSGSGSGTDFTLTITRVEPEDFALYYCQVYGASSYTFGQG | NO: 610 |
| B12 mAb | chain | TKLERKRTVAAPSVFIFPPSDEQLKSGTASVVCLLNNFYPREAKVQWKVDN | |
| (Hu-IgG | | ALQSGNSQESVTEQDSKDSTYSLSSTLTLSKADYEKHKVYACEVTHQGLSS | |
| isotype | | PVTKSFNRGEC | |
| control) | B12 | QVQLVQSGAEVKKPGASVKVSCQASGYRFSNFVIHWVRQAPGQRFEWMGWI | SEQ ID |
| | heavy | NPYNGNKEFSAKFQDRVTFTADTSANTAYMELRSLRSADTAVYYCARVGPY | NO: 611 |
| | chain | SWDDSPQDNYYMDVWGKGTTVIVSSASTKGPSVFPLAPSSKSTSGGTAALG | |
| | | CLVKDYFPEPVTVSWNSGALTSGVHTFPAVLQSSGLYSLSSVVTVPSSSLG | |
| | | TQTYICNVNHKPSNTKVDKKVEPKSCDKTHTCPPCPAPEAAGAPSVFLFPP | |
| | | KPKDTLMISRTPEVTCVVVDVSHEDPEVKFNWYVDGVEVHNAKTKPREEQY | |
| | | NSTYRVVSVLTVLHQDWLNGKEYKCKVSNKALPAPIEKTISKAKGQPREPQ | |
| | | VYTLPPSREEMTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| V491 | TAA | EIVLTQSPATLSLSPGERATLSCRASQSIGTSIHWYQKKPGQAPRLLIYFA | SEQ ID |
| (EIP0491) | light | SESISGIPARFSGSGSGTDFTLTISSLEPEDFAVYYCQQSNSWPLYTFGGG | NO: 612 |
| | chain | TKVEIKRTVAAPSVFIFPPSDEQLKSGTASVVCLLNNFYPREAKVQWKVDN | |
| | | ALQSGNSQESVTEQDSKDSTYSLSSTLRLSKADYEKHKVYACEVTHQGLSS | |
| | | PVTKSFNRGEC | |
| | TAA | QVQLVQSGAEVKKPGASVKVSCKASGYTFTNYDINWVRDAPGQGLEWMGWI | SEQ ID |
| | heavy | YPGDGSTYYNEKFKGRVTMTRDTSTSTVYMELSSLRSEDTAVYYCARSGDY | NO: 613 |
| | chain | GLYYFDYWGQGTLVTVSSASTKGPSVFPLAPSSKSTSGGTAALGCLVDDYF | |
| | | PEPVTVSWNSGALTSGVHTFPAVLQSSGLYSLSSVVTVPSSSLGTQTYICN | |
| | | VNHKPSNTKVDKKVEPKSCDKTHTCPPCPAPEAAGAPSVFLFPPKPKDTLM | |
| | | ISRTPEVTCVVVDVSHEDPEVKFNWYVDGVEVHNAKTKPREEQYNSTYRVV | |
| | | SVLTVLHQDWLNGKEYKCKVSNKALPAPIEKTISKAKGQPREPQVYTLPPC | |
| | | REEMTKNQVSLWCLVKGFYPSDIAVEWESNGQPENNYKTTPPVLDSDGSFF | |
| | | LYSKLTVDKSRWQQGNVFSCSVMHEALHNHYTQKSLSLSPG | |
| | CD3 | ELVVTQEPSLTVSPGGTVTLTCRSSTGAVTTSNYANWVQDKPGQAPRGLIG | SEQ ID |
| | light | GTNKRAPGTPARFSGSLLGGKAALTLSGVQPEDEAEYYCALWYSNLWVFGG | NO: 614 |
| | chain | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 | EVQLLESGGGLVQPGGSLKLSCAASGFTFDTYAMNWVRKAPGKGLEWVAKI | SEQ ID |
| | heavy | RSKYNNYATYYADSVKDRFTISRDDSKNTAYLQMNNLKTEDTAVYYCVRHG | NO: 615 |
| | chain | AFGDSYVSWFAYWGQGTLVTVSSASTKGPSVFPLAPSSKSTSGGTAALGCL | |
| | | VKDYFPEPVTVSWNSGALTSGVHTSPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |
| V577 | TAA | EIVLTQSPATLSLSPGERATLSCRASQSIGTSIHWYQKKPGQAPRLLIYFA | SEQ ID |
| (EIP0577) | light | SESISGIPARFSGSGSGTDFTLTISSLEPEDFAVYYCQQSNSWPLYTFGGG | NO: 616 |
| | chain | TKVEIKRTVAAPSVCIFPPSDEQLKSGTASVVCLLNNFYPREAKVQWKVDN | |
| | | ALQSGNSQESVTEQDSKDSTYSLSSTLRLSKADYEKHKVYACEVTHQGLSS | |
| | | PVTKSFNRGES | |
| | TAA | QVQLVQSGAEVKKPGASVKVSCKASGYTFTNYDINWVRDAPGQGLEWMGWI | SEQ ID |
| | heavy | YPGDGSTYYNEKFKGRVTMTRDTSTSTVYMELSSLRSEDTAVYYCARSGDY | NO: 617 |
| | chain | GLYYFDYWGQGTLVTVSSASTKGPSVFPLAPSSKCTSGGTAALGCLVDDYF | |
| | | PEPVTVSWNSGALTSGVHTFPAVLQSSGLYSLSSVVTVPSSSLGTQTYICN | |
| | | VNHKPSNTKVDKKVEPKSSDKTHTCPPCPAPEAAGAPSVFLFPPKPKDTLM | |
| | | ISRTPEVTCVVVDVSHEDPEVKFNWYVDGVEVHNAKTKPREEQYNSTYRVV | |
| | | SVLTVLHQDWLNGKEYKCKVSNKALPAPIEKTISKAKGQPREPQVYTLPPC | |
| | | REEMTKNQVSLWCLVKGFYPSDIAVEWESNGQPENNYKTTPPVLDSDGSFF | |
| | | LYSKLTVDKSRWQQGNVFSCSVMHEALHNHYTQKSLSLSPG | |
| | CD3 | ELVVTQEPSLTVSPGGTVTLTCRSSTGAVTTSNYANWVQDKPGQAPRGLIG | SEQ ID |
| | light | DTNKRAPGTPARFSGSLLGGKAALTLSGVQPEDEAEYYCALWYSNLWVFGG | NO: 618 |
| | chain | GTKLTVLGQPKANPTVTLFPPSSEELQANKADLVCLISDFYPGAVTVAWKA | |
| | | DGSPVKAGVETCKPSKQSNNKYAASSYLSLTPEQWKSHRSYSCQVTHEGST | |
| | | VEKTVAPTESS | |
| | CD3 | EVQLLESGGGLVQPGGSLKLSCAASGFTFNTYAMNWVRKAPGKGLEWVARI | SEQ ID |
| | heavy | RSKYNNYATYYADSVKDRFTISRDDSKNTAYLQMNNLKTEDTAVYYCVRHG | NO: 619 |
| | chain | NFGDDYVSWFAYWGQGTLVTVSSASTKGPSVFPLAPSSKSTSGGTAALGCL | |
| | | VKDYFPEPVTVSWNSGALTSGVHTSPACLQSSGLYSLSSVVTVPSSSLGTQ | |
| | | TYICNVNHKPSNTKVDKKVEPKSSDKTHTCPPCPAPEAAGAPSVFLFPPKP | |
| | | KDTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQVC | |
| | | TLPPSREEMTKNQVSLSCAVKGFYPSDIAVEWESNGQPENNYKTTPPVLDS | |
| | | DGSFFLVSKLTVDKSRWQQGNVFSCSVMHEALHNHYTQKSLSLSPG | |

### Example 9: In Vivo Characterization of Bispecific Antibodies with Fusion Peptides

### Expansion of T Cells for In Vivo Studies

T-cells were isolated from healthy PBMCs donors using StemCell T-cell isolation kit. OpTmizer media was supplemented with 2% human AB serum, 1× penicillin/streptomycin, 4 mM GlutaMAX, 2 mM Glutamine. Cells were incubated at 37° C. in 5% CO2 in the supplemented OpTmizer media at 5×10⁵ cells/mL and T cells were treated with αCD3 and αCD28 antibody coated Dynabeads at a ratio of 25 μL dynabeads per million cells for 48 hours. Following removal of Dynabeads, T cells were incubated with IL-7 (10 ng/ml) and IL-15 (10 ng/ml) in supplemented OpTmizer media for an additional 6 days. The supplemented media was replaced with freshly thawed cytokines (IL7 and IL15) every 48 hours. On day of adoptive transfer or Co-engraftment, the T-cells are harvested and a hemocytometer was used to determine cell number and viability. T cells were suspended in cold PBS and stored on ice for no longer than 30 minutes until injection.

Tumor Studies with Adoptive Human T Cell Transfer

SiHa tumor cells were initially cultured from frozen stock in an DMEM with 10% fetal bovine serum (FBS) media and incubated at 37° C. in 5% CO2 in treated cell culture flasks. Cells were then split every 2-3 days pending the cell density in the culture flasks. On the days of implantation cells were dissociated from the flask using TrypLE Select and washed twice with PBS. Five million SiHa cells resuspended in PBS and Matrigel at a 1:1 ratio were implanted subcutaneously into flank of NSG mice (NOD.Cg-Prkdc^{scid} Il2rg^{tm1Wjl}/SzJ, Jackson Laboratory) with each treatment cohort containing 8-10 mice. Tumor growth was monitored using electronic calipers and tumor volume were calculated according to the formula: 0.52×(length× width²). When the tumors reached average size of 100 mm³, mice were randomized into following treatment groups where each group had similar mean tumor volumes with each group receiving Control-αCD3 (EIP0546), αULBP2-αCD3 (EIP0205) and αULBP2-αCD3-CD58 (EIP0359) at a dose level of 4 mg/kg) by tail vein infusion on Day 21 after the initial implantation. The following day (Day 22) all mice infused with 5 million activated human T cells. Mice were then dosed with bispecifics and control twice a week for 3 weeks. A second infusion of 5 million human T cells was performed on Day 44 and the following day (Day 45), mice received final dose of antibody.

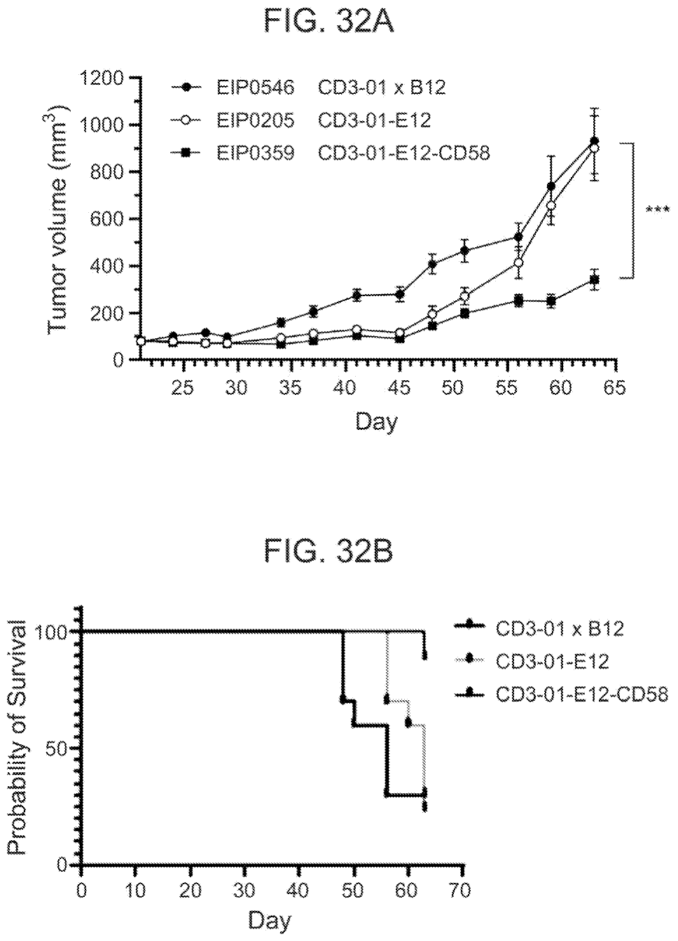

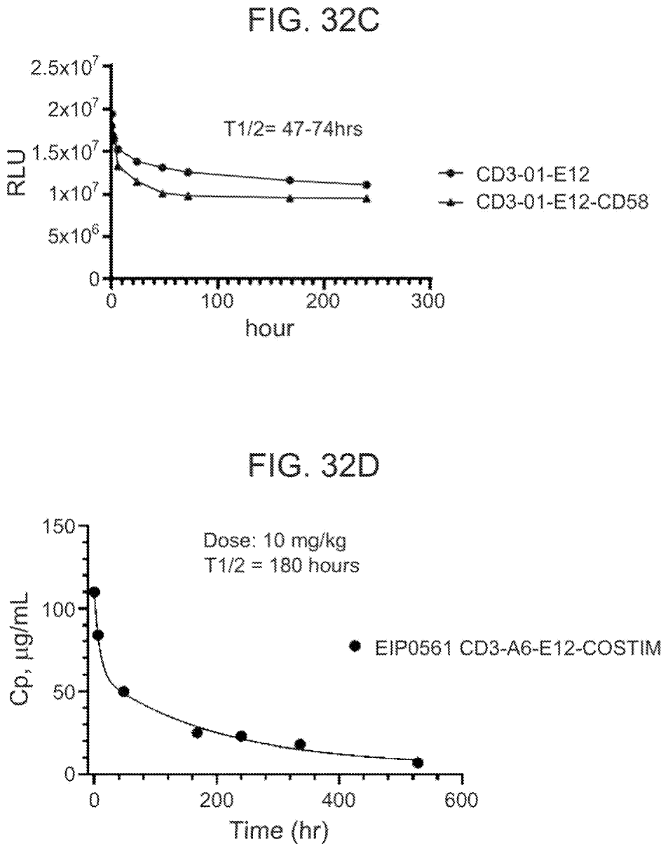

Both the bispecific αULBP2-αCD3 (EIP0205) and the bispecific fusion αULBP2-αCD3-CD58 (EIP0359) were evaluated in NSG mice engrafted with cervical cancer cell line SiHa followed by adoptive transfer of activated human T cells (FIGS. 32A-32C). In comparison to the control antibody, Control-αCD3 (EIP0546), both αULBP2-αCD3 (EIP0205) and αULBP2-αCD3-CD58 (EIP0359) inhibited tumor growth from the beginning of treatment (Day 21) to Day 45. Subsequently, tumors in the αULBP2-αCD3 treatment group expanded rapidly reaching similar size as the control group (900 mm³) whereas in the ULBP2-αCD3-CD58 treated group, tumors remained small and reaching only 350 mm³ at the end of study.

Pharmacodynamic Study with Tumor and Human T Cell Co-Engraftment

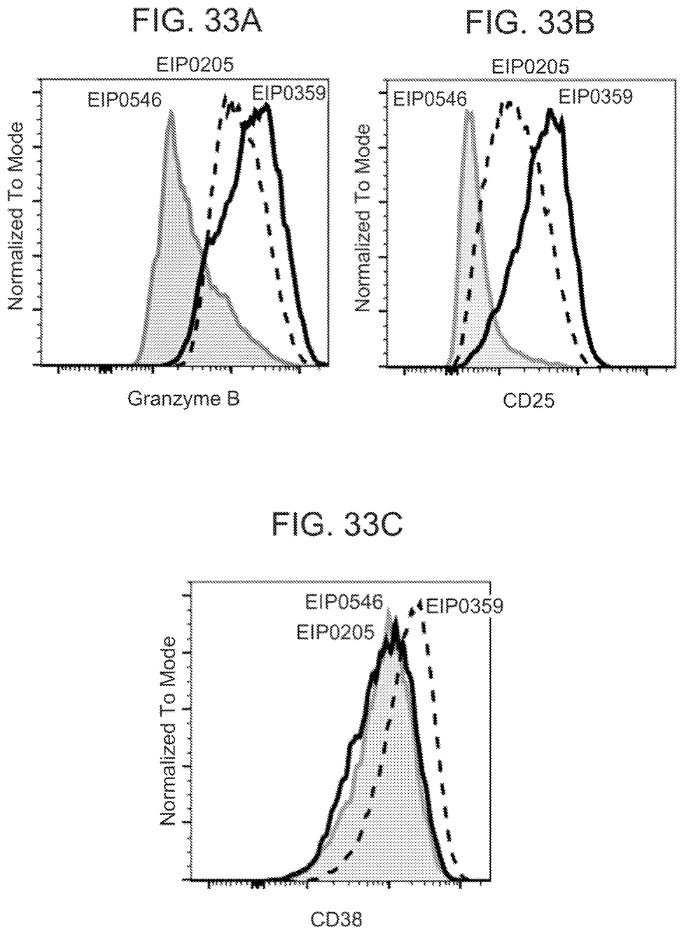

A pharmacodynamic study was performed in which SiHa cells were co-engrafted with activated human T cells into NSG mice and treated with either the bispecific αULBP2-αCD3 (EIP0205) or the bispecific fusion αULBP2-αCD3-CD58 (EIP0359) on the same day. Eight million SiHa cells and 1.6 million activated human T cells were implanted subcutaneously into flank of 9 NSG mice. The mice were then separated into 3 groups and treated with the following agents: Control-αCD3 (EIP0546), αULBP2-αCD3 (EIP0205) and αULBP2-αCD3-CD58 (EIP0359) at a dose of 4 mg/kg by tail vein infusion immediately after co-engraftment. Three days later, all tumors from same treatment group were harvested and pooled prior to tumor dissociation. Single cell suspensions were then subjected to magnetic enrichment using human CD45 beads (Miltenyi) before analysis by flow cytometry using a panel of fluorescently labeled antibodies that bind to markers associated with changes in T cell phenotypes of activation, effector activity and exhaustion. As shown in FIGS. 33A-33C, the bispecific fusion αULBP2-αCD3-CD58

(EIP0359), in comparison to the bispecific αULBP2-αCD3 (EIP0205) induced higher levels of granzyme B (FIG. 33A) and CD25 (FIG. 33B) while maintaining baseline CD3ε (FIG. 33C) levels in CD8 T cells. The difference observed between αULBP2-αCD3 and αULBP2-CD3-CD58 for these 3 markers is similar to what was previously observed in vitro (FIG. 31).

### Pharmacokinetics of EIP0561 in Cynomolgus Monkey

Nine cynomolgus monkeys were infused intravenously with EIP0561 with three dose levels (0.25, 1.0 and 4.0 mg/kg) and three animals per dose. Blood samples were collected at 0.03 0.5, 2, 8, 24, 48, 72 and 168 hours and the concentrations of EIP0561 in the plasma were determined using sandwich ELISA (Table 37). Toxicokinetic parameters were also determined and shown in Table 38.

TABLE 37
Concentration of EIP0561 per animal by time determined by sandwich ELISA.

| | | Concentration of EIP0561 (ng/mL) | | | | | | | | |
|---|---|---|---|---|---|---|---|---|---|---|
| | | Animal | | | | | | | | |
| | | 1 | 2 | 3 | 4 | 5 | 6 | 7 | 8 | 9 |
| | | Dose (mg/kg) | | | | | | | | |
| | | 4 | 4 | 4 | 1 | 1 | 1 | 0.25 | 0.25 | 0.25 |
| Hours | 0.03 | 81464.10 | 68319.68 | 99275.40 | 10589.05 | 8802.04 | 9741.76 | 8142.17 | 11274.85 | 4894.59 |
| | 0.5 | 56244.09 | 47039.75 | 66148.94 | 8545.70 | 7136.46 | 7303.50 | 5548.27 | 6592.56 | 4252.74 |
| | 2 | 48887.20 | 35872.52 | 52869.86 | 5794.66 | 5139.11 | 6280.68 | 4261.61 | 4378.18 | 3430.11 |
| | 8 | 35565.95 | 24675.93 | 34148.58 | 4633.99 | 4218.88 | 4881.90 | 3133.65 | 3275.44 | 2396.57 |
| | 24 | 24764.87 | 17164.71 | 20750.34 | 3467.28 | 2932.91 | 3300.65 | 1994.15 | 1851.72 | 1614.14 |
| | 48 | 16856.31 | 12076.54 | 13603.01 | 2828.00 | 2453.84 | 2626.66 | 1379.33 | 1420.43 | 1325.91 |
| | 72 | 13605.72 | 10020.37 | 11453.64 | 2281.96 | 2150.08 | 2287.76 | 1136.70 | 1093.01 | 1193.81 |
| | 168 | 11432.18 | 7092.89 | 7915.94 | 1683.74 | 1575.06 | 1744.24 | 850.77 | 859.33 | 940.09 |

TABLE 38
Toxicokinetic parameters. Abbreviation and description for each parameter are described below.

| | Dose | AUCLST | C0 | CMAX | CLST | LAMZHLD | CLO | VSSO | VZO |
|---|---|---|---|---|---|---|---|---|---|
| Animal | (mg/kg) | (ug/L*h) | (ug/L) | (ug/L) | (ug/L) | (days) | (L/h) | (L) | (L) |
| 1 | 4 | 2898210 | 83413.4 | 81464.1 | 11432.2 | 10.2079 | 5.76E−07 | 1.91E−04 | 2.04E−04 |
| 2 | 4 | 2026410 | 69966.7 | 68319.7 | 7092.89 | 6.88294 | 1.08E−06 | 2.36E−04 | 2.56E−04 |
| 3 | 4 | 2443040 | 101882 | 99275.4 | 7915.94 | 6.68206 | 9.36E−07 | 1.92E−04 | 2.17E−04 |
| 4 | 1 | 436206 | 10735 | 10589 | 1683.74 | 6.12165 | 1.26E−06 | 2.63E−04 | 2.67E−04 |
| 5 | 1 | 394713 | 8920.68 | 8802.04 | 1575.06 | 8.1006 | 1.20E−06 | 3.22E−04 | 3.35E−04 |
| 6 | 1 | 434449 | 9922.54 | 9741.76 | 1744.24 | 8.9024 | 1.03E−06 | 3.03E−04 | 3.17E−04 |
| 7 | 0.25 | 237956 | 8343.98 | 8142.17 | 850.772 | 7.72519 | 5.37E−07 | 1.32E−04 | 1.44E−04 |
| 8 | 0.25 | 237592 | 11667.7 | 11274.9 | 859.332 | 5.95392 | 6.03E−07 | 1.18E−04 | 1.24E−04 |
| 9 | 0.25 | 224322 | 4938.71 | 4894.59 | 940.091 | 10.4719 | 4.42E−07 | 1.54E−04 | 1.6E−04 |

AUCLST (ug/L*h), area under the curve from the time of dosing to the time of the last observation; C0 (ug/L), concentration at first time point; CMAX (ug/L), maximal concentration, occurring at TMAX; CLST (ug/L), concentration at last time point; LAMZHLD (days), terminal half-life (days); CLO (L/h), total body clearance based on CLST; VSSO (L), estimated volume of distribution at steady state based on CLST; VZO (L), volume of distribution associated with the terminal phase, based on CLST.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody comprising the following structure:
a. a first heavy chain polypeptide (H1) comprising a variable region (VH1), and a constant region (CH1) having a constant region 1 domain (CHI_{H1}), a hinge region (H1H), a constant region 2 domain (CH1_{H2}) and a constant region 3 domain (CH1_{H3}); and a first light chain polypeptide (L1) comprising a variable region (VL1) and a constant region (CL1),
b. a second heavy chain polypeptide (H2) comprising a variable region (VH2), and a constant region (CH2) having a constant region 1 domain (CH2_{H1}), a hinge region (H2H), a constant region 2 domain (CH2_{H2}) and a constant region 3 domain (CH2_{H3}); and second light chain polypeptide (L2) comprising a variable region (VL2) and a constant region (CL2),
wherein
i. the amino acid at positions 39 (Kabat numbering) of the VH1 and VH2 are charged or polar amino acid residues and the amino acid at positions 38 (Kabat numbering) of the VL1 and VL2 are an oppositely charged or polar amino acid residue compared to the amino acids at positions 39 of the VH1 and the VH2; or the amino acid at positions 100 of the VH1 and VH2 (Kabat numbering) are charged or polar amino acid residues and the amino acid at positions 44 (Kabat numbering) of the VL1 and VL2 are an oppositely charged or polar amino acid residue compared to the amino acids at positions 100 of the VH1 and the VH2 (Kabat numbering);
ii. the amino acid at positions 147 of the CHI_{H1} and the CH1_{H2} (EU numbering) are charged or polar amino acid residues and one of the amino acids at positions 131, 179 or 180 of the CL1 or CL2 (EU numbering) is an oppositely charged or polar amino acid residue compared to the amino acids at positions 147 of the CHI_{H1} and the CH1_{H2} (EU numbering);
iii. the amino acid at positions 185 of the CH1_{H1} and the CH1_{H2} (EU numbering) are charged or polar amino acid residues and the amino acid at positions 137 of the CL1 and CL2 (EU numbering) are an oppositely charged or polar amino acid residue compared to the amino acids at positions 185 of the CHI_{H1} and the CH1_{H2} (EU numbering); or the amino acid at positions 187 of the CH1_{H1} and the CH1_{H2} (EU numbering) are charged or polar amino acid residues and one of the amino acids at positions 137 or 138 of the CL1 and CL2 (EU numbering) is an oppositely charged or polar amino acid residue compared to the amino acids at positions 187 of the CHI_{H1} and the CH1_{H2} (EU numbering); and
iv. the amino acid at positions 145 of the CH1_{H1} and the CH1_{H2} (EU numbering) are charged or polar amino acid residues and the amino acids at position 131 of the CL1 and CL2 (EU numbering) are oppositely charged or polar amino acid residues compared to the amino acids at positions 145 of the CHI_{H1} and the CH1_{H2}, (EU numbering).

2. The antibody of claim 1, wherein the charged amino acid residue is a naturally occurring amino acid or a non-naturally occurring amino acid.

3. The antibody of claim 1, wherein the naturally occurring charged amino acid residue is an arginine, a lysine, a histidine, a glutamic acid or an aspartic acid.

4. The antibody of claim 1, wherein the H1 amino acids at position 39, 100, 147, 185, 187 or 145 are positively charged and the L1 amino acids at positions 38, 44, 131, 179, 180, 137 or 138 are negatively charged; and the H2 amino acids at position 39, 100, 147, 185, 187 or 145 are negatively charged and the L2 amino acids at positions 38, 44, 131, 179, 180, 137 or 138 are positively charged.

5. The antibody of claim 1, wherein the HI amino acids at position 39, 100, 147, 185, 187 and 145 are negatively charged and the L1 amino acids at positions 38, 44, 131, 179, 180, 137 or 138 are positively charged; and the H2 amino acids at position 39, 100, 147, 185, 187 or 145 are positively charged and the L2 amino acids at positions 38, 44, 131, 179, 180, 137 or 138 are negatively charged.

6. The antibody of any one of the preceding claims, wherein L1 and L2 are lambda or kappa light chains.

7. The antibody of claim 6, wherein L1 is a lambda light chain and L2 is a kappa light chain; or L1 is a kappa light chain and L2 is a lambda light chain.

8. The antibody of any one of the preceding claims, wherein the antibody has an IgG1, an IgG2, or an IgG4 isotype.

9. The antibody of any one of the preceding claims, wherein the antibody is a chimeric antibody, a human antibody, or a humanized antibody.

10. The antibody of any one of the preceding claims, wherein the antibody is a bispecific antibody.

11. The antibody of claim 10, wherein the bispecific antibody comprises
i) a first antigen binding domain that binds to a cell surface antigen, wherein the cell surface antigen is expressed on a T-cell, a NK cell, a neutrophil, a B cell or a dendritic cell engager; and
ii) a second antigen binding domain that binds to a disease associated antigen (DAA).

12. The antibody of claim 11, wherein the cell surface antigen is expressed on a T-cell.

13. The antibody of claim 12, wherein the cell surface antigen expressed on a T-cell is a CD3.

14. The antibody of claim 11, wherein the DAA is a UL16 Binding Protein 2 (ULBP2) or is encoded by the gene RAET1G (UL16 Binding Protein 5; ULBP5) or is encoded by the gene RAET1L (UL16 Binding Protein 6; ULBP6).

15. The antibody of any one of the preceding claims, wherein a polypeptide is fused to the N-terminus or the C-terminus of H1 and/or H2.

16. The antibody of claim 15, wherein the polypeptide is a CD58, a IL-17 or a fragment thereof.

17. The antibody of claim 15, wherein the polypeptide is fused via a linker peptide.

18. The antibody of claim 17, wherein the linker peptide is at least 5, at least 6, at least 7, at least 8, at least 9, at least 10, at least 11, at least 12, at least 13, at least 14, at least 15, at least 16, at least 17, at least 18, at least 19 or at least 20 amino acid residues in length.

19. The antibody of claim 17, wherein the linker peptide comprises the amino acid sequence of SEQ ID NOS: 52-54.

20. The antibody of any one of the preceding claims, wherein:
i) the amino acid at position 87 (Kabat numbering) of the VH1 and/or VH2 is a G; and
ii) the amino acid at position 45 (Kabat numbering) of the VL1 and/or VL2 is a W.

21. The antibody of any one of the preceding claims, wherein:
i) the CH1_{H3} has a C at position 349, an S at position 366, an A at position 368 and a V at position 407 (EU numbering); and the CH2_{H3} has a C at position 354 and a W at position 366 (EU numbering);
ii) the CH2_{H3} has a C at position 349, an S at position 366, an A at position 368 and a V at position 407 (EU numbering); and the CH1_{H3} has a C at position 354 and a W at position 366 (EU numbering);
iii) the CH1_{H3} has a C at position 354, an S at position 366, an A at position 368 and a V at position 407 (EU numbering); and the CH2_{H3} has a C at position 349 and a W at position 366 (EU numbering); or
iv) the CH2_{H3} has a C at position 354, an S at position 366, an A at position 368 and a V at position 407 (EU numbering); and the CH1_{H3} has a C at position 349 and a W at position 366 (EU numbering).

22. The antibody of any one of the preceding claims, further comprising an amino acid at position 446 (EU numbering) and/or at position 447 (EU numbering) of the CH1_{H3} and/or of the CH2_{H3}; optionally wherein the amino acid at position 446 (EU numbering) is a G; and optionally wherein the amino acid at position 447 (EU numbering) is a K.

23. The antibody of any one of the preceding claims, wherein:
i) the H1H and/or the H2H has an A at positions 234 and 235 (EU numbering);
ii) the H1H and/or the H2H has an A at positions 234, 235 and 237 (EU numbering); or
iii) the H1H and/or the H2H has an A at positions 234 and 235 and G at position 329 (EU numbering).

24. The antibody of any one of the preceding claims, wherein
i) the CH1_{H3} and/or the CH2_{H3} has an A at position 297 (EU numbering);
ii) the CH1_{H3} and/or the CH2_{H3} has a G at position 297 (EU numbering); or
iii) the CH1_{H3} and/or the CH2_{H3} has a S at position 297 (EU numbering).

25. The antibody of any one of the preceding claims, wherein the CH1_{H3} and/or the CH2_{H3} has an S at position 331 (EU numbering).

26. The antibody of any one of the preceding claims, wherein
i) the CH1_{H2} and/or the CH2_{H2} has an C at position 370 (Kabat numbering); and
ii) the CH1_{H2} and/or the CH2_{H2} has an C at position 375 (Kabat numbering).

27. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 185 (EU numbering) of the CH1_{H1} is a K and the amino acid at position 137 (EU numbering) of the CL1 is a D;
iii. the amino acid at position 128 (EU numbering) of the CH1_{H1} is a C and the amino acid at position 118 (EU numbering) of the CL1 is a C; and
iv. the amino acid at position 220 (EU numbering) in the H1H is a S and
the amino acid at position 214 (EU numbering) of the CL1 is a S; and
b) the H2 and the L2 comprise the following:
i. the amino acid at position 39 (Kabat numbering) of the VH2 is a D and the amino acid at position 38 (Kabat numbering) of the VL2 is a K; and
ii. the amino acid at position 147 (EU numbering) of the CH2_{H1} is a D and the amino acid at position 180 (EU numbering) of the CL2 is a R.

28. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D; and
ii. the amino acid at position 185 (EU numbering) of the CH1_{H1} is a K and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 147 (EU numbering) of the CH2_{H1} is a D and the amino acid at position 180 (EU numbering) of the CL2 is a R;
iii. the amino acid at position 134 (EU numbering) of the CH2_{H1} is a C and the amino acid at position 116 (EU numbering) of the CL2 is a C; and
iv. the amino acid at position 220 (EU numbering) in the H2H is a S and
the amino acid at position 214 (EU numbering) of the CL2 is a S.

29. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D; and
ii. the amino acid at position 185 (EU numbering) of the CHI_{H1} is a K and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 147 (EU numbering) of the CH2_{H1} is a D and the amino acid at position 180 (EU numbering) of the CL2 is a R;
iii. the amino acid at position 136 (EU numbering) of the CH2_{H1} is a C and the amino acid at position 114 (EU numbering) of the CL2 is a C; and
iv. the amino acid at position 220 (EU numbering) in the H2H is a S and
the amino acid at position 214 (EU numbering) of the CL2 is a S.

30. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 147 (EU numbering) of the CH1_{H1} is a K and the amino acid at position 131 (EU numbering) of the CL1 is a D;
iii. the amino acid at position 173 (EU numbering) of the CH1_{H1} is a C and the amino acid at position 162 (EU numbering) of the CL1 is a C;
iv. the amino acid at position 220 (EU numbering) in the H1H is a S and
the amino acid at position 214 (EU numbering) of the CL1 is an S; and
b) the H2 and the L2 comprise the following:
i. the amino acid at position 39 (Kabat numbering) of the VH2 is a D and the amino acid at position 38 (Kabat numbering) of the VL2 is a K; and
ii. the amino acid at position 147 (EU numbering) of the CH2_{H1} is a D and the amino acid at position 180 (EU numbering) of the CL2 is a R.

31. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 185 (EU numbering) of the CHI_{H1} is a K and the amino acid at position 137 (EU numbering) of the CL1 is a D;
iii. the amino acid at position 173 (EU numbering) of the CH1_{H1} is a C and the amino acid at position 162 (EU numbering) CL1 is a C; and
iv. the amino acid at position 220 (EU numbering) in the H1H is a S and
the amino acid at position 214 (EU numbering) of the CL1 is a S; and
b) the H2 and the L2 comprise the following:
i. the amino acid at position 39 (Kabat numbering) of the VH2 is a D and the amino acid at position 38 (Kabat numbering) of the VL2 is a K; and
ii. the amino acid at position 147 (EU numbering) of the CH2_{H1} is a D and the amino acid at position 180 (EU numbering) of the CL2 is a R;

32. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D; and
ii. the amino acid at position 185 (EU numbering) of the CH1_{H1} is a K and the amino acid at position 137 (EU numbering) CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 147 (EU numbering) of the CH2_{H1} is a D and the amino acid at position 180 (EU numbering) of the CL2 is a R;
iii. the amino acid at position 131 (EU numbering) of the CH2_{H1} is a C and the amino acid at position 114 (EU numbering) of the CL2 is a C; and
iv. the amino acid at position 220 (EU numbering) in the H2H is a S and
the amino acid at position 214 (EU numbering) of the CL2 is a S.

33. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D; and
ii. the amino acid at position 185 (EU numbering) of the CH1_{H1} is a K and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 138 (EU numbering) of the CL2 is a K;
iii. the amino acid at position 170 (EU numbering) of the CH2_{H1} is a C and the amino acid at position 162 EU numbering) of the CL2 is a C; and
iv. the amino acid at position 220 (EU numbering) in the H2H is a S and
the amino acid at position 214 (EU numbering) of the CL2 is a S.

34. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 185 (EU numbering) of the CH1_{H1} is a E, the amino acid at position 137 (EU numbering) of the CL1 is a K; and
iii. the amino acid at position 179 (EU numbering) of the CL1 is a E; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 138 (EU numbering) of the CL2 is a K;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

35. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D; and
ii. the amino acid at position 185 (EU numbering) of the CHI_{H1} is a K and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 138 (EU numbering) of the CL2 is a K;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

36. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 185 (EU numbering) of the CHI_{H1} is a E and the amino acid at position 137 (EU numbering) of the CL1 is a K; and
iii. the amino acid at position 179 (EU numbering) of the CL1 is a E; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 147 (EU numbering) of the CH2_{H1} is a D and the amino acid at position 180 (EU numbering) of the CL2 is a R;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

37. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D; and
ii. the amino acid at position 185 (EU numbering) of the CH1_{H1} is a K and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 147 (EU numbering) of the CH2_{H1} is a D and the amino acid at position 180 (EU numbering) of the CL2 is a R;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

38. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 147 (EU numbering) of the CHI_{H1} is a K and the amino acid at position 131 (EU numbering) of the CL1 is a D;
iii. the amino acid at position 185 (EU numbering) of the CH1_{H1} is a K and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 147 (EU numbering) of the CH2_{H1} is a D and the amino acid at position 180 (EU numbering) of the CL2 is a R;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and the amino acid at position 214 (EU numbering) of the CL2 is a S.

39. The antibody of claim 1, wherein
a) the H1 and the L1 comprise the following:
i. the amino acid at position 39 (Kabat numbering) of the VH1 is a D and the amino acid at position 38 (Kabat numbering) of the VL1 is a K; and
ii. the amino acid at position 185 (EU numbering) of the CH1_{H1} is a E and the amino acid at position 137 (EU numbering) of the CL1 is a K; and
b) the H2 and the L2 comprise the following:
i. the amino acid at position 39 (Kabat numbering) of the VH2 is a K and the amino acid at position 38 (Kabat numbering) of the VL2 is a D;
ii. the amino acid at position 187 (EU numbering) of the CH2_{H1} is a D and the amino acid at position 138 (EU numbering) of the CL2 is a K;
iii. the amino acid at position 136 (EU numbering) of the CH2_{H1} is a C and the amino acid at position 114 (EU numbering) of the CL2 is a C; and
iv. the amino acid at position 220 (EU numbering) in the H2H is a S and
the amino acid at position 214 (EU numbering) of the CL2 is a S.

40. The antibody of claim 1, wherein
a) the H1 and the L1 comprise the following:
i. the amino acid at position 39 (Kabat numbering) of the VH1 is a D and the amino acid at position 38 (Kabat numbering) of the VL1 is a K; and
ii. the amino acid at position 185 (EU numbering) of the CHI_{H1} is a E and the amino acid at position 137 (EU numbering) of the CL1 is a K; and
b) the H2 and the L2 comprise the following:
i. the amino acid at position 39 (Kabat numbering) of the VH2 is a K and the amino acid at position 38 (Kabat numbering) of the VL2 is a D;
ii. the amino acid at position 187 (EU numbering) of the CH2_{H1} is a D and the amino acid at position 138 (EU numbering) of the CL2 is a K;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

41. The antibody of claim 1, wherein
a) the H1 and the L1 comprise the following:
i. the amino acid at position 39 (Kabat numbering) of the VH1 is a D and the amino acid at position 38 (Kabat numbering) of the VL1 is a K; and
ii. the amino acid at position 185 (EU numbering) of the CH1_{H1} is a K and the amino acid at position 137 (EU numbering) of the CL1 is a D; and
b) the H2 and the L2 comprise the following:
i. the amino acid at position 39 (Kabat numbering) of the VH2 is a K and the amino acid at position 38 (Kabat numbering) of the VL2 is a D;
ii. the amino acid at position 187 (EU numbering) of the CH2_{H1} is a D and the amino acid at position 138 (EU numbering) of the CL2 is a K;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is an S.

42. The antibody of claim 1, wherein
a) the H1 and the L1 comprise the following:
i. the amino acid at position 39 (Kabat numbering) of the VH1 is a D and the amino acid at position 38 (Kabat numbering) of the VL1 is a K;
ii. the amino acid at position 147 (EU numbering) of the CH1_{H1} is a K and the amino acid at position 131 (EU numbering) of the CL1 is a D;
iii. the amino acid at position 185 (EU numbering) of the CH1_{H1} is a E and the amino acid at position 137 (EU numbering) of the CL1 is a K; and
iv. the amino acid at position 145 (EU numbering) of the CH1_{H1} is a S and the amino acid at position 180 (EU numbering) of the CL1 is a E; and
b) the H2 and the L2 comprise the following:
i. the amino acid at position 39 (Kabat numbering) of the VH2 is a K and the amino acid at position 38 (Kabat numbering) of the VL2 is a D;
ii. the amino acid at position 187 (EU numbering) of the CH2_{H1} is a D and the amino acid at position 138 (EU numbering) of the CL2 is a K;
iii. the amino acid at position 170 (EU numbering) of the VH2 is a C and the amino acid at position 162 (EU numbering) of the VL2 is a C; and
iv. the amino acid at position 220 (EU numbering) in the H2H is a S and
the amino acid at position 214 (EU numbering) of the CL2 is a S.

43. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 147 (EU numbering) of the CH1_{H1} is a D and the amino acid at position 131 (EU numbering) of the CL1 is a K; and
iii. the amino acid at position 185 (EU numbering) of the CH1_{H1} is a E and the amino acid at position 137 (EU numbering) of the CL1 is a D; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 137 (EU numbering) of the CL2 is a K;
iii. the amino acid at position 138 (EU numbering) of the CL2 is a R;
iv. the amino acid at position 170 (EU numbering) of the CH2_{H1} is a C and the amino acid at position 162 (EU numbering) of the CL2 is a C; and
V. the amino acid at position 220 (EU numbering) in the H2H is a S and
the amino acid at position 214 (EU numbering) of the CL2 is a S.

44. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 147 (EU numbering) of the CH1_{H1} is a D and the amino acid at position 131 (EU numbering) of the CL1 is a K; and
iii. the amino acid at position 145 (EU numbering) of the CH1_{H1} is a S; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 138 (EU numbering) of the CL2 is a K;
iii. the amino acid at position 170 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

45. The antibody of claim 1, wherein
a) the H1 and the L1 comprise the following:
i. the amino acid at position 39 (Kabat numbering) of the VH1 is a D and the amino acid at position 38 (Kabat numbering) of the VL1 is a K;
ii. the amino acid at position 147 (EU numbering) of the CH1_{H1} is a K and the amino acid at position 131 (EU numbering) of the CL1 is a D; and
iii. the amino acid at position 185 (EU numbering) of the CH1_{H1} is a E and the amino acid at position 137 (EU numbering) of the CL1 is a K; and
b) the H2 and the L2 comprise the following:
i. the amino acid at position 39 (Kabat numbering) of the VH2 is a K and the amino acid at position 38 (Kabat numbering) of the VL2 is a D;
ii. the amino acid at position 147 (EU numbering) of the CH2_{H1} is a D and the amino acid at position 180 (EU numbering) of the CL2 is a R;
iii. the amino acid at position 136 (EU numbering) of the CH2_{H1} is a C and the amino acid at position 114 (EU numbering) of the CL2 is a C; and
iv. the amino acid at position 220 (EU numbering) in the H2H is a S and
the amino acid at position 214 (EU numbering) of the CL2 is an S.

46. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a E;
ii. the amino acid at position 147 (EU numbering) of the CH1_{H1} is a K and the amino acid at position 131 (EU numbering) of the CL1 is a D; and
iii. the amino acid at position 185 (EU numbering) of the CH1_{H1} is a E and the amino acid at position 137 (EU numbering) of the CL1 is a K; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 138 (EU numbering) of the CL2 is a K;
iii. the amino acid at position 136 (EU numbering) of the CH2_{H1} is a C and the amino acid at position 114 (EU numbering) of the CL2 is a C; and
iv. the amino acid at position 220 (EU numbering) in the H2H is a S and
the amino acid at position 214 (EU numbering) of the CL2 is a S.

47. The antibody of claim 1, wherein
a) the H1 and the L1 comprise the following:
i. the amino acid at position 39 (Kabat numbering) of the VH1 is a K and the amino acid at position 38 (Kabat numbering) of the VL1 is a D;
ii. the amino acid at position 147 (EU numbering) of the CHI_{H1} is a D and the amino acid at position 131 (EU numbering) of the CL1 is a K; and
iii. the amino acid at position 185 (EU numbering) of the CH1_{H1} is a D and the amino acid at position 137 (EU numbering) of the CL1 is a K; and
b) the H2 and the L2 comprise the following:
i. the amino acid at position 39 (Kabat numbering) of the VH2 is a D and the amino acid at position 38 (Kabat numbering) of the VL2 is a K;
ii. the amino acid at position 187 (EU numbering) of the CH2_{H1} is a D and the amino acid at position 138 (EU numbering) of the CL2 is a K;
iii. the amino acid at position 171 (EU numbering) of the CH2_{H1} is a C and the amino acid at position 162 (EU numbering) of the CL2 is a C; and
iv. the amino acid at position 220 (EU numbering) in the H2H is a S and
the amino acid at position 214 (EU numbering) of the CL2 is a S.

48. The antibody of any one of claims 27-47, wherein:
i) the amino acid at position 87 (Kabat numbering) of the VH1 and/or VH2 is a G; and
ii) the amino acid at position 45 (Kabat numbering) of the VL1 and/or VL2 is a W.

49. The antibody of any one of claims 27-48, wherein:
i) the CH1_{H3} has a C at position 349, an S at position 366, an A at position 368 and a V at position 407 (EU numbering); and the CH2_{H3} has a C at position 354 and a W at position 366 (EU numbering);
ii) the CH2_{H3} has a C at position 349, an S at position 366, an A at position 368 and a V at position 407 (EU numbering); and the CH1_{H3} has a C at position 354 and a W at position 366 (EU numbering);
iii) the CH1_{H3} has a C at position 354, an S at position 366, an A at position 368 and a V at position 407 (EU numbering); and the CH2_{H3} has a C at position 349 and a W at position 366 (EU numbering); or
iv) the CH2_{H3} has a C at position 354, an S at position 366, an A at position 368 and a V at position 407 (EU numbering); and the CH1_{H3} has a C at position 349 and a W at position 366 (EU numbering).

50. The antibody of any one of claims 27-49, further comprising an amino acid at position 446 (EU numbering) and/or at position 447 (EU numbering) of the CH1_{H3} and/or of the CH2_{H3}; optionally wherein the amino acid at position 446 (EU numbering) is a G; and optionally wherein the amino acid at position 447 (EU numbering) is a K

51. The antibody of any one of claims 27-50, wherein:
i) the H1H and/or the H2H has an A at positions 234 and 235 (EU numbering);
ii) the H1H and/or the H2H has an A at positions 234, 235 and 237 (EU numbering); or
iii) the H1H and/or the H2H has an A at positions 234 and 235 and G at position 329 (EU numbering).

52. The antibody of any one of claims 27-51, wherein
i) the CH1_{H3} and/or the CH2_{H3} has an A at position 297 (EU numbering);
ii) the CH1_{H3} and/or the CH2_{H3} has a G at position 297 (EU numbering); or
iii) the CH1_{H3} and/or the CH2_{H3} has a S at position 297 (EU numbering).

53. The antibody of any one of claims 27-52, wherein the CH1_{H3} and/or the CH2_{H3} has an S at position 331 (EU numbering).

54. The antibody of any one of claims 27-51, wherein
i) the CH1_{H2} and/or the CH2_{H2} has an C at position 370 (Kabat numbering); and
ii) the CH1_{H2} and/or the CH2_{H2} has an C at position 375 (Kabat numbering).

55. The antibody of any one of claims 27-54, wherein:
a) the VH1 comprises the amino acid sequence of SEQ ID NO: 13; and
b) the VL1 comprises the amino acid sequence of SEQ ID NO: 22.

56. The antibody of any one of claims 1-55, wherein:
a) the VH1 comprises the amino acid sequence of
(SEQ ID NO: 440)
EVQLLESGGGLVQPGGSLKLSCAASGFTFNX₁YAMX₂WVRX₃APGKGLEW
VAX₄IRSKYNNYATYYADSVKDRFTISRDDSKNTAYLQMNNLKTEDTAV
YYCVRHGX₅FGNX₆X₇YVSWFAYWGQGTLVTVSS,
wherein X1 of SEQ ID NO: 440 is D or T,
wherein X2 of SEQ ID NO: 440 is D or N,
wherein X3 of SEQ ID NO: 440 is D or K,
wherein X4 of SEQ ID NO: 440 is K or R,
wherein X5 of SEQ ID NO: 440 is N or T,
wherein X6 of SEQ ID NO: 440 is D or N, and
wherein X7 of SEQ ID NO: 440 is D or S; and
b) the VL1 comprises the amino acid sequence of
(SEQ ID NO: 441)
ELVVTQEPSLTVSPGGTVTLTCRSSTGAVTTSNYANWVQX₁KPGQAPRG
LIGX₂TNKRAPGTPARFSGSLLGGKAALTLSGVQPEDEAEYYCALWX₃S
X₄LWVFGGGTKLTVL,
wherein X1 of SEQ ID NO: 441 is E or D,
wherein X2 of SEQ ID NO: 441 is D or G,
wherein X3 of SEQ ID NO: 441 is D or Y, and
wherein X4 of SEQ ID NO: 441 is D, N or Q.

57. The antibody of any one of claims 1-56, wherein:
a) the VH2 comprises:
a complementarity determining region 1 (VH2_{CDR1}) comprising the amino acid sequence of SEQ ID NO: 428;
a complementarity determining region 2 (VH2_{CDR2}) comprising the amino acid sequence of SEQ ID NO: 430; and
a complementarity determining region 3 (VH2_{CDR3}) comprising the amino acid sequence of SEQ ID NO: 432; and
b) the VL2 comprises:
a complementarity determining region 1 (VL2_{CDR1}) comprising the amino acid sequence of SEQ ID NO: 433;
a complementarity determining region 2 (VL2_{CDR2}) comprising the amino acid sequence of SEQ ID NO: 434; and
a complementarity determining region 3 (VL2_{CDR3}) comprising the amino acid sequence of SEQ ID NO: 435.

58. The antibody of any one of claims 1-56, wherein:
a) the VH2 comprises:
a complementarity determining region 1 (VH2_{CDR1}) comprising the amino acid sequence of SEQ ID NO: 5;
a complementarity determining region 2 (VH2_{CDR2}) comprising the amino acid sequence of SEQ ID NO: 7; and
a complementarity determining region 3 (VH2_{CDR3}) comprising the amino acid sequence of SEQ ID NO: 9; and
b) the VL2comprises:
a complementarity determining region 1 (VL2_{CDR1}) comprising the amino acid sequence of SEQ ID NO: 10;
a complementarity determining region 2 (VL2_{CDR2}) comprising the amino acid sequence of SEQ ID NO: 11; and
a complementarity determining region 3 (VL2_{CDR3}) comprising the amino acid sequence of SEQ ID NO: 12.

59. The antibody of any one of claims 16-58, wherein the CD58 comprises the amino acid sequence of SEQ ID NO: 49-50.

60. The antibody of any one of claims 16-58, wherein the IL-7 comprises the amino acid sequence of SEQ ID NO: 51.

61. A polynucleotide comprising a nucleic acid sequence encoding the antibody according to any one of claims 1-58.

62. A vector comprising the polynucleotide of claim 61.

63. A pharmaceutical composition comprising the antibody of any one of claims 1-60, the polynucleotide of claim 61 or the vector of claim 62 and a pharmaceutically acceptable carrier.

64. The pharmaceutical composition of claim 63, wherein the pharmaceutical composition is packaged in a liposome or a lipid nanoparticle.

65. A method of treating cancer in a subject in need thereof comprising administering a therapeutically effective amount of the pharmaceutical composition of claim 63.

66. A method of T-cell re-targeting in a subject in need thereof comprising administering a therapeutically effective amount of the pharmaceutical composition of claim 63.

67. A method of T-cell activation in a subject in need thereof comprising administering a therapeutically effective amount of the pharmaceutical composition of claim 63.

68. The method of any one of claims 66-67, wherein the subject has a cancer.

69. The method of claim 68, wherein the cancer is a ULBP2 positive cancer.

70. The method of claim 68, wherein the cancer is a primary tumor, a metastatic cancer, a multiply resistant cancer, a progressive tumor or recurrent cancer.

71. The method of claim 68, wherein the cancer is a solid tumor.

72. The method of claim 68, wherein the cancer is a bladder cancer, a lung cancer, a brain cancer, a head and neck cancer, a breast cancer, a skin cancer, a melanoma, a liver cancer, a pancreatic cancer, a stomach cancer, a colon cancer, a rectal cancer, a uterine cancer, a cervical cancer, an ovarian cancer, a prostate cancer, a testicular cancer, a skin cancer or an esophageal cancer.

73. The method of any one of claims 65-72, further comprising administering a therapeutically effective amount of a ligand or a cytokine.

74. The method of claim 73, wherein the ligand is CD48, CD58, CD86, TNFSF9, OX40L, 4-1BBL, GITL, CD70, CD80, MR1, TNFSF4, ICOSL, ICOSLG, MICA, MICB, ULBP1, ULBP2, ULBP3, RAET1G (ULBP5) and RAET1L (ULBP6) or the cytokines IL-2, IL-7, IL-10, IL-12, IL-15, IL-18 or IL-21, or agonistic antibodies that bind to the receptors of these ligands and cytokines.

75. The method of any one of claims 65-74, wherein the method further prevents T-cell exhaustion.

76. A method of expanding a population of T-cells comprising contacting the population of T-cells with the pharmaceutical composition of claim 63.

77. The method of claim 76, wherein the contacting of the population of T-cells occurs in vitro, in vivo, ex vivo or a combination thereof.

78. The method of any one of claims 76-77, wherein the expansion of the plurality of T-cells is about 2 to about 50 fold higher than the expansion of a plurality of T cells without the pharmaceutical composition, under the same conditions.

79. A method of expanding a population of T-cells comprising contacting the population of T-cells with the antibody of any one of claims 15-26.

80. The method of claim 79, wherein the contacting of the population of T-cells occurs in vitro, in vivo, ex vivo or a combination thereof.

81. The method of any one of claims 79-80, wherein the expansion of the plurality of T-cells is about 2 to about 50 fold higher than the expansion of a plurality of T cells with an antibody that does not have a polypeptide fused to the N-terminus or the C-terminus of the H1 and/or H2, under the same conditions.
